SEQUENCE LISTING (47 sequences)

SEQ ID NO: 1 - DNA, 3774 bp, Artificial Sequence
  APMV4/duck/Hongkong/D3/75
atttacagttagtttacctgtctatcaagttagaaaaaacacgggtagaagattctggatcccggttggcgccctccaggtgcaagatgaggctatcaaacacaatcttgaccttgattctcatcatacttaccggctatttgataggtgtccactccaccgatgtgaatgagaaaccaaagtccgaagggattaggggtgatcttacaccaggtgcgggtattttcgtaactcaagtccgacagctccagatctaccaacagtctgggtaccatgatcttgtcatcagattgttacctcttctaccaacagagcttaatgattgtcaaagggaagttgtcacagagtacaataacactgtatcacagctgttgcagcctatcaaaaccaacctggatactttgttggcagatggtagcacaagggatgttgatatacagccgcgattcattggggcaataatagccacaggtgccctggctgtagcaacggtagctgaggtaactgcagctcaagcactatctcagtcaaaaacgaatgctcaaaatattctcaagttgagagatagtattcaggccaccaaccaagcagtttttgaaatttcacagggactcgaagcaactgcaaccgtgctatcaaaactgcaaactgagctcaatgagaatatcatcccaagtctgaacaacttgtcctgtgctgccatggggaatcgccttggtgtatcactctcactctatttgaccttaatgaccactctatttggggaccagatcacaaacccagtgctgacgccaatctcttacagcaccctatcggcaatggcgggtggtcacattggtccagtgatgagtaagatattagccggatctgtcacaagtcagttgggggcagaacaactgattgctagtggcttaatacagtcacaggtagtaggttatgattcccagtatcagctgttggttatcagggtcaaccttgtacggattcaggaagtccagaatactagggttgtatcactaagaacactagcagtcaatagggatggtggactttacagagcccaggtgccacccgaggtagttgagcgatctggcattgcagagcggttttatgcagatgattgtgttctaactacaactgattacatctgctcatcgatccgatcttctcggcttaatccagagttagtcaagtgtctcagtggggcacttgattcatgcacatttgagagggaaagtgcattactgtcaactcccttctttgtatacaacaaggcagtcgtcgcaaattgtaaagcagcgacatgtagatgtaataaaccgccatctatcattgcccaatactctgcatcagctctagtaaccatcaccaccgacacttgtgctgaccttgaaattgagggttatcgtttcaacatacagactgaatccaactcatgggttgcaccaaacttcacggtctcaacctcacaaatagtatcggttgatccaatagacatatcctctgacattgccaaaattaacaattctatcgaggctgcgcgagagcagctggaactgagcaaccagatcctttcccgaatcaacccacggattgtgaacgacgaatcactaatagctattatcgtgacaattgttgtgcttagtctccttgtaattggtcttattattgttctcggtgtgatgtacaagaatcttaagaaagtccaacgagctcaagctgctatgatgatgcagcaaatgagctcatcacagcctgtgaccaccaaattggggacacccttctgatgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatgcagggcgacatggagggtagccgtgataacctcacagtagatgatgaattaaagacaacatggaggttagcttatagagttgtatccctcctattgatggtgagtgccttgataatctctatagtaatcctgacgagagataacagccaaagcataatcacggcgatcaaccagtcgtatgacgcagactcaaagtggcaaacagggatagaagggaaaatcacctcaatcatgactgatacgctcgataccaggaatgcagctcttctccacattccactccagctcaatacacttgaggcaaacctgttgtccgccctcggaggttacacgggaattggccccggagatctagagcactgtcgttatccggttcatgactccgcttacctgcatggagtcaatcgattactcatcaatcaaacagctgactacacagcagaaggccccctggatcatgtgaacttcattccggcaccagttacgactactggatgcacaaggatcccatccttttctgtatcatcatccatttggtgctatacacacaatgtgattgaaacaggttgcaatgaccactcaggtagtaatcaatatatcagtatgggggtgattaagagggctggcaacggcttaccttacttctcaacagtcgtgagtaagtatctgaccgatgggttgaatagaaaaagctgttccgtagctgcgggatccgggcattgttacctcctttgtagcctagtgtcagagcccgaacctgatgactatgtgtcaccagatcccacaccgatgaggttaggggtgctaacaagggatgggtcttacactgaacaggtggtacccgaaagaatatttaagaacatatggagcgcaaactaccctggggtagggtcaggtgctatagcaggaaataaggtgttattcccattttacggcggagtgaagaatggatcaacccctgaggtgatgaataggggaagatattactacatccaggatccaaatgactattgccctgacccgctgcaagatcagatcttaagggcagaacaatcgtattatcctactcgatttggtaggaggatggtaatgcagggagtcctaacatgtccagtatccaacaattcaacaatagccagccaatgccaatcttactatttcaacaactcattaggattcatcggggcggaatctaggatctattacctcaatggtaacatttacctttatcaaagaagctcgagctggtggcctcacccccaaatttacctacttgattccaggattgcaagtccgggtacgcagaacattgactcaggcgttaacctcaagatgttaaatgttactgtcattacacgaccatcatctggcttttgtaatagtcagtcaagatgccctaatgactgcttattcggggtttattcagatgtctggcctcttagccttacctcagacagcatatttgcatttacaatgtacttacaagggaagacgacacgtattgacccagcttgggcgctattctccaatcatgtaattgggcatgaggctcgtttgttcaacaaggaggttagtgctgcttattctaccaccacttgtttttcggacaccatccaaaaccaggtgtattgtctgagtatacttgaagtcagaagtgagctcttgggggcattcaagatagtgccattcctctatcgtgtcttatagttgagtcaattataaaggagttggaaagatggcattgtatcacctatcttctgcgacatcaagaatca

SEQ ID NO: 2 - DNA, 3720 bp, Artificial Sequence
  APMV17/Antarctica/107/13
atttacagttagtttacctgtctatcaagttagaaaaaacacgggtagaagattctggatcccggttggcgccctccaggtgcaagatgcaattgtactcagtcctggctctggccttactggcagttcaagcggagctcggtataatcccctccctgagcaacacacactcaatagatgcaggatttgtgttccagtctgagcgagcagtcaatatctacaccaattcattaaccgggagtgtggtagttaaactgctccctaacttaccagatcacctcaaaagctgccaccttgatgtcctaagcagttacaaccgcacccttacatctatcttccaaccgctaggtgagagtataaagcatatatggggtaacactacaggagggtcagccgcaggaggaatccagtctcggatagtgggtgccatacttggaggtgtagccttaggtgtagcaacatcagcacagattacagcaggagttgccttagcacaatccaggcagaatgcagagaatatcttaaagttgaaacagtctattgctgcaacaaatgatgctgtgcaagaggttattgcaggacaacgagagcttgtgattgcaataggtaaaatgcaggattacatcaatcaagcgcttaacagcactatccagcaaatcgactgtgttactgctgcaaatcggctcggagtggaacttagcttgtatctgactcagcttaccaccgccttcagtaatcaaatccagaaccctgcactcactccgttgtctattcaggcgttatataacctagctggaggtaatttggataggttcctcaatcgcattggagctaccacatctaatctacagtccatcatatcaagtgggctaattcaggggcaacctattggctacgactctgagaagcagctattgatcctgtctgtatctgtaccaagcataaatgcagtggataatttgcgcatggcgcagctgacccccatagtggtgtccactagccaggggctgggagctgtagttatccccaaatatatcattgcaatcgcagacttaatagaggagtttgtggctgatgactgtatcttcacaacatctgatgcatattgtaccagccttaccacactacctctcagcaattcactgcagcaatgcatcaggggcaatgtgtcagcatgctcgtactcgctggtgaggggagtactatcgaccaagttcatcacccttgatggctctgttatagccaactgccaagcagtgacatgtagatgcattgatccccccaaaatcatatcacaatttgccgggaagccgctcaccattatcaatagcaagatatgcaacattatcaatattgagcaagttacgctaaggctgtctggtcatttcatgtcacaatatggtgctaatcttagtatcagcgaagggcaaattgtggttactggacccctggacattagcaatgagcttggtcgagtcaaccaaagtatcacgaatgcgcaagcatccatagataagagtaaccagattctggaaggtgtcaatgtcaggctgatacaggtgccagccttagcaacgtcacttgctctggcaattgcagggactgtattgggcgcgctggcaataattgggatcttagtgttatgggcagctaataagaagcagagcaaaaagatggagtggctgctcgcatcaaaggcatcaaggatgtgatgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatgccatccatcatgacatccacctcatcgcaaagtcgggagcatcttgcatcacgggatgatgacgatgacagcaagtgtacctggcggctggtgttcagggtgtcggccatagccctacttcttaccattcttggtctctcgattgcaacattctgtaaaataccttcaaaggattttgaacccataatagaagaagctgtacatgagatcacttcgatattgacaccgctaggtgcagggattactgcaatcttggattactgccaaaagatataccgccagagtgtgctagagactcccctgcagttgtcagccatgcaaactagtatacttcagagcctcagtgcgttgtcttatcaaattagtctggaggccaacgggagtaattgcggcgcacccattcatgatgaggcctttgcaggaggtattgagacacctcttttctctgggaaattcactaatggtaagcaattcagggtaagtaagtatatagaacaccttaacttcataccggcacctaccactgggagaggctgcaccagaataccatcgttctctctgtccactagtcacggatgctacacacacaatgtgatcttggacggatgtgcagatcatggcgcgtcccaccagtatatatcaatcggaactctaagagtgtccccatctgggagaatatacttctcaaccctccggagtgtcaacttggatgatggggtgaaccggaagtcatgcagcatcgcagctacccgctatgggtgtgacctgttatgctcagtggtaacagaaacggaaaggagcgactatgcgtcaaacccacctacccggatgatacacggaaggttggatttcgggggttcttatagcgagactgatatcaatagtcaggtgctcttctctgactgggcagctaactacccaggtgtggggtcaggagttgcagtagatgataggattctgttcccgatatatgggggactcagagcgggaaccccttcctataacaggaactatggttcatatgccatctatcagagaagcggtgatgtgtgtcctgacaacaatgctactcaagtaagaaatgcaaaagcatcttacattgtgcctctattctccaatcgattgatacaacaggccatcctatctatcaagttagatcccggtctagggaaggatactacacttcacatttcatctaataatgtgacattgatgggtgcagaggctagactagtagccattgatggccaggtgtacatgtatcaaagaggcagctcatggttcccagctgcagtcctgtaccccatacacagaaagaatggcacattcgcatttgggagaccatacatatatgataactttacgaggcctggcacaggcttctgttctgcagctagcagatgcccaaacacatgtattacaggggtttacacggatgctttcccaattgtcttttctgcggacaaaaagccgataggggtgttcgggacttatctcaaccatcgtagtgatcgtcaaaatcctagatctgctgtgttctttgatgtctccatgagcaatgcaactaatgtctccacaccccccgtaggtgctgcatacactacatccacatgtttcaaaatgacctcaaccggacggcggtactgcatatcaatagcagaaattaggaacaccattttcggggaatacagaattgtgcctctccttgttgaaatagaacaggtgtagttgagtcaattataaaggagttggaaagatggcattgtatcacctatcttctgcgacatcaagaatca

SEQ ID NO: 3 - DNA, 3756 bp, Artificial Sequence
  APMV9/duck/New York/22/78
atttacagttagtttacctgtctatcaagttagaaaaaacacgggtagaagattctggatcccggttggcgccctccaggtgcaagatggggtacttccacctattacttatactaacagcgattgccatatctgcgcacctctgctataccacgacattggatggtagaaaactgcttggtgcaggcatagtgataacagaagagaagcaagttagggtgtacacagctgcgcaatcaggaacaattgtcttaaggtctttccgtgtggtctccttagacagatactcgtgcatggaatccactattgagtcatataacaagactgtatataacatacttgcacctctgggcgatgcaatccgccgaatacaggcaagtggtgtatcggttgagcgtatccgagagggccgcatatttggtgccatccttgggggagttgccttaggtgtagccaccgcagcacagataacagctgcaattgctttgattcaggctaacgagaacgcaaaaaacatcctgcgtattaaagacagtataactaagaccaacgaggcagtgagagatgtaactaatggcgtgtcgcagttaactatcgctgtaggtaaattacaggacttcgtcaataaggaattcaataagacaactgaggccattaattgtgtacaggcagctcaacaattaggtgtggagctaagcctctatctgaccgagatcactacagtcttcggacctcagataacctctcctgctttaagcaaattgactatccaagcgctgtataatttggcgggcgtaagcttggatgtactactgggaaggctcggagcagacaattcacagttatcatctttggttagtagtggtcttattaccggacagcccattctctacgactcggaatctcaaatattggcactgcaagtgtcactaccctccattagtgacttaaggggagtgagagcgacatacttagacacgttggctgtcaacactgcagcaggacttgcatctgctatgattccaaaggtagtaatccaatctaataatatagttgaagaattagatactacagcatgtatagcagcagaagctgacttatactgtacgaggattactacattccccattgcgtcggctgtatcagcctgcattcttggggatgtatcgcaatgcctttattcaaagactaatggcgtcttaaccactccatatgtagcagtaaaggggaaaattgtagccaattgtaagcatgtcacatgtaggtgtgtagatcctacatccatcatatctcaaaattacggtgaagcagcgactcttatcgatgatcagctatgcaaggtaatcaacttagatggtgtgtccatacagctgagcggcacatttgaatcgacttatgtgcgcaacgtctcgataagtgcaaacaaggtcattgtctcaagcagtatagatatatctaatgagctggagaatgttaacagctctttaagttcggctctggaaaaactggatgaaagtgacgctgcgctaagcaaagtaaatgttcacttaactagcacctcagctatggccacatacattgttctaactgtaattgctcttatcttggggtttgtcggcctaggattgggttgctttgctatgataaaagtaaagtctcaagcaaagacactactatggcttggtgcacatgctgaccgatcatatatactccagagtaagccggctcaatcgtccacatgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaatcaggaatcagccaggcatctcttgtcaatgacaacatagaattaaggaatacgtggcgcacggccttccgtgtggtctccttattactcggcttcaccagcttggtgctcactgcttgcgctttacacttcgctttgaatgccgctacccctgcggatctctctagtatcccagtcgctgttgaccaaagtcatcatgaaattctacaaaccttgagtctgatgagcgacattggcaataagatttacaagcaggtagcactagatagtccagtggcgctgctcaacactgaatcaaccttaatgagcgcaattacatcactatcttatcagattaacaatgcagcgaataactcaggttgtggcgcccctgtgcatgataaggattttatcaatggagtggcaaaggaattatttgtagggtctcaatacaatgcctcgaactatcgaccctccaggttccttgagcatctaaatttcatccccgcccctactacgggaaaaggttgcaccagaattccgtcctttgatctagctgcaacacattggtgttatactcacaatgtgattcttaatggttgtaatgatcatgctcaatcttatcaatacatatccctcgggatactcaaggtgtcagccacgggaaacgtgttcttatctactctcagatctatcaacctggatgatgatgaaaaccggaaatcatgtagcatatcagcaacgccactagggtgtgacttactttgtgctaaagtcactgagagagaagaggcagattacaattcagatgcagcgacgagattagttcatggcaggttaggttttgatggggtataccatgagcaggccctgcctgtagaatcattgttcagtgactgggttgcaaactatccgtcagtcggcggaggcagttactttgataatagggtatggtttggcgtgtatggggggatcagacctggctctcagactgatctgctccagtctgagaagtacgcgatatatcgtaggtacaataatacctgccctgataataatcccacccagattgagcgggccaaatcatcttatcgtccgcagcggtttggccagcggcttgtacaacaagcaattctatcaattagagtggagccatctttgggtaatgatcctaaactatctgtgttagataatacagtcgtgttgatgggggcggaagcaaggataatgacatttggccacgtggcattaatgtatcaaagagggtcatcatattttccttctgcactattataccctctcagtttaacaaatggtagtgcagcagcatccaagcctttcatattcgagcaatatacaaggccaggtagcccaccttgtcaggccactgcaagatgtccaaattcatgtgttactggtgtctacacagacgcatacccgttattttggtctgaagatcataaagtgaatggtgtatatggtatgatgttagatgacatcacatcacggttaaacccggtagcagctatatttgataggtatggtaggagtagagtgactagggttagcagtagcagcacgaaggcagcttacactacaaatacatgctttaaggttgtcaaaacaaagagagtatactgcttgagcattgccgagatagagaatacactgtttggagaattcagaataacccctttactctccgagataatatttgacccaaaccttgaaccctcagacacgagccgtaactagttgagtcaattataaaggagttggaaagatggcattgtatcacctatcttctgcgacatcaagaatca

SEQ ID NO: 4 - DNA, 3690 bp, Artificial Sequence
  APMV7/Dove/Tennessee/4/75
atttacagttagtttacctgtctatcaagttagaaaaaacacgggtagaagattctggatcccggttggcgccctccaggtgcaagatgagagtacgacctttaataataatcctggtgcttttagtgttgctgtggttaaatattctacccgtaattggcttagacaattcaaagattgcacaagcaggtattatcagtgcacaagaatatgcagttaatgtgtattcacagagtaatgaggcttacattgcactgcgcactgtgccatatatacctccacacaatctctcttgtttccaggatttaatcaacacatacaatacaacgattcaaaacatattctcaccaattcaggatcaaatcacatctataacatcggcgtcaacgctcccctcatcaagatttgcaggattagtagtcggtgcaatcgctctcggagtagcgacatctgcacaaataactgcagccgtggcactcacaaaggcacagcagaacgctcaagaaataatacgattacgtgattctatccaaaatactatcaatgctgtgaatgacataacagtagggttaagttcaataggagtagcactaagcaaggtccaaaactacttgaatgatgtgataaaccctgctctgcagaacctgagctgccaggtttctgcattaaacttagggatccaattaaatctttatttaaccgaaattacaactatctttggaccgcaaattacaaatccatcattgaccccattgtcaattcaggcattatacaccctagcaggagataacctgatgcaatttcttaccaggtatggctatggagagacaagtgttagcagtattctcgagtcaggactaatatcagcacaaattgtatcttttgataaacagacaggcattgcaatattgtatgtcacattaccatcaattgcgactctttccggttctagagttaccaaattgatgtcagttagtgtccaaactggagttggagagggttctgctattgtaccatcatacgttattcagcagggaacagtaatagaagaatttattcctgacagttgcatcttcacaagatcagatgtttattgtactcaattgtacagtaaattattgcctgatagcatattgcaatgcctccagggatcaatggcagattgccaatttactcgctcattgggttcatttgcaaacagattcatgaccgttgcaggtggggtgatagcaaattgtcagacagtcctgtgccgatgctataatccagttatgattattccccagaacaatggaattgctgtcactctgatagatggtagtttatgtaaagaacttgaattggaggggataagactaacaatggcagacccagtatttgcttcatactctcgtgatctgattataaatgggaatcaatttgctccgtctgatgctttagacattagtagcgaattaggtcaactgaataactcaattagctcagcaactgataatttacagaaggcacaggaatcattgaataagagtatcattccagctgcgacttccagctggttaattatattactatttgtattagtatcaatctcattagtgataggatgtatctccatttattttatatataaacattcaaccacaaatagatcacgaaatctctcaagtgacatcatcagtaatccttatatacagaaagctaattgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggagtcaatcgggaaaggaacctggagaactgtgtatagagtccttacgattctattagatgtagtgatcattattctctctgtgattgctctgatttcattgggtctgaagccaggtgagaggatcatcaatgaagtcaatggatctatccataatcaacttgttcccttatcggggattacttccgatattcaggcaaaagtcagcagcatatatcggagcaacttgctaagtatcccactacaacttgatcaaatcaaccaggcaatatcatcatctgctaggcaaattgctgatacaatcaactcgtttctcgctctgaatggcagtggaacttttatttatacaaattcacctgagtttgcaaatggtttcaatagagcaatgttcccaaccctaaatcaaagcttaaatatgctaacacctggtaatctaattgaatttactaattttattccaactccaacaacaaaatcaggatgtatcagaataccatcattttcaatgtcatcaagtcactggtgttatacccataatatcattgctagtggatgtcaggatcattcaaccagtagtgaatacatatcgatgggggttgttgaagtgactgatcaggcttacccgaactttcggacaactctttctattacattagctgataatctaaacagaaagtcatgtagcattgcagcaactgggttcgggtgtgatatattatgtagtgttgtcactgagacagaaaatgatgattatcaatcaccagaaccgactcagatgatctatggaagattattttttaatggcacatattcagagatgtcattgaatgtgaaccaaatgttcgcagattgggttgcaaattatccagcagttggatcaggagtagagttagcagattttgtcattttcccactctatggaggtgttaaaatcacttcaaccctaggagcatctttaagccagtattactatattcccaaggtgcccacagtcaattgctctgagacagatgcacaacaaatagagaaggcaaaagcatcctattcaccacctaaagtggctccaaatatctgggctcaggcagtcgttaggtgcaataaatctgttaatcttgcaaattcatgtgaaattctgacatttaacactagcactatgatgatgggtgctgagggaagactcttgatgataggaaagaatgtatacttttatcaacgatctagttcgtattggccagtgggaattatatataaattagatctacaagaattgacaacattttcatcaaatcaattgctgtcaacaataccaattccatttgagaaattccctagacctgcatctactgctggtgtatgttcaaaaccaaatgtgtgtcctgcagtatgccagactggtgtttatcaagatctctgggtactatatgatcttggcaaattagaaaataccacagcagtaggattgtatctaaactcagcagtaggccgaatgaacccttttattgggattgcaaatacgctatcttggtataatacaactagattattcgcacagggtactccagcatcatattcaacaacgacctgcttcaaaaatactaagattgacacggcatactgcttatcaatattagaattaagtgattctttgttaggatcatggagaattacaccattattgtacaatatcactttaagtattatgagctagttgagtcaattataaaggagttggaaagatggcattgtatcacctatcttctgcgacatcaagaatca

SEQ ID NO: 5 - DNA, 3702 bp, Artificial Sequence
  APMV21/pigeon/Taiwan/AHRI128/17
atttacagttagtttacctgtctatcaagttagaaaaaacacgggtagaagattctggatcccggttggcgccctccaggtgcaagatgacaaagaggatcaatctaacgttgtccttatatatactggtcacaatctcagtgtgtctcccaacaactcgatgtctagacaatagcaaattggctcgagccgggataataagttcagccgaatatgcagtgagtgtgtatgctcagacaaatgaagcatacatagcacttagaaccattccatatttaccagccaatccgaataattgcttcacaccaactataaccacatataacactacaatacaatctattttctcacctattgtctcacagattaatgcaattacttcgagcacctcgacccaacaagagagactcttcggagtgatcataggtactgttgctttgggagtggccactgcagcacaagtgactgctgcagttgcgctaacacaagctcaaagcaatgcaaaagcaatcctacaactcaaatcatcaatacaaaatactattgctgctgtgtcagaagtgaaagacgggttaagcacaattgggattgctctaggaaaaatccaagtttatgtcaatgaagtgataaatccccaacttgctaacctgacttgccaaacagctgctgcgaatttaggagtccaacttagtctatatctaacagaattgacaactgtgtttgggccccaaatcacaaaccctgcattatcaccactgactattcaagcactttataatctggcaggatcgaacttagatacattctttgagaaatatggttataagcaagcaactgcaacaagtgtgctagaggctggactagtaactggccagattgtatcttttgatccagctacaggtattggaattattcgagtttcccttcctagcatagcaacactatcttctgctcgtgtcaccaaacttgaaactgtgagtgttagtacctcaacaggagaagctgtagccattgtcccgtcattcataatacagcaagggacagttatagaagaattcataattgacggctgtataagaacaagtgctgatatatattgcactcggctgtttacaaaaatactgcctgatagcgtactgaattgcttgcaaggattagttaatgagtgtcaatttacccgtggcttagggacctatgcaaataggtttgtaacaatcaatggtggaattgttgcaaattgtcaaacattactctgccgctgctatagcccgtcatatattattacgcaaaattccaatatagcagtcaccttaatcgactcaagtacctgtcgtgacttagacttggacggcataagattagctttaggaaatactgaattctcagagtatgccaaaaatctaacaatagcagagtcccaattcgcaccctctgatgcattggatatcagcagtgaaatagggaaactaaatgctacgatatcaagagtggaagactacctcaaccaagcaacaaaagatgtcactgctatatcaattaataagtcagcagcagacataattctgattgtgacattaatacttaccatccttctaataattactgtcatagtcatagttgttatcatcataaaacaaaggagggtaattacacacaaaacgacaaatgaggatatgatttcgaatccatacgttacaaatgccaagtgatgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggactcacatatcccagccaagaacacatggcggacagtatatcgcgtggtgaccatactgttagatatcgtgataattgtccttgccatcatatctcttgtgtctttaggcttaaaacctggtgagaagatcttgacaggggtaaatgattcagtgcatgcagagttaggcatgatgagaccagcattattagatatagatagtaaggtgagtacaatttacaggtacaatctcatcaacctgcctctacagttggatgatatccagacagccatagtgtcttcttcaaggcaattagctgacactataaatagcttcttagcaatcaatggttcatctgcagtgctatacacaacaggtcccgagttctcaaacggatttaataaggagctttatcccaactttaatcagactcgagattctatatcaattgggcagctagttgagttcacaaacttcataccaacaccaacaactaagccggggtgcataagaatacccacatttgctgcaggacaatctcattggtgttatagtcacaatataattgcatccggatgtcaagaccattcaacaagtagtcagtacatagccatgggagtaatcatcattaatcaacaacaatcacctgacttcagaacaacaacttctatcaccctgtcggataataagaatagaaaatcatgtagtgtcggagtgtcggaatacggatgtgatcttttgtgcagtgtggtaacagaaacagagaatgaagattacaagtcagaacccccgacagacatgatatacgggaggttattctttaacgggacatacagtgaagttgatctccctgtgtctacactgttctctgaatgggttgcgaattacccaggagtagggtcaggggttgtgtatcgcaggaagatgtatttccctatttatggaggaattaagatttcctctaatttgggaaactacttgtctcacttttactatataccacaagttcccactgtcaattgtacagacagtgatgagatacagattaccaatgcaaaggcgtcatactctcctcctaaagttgctccgaatttatgggggcaagcattgcttgcttgtaatatcagcgttaatttgccgagttcctgtagattactagtcttcaatacgtcatcaatgatgatgggtgctgaaggtagaatatataacatcaatgagcagtattatttctaccaaagatcaagcagttattggccagtgggcctcatctataggctggaaatgacaagccttgacagcatgactgattcaggtattatcaacactactcccatcccctttgagaaattcccacggccagcatcacaggctggtgtgtgctcaattccaagcgtttgtcctcgagtctgtcaaactggcgtttatcaggacatctgggtgttatcacaaccaagtgttgaaatgaatactacagcaataggtatctatcttaactcagctgtcggtaggacaaatccgaaaattggcgttgcaaataccttaagctggatagacgctgttcaattgtttcaaccaactactcctgctagttactccacaactacttgttttaagaatacagcaagagatatatcttattgtctgtcaatcctggaactaagcgattcactacttggttcttggaggatagctccattgctgtataatcttacactggttcctaattcatagttgagtcaattataaaggagttggaaagatggcattgtatcacctatcttctgcgacatcaagaatca

SEQ ID NO: 6 - DNA, 3870 bp, Artificial Sequence
  APMV6/duck/HongKong/18/199/77
atttacagttagtttacctgtctatcaagttagaaaaaacacgggtagaagattctggatcccggttggcgccctccaggtgcaagatgggagcccgactggggccctttacaatggcacccggccggtatgtgattattttcaacctcatccttctccacaaggttgtgtcactagacaattcaagattactacagcaggggattatgagtgcaaccgaaagagaaatcaaagtgtacacaaactccataactggaagcattgctgtgagattgattcccaacctacctcaagaagtgcttaaatgttctgctgggcagatcaaatcatacaatgacacccttaatcgaattttcacacctatcaaggcgaatcttgagaggttactggctacaccgagtatgcttgaacacaaccagaaccctgccccagaacctcgcctgattggagcaattataggcacagcagcactggggctggcaacagcagctcaggttacagctgcactcgcccttaaccaggcccaggataatgctaaggccatcttaaacctcaaagagtccataacaaaaacaaatgaagctgtgcttgagcttaaggatgcaacagggcaaattgcgatagcgctagataagactcaaagattcataaatgacaatatcttaccggcaatcaataatctgacatgtgaagtagcaggtgctaaagtaggtgtggaactatcattatacttgaccgagttaagcactgtgtttgggtcgcagataaccaatccagcactctccactctatccattcaagccctcatgtcactctgcggtaatgattttaattacctcctgaacctaatgggggccaaacactccgatctgggtgcactttatgaggcaaacttaatcaatggcagaatcattcaatatgaccaagcaagccaaatcatggttatccaggtctccgtgcctagcatatcatcgatttcggggttgcgactgacagaattgtttactctgagcattgaaacacctgtcggtgagggcaaggcagtggtacctcagtttgttgtagaatctggccagcttcttgaagagatcgacacccaggcatgcacactcactgacaccaccgcttactgtactatagttagaacaaaaccattgccagaactagtcgcacaatgtctccgaggggatgagtctagatgccaatatacgactggaatcggtatgcttgaatctcgatttggggtatttgatggacttgttattgctaattgtaaggccaccatctgccgatgtctagcccctgagatgataataactcaaaacaagggactcccccttacagtcatatcacaagaaacttgcaagagaatcctgatagatggggttactctgcagatagaagctcaagttagcggatcgtattccaggaatataacggtcgggaacagccaaattgccccatctggaccccttgacatctcaagcgaactcggaaaggtcaaccagagtctatctaatgtcgaggatcttattgaccagagcaatcagctcttgaatagggtgaatccaaacatagtaaacaacaccgcaattatagtcacaatagtattgctagttatcctggtattatggtgtttggccctaacgattagtatcttgtatgtatcaaaacatgctgtgcgaatgataaagacagttccgaatccgtatgtaatgcaagcaaagtcgccgggaagtgccacacagttctgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggcttcctcaggcgatatgagacagagtcaggcaactctatatgagggtgaccctaacagcaaaaggacatggaggactgtgtaccgggttgtcaccatattgctagatataaccgtcctttgtgttggcatagtggcaatagttaggatgtcaaccattacaacaaaagatattgataacagtatctcatcatctattacatccctgagtgccgattaccagccaatatggtcagatacccatcagaaagttaacagtattttcaaggaagttggaatcactatccctgtcacactcgacaagatgcaagtagaaatgggaacagcggttaacataatcactgatgctgtaagacaactacaaggagtcaatgggtcagcaggatttagcattaccaattccccagagtatagtggagggatagacacactgatataccctcttaattcacttaatggaaaggctctagctgtatcagacttactagaacacccgagcttcataccgacgcctaccacctctcacggttgtacccgcattcctacattccacctagggtaccgtcattggtgttatagtcacaacacgatagagtctggttgtcacgatgcaggagaaagcattatgtacgtatccatgggtgcggtaggggtcggccatcgcgggaaacctgtgtttacgacaagtgcagcgacaatcctagatgatggaaggaacaggaaaagttgtagcatcatagcaaaccctaatgggtgtgatgtcttatgcagcttggttaagcagacagaaaatgaaggctacgctgaccctacaccgaccccaatgatccacggtaggctccacttcaatggcacatacactgagtctgaacttgaccctggcctatttaataaccattgggtcgctcaatatccagcagttggtagcggtgtcgtcagccacagaaaactatttttcccgctctacggagggatatcaccgaagtcaaaactgttcaatgagctcaagtcatttgcttactttactcataatgctgaattgaaatgtgagaacctgacagagagacagaaggaagacctttataacgcatataggcctgggaaaatagcaggatctctctgggctcaaggggttgtaacatgtaatctgaccaatttagctgattgcaaagttgcaattgcgaacacgagcaccatgatgatggctgccgaggggaggttacagcttgtgcaagataagattgtcttctaccaaagatcctcatcatggtggccagtcctaatatattatgatatccctattagtgaccttatcagtgccgatcatttagggatagtgaactggactccgtatccacagtctaagtttccgaggcccacctggacaaagggcgtatgtgagaaaccggcgatatgccccgctgtatgtgtaacgggtgtttaccaagatgtttgggtagttagtatagggtcacagagcaatgagactgttgtggttggcgggtacttagatgctgcagcagcccgtcaggatccatggattgcagcagctaaccagtacaactggctggttaggcgtcgcctctttacatcccaaactaaagcagcatactcatcaaccacttgcttcagaaacacgaagcaggatagagtgttctgcctgactataatggaagtcacagacaacctactcggagactggaggatcgccccgctgttgtatgaagttactgtggctgataagcagcagggcaatcgcaattacgtgcctatggggagggtggggacagataagttccaatattataccccaggtgacagatatactcctcagcattagttgagtcaattataaaggagttggaaagatggcattgtatcacctatcttctgcgacatcaagaatca

SEQ ID NO: 7 - DNA, 3804 bp, Artificial Sequence
  APMV11/common_snipe/France/100212/10
atttacagttagtttacctgtctatcaagttagaaaaaacacgggtagaagattctggatcccggttggcgccctccaggtgcaagatgggcacatgcctaaacaaccgtctgtcgactattccctctatcaaaactgtacaatgtattttgattatcttatcttacataataccatactctgcaacagacaacccaattgccgaccggtcattactgcgcgccggaattgtacccatatattctaagagtctcagcgtatacactaattcaatctcaggttatctgacagtgcgcatgttaccccctcttccaaagaatctaactgaatgtagtcaagaagttgttagtaactataataaaacaattacaagaatgtttcaacctatatctgataatctaatgcgaattcaagaggggacagatagtgggacaaaaagatttgtgggtgccgtgataggatcagttgctctcggtgttgctacttctgcccaaattactgctgctttggcgatggtacaggctcaggataatgcaaaagccatctggaaactcaaggaagcgatttcttcaacaaatcaggctgtattagagttaaaggagggcgtaaatacattgggagtcgcagtagacaagatccaaggatatataaataatgaaatactcccctcattgtcagagctagagtgtcgagttaatgcaaataagttggcctctcagttaaatctatatttgattgagttaaccactatatttggggatcagataacgaatccagcattaacacccttaagccttcaggcattgtatactcttgcaggagacacaatgggaagcttcttacaatatattggtgcacaggataacgaaattgagtcactatatgatagtggattaattaatgggcagattgtgtcatatgatgcatcaatccagaccataattattaaggtatccattccatccatatcatctctctcacgattttctattatgaggctggcaacagttagctcatcagttggaggttttgagaaaactcccttggtgcctgagtacttgctcataagtgacaatcacattgaagagttcagtattgttgattgcaaagagtcatcagatattttttattgccctcaaattttgtcaatgccaatatcaactgccactgtagaatgcctcaagggtagaattgatcagtgcatatatacatcacagctaactatactttctcatcgcatagtaacatacaatggtgtcgttgttgccaattgttttgcagaattatgtagatgtactaatcctagttacataattagacaagaccgggatgttgctgtcacagtaattgataaagatttgtgtaaacgagttcagataggagatatagaactaattgtacaagcatctattgctaatgaatacaaggttaactttactgtatcagaggaccaacttgcgccctctacccctatcgatatcagtaatgaattaaattcattgaatcaaactttagataaggtaggacaattgatcaatacgagtaaccaaattctggcatcactgaacccaaaattggtgaacaatacatctattattgtattaattgtaatgggagtggtgctaattttatggctgttggctcttaccatctactcgatatatgctgcaagaaatctaaactctataggacgactagctaaatctgcatatgcatcttatgtagctgataaaaatgtatataaaaacgaatcaactagctctagcagtatttgatgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgatctaggagccttgattacttagctgattcacctgaaatcaaaaatacatggcgacagttcttcagagtggtccttattatactccaaattaccatgctgtgtatcagcatttctgcactggctattacaattcaagttcgtgatcaacatcttccttctctaataaaagagaatcctaaaaccacctcatcacttatatcaagtgaactcaacccgctgctatcctatcttcctggtataaatagagaagttcagttgaatatacctattcaattagataaaattcaacaatctgcgacctcagaaatcaatcggcttactgctgctattaatcagatggcatttggcactctttcacctggactcctgctaaagaactcaaaggattatgtaggaggtattaataagccgcttattccttctgacaaactcaattggacaaatgcaacaatatcaggatttattgaacaccctagtttcatacccggaccaaccaccaaaaaggggtgtacacgaataccatctttccatttggccgagtctcattggtgctatacacataatactatagcttcagggtgtgaagaccatggtgtgtcttctatgtatatctctggtgggattttatataagggctctaataaagagccttctcttctaacgacagtaagtatcttgttggcagacgagcttaataggaaaagctgcagtataattgcttcttcttatggatgtgatgtcctctgttctctagtcacagaaagcgagagtcaagattataagtcagttaatccgacacccatggtacatggtcggttgttttttaatggatcatattctgaacaggaacttgatcccaggatttttggagatttgtggactgcgaactaccctggagtcggatcagggatacttttaaaagatagacttgtgttcccaatttatggaggtctagatgagacaaagctgaatctaacatcttatcttaaccatcccttgtacacaaaaaatgaatgggtgtcatgtaataaatcatatgacgaggttgtacagacattaagagctgcatatcggccttcttggttcgctgggagagttgttactcaaggagtgatggtctgccattatgatagagaattgctagggaggtgtctcatagcacgctttaatacatctacagtaatgatgggagcagaaagtagattagtgatgcaaggtgactcactccttctataccaacgatcaagttcatggtggccagttggaattgtttatttagttccggaatccatcatctcaattaatgagacaaattcggtattcgacttatcgcctatcccattgtccaaattcccgagacctactaataagaaaggatattgtgaacgacctgctgtttgtcctgcagtttgtgtgaccggtgtgtatcaagatctttggccgttatcaccattagctattgagaacaggacagccaccaaccctacttttgcaggtgcattccttaatgcatttacaacaagaacagctccttactttggagtagcagggccaaacaagtgggctcggtcagtacagttatttactgaccagactccagcatcatattcaacaactacttgctttaaggacaccataactacacaaacttattgcttaattatcattgaactacaagagaatcttttgggtacctggaaaattgtaccccttttggttaaagtatctttagtttactcgtagttgagtcaattataaaggagttggaaagatggcattgtatcacctatcttctgcgacatcaagaatca

SEQ ID NO: 8 - DNA, 3732 bp, Artificial Sequence
  APMV15/calidris_fuscicollis/Brazil/RS-1177/12
atttacagttagtttacctgtctatcaagttagaaaaaacacgggtagaagattctggatcccggttggcgccctccaggtgcaagatgtatgtaccaggtgtaattctagctctcttgatgatcaatccctgtctaactctagacaattctaagctagcacccgtgggtattattagtgctgcggagcatgagttggctatatatacgaatacactctctggctctattgctgtcagattcctgcccaatttgccagctaatcttacacattgtcagaagacaatcttagataactacaatgtaactgtgacacgcattttgaagccgattgcggataatctgaatatactcaagcatggattagaagttccaaaagagagattagtaggtgctatcataggcactgttgctcttggtgtggccacatcagctcaaatcacagctgcagttgcagttgcccaagctcagcagaatgcaaaagatatctggaagcttaaaaatgcaatccttagtactaatgaggctgtactagaattaaagacaggcttgcaacaaactgccatcgcactagacaaaatacaggattatatcaataatgagattataccaacagttaataatttaacctgtgaagtgatggcaaacagacttggtgtatatttgtccttgtatttgacggagctaaccacagtatttgggaatcaaataaccaatcctgccctcagcacaatttcatatcaaggactgacgaatttgtgtgggaataatattggagcactgacaaaattaatagggttaaaagatgataatgtagaatcaatatatgaagcgggattaataactggtcaagtagttgactatgaccctgcaagtcaaatcttaatcatccaggttagttatccaagtatatcaagattgagtgatataagagctactgagttaatcactgttggtgtgacaactccttttggtgaaggaagggcaattgtcccgaagtatgtagcacagagcactgtattaattgaggagttggacatctcatcttgtaaattcagttcaactacattatactgtactcagattaatactcgcccgttacctccgagagtatcaagctgccttaaaggtgattatgagaattgtcagtttacaacagaagtgggggtgcttgcatctaggtatgcatctatagggaagggagtagtagtcaattgcagatcaattatatgtaaatgtttagagcctcctagaattatacctcagaatagcttggcatctataacagtcatagatagcaagatctgtaagaagctccaattacctgatgttatattgcgtctagatggtaacctcgaatctcagtatttcactaatatatcaatcaatggtgggcaagtgaccccttctgggccacttgatattagcagtgagatagggaacatcaatcaaactgtaaatcgagttgaggatttgattcatgaatctgaaagctggctgtctcgagtcaatcccaagctaatatcaaacacagcaatcattgttctctgtgtcttatcatcgctgtgtgtgctttggcttatattaatcactgcattcatggctaaattactaagtaatgttaaaaagatagaaaggaaggtagcggtatcttccctaataggtaatccttatgtttataccaatcctggctattcaggttctaagagcgcatgatgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatgaattcaagttactctcaagataatttatatacaaatcaaactgctgctcaacctagaggaacttggagagttctttatagggccgtatcattgatattccagatattaatcttctcactagtactgacaaatgtcatccaatattcaaatctccatagcccctctgtgtcagagatttctgcagccactacaactgagaccattgatgggttaaaaccacacttagaaactccacttaaccagataaatgacatctttcgcttaactgcccttgacttgcccatacagatgaacacgatgactcgagaaatcacaagtcaactcaatatcctgaccagtggaattaatgagcttgttacctcgaataattcgggaagacttctccagactacagaccctgcatatacaggtggtattggagtctttgtgctcaataattacttagattatccaccgaatctgcagaacatgtcattattagagcagcctaattttgtacccggttctaccaccactgggggttgtacacggattcccactttccatttgtcatcaactcattggtgttattctcataacatcattgagaaaggctgccatgatgcaggacactcgagtatgtacatatctattggtgtggtccaagtatcctcacgcggtgttccagtgtttctgacgactcagagtgtaattgttgacgacgaaaccaaccgaaaatcctgtagcattgtgtcaactgaatatggttgtgatatattatgcagcatagttactgagcgtgagtcagatgactataaatcagatccacctactagaatgctacatggtagactcttgttcaacggctcttatgtagaagctgctgtcaaattcaccaatgacatcaataaattctccgcaaattacccgggagtcggttcaggaatcctcctaggcaataaaattctattccctctgtatggaggcataaagcagagtacagatttgtttaattacttacacaacaggactgctcaagtatcaaacaataaaacagtatgtagcaccggttatgataaaaagaaactagaagctgcatatcgaccgccactaattggagggagattttgggcaatcgggatagttatatgtaagttcagcataaattcacttggagattgcagatacaaaatatatgacagcagcgtagtcatgatgggttcagaaaatcgcctcatgaaggtaggcaatcaggtattcttgtatcagagatccagctcatggtggcctattggattgacctacatactcaatagcactgacttgcttaacaccgattctgacatagtcagcagtataatccccatatatcatacaaaattcccgcgccctacttatgataggaatgcatgcactagaccaaacgtttgtcctgccacatgcatagaaggtgtttatgcagatatttggccacttaataatccggcagaaccgagtaaaattatatgggtcagtcattatctcaattcagaagtagggagagaattccctgctatcggtgttgccaaccaatatgaatgggtaaaagaatttcgtccacttccacccacaacaggtgcagcgtatgcaactacttcttgttttaagaatacaataagcaaccgcatcttctgtgttagtgtagctgaattcaaggacaatttatttgggcaattcagaatcgtaccgcttctatatgagattaaagtaatcaactagttgagtcaattataaaggagttggaaagatggcattgtatcacctatcttctgcgacatcaagaatca

SEQ ID NO: 9 - DNA, 3726 bp, Artificial Sequence
  APMV8/Goose/Delaware/1053/76
atttacagttagtttacctgtctatcaagttagaaaaaacacgggtagaagattctggatcccggttggcgccctccaggtgcaagatgggtaaaatatcaatatatctaattaatagcgtgctattattgctggtatatcctgtgaattcgattgacaatacactcgttgccccaatcggagtcgccagcgcaaatgaatggcagcttgctgcatatacaacatcactttcagggacaattgccgtgcgattcctacctgtgctcccggataatatgactacctgtcttagagaaacaataactacatataataatactgtcaacaacatcttaggcccactcaaatccaatctggatgcactgctctcatctgagacttatccccagacaagattaattggggcagttataggttcaattgctcttggtgttgcaacatcggctcaaatcactgctgcagtcgctctcaagcaagcacaagataatgcaagaaacatactggcactcaaagaggcactgtccaaaactaatgaggcggtcaaggagcttagcagtggattgcaacaaacagctattgcacttggtaagatacagagctttgtgaatgaggaaattctgccatctatcaaccaactgagctgcgaggtgacagccaataaacttggggtgtatttatctctgtatctcacagaactgaccactatattcggtgcacagttgactaaccctgcattgacttcattatcatatcaagcgctgtacaacctgtgtggtggcaacatggcaatgcttactcagaagattggaattaaacagcaagacgttaattcgctatatgaagccggactaatcacaggacaagtcattggttatgactctcagtaccagctgctggtcatccaggtcaattatccaagcatttctgaggtaactggtgtgcgtgcgacagaattagtcactgttagtgtaacaacagacaagggtgaagggaaagcaattgtaccccaatttgtagctgaaagtcgggtgactattgaggagcttgatgtagcatcttgtaaattcagcagcacaaccctatactgcaggcaggtcaacacaagggcacttcccccgctagtggctagctgtctccgaggtaactatgatgattgtcaatataccacagagattggagcattatcatcccggtatataacactagatggaggggtcttagtcaattgtaagtcaattgtttgtaggtgccttaatccaagtaagatcatctctcaaaatacaaatgctgcagtaacatatgttgatgctacaatatgcaaaacaattcaattggatgacatacaactccagttggaagggtcactatcatcagtttatgcaaggaacatctcaattgagatcagtcaggtgactacctccggttctttggatatcagcagtgagatagggaacatcaataatacggtgaatcgtgtggaggatttaatccaccaatcggaggaatggctggcaaaagttaacccacacattgttaataatactacactaattgtactctgtgtgttaagtgcgcttgctgtgatctggctggcagtattaacggctattataatatacttgagaacaaagttgaagactatatcggcattggctgtaaccaatacaatacagtctaatccctatgttaaccaaacgaaacgtgaatctaagttttgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatgagtaacattgcatccagtttagaaaatattgtggagcaggatagtcgaaaaacaacttggagggccatctttagatggtccgttcttcttattacaacaggatgcttagccttatccattgttagcatagttcaaattgggaatttgaaaattccttctgtaggggatctggcggacgaggtggtaacacctttgaaaaccactctgtctgatacactcaggaatccaattaaccagataaatgacatattcaggattgttgcccttgatattccattgcaagtaactagtatccaaaaagacctcgcaagtcaatttagcatgttgatagatagtttaaatgctatcaaattgggcaacgggaccaaccttatcatacctacatcagataaggagtatgcaggaggaattggaaaccctgtctttactgtcgatgctggaggttctataggattcaagcaatttagcttaatagaacatccgagctttattgctggacctacaacgacccgaggctgtacaagaatacccacttttcacatgtcagaaagtcattggtgctactcacacaacatcatcgctgctggctgtcaagatgccagtgcatctagtatgtatatctcaatgggggttctccatgtgtcttcatctggcactcctatctttcttactactgcaagtgaactgatagacgatggagttaatcgtaagtcatgcagtattgtagcaacccaattcggctgtgacattttgtgcagtattgtcatagagaaggagggagatgattattggtctgatactccgactccaatgcgccacggccgtttttcattcaatgggagttttgtagaaaccgaactacccgtgtccagtatgttctcgtcattctctgccaactaccctgctgtgggatcaggcgaaattgtaaaagatagaatattattcccaatttacggaggtataaagcagacttcaccagagtttaccgaattagtgaaatatggactctttgtgtcaacacctacaactgtatgtcagagtagctggacttatgaccaggtaaaagcagcgtataggccagattacatatcaggccggttctgggcacaagtgatactcagctgcgctcttgatgcagtcgacttatcaagttgtattgtaaagattatgaatagcagcacagtgatgatggcagcagaaggaaggataataaagatagggattgattacttttactatcagcggtcatcttcttggtggccattggcatttgttacaaaactagacccgcaagagttagcagacacaaactcgatatggctgaccaattccataccaatcccacaatcaaagttccctcggccttcatattcagaaaattattgcacaaagccagcagtttgccctgctacttgtgtcactggtgtatactctgatatttggcccttgacctcatcttcatcactcccgagcataatttggatcggccagtaccttgatgcccctgttggaaggacttatcccagatttggaattgcaaatcaatcacactggtaccttcaagaagatattctacccacctccactgcaagtgcgtattcaaccactacatgttttaagaatactgccaggaatagagtgttctgcgtcaccattgctgaatttgcagatgggttgtttggagagtacaggataacacctcagttgtatgaattagtgagaaataattagttgagtcaattataaaggagttggaaagatggcattgtatcacctatcttctgcgacatcaagaatca

SEQ ID NO: 10 - DNA, 3714 bp, Artificial Sequence
  APMV2/Chicken/California/Yucaipa/56
atttacagttagtttacctgtctatcaagttagaaaaaacacgggtagaagattctggatcccggttggcgccctccaggtgcaagatgaatcaagcactcgtgattttgttggtatctttccagctcggcgttgccttagataactcagtgttggctccaataggagtagctagcgcacaggagtggcaactggcggcatatacaacgaccctcacagggaccatcgcagtgagatttatcccggtcctgcctgggaacctatcaacatgtgcacaggagacgctgcaggaatataatagaactgtgactaatatcttaggcccgttgagagagaacttggatgctctcctatctgacttcgataaacctgcatcgaggttcgtgggcgccatcattgggtcggtggccttgggggtagcaacagctgcacaaatcacagccgccgtggctctcaatcaagcacaagagaatgcccggaatatatggcgtctcaaggaatcgataaagaaaaccaatgcggctgtgttggaattgaaggatggacttgcaacgactgctatagctttggacaaagtgcaaaagtttatcaatgatgatattataccacagattaaggacattgactgccaggtagttgcaaataaattaggcgtctacctctccttatacttaacagagcttacaactgtatttggttctcagatcactaatcctgcattatcaacgctctcttaccaggcgctgtacagcttatgtggaggggatatgggaaagctaactgagctgatcggtgtcaatgcaaaggatgtgggatccctctacgaggctaacctcataaccggccaaatcgttggatatgaccctgaactacagataatcctcatacaagtatcttacccaagtgtgtctgaagtgacaggagtccgggctactgagttagtcactgtcagtgtcactacaccaaaaggagaagggcaggcaattgttccgagatatgtggcacagagtagagtgctgacagaggagttggatgtctcgacttgtaggtttagcaaaacaactctttattgtaggtcgattctcacacggcccctaccaactttgatcgccagctgcctgtcagggaagtacgacgattgtcagtacacaacagagataggagcgctatcttcgagattcatcacagtcaatggtggagtccttgcaaactgcagagcaattgtgtgtaagtgtgtctcacccccgcatataataccacaaaacgacattggctccgtaacagttattgactcaagtatatgcaaggaagttgtcttagagagtgtgcagcttaggttagaaggaaagctgtcatcccaatacttctccaacgtgacaattgacctttcccaaatcacaacgtcagggtcgctggatataagcagtgaaattggtagcattaacaacacagttaatcgggtcgacgagttaatcaaggaatccaacgagtggctgaacgctgtgaacccccgccttgtgaacaatacgagcatcatagtcctctgtgtccttgccgccctgattattgtctggctaatagcgctgacagtatgcttctgttactccgcaagatactcagctaagtcaaaacagatgaggggcgctatgacagggatcgataatccatatgtaatacagagtgcaactaagatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggatttcccatctagggagaacctggcagcaggtgacatatcggggcggaagacttggagattactgttccggatcctcacattgagcataggtgtggtctgtcttgccatcaatattgccacaattgcaaaattggatcacctggataacatggcttcgaacacatggacaacaactgaggctgaccgtgtgatatctagcatcacgactccgctcaaagtccctgtcaaccagattaatgacatgtttcggattgtagcgcttgacctacctctgcagatgacatcattacagaaagaaataacatcccaagtcgggttcttggctgaaagtatcaacaatgttttatccaagaatggatctgcaggcctggttcttgttaatgaccctgaatatgcaggggggatcgctgtcagcttgtaccaaggagatgcatctgcaggcctaaatttccagcccatttctttaatagaacatccaagttttgtccctggtcctactactgctaagggctgtataaggatcccgaccttccatatgggcccttcacattggtgttactcacataacatcattgcatcaggttgccaggatgcgagccactccagtatgtatatctctctgggggtgctgaaagcatcgcagaccgggtcgcctatcttcttgacaacggccagccatctcgtggatgacaacatcaaccggaagtcatgcagcatcgtagcctcaaaatacggttgtgatatcctatgcagtattgtgattgaaacagagaatgaggattataggtctgatccggctactagcatgattataggtaggctgttcttcaacgggtcatacacagagagcaagattaacacagggtccatcttcagtctattctctgctaactaccctgcggtggggtcgggtattgtagtcggggatgaagccgcattcccaatatatggtggggtcaagcagaacacatggttgttcaaccagctcaaggattttggttacttcacccataatgatgtgtacaagtgcaatcggactgatatacagcaaactatcctggatgcatacaggccacctaaaatctcaggaaggttatgggtacaaggcatcctattgtgcccagtttcactgagacctgatcctggctgtcgcttaaaggtgttcaataccagcaatgtgatgatgggggcagaagcgaggttgatccaagtaggctcaaccgtgtatctataccaacgctcatcctcatggtgggtggtaggactgacttacaaattagatgtgtcagaaataacttcacagacaggtaacacactcaaccatgtagaccccattgcccatacaaagttcccaagaccatctttcaggcgagatgcgtgtgcgaggccaaacatatgccctgctgtctgtgtctccggagtttatcaggacatttggccgatcagtacagccaccaataacagcaacattgtgtgggttggacagtacttagaagcattctattccaggaaagacccaagaatagggatagcaacccagtatgagtggaaagtcaccaaccagctgttcaattcgaatactgagggagggtactcaaccacaacatgcttccggaacaccaaacgggacaaggcatattgtgtagtgatatcagagtacgctgatggggtgttcggatcatacaggatcgttcctcagcttatagagattagaacaaccaccggtaaatctgagtagttgagtcaattataaaggagttggaaagatggcattgtatcacctatcttctgcgacatcaagaatca

SEQ ID NO: 11 - DNA, 3714 bp, Artificial Sequence
  APMV3/Turkey/Wisconsin/68
atttacagttagtttacctgtctatcaagttagaaaaaacacgggtagaagattctggatcccggttggcgccctccaggtgcaagatggcctccccaatggtcccactactcatcataacggtagtacccgcactcatttcaagtcaatcagctaatattgataagctcattcaagcagggattatcatgggctcagggaaggaactccacatttatcaagaatctggctctcttgatttgtatcttagactattgccagttatcccttcaaatctttctcattgccagagtgaagtaataacacaatataactcgactgtaacgagactattatcaccaattgcaaaaaatctaaaccatttgctacaaccgagaccgtctggcaggttatttggcgctgtaattggatcgattgccttaggggtagctacatccgcacagatttcagctgctatagcattggtccgtgctcaacagaatgcaaacgatatcctcgctcttaaagctgcaatacaatctagtaatgaggcaataaaacaacttacttatggccaagaaaagcaactactagcaatatcaaaaatacaaaaagccgtaaatgaacaagtaatccctgcattgactgcacttgactgtgcagttcttggaaataaactagctgcacaactgaacctctacctcattgaaatgacgactatttttggtgaccaaataaataacccagtcctaactccaataccactcagttatctcctgcggttgacaggctctgagttaaatgatgtattattacaacagactcgatcctctttgagcctaatccaccttgtctctaaaggcttattaagtggtcagattataggatatgacccttcagtacaaggcatcattatcagaataggactgatcaggactcaaagaatagatcggtcactagttttccaaccttacgtattaccaattactattagttctaacatagccacaccaattatacccgactgtgtggtcaagaagggagtaataattgagggaatgcttaagagtaattgtatagaattggaacgagatataatttgcaagactatcaacacataccaaataactaaggaaactagagcatgcttacaaggtaatataacaatgtgtaagtaccagcagtccaggacacagttgagcaccccctttattacatataatggagttgtaattgcaaattgtgatttggtatcatgccgatgcataagaccccctatgattatcacacaagtaaaaggttaccctctgacaattataaataggaatttatgtaccgagttgtcggtggataatttaattttaaatattgaaacaaaccataacttttcattaaaccctactattatagattcacaatcccggcttatagctactagtccattagaaatagatgcccttattcaagatgcgcaacatcacgcggctgcggcccttcttaaagtagaagaaagcaatgctcacttattaagagttacagggctgggctcatcaagttggcacatcatacttatattaacattgcttgtatgcaccatagcatggctcattggtttatctatttatgtctgccgcattaaaaatgatgactcgaccgacaaagaacctacaacccaatcatcgaaccgaggcattggggttggatctatacaatatatgacatgatgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggagccgacaggatcaaaagttgacattgtcccttcccaaggtaccaagagaacatgtcgaaccttttatcgcctcttaattcttattttgaatcttattataattatattaacaattatcagtatttatgtctctatctcaacagatcaacacaaattgtgcaataatgaggctgactcacttttacactcaatagtagaacccataacagtccccctaggaacagactcggatgttgaggatgaattacgtgagattcgacgtgatacaggcataaatattcctatccaaattgacaacacagagaacatcatattaactacattagcaagtatcaactctaacattgcacgccttcataacgccaccgatgaaagcccaacatgcctgtcaccagttaatgatcccaggtttatagcagggattaataagataaccaaagggtcgatgatatataggaatttcagcaatttgatagaacatgttaactttataccatctccaacgacattatcaggctgtacaagaattccatctttttcactatctaaaacacattggtgttactcgcataatgtaatatctactggttgtcaagaccatgctgcgagttcacagtatatttccataggaatagtagatacaggattgaataatgagccctatttgcgtacaatgtcttcacgcttgctaaatgatggcctaaatagaaagagctgctctgtcacagccggcgctggtgtctgttggctattgtgtagtgttgtaacagaaagtgaatcagctgactacagatcaagagcccccactgcaatgattctcggaaggttcaatttttatggtgattacactgaatcccctgttcctgcatctttgttcagcggtcgtttcactgctaattaccctggagttggctcaggaacccaattaaatgggaccctttattttccaatatatgggggtgttgttaacgactctgatattgagttatcgaaccgagggaagtcattcagacctaggaaccctacaaacccatgtccagatcctgaggtgacccaaagtcagagggctcaggcaagttactatccgacaaggtttggcaggctgctcatacaacaagcaatactagcttgtcgtattagtgacactacatgcactgattattatcttctatactttgataataatcaagtcatgatgggtgcagaagcccgaatttattatttaaacaatcagatgtacttatatcaaagatcttcgagttggtggccgcatccgcttttttacagattctcactgcctcattgtgaacctatgtctgtctgtatgatcaccgatacacacttaatattgacatatgctacctcacgccctggcacttcaatttgtacaggggcctcgcgatgtcctaataactgtgttgatggtgtctatacagacgtttggcccttgactgagggtacaacacaagatccagattcctactacacagtattcctcaacagtcccaaccgcaggatcagtcctacaattagcatttacagctacaaccagaagattagctctcgtctggctgtaggaagtgaaataggagctgcttacacgaccagtacatgttttagcaggacagacactggggcactatactgcatcactataatagaagctgtaaacacaatctttggacaataccgaatagtaccgatccttgttcaactaattagtgactagttgagtcaattataaaggagttggaaagatggcattgtatcacctatcttctgcgacatcaagaatca

SEQ ID NO: 12 - DNA, 3846 bp, Artificial Sequence
  APMV12/Wigeon/Italy/3920_1/05
atttacagttagtttacctgtctatcaagttagaaaaaacacgggtagaagattctggatcccggttggcgccctccaggtgcaagatggccatcccagtgccctcttcgaccgctctgatgatcttcaacattctagtgtccctcgcccccgcctccgctctggatggcagactgttgttaggagcaggtatcgtacctacgggagacagacaggtaaatgtgtatacttcatctcaaaccggtataattgccttaaaattgctgcccaacctcccaaaggataaggagaattgcgctgaggtgtctatcagatcctacaacgagactctgacccgcatcctcacccctctcgctcaatccatggcagccataaggggtaattcaacagtatcaactcgtggaagagagccaagactagtgggtgccatcataggaggcgtagctctaggtgtagctacggcagcacagatcacagcggcaacggcccttatccaagccaatcaaaatgcagagaacattgcaagacttgccaaaggtctagcagctaccaatgaggcagtgacggatttaacgaaaggagtgggctctcttgctattggggttggaaagttacaggattatgtaaatgagcaatttaataggacgggagaggcaatcgaatgtttgacgatcgaatctagagtaggtgtccagctcagtctctatctaacagaggttattggagtcttcggtgatcagatcacatctccagcattatctgatatcagtattcaggcattatacaatctggctggagggaacttgaacgtcttgctgcagaagatgggtattgaagggacacagctaggctccttaatcaacagcggattgataaaaggcagaccaatcatgtatgatgatggtaacaaaattttaggtatccaagtaactcttccatcagtgggtaggatcaatggcgcacgagcaactctacttgaggcaattgcggtggctactcctaaagggaatgctagcccattaatacctagagctgttatctcagtgggatcgctagtggaagaattagatatgactccatgcgtgctgactccaacagacatcttttgcaccaggatcttgtcttatccattaagtgattctctcaccacttgtctcaaagggaatctttcgtcttgcgtcttctcacgtacggaaggggcattatcgacaccttatgtttctgtgcatggtaagattgttgccaattgtaagtctgtggtttgccgatgtgtggagccacaacaaatcatatcccaaaactatggggaggcccttagcctgatagatgagtccctatgtaggatcttagaactaaacggagtgatccttaagatggacggacagttcacatcagaatacacaaaaaacataactatagatcctgtgcaggtcataatatctggaccgatcgatatatcttctgagctttcgcaggtcaaccaatcactagatagcgcactggaaaatataaaagagagcaattcatacctgtcaaaagtgaatgtgaagctgatcagctcctcggccatgatcacgtacattgtgataactgtgatttgcctgattttgactttcgtagcgttagtccttgggatatactcatatacaaaaatcaggtctcaacagaagactctgatatggatgggtaataacattgcgaggtcaaaagaggggaaccggttttgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggagagtgcaaccagccaagtgtcctttgaaaatgacaaaacctctgatcgtcggacttggcgagcagtatttagagtactgatgataatactcgctcttagtagcctatgtgtaactgtagcagctcttatatactcagcgaaggccgcaatccctgggaacatcgatgcatctgaacaaaggatattatcatccgttgaggccgttcaggtgcccgtatcaaggttagaagacaccagtcagaagatataccgccaggtcattctcgaggcgccggtaactcagctcaacatggagacgaatattctaaatgctattacatccctttcatatcaaattgatgcttcagccaactcttctggttgcggtgcccctgtccatgactctgacttcacagggggtgtcggtcgagagctacttcaagaggcagaagttaatctgaccataatcaggccctccaaattcttggagcacttaaacttcataccagccccgacaacagggaatggctgcacaaggataccatcgtttgatctaggccaaactcattggtgctacacacacaacgtcgtgctcaatggctgcagagaccgaggccactcttttcaatatgttgcactaggcatactcaggacatcagctacagggtcagtattcttatcaacactccgatctgtaaatttagacgacgaccgtaacagaaagtcatgtagtgtaagtgcaaccccgataggctgcgagatgctctgttctcttgtcacagagactgaagaaggagattatgatagcatcgacccgacccctatggtgcatggcaggttaggatttgatggcaaatatagggaagtggaccttagcgaaaaggagatattcgctgactggcgcgccaattatcctgctgttggcggtggcgctttttttggtaatcgtgtatggttccctgtttatggaggtctgaaggaaggaacccaaagtgagagagatgcagagaaaggttatgcaatatataaacgcttcaataacacttgccctgacgataatacaactcaaatcgcgaatgctaaagcatcatatcggccatctcgatttggcggacgatttatccaacagggtatcctctcttttaaagttgaagggaacttaggatcagatccgatcctcagcctgactgacaactcaatcacattgatgggtgccgaggcacgtgtgatgaatattgagaataaactatacctctatcagagaggtacttcttggtttccatctgccttagtataccccttggatgtagctaatacagctgtaaaagtgcgggcgccatacatttttgacaaattcactaggcccggaggacatccatgcagcgccagttcacggtgccctaacgtatgcgtcacaggggtttatacggatgcctatccacttgtattttcaaggagtcatgacattgtggcagtctacggtatgcagttggcggcgggcactgcacgacttgatcctcaggcagcaatatggtatgggaacgagatgagtacacctactaaagtaagtagctcaactactaaagctgcctatactacttccacatgttttaaggtgacaaaaactaagagaatctactgtataagtatagcagaaatagggaacacactctttggcgagtttaggatagtgccactattaatcgaagtacaaaagactcctctcactaggagaagcgagctccggcaacaaatgccccaacctcccatcgatttggttattgacaatccgttctgtgcgccctctggtaacttgagcagaaagaatgccattgacgagtatgccaattcatggccatagttgagtcaattataaaggagttggaaagatggcattgtatcacctatcttctgcgacatcaagaatca

SEQ ID NO: 13 - DNA, 3720 bp, Artificial Sequence
  APMV5/budgerigar/Japan/TI/75
atttacagttagtttacctgtctatcaagttagaaaaaacacgggtagaagattctggatcccggttggcgccctccaggtgcaagatgttccaacttcctttgaccattcttcttagcattcttagtgttcaccagtcgctttgtctagacaacagtaagctcattcatgcaggaatcatgagtactactgagagagaagttaatgtttatgcacaatctattactgggtcaatagtggtgaggttgattccaaatatcccaagtaaccataaatcttgtgcaactagccaaatcaaactatacaatgacacgttaacaagattgttgaccccaattagagctaatctagaaggacttattagtgctgtttctcaggaccaatcgcagaattctgggataagagagccgcgttttgtaggcgcagtaattggagcagctgccctcggcttggcaaccgctgcacaggtgactgccactgtagcgttaaatcaagcgcaagaaaacgctcggaatatcctaaagcttaaaaactcgattcagaagacaaacgaggcggtgatggaacttaaagatgctgtgggccaaacagcagtagctattgacaaaactcaggccttcataaataatcaaatcttgcctgcaatttcaaatctctcatgtgaggtcctagggaataaaattggggtccaattatctttgtaccttactgaattaacaacagtattcggcaatcaactgacaaacccagcccttaccacactgtcattacaagccttgtacaatctttgtggagatgacttcaattacttaatcaacctattaaatgcaaaaaatcgtaacttagcctcactttatgaagcaaacctaattcaggggagaatcactcaatatgactcaatgaatcagttattaattattcaggtacaaataccaagcatctccatagtgtcaggaatgagggtcacagaattattcacacttagtgttgatacacctataggagagggaaaggccctagtaccaaaatatgtcctgtcctcagggagaataatggaagaggttgacctaagcagttgcgctataacatcaacatcagttttctgttcctctatcatctctagaccccttccacttgaaacaataaattgcctgaatgggaatgttacacagtgtcaatttaccgccaacacaggaacccttgaatcgagatacgctgttataggaggcttggtgattgctaactgtaaggctatagtatgcaggtgcctaaatccaccaggtgtcattgcgcaaaatcttggcttaccaattacaatcatctcatccaatacttgtcagcgaattaatttagaacaaatcactttgtctcttgggaacagcatattatctacatacagtgccaatttatcccaagttgagatgaatttagctccatcaaatcctctggatatctcagttgaattgaatcgagtcaacaccagtctctctaaagtggaatctctaataaaagaaagcaatagtatcctggactcagttaaccctcaaattttaaatgtcaagacagtaattatcctggccttcataataggactcattgttgtgtggtgtttcatattgacatgtctaataattagaggatttatgcttcttgtaaaacaacaaaagtttaaaggactctctgttcagaataatccgtatgtttctaacaattctcattgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggacaaatcatattacatagagcctgaagatcaaagaggtaactctcgaacatggagactattatttaggttgattgtattaacgttgctctgtctgatcgcatgtatcttagtaagtcaattgttctacccttggctcccccaagtcttgtccactctgatcagcctaaataactcaattatcacaagcagcaatggtctcaaaaaggaaatcctgaaccagaacataaaagaggacctcatatatagagaagttgctataaatatacctttaacattagatagggttactgttgaggtagggactgcagtaaaccagattactgatgcactcaggcaactccagtcagttaatggatctgctgcattcgcctcatcaaactctcctgattatagtgggggaatagaacacctgattttccaaaggaatacgcttattaatcgctcagtgagtgtctcagatttaatagaacaccccagtttcataccaactcctactacacagcatggttgtaccagaatccccacattccacctaggaactcgccactggtgctatagtcacaatataataggtcagggatgtgctgattctagagctagtgtgatgtatatttcaatgggagcactgggtgtcagttcattgggaaccccgaccttcacaacatctgctgcatcaatattatctgatagcctcaatcggaagagttgcagtatagtagcaacaactgagggttgtgacgtactctgcagtatagttacacaaacagaagaccaagattatgctgatcacactcctactccaatgatacatggtagattatggtttaatggcacatacacagagagatccttatcccagagtttattccttggaacatgggctgcgcaatatccggctgtaggatctggtataatgacacctgggcgagttatattccctttctatggaggtgtgatccctaactctcctctcttcttggatctcgaaagattcgctttattcacacataatggagacttagaatgcatgaacttaacacaatatcagaaagaagcaatttactctgcatataagcctcccaagattagaggatcactgtgggcacaaggcttcatagtatgttcagtaggagacatggggaattgctctcttaaagtgatcaatacaagcacagttatgatgggtgcagaaggtcggctacaattagttggggactccgttatgtactatcagagatcatcatcctggtggcctgtaggaattctttatcggttgagtcttgtagacatcatcgccggagatatacaggtcgtcataaacagtgaaccactccctctgagcaagttcccacggccaacctggactccaggagtgtgtcaaaaaccaaatgtatgccctgcagtttgtgtaactggggtctatcaagacctttgggcaatttccgcaggggagacactatctgaaatgacattctttggaggatatttagaggcatccacccaacgaaaagatccatggataggcgttgctaatcaatatagttggttcatgagaagaagattattcaagacaagcactgaagctgcatattcgtcatcaacgtgttttaggaacactagactggatcgaaatttctgcctattagtctttgaattaactgataacttacttggagactggagaattgtccccctcttatttgaattaaccatcgtatagttgagtcaattataaaggagttggaaagatggcattgtatcacctatcttctgcgacatcaagaatca

SEQ ID NO: 14 - DNA, 3702 bp, Artificial Sequence
  APMV10/penguin/Falkland Islands/324/07
atttacagttagtttacctgtctatcaagttagaaaaaacacgggtagaagattctggatcccggttggcgccctccaggtgcaagatgactcgtactcggttgctctttctcctcacttgttacattccaggtgctgtctcacttgacaactctatattagctccagctgggataataagcgcttcggagagacagattgctatatatactcaaactctgcagggaactattgcacttcgatttatacctgtacttccgcagaacttatcatcatgtgctaaagacaccctggaatcctataactccactgtttcaaatcttttattgcctattgctgagaacctaaatgccttgttaaaagacgccgataagccatcccaacgaattattggggctatcataggatcagtagcgctaggcgtagcaacaactgcacaagtgactgcagcccttgcaatgacgcaggcacaacaaaatgcacgaaatatatggaagttgaaagagtctatcaaaaatacaaatcaagcagtattggaactaaaagacgggctacaacaatctgctattgcacttgacaaggtccagtccttcatcaactcggaaatattacctcaaataaatcagttaggatgcgaggttgcagcaaacaaattgggaatatttctatctctctacctaactgaaatcactacagtgttcaaaaatcaaatcacgaatcccgctctttccacattatcataccaagccctttataatttgtgtgggggcaatatggctgctttaacaaaacaaataggaattaaagatacagaaattaactcattatatgaagcagaattgatcacaggacaagttatagggtatgattcagcagaccagatactgttgattcaagtatcatatccaagtgtttcaagggtccaaggggttagggcagtagaactcttgacagtcagtgtggcaacaccaaaaggtgaggggaaagcaattgctccaagctttatagctcagagcaatataattgctgaagagttagatacacaaccatgtaagtttagtaagacaacactctactgtagacaagttaacactaggacattaccagttagggtagcaaactgccttaaaggcaaatataatgactgccaatataccacagaaataggtgcattggcatcacgatatgtcacgattacaaatggggttgttgccaactgcagatctatcatctgtaggtgcctggacccggagggaatagttgcccaaaattctgacgcagcaatcactgttattgataggtccacttgcaagttgatccagttaggtgatattaccctcagattagaaggcaaattatcctcatcatactcaaagaatataaccattgatatatctcaagtaactacatctggttctttagatataagtagcgaattaggctctattaataatactataaccaaagtagaagatttgataagtaagtccaatgattggttgagtaaagtaaatcctaccctaatatcgaatgacactatcattgccctctgtgtgattgctggtattgtcgttatctggttagttataatcacaatactatcgtattatatactcataaaacttaaaaatgtagcattgctttcaaccatgccaaagaaagatctaaacccgtatgttaacaacactaaattttgatgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggactcatcacaaatgaatattttggatgctatggatagggaaagtagtaaaaggacatggaggggagtattccgagttactactataatcatggttgtcacatgtgtcgtcctatctgcaatcacactatcgaaggttgcccatcctcaggggttcgacaccaatgagctgggtaatggcatcgtggatcgagtcagcgataaaataactgaggcgttaacagtgccaaacaatcaaataggtgaaatattcaaaattgtggcactggacttgcatgttctggtcagctcatcgcaacaggcaattgcaggacaaattggcatgcttgctgagagtatcaacagtatattaagccaaaatggatctgcatcgaccattctatcatccagcccagaatatgcagggggtataggtgtccctctctttagcaataagttaacaaatggaactgtaataaaacccataaccttaattgaacaccctagtttcatcccgggcccaactactataggaggttgcactcggattcccacattccatatggcctcgtctcattggtgttactctcataatatcatagagaaaggttgtaaggatagtgggatatcatccatgtatatatcattaggagtattacaagtattgaagaaaggaactcctgtatttttggtaactgcaagcgctgtactatctgatgataggaaccggaaatcgtgcagtattataagttcaaggtttgggtgtgagatactatgcagccttgtaacagaagcagagtctgacgattacaaatctgatacgccaactggaatggtgcacggcagattatatttcaatgggacatacagagaagggctggtagatacagagactatattccgagacttttctgctaattatcccggggtcggatcaggggaaattgtagaggggcacatacattttcccatatacggaggagtgaagcagaatactggcctatacaacagtcttaccccttattggctcgatgcaaagaacaaatatgactattgcaagttgccatatacaaatcaaacaatccagaactcgtataaacccccattcatccacggaagattttgggcacaaggaatactatcatgtgaattagatctattcaatttggggaattgcaatctcaagataatccgaagtgataaagtcatgatgggagcagaaagtcggttaatgcttgtggggtcaaaattgctaatgtatcagagggcatcgtcctggtggccgctggggattacacaggagatagatatagctgagctacactcaagtaatactaccatattaagagaagttaaacccatactgtcatcgaagttcccacggccgtcctatcagccgaattattgcacgaagccaagtgtatgtcctgcagtgtgtgtcacaggagtatacactgacatgtggcctatttcaatcactggcaacatatctgattatgcttggattagccattatttggatgcaccaacatcaagacaacagccgaggattgggattgcaaaccaatatttctggatccatcagacgactatatttccaaccaatactcagagctcctactccactaccacatgctttaggaatcaggtgagaagtaggatgttttgtttatccattgcagagtttgcagatggagtgtttggggaatttaggatcgtgcccttattatatgagttgagagtatagttgagtcaattataaaggagttggaaagatggcattgtatcacctatcttctgcgacatcaagaatca

SEQ ID NO: 15 - DNA, 15186 bp, Artificial Sequence
  cDNA of genomic sequence of NDV strain LaSota
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 16 - DNA, 21 bp, Mus
  Mouse IFN-B Forward Primer Sequence
cagctccaagaaaggacgaac

SEQ ID NO: 17 - DNA, 21 bp, Mus
  Mouse IFN-B Reverse Primer Sequence
ggcagtgtaactcttctgcat

SEQ ID NO: 18 - DNA, 22 bp, Homo sapiens
  Human IFN-B Forward Primer Sequence
tctggcacaacaggtagtaggc

SEQ ID NO: 19 - DNA, 22 bp, Homo sapiens
  Human IFN-B Reverse Primer Sequence
gagaagcacaacaggagagcaa

SEQ ID NO: 20 - DNA, 21 bp, Mus
  Mouse IL-6 Forward Primer Sequence
ctgcaagagacttccatccag

SEQ ID NO: 21 - DNA, 23 bp, Mus
  Mouse IL-6 Reverse Primer Sequence
agtggtatagacaggtctgttgg

SEQ ID NO: 22 - DNA, 22 bp, Homo sapiens
  Human IL-6 Forward Primer Sequence
agaggcactggcagaaaacaac

SEQ ID NO: 23 - DNA, 23 bp, Homo sapiens
  Human IL-6 Reverse Primer Sequence
aggcaagtctcctcattgaatcc

SEQ ID NO: 24 - DNA, 21 bp, Homo sapiens
  Human IL-1B Forward Primer Sequence
ctcgccagtgaaatgatggct

SEQ ID NO: 25 - DNA, 21 bp, Homo sapiens
  Human IL-1B Reverse Primer Sequence
gtcggagattcgtagctggat

SEQ ID NO: 26 - DNA, 22 bp, Mus
  Mouse IL-1B Forward Primer Sequence
tgggctggactgtttctaatgc

SEQ ID NO: 27 - DNA, 21 bp, Mus
  Mouse IL-1B Reverse Primer Sequence
tgtcttggccgaggactaagg

SEQ ID NO: 28 - DNA, 21 bp, Mus
  Mouse ISG-15 Forward Primer Sequence
ggtgtccgtgactaactccat

SEQ ID NO: 29 - DNA, 21 bp, Mus
  Mouse ISG-15 Reverse Primer Sequence
tggaaagggtaagaccgtcct

SEQ ID NO: 30 - DNA, 22 bp, Homo sapiens
  Human ISG-15 Forward Primer Sequence
tcctggtgaggaataacaaggg

SEQ ID NO: 31 - DNA, 20 bp, Homo sapiens
  Human ISG-15 Reverse Primer Sequence
gtcagccagaacaggtcgtc

SEQ ID NO: 32 - DNA, 21 bp, Mus
  Mouse MX1 Forward Primer Sequence
gaccataggggtcttgaccaa

SEQ ID NO: 33 - DNA, 23 bp, Mus
  Mouse MX1 Reverse Primer Sequence
agacttgctctttctgaaaagcc

SEQ ID NO: 34 - DNA, 21 bp, Homo sapiens
  Human MX1 Forward Primer Sequence
gtttccgaagtggacatcgca

SEQ ID NO: 35 - DNA, 20 bp, Homo sapiens
  Human MX1 Reverse Primer Sequence
gaagggcaactcctgacagt

SEQ ID NO: 36 - DNA, 21 bp, Mus
  Mouse STAT-1 Forward Primer Sequence
tcacagtggttcgagcttcag

SEQ ID NO: 37 - DNA, 21 bp, Mus
  Mouse STAT-1 Reverse Primer Sequence
gcaaacgagacatcataggca

SEQ ID NO: 38 - DNA, 23 bp, Homo sapiens
  Human STAT-1 Forward Primer Sequence
atgtctcagtggtacgaacttca

SEQ ID NO: 39 - DNA, 21 bp, Homo sapiens
  Human STAT-1 Reverse Primer Sequence
tgtgccaggtactgtctgatt

SEQ ID NO: 40 - DNA, 20 bp, Artificial Sequence
  LS-L289A N Forward Primer Sequence
gaaccatgttgcccttgcag

SEQ ID NO: 41 - DNA, 20 bp, Artificial Sequence
  LS-L289A N Reverse Primer Sequence
cctctccagggtatcggtga

SEQ ID NO: 42 - DNA, 20 bp, Artificial Sequence
  APMV-4 N Forward Primer Sequence
atcggtccttagcaggagga

SEQ ID NO: 43 - DNA, 20 bp, Artificial Sequence
  APMV-4 N Reverse Primer Sequence
gggtccagtcgttgacactt

SEQ ID NO: 44 - DNA, 19395 bp, Artificial Sequence
  NDV-APMV2-GFP APMV2/Chicken/California/ Yucaipa/56
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttaatacgactcactatagggagattggtctgatgagtccgtgaggacgaaacggagtctagactccgtcaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggttagaaaaaatacgggtagaaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgaatcaagcactcgtgattttgttggtatctttccagctcggcgttgccttagataactcagtgttggctccaataggagtagctagcgcacaggagtggcaactggcggcatatacaacgaccctcacagggaccatcgcagtgagatttatcccggtcctgcctgggaacctatcaacatgtgcacaggagacgctgcaggaatataatagaactgtgactaatatcttaggcccgttgagagagaacttggatgctctcctatctgacttcgataaacctgcatcgaggttcgtgggcgccatcattgggtcggtggccttgggggtagcaacagctgcacaaatcacagccgccgtggctctcaatcaagcacaagagaatgcccggaatatatggcgtctcaaggaatcgataaagaaaaccaatgcggctgtgttggaattgaaggatggacttgcaacgactgctatagctttggacaaagtgcaaaagtttatcaatgatgatattataccacagattaaggacattgactgccaggtagttgcaaataaattaggcgtctacctctccttatacttaacagagcttacaactgtatttggttctcagatcactaatcctgcattatcaacgctctcttaccaggcgctgtacagcttatgtggaggggatatgggaaagctaactgagctgatcggtgtcaatgcaaaggatgtgggatccctctacgaggctaacctcataaccggccaaatcgttggatatgaccctgaactacagataatcctcatacaagtatcttacccaagtgtgtctgaagtgacaggagtccgggctactgagttagtcactgtcagtgtcactacaccaaaaggagaagggcaggcaattgttccgagatatgtggcacagagtagagtgctgacagaggagttggatgtctcgacttgtaggtttagcaaaacaactctttattgtaggtcgattctcacacggcccctaccaactttgatcgccagctgcctgtcagggaagtacgacgattgtcagtacacaacagagataggagcgctatcttcgagattcatcacagtcaatggtggagtccttgcaaactgcagagcaattgtgtgtaagtgtgtctcacccccgcatataataccacaaaacgacattggctccgtaacagttattgactcaagtatatgcaaggaagttgtcttagagagtgtgcagcttaggttagaaggaaagctgtcatcccaatacttctccaacgtgacaattgacctttcccaaatcacaacgtcagggtcgctggatataagcagtgaaattggtagcattaacaacacagttaatcgggtcgacgagttaatcaaggaatccaacgagtggctgaacgctgtgaacccccgccttgtgaacaatacgagcatcatagtcctctgtgtccttgccgccctgattattgtctggctaatagcgctgacagtatgcttctgttactccgcaagatactcagctaagtcaaaacagatgaggggcgctatgacagggatcgataatccatatgtaatacagagtgcaactaagatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggatttcccatctagggagaacctggcagcaggtgacatatcggggcggaagacttggagattactgttccggatcctcacattgagcataggtgtggtctgtcttgccatcaatattgccacaattgcaaaattggatcacctggataacatggcttcgaacacatggacaacaactgaggctgaccgtgtgatatctagcatcacgactccgctcaaagtccctgtcaaccagattaatgacatgtttcggattgtagcgcttgacctacctctgcagatgacatcattacagaaagaaataacatcccaagtcgggttcttggctgaaagtatcaacaatgttttatccaagaatggatctgcaggcctggttcttgttaatgaccctgaatatgcaggggggatcgctgtcagcttgtaccaaggagatgcatctgcaggcctaaatttccagcccatttctttaatagaacatccaagttttgtccctggtcctactactgctaagggctgtataaggatcccgaccttccatatgggcccttcacattggtgttactcacataacatcattgcatcaggttgccaggatgcgagccactccagtatgtatatctctctgggggtgctgaaagcatcgcagaccgggtcgcctatcttcttgacaacggccagccatctcgtggatgacaacatcaaccggaagtcatgcagcatcgtagcctcaaaatacggttgtgatatcctatgcagtattgtgattgaaacagagaatgaggattataggtctgatccggctactagcatgattataggtaggctgttcttcaacgggtcatacacagagagcaagattaacacagggtccatcttcagtctattctctgctaactaccctgcggtggggtcgggtattgtagtcggggatgaagccgcattcccaatatatggtggggtcaagcagaacacatggttgttcaaccagctcaaggattttggttacttcacccataatgatgtgtacaagtgcaatcggactgatatacagcaaactatcctggatgcatacaggccacctaaaatctcaggaaggttatgggtacaaggcatcctattgtgcccagtttcactgagacctgatcctggctgtcgcttaaaggtgttcaataccagcaatgtgatgatgggggcagaagcgaggttgatccaagtaggctcaaccgtgtatctataccaacgctcatcctcatggtgggtggtaggactgacttacaaattagatgtgtcagaaataacttcacagacaggtaacacactcaaccatgtagaccccattgcccatacaaagttcccaagaccatctttcaggcgagatgcgtgtgcgaggccaaacatatgccctgctgtctgtgtctccggagtttatcaggacatttggccgatcagtacagccaccaataacagcaacattgtgtgggttggacagtacttagaagcattctattccaggaaagacccaagaatagggatagcaacccagtatgagtggaaagtcaccaaccagctgttcaattcgaatactgagggagggtactcaaccacaacatgcttccggaacaccaaacgggacaaggcatattgtgtagtgatatcagagtacgctgatggggtgttcggatcatacaggatcgttcctcagcttatagagattagaacaaccaccggtaaatctgagtagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtgcgcgcggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatatgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 45 - DNA, 19395 bp, Artificial Sequence
  NDV-APMV3-GFP APMV3/Turkey/Wisconsin/68
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttaatacgactcactatagggagattggtctgatgagtccgtgaggacgaaacggagtctagactccgtcaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggttagaaaaaatacgggtagaaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatggcctccccaatggtcccactactcatcataacggtagtacccgcactcatttcaagtcaatcagctaatattgataagctcattcaagcagggattatcatgggctcagggaaggaactccacatttatcaagaatctggctctcttgatttgtatcttagactattgccagttatcccttcaaatctttctcattgccagagtgaagtaataacacaatataactcgactgtaacgagactattatcaccaattgcaaaaaatctaaaccatttgctacaaccgagaccgtctggcaggttatttggcgctgtaattggatcgattgccttaggggtagctacatccgcacagatttcagctgctatagcattggtccgtgctcaacagaatgcaaacgatatcctcgctcttaaagctgcaatacaatctagtaatgaggcaataaaacaacttacttatggccaagaaaagcaactactagcaatatcaaaaatacaaaaagccgtaaatgaacaagtaatccctgcattgactgcacttgactgtgcagttcttggaaataaactagctgcacaactgaacctctacctcattgaaatgacgactatttttggtgaccaaataaataacccagtcctaactccaataccactcagttatctcctgcggttgacaggctctgagttaaatgatgtattattacaacagactcgatcctctttgagcctaatccaccttgtctctaaaggcttattaagtggtcagattataggatatgacccttcagtacaaggcatcattatcagaataggactgatcaggactcaaagaatagatcggtcactagttttccaaccttacgtattaccaattactattagttctaacatagccacaccaattatacccgactgtgtggtcaagaagggagtaataattgagggaatgcttaagagtaattgtatagaattggaacgagatataatttgcaagactatcaacacataccaaataactaaggaaactagagcatgcttacaaggtaatataacaatgtgtaagtaccagcagtccaggacacagttgagcaccccctttattacatataatggagttgtaattgcaaattgtgatttggtatcatgccgatgcataagaccccctatgattatcacacaagtaaaaggttaccctctgacaattataaataggaatttatgtaccgagttgtcggtggataatttaattttaaatattgaaacaaaccataacttttcattaaaccctactattatagattcacaatcccggcttatagctactagtccattagaaatagatgcccttattcaagatgcgcaacatcacgcggctgcggcccttcttaaagtagaagaaagcaatgctcacttattaagagttacagggctgggctcatcaagttggcacatcatacttatattaacattgcttgtatgcaccatagcatggctcattggtttatctatttatgtctgccgcattaaaaatgatgactcgaccgacaaagaacctacaacccaatcatcgaaccgaggcattggggttggatctatacaatatatgacatgatgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggagccgacaggatcaaaagttgacattgtcccttcccaaggtaccaagagaacatgtcgaaccttttatcgcctcttaattcttattttgaatcttattataattatattaacaattatcagtatttatgtctctatctcaacagatcaacacaaattgtgcaataatgaggctgactcacttttacactcaatagtagaacccataacagtccccctaggaacagactcggatgttgaggatgaattacgtgagattcgacgtgatacaggcataaatattcctatccaaattgacaacacagagaacatcatattaactacattagcaagtatcaactctaacattgcacgccttcataacgccaccgatgaaagcccaacatgcctgtcaccagttaatgatcccaggtttatagcagggattaataagataaccaaagggtcgatgatatataggaatttcagcaatttgatagaacatgttaactttataccatctccaacgacattatcaggctgtacaagaattccatctttttcactatctaaaacacattggtgttactcgcataatgtaatatctactggttgtcaagaccatgctgcgagttcacagtatatttccataggaatagtagatacaggattgaataatgagccctatttgcgtacaatgtcttcacgcttgctaaatgatggcctaaatagaaagagctgctctgtcacagccggcgctggtgtctgttggctattgtgtagtgttgtaacagaaagtgaatcagctgactacagatcaagagcccccactgcaatgattctcggaaggttcaatttttatggtgattacactgaatcccctgttcctgcatctttgttcagcggtcgtttcactgctaattaccctggagttggctcaggaacccaattaaatgggaccctttattttccaatatatgggggtgttgttaacgactctgatattgagttatcgaaccgagggaagtcattcagacctaggaaccctacaaacccatgtccagatcctgaggtgacccaaagtcagagggctcaggcaagttactatccgacaaggtttggcaggctgctcatacaacaagcaatactagcttgtcgtattagtgacactacatgcactgattattatcttctatactttgataataatcaagtcatgatgggtgcagaagcccgaatttattatttaaacaatcagatgtacttatatcaaagatcttcgagttggtggccgcatccgcttttttacagattctcactgcctcattgtgaacctatgtctgtctgtatgatcaccgatacacacttaatattgacatatgctacctcacgccctggcacttcaatttgtacaggggcctcgcgatgtcctaataactgtgttgatggtgtctatacagacgtttggcccttgactgagggtacaacacaagatccagattcctactacacagtattcctcaacagtcccaaccgcaggatcagtcctacaattagcatttacagctacaaccagaagattagctctcgtctggctgtaggaagtgaaataggagctgcttacacgaccagtacatgttttagcaggacagacactggggcactatactgcatcactataatagaagctgtaaacacaatctttggacaataccgaatagtaccgatccttgttcaactaattagtgactagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtgcgcgcggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatatgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 46 - protein, 5 aa, Artificial Sequence
  linker
GGGGS

SEQ ID NO: 47 - protein, 5 aa, Artificial Sequence
  linker
  MISC_FEATURE (1)..(5): GGGGS can be repeated n times, wherein n is 1, 2, 3, 4, 5 or more
GGGGS

(19) United States
(12) Patent Application Publication
(10) Pub. No.: US 2024/0199705 A1
(43) Pub. Date: Jun. 20, 2024

(54) CHIMERIC NEWCASTLE DISEASE VIRUS EXPRESSING APMV HN AND F PROTEINS

(71) Applicant: ICAHN SCHOOL OF MEDICINE AT MOUNT SINAI, New York, NY (US)

(72) Inventors: Adolfo GARCIA-SASTRE, New York, NY (US); Ignacio MENA, New York, NY (US); Peter PALESE, New York, NY (US); Florian KRAMMER, New York, NY (US); Weina SUN, New York, NY (US)

(73) Assignee: ICAHN SCHOOL OF MEDICINE AT MOUNT SINAI, New York, NY (US)

(21) Appl. No.: 18/556,458

(22) PCT Filed: Apr. 25, 2022
(86) PCT No.: PCT/US2022/026185

§ 371 Date: Oct. 20, 2023

Related U.S. Application Data
(60) Provisional application No. 63/302,434, filed on Jan. 24, 2022.
(60) Provisional application No. 63/179,994, filed on Apr. 26, 2021.

(51) Int. Cl. C07K 14/005; A61K 39/00; A61K 39/17; A61P 35/00; C12N 15/86

(52) U.S. Cl. CPC C07K 14/005; A61K 39/17; A61P 35/00; C12N 15/86; A61K 2039/525; C12N 2760/18122; C12N 2760/18134; C12N 2760/18143

(57) ABSTRACT

In one aspect, described herein are recombinant Newcastle disease virus (“NDV”) comprising a packaged genome, wherein the packaged genome comprises a nucleotide sequence of a Newcastle disease virus genome in which the nucleotide sequence encoding the NDV HN protein has been replaced with a nucleotide sequence encoding the HN protein of an avian paramyxovirus (APMV) other than NDV or a variant of the non-NDV-APMV HN protein, and the nucleotide sequence encoding the NDV F protein has been replaced with a nucleotide sequence encoding the F protein of an APMV other than NDV or a variant of the non-NDV-APMV F protein. In some embodiments, the packaged genome further comprises a transgene comprising a nucleotide sequence encoding an antigen. Also described herein are compositions comprising such recombinant NDV and the use of such recombinant NDV to induce an immune response in a subject.

### CROSS-REFERENCE TO RELATED APPLICATIONS

This application claims the benefit of U.S. Provisional Application No. 63/302,434, filed Jan. 24, 2022, and U.S. Provisional Application No. 63/179,994, filed Apr. 26, 2021, the disclosure of each of which is incorporated by reference herein in its entirety.

### STATEMENT REGARDING FEDERALLY SPONSORED RESEARCH

This invention was made with government support under AI097092 awarded by The National Institutes of Health. The government has certain rights in the invention.

### SEQUENCE LISTING

This application incorporates by reference a Sequence Listing submitted with this application as a text file entitled “06923-382-228_SEQ_LISTING.txt,” was created on Apr. 22, 2022, and is 147,499 bytes in size.

### 1. INTRODUCTION

In one aspect, described herein are recombinant Newcastle disease virus (“NDV”) comprising a packaged genome, wherein the packaged genome comprises a nucleotide sequence of a Newcastle disease virus genome in which the nucleotide sequence encoding the NDV HN protein has been replaced with a nucleotide sequence encoding the HN protein of an avian paramyxovirus (APMV) other than NDV or a variant of the non-NDV-APMV HN protein, and the nucleotide sequence encoding the NDV F protein has been replaced with a nucleotide sequence encoding the F protein of an APMV other than NDV or a variant of the non-NDV-APMV F protein. In some embodiments, the packaged genome further comprises a transgene comprising a nucleotide sequence encoding an antigen. Also described herein are compositions comprising such recombinant NDV and the use of such recombinant NDV to induce an immune response in a subject.

### 2. BACKGROUND

Newcastle disease virus (NDV) is a member of the Avulavirinae subfamily in the Paramyxoviridae family, which has been shown to infect a number of avian species (Alexander, D J (1988). Newcastle disease, Newcastle disease virus—an avian paramyxovirus. Kluwer Academic Publishers: Dordrecht, The Netherlands. pp 1-22). NDV possesses a single-stranded RNA genome in negative sense and does not undergo recombination with the host genome or with other viruses (Alexander, D J (1988). Newcastle disease, Newcastle disease virus—an avian paramyxovirus. Kluwer Academic Publishers: Dordrecht, The Netherlands. pp 1-22). The genomic RNA contains genes in the order of 3′-NP-P-M-F-HN-L-5′, described in further detail below. Two additional proteins, V and W, are produced by NDV from the P gene by alternative mRNAs that are generated by RNA editing. The genomic RNA also contains a leader sequence at the 3′ end.

The structural elements of the virion include the virus envelope which is a lipid bilayer derived from the cell plasma membrane. The glycoprotein, hemagglutinin-neuraminidase (HN) protrudes from the envelope allowing the virus to contain both hemagglutinin (e.g., receptor binding/fusogenic) and neuraminidase activities. The fusion glycoprotein (F), which also interacts with the viral membrane, is first produced as an inactive precursor, then cleaved post-translationally to produce two disulfide linked polypeptides. The active F protein is involved in penetration of NDV into host cells by facilitating fusion of the viral envelope with the host cell plasma membrane. The matrix protein (M), is involved with viral assembly, and interacts with both the viral membrane as well as the nucleocapsid proteins.

The main protein subunit of the nucleocapsid is the nucleocapsid protein (NP) which confers helical symmetry on the capsid. In association with the nucleocapsid are the P and L proteins. The phosphoprotein (P), which is subject to phosphorylation, is thought to play a regulatory role in transcription, and may also be involved in methylation, phosphorylation and polyadenylation. The L gene, which encodes an RNA-dependent RNA polymerase, is required for viral RNA synthesis together with the P protein. The L protein, which takes up nearly half of the coding capacity of the viral genome is the largest of the viral proteins, and plays an important role in both transcription and replication. The V protein has been shown to inhibit interferon-alpha and to contribute to the virulence of NDV (Huang et al. (2003). Newcastle disease virus V protein is associated with viral pathogenesis and functions as an Alpha Interferon Antagonist. Journal of Virology 77: 8676-8685).

### 3. SUMMARY

In one aspect, provided herein is a nucleic acid sequence comprising a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV APMV HN protein coding sequence; and (2) the nucleotide sequence encoding the NDV F protein has been replaced with a nucleotide sequence encoding a non-NDV APMV F protein, wherein NDV intergenic regions are before and after the non-NDV APMV F protein coding sequence. In specific embodiments, the term “non-NDV APMV” is used to refer to an APMV other than NDV. In specific embodiments, the non-NDV APMV F protein and non-NDV APMV HN protein are immunologically distinct from the NDF F protein and NDV HN protein, respectively. In some embodiments, the non-NDV APMV F protein and non-NDV APMV HN protein are from a different genus than NDV. In some embodiments, the non-NDV APMV F protein and non-APMV HN protein are an F protein and an HN protein from a member of the subfamily Avulavirinae, but not NDV. In some embodiments, the non-NDV APMV F protein and non-APMV HN protein are an F protein and an HN protein from a member of the subfamily Avulavirinae and the genus metaavulavirus. In some embodiments, the non-NDV APMV F protein and non-APMV HN protein are an F protein and an HN protein from a member of the subfamily Avulavirinae and genus paraavulavirus. In some embodiments, the non-NDV APMV F protein and non-APMV HN protein are an F protein and an HN protein from a member of the subfamily Avulavirinae and the genus orthoavulavirus but is not NDV. In some embodiments, the NDV genome comprises the NP gene, P gene, M gene, and L gene of NDV LaSota strain.

In some embodiments, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV large polymerase (L), and (5) the nucleotide sequence of any one of SEQ ID NOS:1-14, or a nucleotide sequence that is at least 80%, at least 85%, at least 90%, at least 95%, or at least 98% identical to the nucleotide sequence of any one of SEQ ID NOS:1-14. In some embodiments, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV large polymerase (L), and (5) a negative sense RNA sequence corresponding to the nucleotide sequence of any one of SEQ ID NOS:1-14, or a negative sense RNA sequence corresponding to a nucleotide sequence that is at least 80%, at least 85%, at least 90%, at least 95%, or at least 98% identical to the nucleotide sequence of any one of SEQ ID NOS:1-14. In some embodiments, the NDV nucleocapsid protein, NDV phosphoprotein, NDV matrix protein, and NDV large polymerase are of the NDV LaSota strain.

In some embodiments, provided herein is a nucleic acid sequence comprising the nucleotide sequence of SEQ ID NO:44, or SEQ ID NO:44 without the GFP coding sequence. In some embodiments, provided herein is a nucleic acid sequence comprising a nucleotide sequence that is at least 80%, at least 85%, at least 90%, at least 95%, or at least 98% identical to the nucleotide sequence of SEQ ID NO:44, or SEQ ID NO:44 without the GFP coding sequence. In some embodiments, provided herein is a nucleic acid sequence comprising the nucleotide sequence of SEQ ID NO:45, or SEQ ID NO:45 without the GFP coding sequence. In some embodiments, provided herein is a nucleic acid sequence comprising a nucleotide sequence that is at least 80%, at least 85%, at least 90%, at least 95%, or at least 98% identical to the nucleotide sequence of SEQ ID NO:45, or SEQ ID NO:45 without the GFP coding sequence.

In some embodiments, provided herein is a nucleic acid sequence comprising a negative sense RNA sequence corresponding to the nucleotide sequence of SEQ ID NO:44, or SEQ ID NO:44 without the GFP coding sequence. In some embodiments, provided herein is a nucleic acid sequence comprising a negative sense RNA sequence corresponding to a nucleotide sequence that is at least 80%, at least 85%, at least 90%, at least 95%, or at least 98% identical to the nucleotide sequence of SEQ ID NO:44, or SEQ ID NO:44 without the GFP coding sequence. In some embodiments, provided herein is a nucleic acid sequence comprising a negative sense RNA sequence corresponding to the nucleotide sequence of SEQ ID NO:45, or SEQ ID NO:45 without the GFP coding sequence. In some embodiments, provided herein is a nucleic acid sequence comprising a negative sense RNA sequence corresponding to a nucleotide sequence that is at least 80%, at least 85%, at least 90%, at least 95%, or at least 98% identical to the nucleotide sequence of SEQ ID NO:45, or SEQ ID NO:45 without the GFP coding sequence.

In some embodiments, the nucleic acid sequence further comprises a transgene. In some embodiments, the nucleic acid sequence further comprises a transgene encoding an antigen. In some embodiments, the antigen is viral, bacterial, fungal or protozoan antigen. In some embodiments, the antigen comprises a SARS-CoV-2 spike protein or a fragment thereof. In some embodiments, the fragment comprises the receptor binding domain of the SARS-CoV-2 spike protein. In some embodiments, the fragment comprises the ectodomain of the SARS-CoV-2 spike protein. In some embodiments, the antigen comprises a MERS-CoV antigen, respiratory syncytial virus antigen, human metapneumovirus antigen, a Lassa virus antigen, Ebola virus antigen, or Nipah virus antigen. In some embodiments, the antigen is a cancer or tumor antigen.

In some embodiments, the non-NDV APMV F protein and non-NDV APMV HN protein are immunologically distinct from the NDF F protein and NDV HN protein, respectively. In some embodiments, the non-NDV APMV HN is the HN protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07. In some embodiments, the non-NDV APMV F is the F protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07.

In some embodiments, the nucleic acid sequence is a cDNA sequence. In some embodiments, the nucleic acid sequence is a negative-sense stranded RNA sequence.

In some embodiments, provided herein is a recombinant NDV comprising a nucleic acid sequence described herein. In some embodiments, provided herein is a recombinant NDV comprising a non-APMV F protein described herein, a non-APMV-HN protein described herein, or a non-APMV F protein described herein and a non-APMV-HN protein described herein. In some embodiments, the non-APMV F protein is encoded by a nucleotide sequence of any one of SEQ ID Nos: 1-14. In some embodiments, the non-APMV F protein is encoded by a nucleotide sequence that is at least 80%, at least 85%, at least 90%, at least 95%, at least 98%, or at least 99% identical to the nucleotide sequence of any one of SEQ ID Nos:1-14. In some embodiments, the non-APMV HN protein is encoded by a nucleotide sequence of any one of SEQ ID Nos: 1-14. In some embodiments, the non-APMV HN protein is encoded by a nucleotide sequence that is at least 80%, at least 85%, at least 90%, at least 95%, at least 98%, or at least 99% identical to the nucleotide sequence of any one of SEQ ID Nos:1-14.

In another aspect, provided herein are recombinant Newcastle disease virus (NDV), comprising a packaged genome in which the coding sequence for NDV F protein has been replaced with the coding sequence for an F protein of an avian paramyxovirus (APMV) other than NDV (non-NDV APMV F protein) or a variant thereof and/or the coding sequence for NDV HN protein has been replaced with the coding sequence for an HN protein of an APMV other than NDV (non-NDV APMV HN protein) or a variant thereof. In a specific embodiment, provided herein is a recombinant NDV comprising a packaged genome, wherein the packaged genome comprises a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV AMPV HN protein coding sequence; and (2) the nucleotide sequence encoding the NDV F protein has been replaced with a nucleotide sequence encoding a non-NDV APMV F protein, wherein NDV intergenic regions are before and after the non-NDV AMPV F protein coding sequence. In specific embodiments, the NDV intergenic regions before and after the non-NDV APMV HN protein coding sequence are NDV HN intergenic regions. In specific embodiments, the NDV intergenic regions before and after the non-NDV APMV F protein coding sequence are NDV F intergenic regions. In specific embodiments, the non-NDV APMV F protein and non-NDV APMV HN protein are immunologically distinct from the NDF F protein and NDV HN protein, respectively. In some embodiments, the non-NDV APMV F protein and non-NDV APMV HN protein are from a different genus than NDV. In some embodiments, the non-NDV APMV F protein and non-APMV HN protein are an F protein and an HN protein from a member of the subfamily Avulavirinae, but not NDV. In some embodiments, the non-NDV APMV F protein and non-APMV HN protein are an F protein and an HN protein from a member of the subfamily Avulavirinae and the genus Metaavulavirus. In some embodiments, the non-NDV APMV F protein and non-APMV HN protein are an F protein and an HN protein from a member of the subfamily Avulavirinae and genus paraavulavirus. In some embodiments, the non-NDV APMV F protein and non-APMV HN protein are an F protein and an HN protein from a member of the subfamily Avulavirinae and the genus orthoavulavirus but is not NDV. In specific embodiments, the NDV genome comprises the NP gene, P gene, M gene, and L gene of NDV LaSota. In certain embodiments, the packaged genome further comprises a transgene. In some embodiments, the transgene comprises a nucleotide sequence encoding a viral, bacterial, fungal or protozoan antigen. In specific embodiments, the transgene comprises a nucleotide sequence encoding a SARS-CoV-2 antigen. In specific embodiments, the SARS-CoV-2 antigen is the SARS-CoV-2 spike protein or a fragment thereof. In specific embodiments, the SARS-CoV-2 antigen comprises a SARS-CoV-2 spike protein or a fragment thereof. In specific embodiments, the fragment comprises the receptor binding domain of the SARS-CoV-2 spike protein. In some embodiments, the fragment of a SARS-CoV-2 spike protein is the ectodomain of the SARS-CoV-2 spike protein. In specific embodiments, the transgene comprises a nucleotide sequence encoding a MERS-CoV antigen. In specific embodiments, the transgene comprises a nucleotide sequence encoding a respiratory syncytial virus antigen or human metapneumovirus antigen. In specific embodiments, the transgene comprises a nucleotide sequence encoding a Lassa virus antigen, Ebola virus antigen or Nipah virus antigen. In specific embodiments, the transgene comprises a nucleotide sequence encoding a cancer or tumor antigen.

In certain embodiments, a non-NDV APMV F protein or a variant thereof is immunologically distinct from an NDV F protein if antibodies directed to the NDV F protein do not cross-react with the non-NDV APMV F protein or variant thereof. In some embodiments, a non-NDV APMV F protein or a variant thereof is immunologically distinct from an NDV F protein if antibodies directed to the NDV F protein bind to the non-NDV APMV F protein or variant thereof with a 2-fold, 5-fold, 10-fold, 15-fold, 20-fold or lower affinity than to NDV F protein in an assay known to one of skill in the art or described herein. In certain embodiments, a non-NDV APMV F protein or a variant thereof is immunologically distinct from an NDV F protein if antibodies directed to the NDV F protein bind to the non-NDV APMV F protein or variant thereof with a 0.5 log, 1 log, 1.5 log, 2 log, 2.5 log, 3 log or lower affinity than to NDV F protein in an assay known to one of skill in the art or described herein. In certain embodiments, a non-NDV APMV F protein or a variant thereof is immunologically distinct from an NDV F protein if anti-NDV F antibodies do not substantially inhibit replication of NDV expressing the non-NDV APMV F protein or a variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or as described herein. In a specific embodiment, a non-NDV APMV F protein or a variant thereof is immunologically distinct from an NDV F protein if anti-NDV F antibodies inhibit replication of NDV expressing the non-NDV APMV F protein or variant thereof in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs.

In certain embodiments, a non-NDV APMV HN protein or a variant thereof is immunologically distinct from an NDV HN protein if antibodies directed to the NDV HN protein do not cross-react with the non-NDV APMV HN protein or variant thereof. In some embodiments, a non-NDV APMV HN protein or a variant thereof is immunologically distinct from an NDV NH protein if antibodies directed to the NDV HN protein bind to the non-NDV APMV NH protein or variant thereof with a 2-fold, 5-fold, 10-fold, 15-fold, 20-fold or lower affinity than to NDV HN protein in an assay known to one of skill in the art or described herein. In certain embodiments, a non-NDV APMV HN protein or a variant thereof is immunologically distinct from an NDV HN protein if antibodies directed to the NDV HN protein bind to the non-NDV APMV HN protein or variant thereof with a 0.5 log, 1 log, 1.5 log, 2 log, 2.5 log, 3 log or lower affinity than to NDV HN protein in an assay known to one of skill in the art or described herein. In certain embodiments, a non-NDV APMV HN protein or a variant thereof is immunologically distinct from an NDV HN protein if anti-NDV HN antibodies do not substantially inhibit replication of NDV expressing the non-NDV APMV HN protein or a variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a non-NDV APMV HN protein or a variant thereof is immunologically distinct from an NDV HN protein if anti-NDV HN antibodies inhibit replication of NDV expressing the non-NDV APMV HN protein or variant thereof in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs.

In some embodiments, a non-NDV APMV HN protein is an HN protein from a different genus than NDV. In some embodiments, a non-NDV APMV HN protein is an HN protein from a member of the subfamily Avulavirinae, but not NDV. In some embodiments, a non-NDV APMV HN protein is an HN protein from a member of the subfamily Avulavirinae and the genus Metaavulavirus. In some embodiments, a non-NDV APMV HN protein is an HN protein from a member of the subfamily Avulavirinae and genus paraavulavirus. In some embodiments, a non-NDV APMV HN protein is an HN protein from a member of the subfamily Avulavirinae and the genus orthoavulavirus but is not NDV.

In specific embodiments, the non-NDV APMV HN is the HN protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07. In specific embodiments, the non-NDV APMV F is the F protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07. In specific embodiments, the non-NDV APMV F protein and the non-NDV AMPV HN protein are from or derived from the same APMV strain. In other embodiments the non-NDV APMV F protein and the non-NDV AMPV HN protein are from or derived from different APMV strains.

In a specific embodiment, provided herein is a recombinant Newcastle disease virus (NDV) comprising a packaged genome, wherein the packaged genome comprises a nucleotide sequence of a Newcastle disease virus genome in which the nucleotide sequences encoding the NDV HN protein and NDV F protein are replaced with a nucleotide sequence comprising a negative sense RNA sequence transcribed from the cDNA sequence set forth in any one of SEQ ID NOs:1-14. In a specific embodiment, provided herein is a recombinant Newcastle disease virus (NDV) comprising a packaged genome, wherein the packaged genome comprises a nucleotide sequence of a Newcastle disease virus genome in which the nucleotide sequences encoding the NDV HN protein and NDV F protein are replaced with a nucleotide sequence comprising a negative sense RNA sequence transcribed from a cDNA sequence that is at least 80%, at least 85%, at least 90%, at least 95%, at least 98%, or at least 99% identical to the cDNA sequence set forth in any one of SEQ ID NOs:1-14. In specific embodiments, the NDV genome comprises the NP gene, P gene, M gene, and L gene of NDV LaSota. In certain embodiments, the packaged genome further comprises a transgene. In some embodiments, the transgene comprises a nucleotide sequence encoding a viral, bacterial, fungal or protozoan antigen. In specific embodiments, the transgene comprises a nucleotide sequence encoding a SARS-CoV-2 antigen. In specific embodiments, the SARS-CoV-2 antigen is the SARS-CoV-2 spike protein or a fragment thereof. In specific embodiments, the SARS-CoV-2 antigen comprises a SARS-CoV-2 spike protein or a fragment thereof. In specific embodiments, the fragment comprises the receptor binding domain of the SARS-CoV-2 spike protein. In some embodiments, the fragment of a SARS-CoV-2 spike protein is the ectodomain of the SARS-CoV-2 spike protein. In specific embodiments, the transgene comprises a nucleotide sequence encoding a MERS-CoV antigen. In specific embodiments, the transgene comprises a nucleotide sequence encoding a respiratory syncytial virus antigen or human metapneumovirus antigen. In specific embodiments, the transgene comprises a nucleotide sequence encoding a Lassa virus antigen, Ebola virus antigen or Nipah virus antigen. In specific embodiments, the transgene comprises a nucleotide sequence encoding a cancer or tumor antigen.

In another aspect, provide herein is an immunogenic composition comprising a recombinant NDV described herein. The immunogenic composition may further comprise a pharmaceutically acceptable carrier. The composition may comprise 10⁴ to 10¹² PFU of a recombinant NDV described herein.

In another aspect, provided herein is a method for inducing an immune response to an antigen, comprising administering a recombinant NDV described herein or an immunogenic composition described herein a subject (e.g., a human subject). In another aspect, provided herein is a method for preventing an infectious disease, comprising administering a recombinant NDV described herein or an immunogenic composition described herein a subject (e.g., a human subject). In another aspect, provided herein is a method for immunizing a subject against an infectious disease, comprising administering a recombinant NDV described herein or an immunogenic composition described herein a subject (e.g., a human subject). In another aspect, provided herein is a method for treating cancer, comprising administering a recombinant NDV described herein or an immunogenic composition described herein a subject (e.g., a human subject). In some embodiments, the recombinant NDV or composition is administered to the subject intranasally. In certain embodiments, the method further comprises administering a second recombinant NDV comprising a packaged genome, wherein the packaged genome of the second recombinant NDV comprises a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV AMPV HN protein coding sequence; and (2) the nucleotide sequence encoding the NDV F protein has been replaced with a nucleotide sequence encoding a non-NDV APMV F protein, wherein NDV intergenic regions are before and after the non-NDV AMPV F protein coding sequence, and wherein the second recombinant NDV is immunologically distinct than the first recombinant NDV administered to the subject. In specific embodiments, the NDV intergenic regions before and after the non-NDV APMV HN protein coding sequence are NDV HN intergenic regions. In specific embodiments, the NDV intergenic regions before and after the non-NDV APMV F protein coding sequence are NDV F intergenic regions. In some embodiments, the recombinant NDV described herein or a composition thereof is administered to a subject that has previously been vaccinated or administered NDV composition (e.g., a vaccine). In certain embodiments, the recombinant NDV described herein or a composition thereof is administered to a subject that has previously been vaccinated or administered an APMV-based composition (e.g. a vaccine). In some embodiments, the recombinant NDV described herein or a composition thereof is administered to a subject that has previously been vaccinated or administered NDV composition (e.g., a vaccine) and an APMV-based composition (e.g. a vaccine).

In certain embodiments, the first recombinant NDV is immunologically distinct from the second recombinant NDV if the first recombinant NDV and second recombinant NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771. In a specific embodiment, a first recombinant NDV is considered immunologically distinct from a second recombinant NDV if the first recombinant NDV and the second recombinant NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, by less than about 0.5 logs, less than about 1 log, less than about 1.5 logs, or less than about 2 logs.

In another aspect, provided herein is a kit comprising a recombinant NDV described herein. In another aspect, provided herein is an in vitro or ex vivo cell comprising the recombinant NDV. In another aspect, provided herein is a cell line or chicken embryonated egg comprising a recombinant NDV described herein.

In another aspect, provided herein is a kit comprising a nucleic acid sequence that comprises a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV APMV HN protein coding sequence; and (2) the nucleotide sequence encoding the NDV F protein has been replaced with a nucleotide sequence encoding a non-NDV APMV F protein, wherein NDV intergenic regions are before and after the non-NDV APMV F protein coding sequence. In some embodiments, the NDV genome comprises the NP gene, P gene, M gene, and L gene of NDV LaSota strain.

In some embodiments, provided herein is a kit comprising a nucleic acid sequence that comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV large polymerase (L), and (5) the nucleotide sequence of any one of SEQ ID NOS:1-14, or a nucleotide sequence that is at least 80%, at least 85%, at least 90%, at least 95%, or at least 98% identical to the nucleotide sequence of any one of SEQ ID NOS:1-14. In some embodiments, is a kit comprising a nucleic acid sequence that comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV large polymerase (L), and (5) a negative sense RNA sequence corresponding to the nucleotide sequence of any one of SEQ ID NOS:1-14, or a negative sense RNA sequence corresponding to a nucleotide sequence that is at least 80%, at least 85%, at least 90%, at least 95%, or at least 98% identical to the nucleotide sequence of any one of SEQ ID NOS:1-14. In some embodiments, the NDV nucleocapsid protein, NDV phosphoprotein, NDV matrix protein, and NDV large polymerase are of the NDV LaSota strain.

In another aspect, provided herein is a method for propagating the recombinant NDV described herein, the method comprising culturing the cell or embryonated egg comprising a recombinant NDV described herein. In some embodiments, the method further comprises isolating the recombinant NDV from the egg or embryonated egg.

### 3.1 Terminology

As used herein, the term “about” or “approximately” when used in conjunction with a number refers to any number within 1, 5 or 10% of the referenced number.

As used herein, the terms “antibody” and “antibodies” refer to molecules that contain an antigen binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “heterologous” in the context of a NDV refers to an entity not found in nature to be associated with (e.g., encoded by, expressed by the genome of, or both) a naturally occurring NDV. In a specific embodiment, a heterologous sequence encodes a protein that is not found associated with naturally occurring NDV.

As used herein, the term “heterologous” in the context of a nucleic acid or nucleotide sequence, or amino acid sequence refers to a second nucleic acid or nucleotide sequence, or second amino acid sequence not found in nature to be associated with a first nucleic acid or nucleotide sequence, or first amino acid sequence.

As used herein, the phrases “IFN deficient systems” or “IFN-deficient substrates” refer to systems, e.g., cells, cell lines and animals, such as mice, chickens, turkeys, rabbits, rats, horses etc., which do not produce one, two or more types of interferon (IFN), or do not produce any type of IFN, or produce low levels of one, two or more types of IFN, or produce low levels of any IFN (i.e., a reduction in any IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to one, two or more types of IFN, or do not respond to any type of IFN, have a delayed response to one, two or more types of IFN, are deficient in the activity of antiviral genes induced by one, two or more types of IFN, or induced by any type of IFN, or any combination thereof.

As used herein, the terms “subject” or “patient” are used interchangeably. As used herein, the terms “subject” and “subjects” refers to an animal. In some embodiments, the subject is a mammal including a non-primate (e.g., a camel, donkey, zebra, bovine, horse, horse, cat, dog, rat, and mouse) and a primate (e.g., a monkey, chimpanzee, and a human). In some embodiments, the subject is a non-human mammal. In certain embodiments, the subject is a pet (e.g., dog or cat) or farm animal (e.g., a horse, pig or cow). In specific embodiments, the subject is a human. In other specific embodiments, the subject is a bovine. In certain embodiments, the mammal (e.g., human) is 4 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, the subject is an animal that is not avian.

As used herein, the term “in combination” in the context of the administration of (a) therapy(ies) to a subject, refers to the use of more than one therapy. The use of the term “in combination” does not restrict the order in which therapies are administered to a subject. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. For example, a recombinant NDV described herein may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of another therapy.

As used herein, the term “wild-type” in the context of nucleotide and amino acid sequences of viruses refers to the nucleotide and amino acid sequences of viral strains found in nature. In particular, the sequences described as wild-type herein are sequences that have been reported in public databases as sequences from natural viral isolates.

### 4. BRIEF DESCRIPTION OF THE FIGURES

FIG. 1. Strategy for the construction of recombinant chimeric NDV-APMV vectors. Sequences corresponding to Newcastle Disease Virus (NDV or APMV-1) are shown in white boxes and the sequences corresponding to an antigenically different avian paramyxovirus (e.g., APMV-4) are shown in gray boxes.

FIGS. 2A-2C. Generation of the acceptor plasmid pNDV-F-HNless. The F and HN genes of a rescue plasmid pNDV-LaSota (FIG. 2A) were replaced by a short sequence containing 2 unique restriction sites, Pmel and NruI, to generate an acceptor plasmid pNDV-F-HNless (FIG. 2B). FIG. 2C shows a functional rescue plasmid in which the F and HN genes from NDV were reinserted into the acceptor plasmid of FIG. 2B to generate a functional rescue plasmid pNDV-LaSota. FIGS. 2A-2C were not drawn to scale.

FIGS. 3A-3B. Maximum likelihood phylogenetic trees. The phylogenetic trees of the F and HN amino acid sequences of all the avian paramyxoviruses (excluding NDV) with a full genome sequence available are shown in FIG. 3A and FIG. 3B, respectively. The F and HN proteins of 14 viruses that were selected for sequence synthesis are in bold.

FIGS. 4A-4C. Construction of a rescue plasmid chimeric NDV-APMV. Synthetic inserts containing F and HN coding sequences from different APMVs and NDV non-coding flanking regions are amplified by PCR with primers designed for the seamless reconstitution of the NDV sequences flanking the F and HN open reading frames. The white boxes in FIG. 4A represent NDV non-coding flanking regions and the gray boxes represent F and HN coding sequences from different APMVs (not drawn to scale). FIG. 4B shows the acceptor plasmid pNDV-F-HNless and FIG. 4C shows a rescue plasmid chimeric NDV-APMV in which the synthetic inserts were inserted between the M and L genes of the acceptor plasmid pNDV-F-HNless.

FIGS. 5A-5H. Transcription analysis of viral replication and proinflammatory genes by qPCR. Cancer cells were infected at a MOI of 1 or mock-infected and subjected to RNA extraction at 8- and 16-hours post-infection. FIGS. 5A-5D, Viral replication levels measured as mRNA expression of the N protein. Bars represent the average of three independent biological samples ±SD, shown in the order of LS-L289A, APMV-4, and rAPMV-4. FIGS. 5E-5H, Heat maps showing levels of induction of IFN-b, ISGs (STAT1, ISG15, MX, OAS-1) and proinflammatory cytokines (IL-6 and IL-1B) for each independent biological sample (1, 2, 3) corresponding to FIGS. 5A-5D. Expression levels for each individual gene were calculated as Log 10 of Fold induction over mock infected cells. Two-way ANOVA analysis: *p<0.05; ***p<0.001; ****p<0.0001; ns: non-significant.

FIGS. 6A-6B. FIG. 6A depicts the phylogenetic tree of the Avulavirinae subfamily of avian paramyxoviruses. The figure has been adapted from Rima et al., 2019, J. Gen. Virol. 100(12):1593-1594. FIG. 6B is a schematic depicting the removal of the NDV F protein and NDV HN protein coding sequences from the NDV genomic sequence, the insertion of F protein and HN protein coding sequences of distant avian paramyxoviruses into the NDV genome in which the NDV F protein and NDV HN protein coding sequences have been removed, and the insertion of a transgene, such as a transgene encoding green fluorescent protein (GFP) into the NDV genome.

FIG. 7 depicts the location of APMV-2 and APMV-3 in the phylogenetic tree and schematics of the NDV genome with a transgene encoding GFP and the NDV F protein and NDV HN protein coding sequences replaced with either APMV-2 F protein and HN protein coding sequences (chimeric NDV-APMV-2-GFP), or APMV-3 F protein and HN protein coding sequences (chimeric NDV-APMV-3-GFP). Also depicted is a schematic of the NDV genome with a transgene encoding GFP (NDV-GFP).

FIGS. 8A-8B. FIG. 8A shows the expression of GFP by chicken embryo fibroblasts (CEF) cells infected with chimeric NDV-APMV2-GFP and chimeric NDV-APMV3-GFPs. FIG. 8B shows the results of a hemagglutination inhibition (HI) assay using rabbit sera raised against the wild-type (WT) NDV viruses. HI activity of the rabbit serum was significantly reduced against both chimeric NDV-APMV-2-GFP and chimeric NDV-APMV-3-GFP as compared to that against the NDV-GFP.

### 5. DETAILED DESCRIPTION

### 5.1 Recombinant Newcastle Disease Virus

In one aspect, provided herein is a recombinant NDV a packaged genome, wherein the packaged genome comprises a nucleic acid sequence described in Section 5.1.1. In a specific embodiment, provided herein is a recombinant NDV comprising a nucleic acid sequence described in Section 5.1.1.

In another aspect, provided herein is a recombinant NDV comprising a packaged genome, wherein the packaged genome comprises a nucleotide sequence of a Newcastle disease virus genome in which the nucleotide sequence encoding the NDV HN protein has been replaced with a nucleotide sequence encoding the HN protein of an avian paramyxovirus (APMV) other than NDV or a variant of the non-NDV-APMV HN protein, or the nucleotide sequence encoding the NDV F protein has been replaced with a nucleotide sequence encoding the F protein of an APMV other than NDV or a variant of the non-NDV-APMV F protein. In certain instances herein, the term “non-NDV APMV” is used to refer to an APMV other than NDV. In one embodiment, provided herein is a recombinant NDV comprising a packaged genome, wherein the packaged genome comprises a nucleotide sequence of a Newcastle disease virus genome in which (1) the nucleotide sequence encoding the NDV HN protein has been replaced with a nucleotide sequence encoding a non-NDV APMV HN protein or a variant thereof, wherein NDV intergenic regions are before and after the non-NDV APMV HN protein coding sequence or variant HN protein coding sequence; or (2) the nucleotide sequence encoding the NDV F protein has been replaced with a nucleotide sequence encoding a non-NDV APMV F protein or a variant thereof, wherein NDV intergenic regions are before and after the non-NDV AMPV F protein coding sequence or variant F protein coding sequence. In specific embodiments, the NDV intergenic regions before and after the non-NDV APMV HN protein coding sequence are NDV HN intergenic regions. In specific embodiments, the NDV intergenic regions before and after the non-NDV APMV F protein coding sequence are NDV F intergenic regions. In another embodiment, provided herein is a recombinant NDV comprising a packaged genome, wherein the packaged genome comprises a nucleotide sequence of a Newcastle disease virus genome in which (1) the transcription unit encoding the NDV HN protein has been replaced with a transcription unit comprising a nucleotide sequence encoding a non-NDV APMV HN protein or a variant thereof; or (2) the transcription unit encoding the NDV F protein has been replaced with a transcription unit comprising a nucleotide sequence encoding a non-NDV APMV F protein or a variant thereof. In specific embodiments, the non-NDV APMV F protein or variant thereof has one or more, or all of the functions of NDV F protein required for NDV to replicate in vitro, in vivo or both. In specific embodiments, the non-NDV APMV HN protein or variant thereof has one or more, or all of the functions of NDV HN protein required for NDV to replicate in vitro, in vivo or both. The NDV genome typically comprises the N gene, P gene, L gene, M gene, HN gene, and F gene.

In another embodiment, provided herein is a recombinant NDV comprising a packaged genome, wherein the packaged genome comprises a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV APMV HN protein coding sequence; or (2) the nucleotide sequence encoding the NDV F protein has been replaced with a nucleotide sequence encoding a non-NDV APMV F protein, wherein NDV intergenic regions are before and after the non-NDV APMV F protein coding sequence. In specific embodiments, the NDV intergenic regions before and after the non-NDV APMV HN protein coding sequence are NDV HN intergenic regions. In specific embodiments, the NDV intergenic regions before and after the non-NDV APMV F protein coding sequence are NDV F intergenic regions. In another embodiment, provided herein is a recombinant NDV comprising a packaged genome, wherein the packaged genome comprises a nucleotide sequence of a Newcastle disease virus genome in which (1) the transcription unit encoding the NDV HN protein has been replaced with a transcription unit comprising a nucleotide sequence encoding a non-NDV APMV HN protein; or (2) the transcription unit encoding the NDV F protein has been replaced with a transcription unit comprising a nucleotide sequence encoding a non-NDV APMV F protein. In specific embodiments, the non-NDV APMV F protein has one or more, or all of the functions of NDV F protein required for NDV to replicate in vitro, in vivo or both. In specific embodiments, the non-NDV APMV HN protein has one or more, or all of the functions of NDV HN protein required for NDV to replicate in vitro, in vivo or both.

In another embodiment, provided herein is a recombinant NDV comprising a packaged genome, wherein the packaged genome comprises a nucleotide sequence of a Newcastle disease virus genome in which (1) the nucleotide sequence encoding the NDV HN protein has been replaced with a nucleotide sequence encoding a variant of a non-NDV APMV HN protein, wherein NDV intergenic regions are before and after the variant HN protein coding sequence; or (2) the nucleotide sequence encoding the NDV F protein has been replaced with a nucleotide sequence encoding a variant of a non-NDV APMV F protein, wherein NDV intergenic regions are before and after the variant F protein coding sequence. In specific embodiments, the NDV intergenic regions before and after the variant of the non-NDV APMV HN protein coding sequence are NDV HN intergenic regions. In specific embodiments, the NDV intergenic regions before and after the variant of the non-NDV APMV F protein coding sequence are NDV F intergenic regions. In another embodiment, provided herein is a recombinant NDV comprising a packaged genome, wherein the packaged genome comprises a nucleotide sequence of a Newcastle disease virus genome in which (1) the transcription unit encoding the NDV HN protein has been replaced with a transcription unit comprising a nucleotide sequence encoding a variant of a non-NDV APMV HN protein; or (2) the transcription unit encoding the NDV F protein has been replaced with a transcription unit comprising a nucleotide sequence encoding a variant of a non-NDV APMV F protein. In specific embodiments, the variant of the non-NDV F protein has one or more, or all of the functions of NDV F protein required for NDV to replicate in vitro, in vivo or both. In specific embodiments, the variant of the non-NDV HN protein has one or more, or all of the functions of NDV HN protein required for NDV to replicate in vitro, in vivo or both.

In another aspect, provided herein is a recombinant NDV comprising a packaged genome, wherein the packaged genome comprises a nucleotide sequence of a Newcastle disease virus genome in which the nucleotide sequence encoding the NDV HN protein has been replaced with a nucleotide sequence encoding the HN protein of an avian paramyxovirus (APMV) other than NDV or a variant of the non-NDV-APMV HN protein, and the nucleotide sequence encoding the NDV F protein has been replaced with a nucleotide sequence encoding the F protein of an APMV other than NDV or a variant of the non-NDV-APMV F protein. In one embodiment, provided herein is a recombinant NDV comprising a packaged genome, wherein the packaged genome comprises a nucleotide sequence of a Newcastle disease virus genome in which (1) the nucleotide sequence encoding the NDV HN protein has been replaced with a nucleotide sequence encoding a non-NDV APMV HN protein or a variant thereof; and (2) the nucleotide sequence encoding the NDV F protein has been replaced with a nucleotide sequence encoding a non-NDV APMV F protein or a variant thereof, wherein NDV intergenic regions are before, in between and after the non-NDV APMV HN and F protein coding sequences or variant HN and F protein coding sequences. In specific embodiments, the NDV intergenic regions before and after the nucleotide sequence encoding the non-NDV APMV HN protein or a variant thereof are NDV HN intergenic regions. In specific embodiments, the NDV intergenic regions before and after the nucleotide sequence encoding the non-NDV APMV F protein or a variant thereof are NDV F intergenic regions. In another embodiment, provided herein is a recombinant NDV comprising a packaged genome, wherein the packaged genome comprises a nucleotide sequence of a Newcastle disease virus genome in which (1) the transcription unit encoding the NDV HN protein has been replaced with a transcription unit comprising a nucleotide sequence encoding a non-NDV APMV HN protein or a variant thereof, and (2) the transcription unit encoding the NDV F protein has been replaced with a transcription unit comprising a nucleotide sequence encoding a non-NDV APMV F protein or a variant thereof. In specific embodiments, the non-NDV F protein or variant thereof has one or more, or all of the functions of NDV F protein required for NDV to replicate in vitro, in vivo or both. In specific embodiments, the non-NDV HN protein or variant thereof has one or more, or all of the functions of NDV HN protein required for NDV to replicate in vitro, in vivo or both.

In another embodiment, provided herein is a recombinant NDV comprising a packaged genome, wherein the packaged genome comprises a nucleotide sequence of a Newcastle disease virus genome in which (1) the nucleotide sequence encoding the NDV HN protein has been replaced with a nucleotide sequence encoding a non-NDV APMV HN protein; and (2) the nucleotide sequence encoding the NDV F protein has been replaced with a nucleotide sequence encoding a non-NDV APMV F protein, wherein NDV intergenic regions are before, in between and after the non-NDV APMV HN and F protein coding sequences. In specific embodiments, the NDV intergenic regions before and after the nucleotide sequence encoding the non-NDV APMV HN protein are NDV HN intergenic regions. In specific embodiments, the NDV intergenic regions before and after the nucleotide sequence encoding the non-NDV APMV F protein are NDV F intergenic regions. In another embodiment, provided herein is a recombinant NDV comprising a packaged genome, wherein the packaged genome comprises a nucleotide sequence of a Newcastle disease virus genome in which (1) the transcription unit encoding the NDV HN protein has been replaced with a transcription unit comprising a nucleotide sequence encoding a non-NDV APMV HN protein; and (2) the transcription unit encoding the NDV F protein has been replaced with a transcription unit comprising a nucleotide sequence encoding a non-NDV APMV F protein. In specific embodiments, the non-NDV APMV HN and F proteins are found in nature in the same strain of APMV. For example, the non-NDV APMV HN and F proteins may both be found in nature in the same APMV-15 strain. In other embodiments, the non-NDV APMV HN and F proteins are found in nature in the different strains of APMV. In specific embodiments, the non-NDV F protein has one or more, or all of the functions of NDV F protein required for NDV to replicate in vitro, in vivo or both. In specific embodiments, the non-NDV HN protein has one or more, or all of the functions of NDV HN protein required for NDV to replicate in vitro, in vivo or both.

In another embodiment, provided herein is a recombinant NDV comprising a packaged genome, wherein the packaged genome comprises a nucleotide sequence of a Newcastle disease virus genome in which (1) the nucleotide sequence encoding the NDV HN protein has been replaced with a nucleotide sequence encoding a variant of a non-NDV APMV HN protein; and (2) the nucleotide sequence encoding the NDV F protein has been replaced with a nucleotide sequence encoding a variant of a non-NDV APMV F protein, wherein NDV intergenic regions are before, in between and after the variant HN and F protein coding sequences. In specific embodiments, the NDV intergenic regions before and after the nucleotide sequence encoding the variant of the non-NDV APMV HN protein are NDV HN intergenic regions. In specific embodiments, the NDV intergenic regions before and after the nucleotide sequence encoding the variant of the non-NDV APMV F protein are NDV F intergenic regions. In another embodiment, provided herein is a recombinant NDV comprising a packaged genome, wherein the packaged genome comprises a nucleotide sequence of a Newcastle disease virus genome in which (1) the transcription unit encoding the NDV HN protein has been replaced with a transcription unit comprising a nucleotide sequence encoding a variant of a non-NDV APMV HN protein; and (2) the transcription unit encoding the NDV F protein has been replaced with a transcription unit comprising a nucleotide sequence encoding a variant of a non-NDV APMV F protein. In specific embodiments, the variant HN and F proteins are derived from the same strain of APMV. For example, the variant HN and F proteins may both be derived from the same APMV-15 strain. In other embodiments, the variant HN and F proteins are derived from different strains of APMV. In specific embodiments, the variant of the non-NDV F protein has one or more, or all of the functions of NDV F protein required for NDV to replicate in vitro, in vivo or both. In specific embodiments, the variant of the non-NDV HN protein has one or more, or all of the functions of NDV HN protein required for NDV to replicate in vitro, in vivo or both.

In another aspect, provided herein is a recombinant NDV comprising a packaged genome, wherein the packaged genome comprises a nucleotide sequence of a Newcastle disease virus genome in which the nucleotide sequence encoding the NDV HN protein has been replaced with a nucleotide sequence encoding a chimeric HN protein, or the nucleotide sequence encoding the NDV F protein has been replaced with a nucleotide sequence encoding a chimeric F protein. See, e.g., Park et al., 2006, PNAS May 23, 2006 103 (21) 8203-8208, International Patent Application No. WO 2007/064802, and U.S. Pat. No. 9,387,242 B2 regarding methods for producing chimeric F or chimeric HN proteins. In one embodiment, provided herein is a recombinant NDV comprising a packaged genome, wherein the packaged genome comprises a nucleotide sequence of a Newcastle disease virus genome in which (1) the nucleotide sequence encoding the NDV HN protein has been replaced with a nucleotide sequence encoding a chimeric HN protein, wherein NDV intergenic regions are before and after the chimeric HN protein coding sequence; or (2) the nucleotide sequence encoding the NDV F protein has been replaced with a nucleotide sequence encoding a chimeric F protein, wherein NDV intergenic regions are before and after the chimeric F protein coding sequence. In specific embodiments, the NDV intergenic regions before and after the nucleotide sequence encoding the chimeric HN protein are NDV HN intergenic regions. In specific embodiments, the NDV intergenic regions before and after the nucleotide sequence encoding the chimeric F protein are NDV F intergenic regions. In another embodiment, provided herein is a recombinant NDV comprising a packaged genome, wherein the packaged genome comprises a nucleotide sequence of a Newcastle disease virus genome in which (1) the transcription unit encoding the NDV HN protein has been replaced with a transcription unit comprising a nucleotide sequence encoding a chimeric HN protein; or (2) the transcription unit encoding the NDV F protein has been replaced with a transcription unit comprising a nucleotide sequence encoding a chimeric F protein. In specific embodiments, the chimeric HN protein comprises a non-NDV APMV HN protein ectodomain and NDV HN protein transmembrane and cytoplasmic domains. In other words, the NDV HN protein transmembrane and cytoplasmic domains replace the non-NDV APMV HN protein transmembrane and cytoplasmic domains so that the chimeric HN protein does not include the non-NDV APMV HN protein transmembrane and cytoplasmic domains. The ectodomain, transmembrane and cytoplasmic domains of the non-NDV APMV HN protein and NDV HN protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the non-NDV HN protein and NDV HN protein. In specific embodiments, the chimeric HN protein comprises an ectodomain of a variant of a non-NDV APMV HN protein and NDV HN protein transmembrane and cytoplasmic domains. In specific embodiments, the chimeric HN protein has one or more, or all of the functions of NDV HN required for NDV to replicate in vitro, in vivo or both. In specific embodiments, the chimeric F protein comprises a non-NDV APMV F protein ectodomain and NDV F protein transmembrane and cytoplasmic domains. In other words, the NDV F protein transmembrane and cytoplasmic domains replace the non-NDV APMV F protein transmembrane and cytoplasmic domains so that the chimeric F protein does not include the non-NDV APMV F protein transmembrane and cytoplasmic domains. The ectodomain, transmembrane and cytoplasmic domains of the non-NDV APMV F protein and NDV F protein may be determined using techniques known to one of skill in the art. For example, published information, GenBank or websites such as VIPR virus pathogen website (www.vibpbre.org), DTU Bioinformatics domain website (www.cbs.dtu.dk/services/TMHMM/) or programs available to determine the transmembrane domain may be used to determine the ectodomain, transmembrane and cytoplasmic domains of the non-NDV F protein and NDV F protein. In specific embodiments, the chimeric F protein comprises an ectodomain of a variant of a non-NDV APMV F protein and NDV F protein transmembrane and cytoplasmic domains. In specific embodiments, the chimeric F protein has one or more, or all of the functions of NDV F protein required for NDV to replicate in vitro, in vivo or both.

In another aspect, provided herein is a recombinant NDV comprising a packaged genome, wherein the packaged genome comprises a nucleotide sequence of a Newcastle disease virus genome in which the nucleotide sequence encoding the NDV HN protein has been replaced with a nucleotide sequence encoding a chimeric HN protein, and the nucleotide sequence encoding the NDV F protein has been replaced with a nucleotide sequence encoding a chimeric F protein. In one embodiment, provided herein is a recombinant NDV comprising a packaged genome, wherein the packaged genome comprises a nucleotide sequence of a Newcastle disease virus genome in which (1) the nucleotide sequence encoding the NDV HN protein has been replaced with a nucleotide sequence encoding a chimeric HN protein, wherein NDV intergenic regions are before and after the chimeric HN protein coding sequence; and (2) the nucleotide sequence encoding the NDV F protein has been replaced with a nucleotide sequence encoding a chimeric F protein, wherein NDV intergenic regions are before and after the chimeric F protein coding sequence. In specific embodiments, the NDV intergenic regions before and after the nucleotide sequence encoding the chimeric HN protein are NDV HN intergenic regions. In specific embodiments, the NDV intergenic regions before and after the nucleotide sequence encoding the chimeric F protein are NDV F intergenic regions. In another embodiment, provided herein is a recombinant NDV comprising a packaged genome, wherein the packaged genome comprises a nucleotide sequence of a Newcastle disease virus genome in which (1) the transcription unit encoding the NDV HN protein has been replaced with a transcription unit comprising a nucleotide sequence encoding a chimeric HN protein; and (2) the transcription unit encoding the NDV F protein has been replaced with a transcription unit comprising a nucleotide sequence encoding a chimeric F protein. In specific embodiments, the chimeric HN protein comprises a non-NDV APMV HN protein ectodomain and NDV HN protein transmembrane and cytoplasmic domains. In other words, the NDV HN protein transmembrane and cytoplasmic domains replace the non-NDV APMV HN protein transmembrane and cytoplasmic domains so that the chimeric HN protein does not include the non-NDV APMV HN protein transmembrane and cytoplasmic domains. The ectodomain, transmembrane and cytoplasmic domains of the non-NDV APMV HN protein and NDV HN protein may be determined using techniques known to one of skill in the art or described herein. In specific embodiments, the chimeric HN protein comprises an ectodomain of a variant of a non-NDV APMV HN protein and NDV HN protein transmembrane and cytoplasmic domains. In specific embodiments, the chimeric HN protein has one or more, or all of the functions of NDV HN required for NDV to replicate in vitro, in vivo or both. In specific embodiments, the chimeric F protein comprises a non-NDV APMV F protein ectodomain and NDV F protein transmembrane and cytoplasmic domains. In other words, the NDV F protein transmembrane and cytoplasmic domains replace the non-NDV APMV F protein transmembrane and cytoplasmic domains so that the chimeric F protein does not include the non-NDV APMV F protein transmembrane and cytoplasmic domains. The ectodomain, transmembrane and cytoplasmic domains of the non-NDV APMV F protein and NDV F protein may be determined using techniques known to one of skill in the art or described herein. In specific embodiments, the chimeric F protein comprises an ectodomain of a variant of a non-NDV APMV F protein and NDV F protein transmembrane and cytoplasmic domains. In specific embodiments, the chimeric F protein has one or more, or all of the functions of NDV F protein required for NDV to replicate in vitro, in vivo or both. In specific embodiments, the ectodomains of the non-NDV APMV HN and F proteins are found in nature in the same strain of APMV. For example, the ectodomains of the non-NDV APMV HN and F proteins may both be found in nature in the same APMV-15 strain. In other embodiments, the ectodomains of the non-NDV APMV HN and F proteins are found in nature in the different strains of APMV.

In another aspect, provided herein is a recombinant NDV comprising a packaged genome, wherein the packaged genome comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV fusion (F) protein, (5) a transcription unit encoding a non-NDV APMV hemagglutinin-neuraminidase (HN) or a variant thereof, and (6) a transcription unit encoding a NDV large polymerase (L). In another aspect, provided herein is a recombinant NDV comprising a packaged genome, wherein the packaged genome comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a non-NDV APMV fusion (F) protein or a variant thereof, (5) a transcription unit encoding a NDV hemagglutinin-neuraminidase (HN), and (6) a transcription unit encoding a NDV large polymerase (L). In specific embodiments, the non-NDV F protein or variant thereof has one or more, or all of the functions of NDV F protein required for NDV to replicate in vitro, in vivo or both. In specific embodiments, the non-NDV HN protein or variant thereof has one or more, or all of the functions of NDV HN protein required for NDV to replicate in vitro, in vivo or both.

In another aspect, provided herein is a recombinant NDV comprising a packaged genome, wherein the packaged genome comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a non-NDV APMV fusion (F) protein or a variant thereof, (5) a transcription unit encoding a non-NDV APMV hemagglutinin-neuraminidase (HN) or a variant thereof, and (6) a transcription unit encoding a NDV large polymerase (L). In specific embodiments, the non-NDV F protein or variant thereof has one or more, or all of the functions of NDV F protein required for NDV to replicate in vitro, in vivo or both. In specific embodiments, the non-NDV HN protein or variant thereof has one or more, or all of the functions of NDV HN protein required for NDV to replicate in vitro, in vivo or both.

In one embodiment, provided herein is a recombinant NDV comprising a packaged genome, wherein the packaged genome comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a non-NDV APMV fusion (F) protein, (5) a transcription unit encoding a non-NDV APMV hemagglutinin-neuraminidase (HN), and (6) a transcription unit encoding a NDV large polymerase (L). In specific embodiments, the non-NDV APMV HN and F proteins are found in nature in the same strain of APMV. For example, the non-NDV APMV HN and F proteins may both be found in nature in the same APMV-15 strain. In other embodiments, the non-NDV APMV HN and F proteins are found in nature in the different strains of APMV. In specific embodiments, the non-NDV F protein has one or more, or all of the functions of NDV F protein required for NDV to replicate in vitro, in vivo or both. In specific embodiments, the non-NDV HN protein has one or more, or all of the functions of NDV HN protein required for NDV to replicate in vitro, in vivo or both.

In another embodiment, provided herein is a recombinant NDV comprising a packaged genome, wherein the packaged genome comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a variant of a non-NDV APMV fusion (F) protein, (5) a transcription unit encoding a variant of a non-NDV APMV hemagglutinin-neuraminidase (HN), and (6) a transcription unit encoding a NDV large polymerase (L). In specific embodiments, the variants of the non-NDV APMV HN and F proteins are derived from the same strain of APMV. For example, the variants of the non-NDV APMV HN and F proteins may both be derived from the same APMV-15 strain. In other embodiments, the variants of the non-NDV APMV HN and F proteins are derived from the different strains of APMV. In specific embodiments, the variant of the non-NDV F protein has one or more, or all of the functions of NDV F protein required for NDV to replicate in vitro, in vivo or both. In specific embodiments, the variant of the non-NDV HN protein has one or more, or all of the functions of NDV HN protein required for NDV to replicate in vitro, in vivo or both.

In another aspect, provided herein is a recombinant NDV comprising a packaged genome, wherein the packaged genome comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV fusion (F) protein, (5) a transcription unit encoding a chimeric hemagglutinin-neuraminidase (HN), and (6) a transcription unit encoding a NDV large polymerase (L). In another aspect, provided herein is recombinant NDV comprising a packaged genome, wherein the packaged genome comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a chimeric fusion (F) protein, (5) a transcription unit encoding a NDV hemagglutinin-neuraminidase (HN), and (6) a transcription unit encoding a NDV large polymerase (L). In another aspect, provided herein is a recombinant NDV comprising a packaged genome, wherein the packaged genome comprises: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a chimeric fusion (F) protein, (5) a transcription unit encoding a chimeric hemagglutinin-neuraminidase (HN), and (6) a transcription unit encoding a NDV large polymerase (L). In specific embodiments, the chimeric HN protein comprises a non-NDV APMV HN protein ectodomain and NDV HN protein transmembrane and cytoplasmic domains. In other words, the NDV HN protein transmembrane and cytoplasmic domains replace the non-NDV APMV HN protein transmembrane and cytoplasmic domains so that the chimeric HN protein does not include the non-NDV APMV HN protein transmembrane and cytoplasmic domains. The ectodomain, transmembrane and cytoplasmic domains of the non-NDV APMV HN protein and NDV HN protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the non-NDV HN protein and NDV HN protein. In specific embodiments, the chimeric HN protein has one or more, or all of the functions of NDV HN required for NDV to replicate in vitro, in vivo or both. In specific embodiments, the chimeric F protein comprises a non-NDV APMV F protein ectodomain and NDV F protein transmembrane and cytoplasmic domains. In other words, the NDV F protein transmembrane and cytoplasmic domains replace the non-NDV APMV F protein transmembrane and cytoplasmic domains so that the chimeric F protein does not include the non-NDV APMV F protein transmembrane and cytoplasmic domains. The ectodomain, transmembrane and cytoplasmic domains of the non-NDV APMV F protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the non-NDV F protein and NDV F protein. In specific embodiments, the chimeric F protein has one or more, or all of the functions of NDV F protein required for NDV to replicate in vitro, in vivo or both. In specific embodiments, the ectodomains of the non-NDV APMV HN and F proteins are found in nature in the same strain of APMV. For example, the ectodomains of the non-NDV APMV HN and F proteins may both be found in nature in the same APMV-15 strain. In other embodiments, the ectodomains of the non-NDV APMV HN and F proteins are found in nature in the different strains of APMV.

In specific embodiments, the non-NDV APMV is immunologically distinct from NDV. In certain embodiments, a non-NDV APMV is immunologically distinct from NDV if the non-NDV APMV and NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a non-NDV APMV is considered immunologically distinct from NDV if the non-NDV APMV and NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In a specific embodiment, a non-NDV APMV is considered immunologically distinct from NDV if NDV antiserum HI activity is significantly reduced against the non-NDV APMV in an HI assay, such as described below (e.g., in Example 3). In a specific embodiment, a non-NDV APMV is considered immunologically distinct from NDV if NDV antiserum HI activity is reduced by at least 2 fold, 3 fold, 4 fold, 5 fold, 10 fold, or more against the non-NDV APMV in an HI assay, such as described below (e.g., in Example 3), relative the NDV antiserum HI activity against NDV. In certain embodiments, the non-NDV APMV is AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, the non-NDV APMV is an APMV-2, such as, e.g., Chicken/California/Yucaipa/56 (Accession No. EU338414). In certain embodiments, the non-NDV APMV is APMV-3, such as, e.g., APMV3/Turkey/Wisconsin/68 (Accession No. EU782025). In some embodiments, the non-NDV APMV is APMV-4, such as, e.g., APMV4/duck/Hongkong/D3/75 (Accession No. FJ177514), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, the non-NDV APMV is an APMV-5, such as, e.g., APMV-5 budgerigar/Kunitachi/74 (Accession No. GU206351) or APMV5/budgerigar/Japan/TI/75 (Accession No. LC168750). In some embodiments, the non-NDV APMV is APMV-6, such as, e.g., APMV-6 Goose/FarEast/4440/2003 (Accession No. EF569970) or APMV6/duck/HongKong/18/199/77 (Accession No. EU622637). In certain embodiments, the non-NDV APMV is APMV-7, such as, e.g., APMV-7 dove/Tennessee/4/75 (Accession No. FJ231524). In some embodiments, the non-NDV APMV is APMV-8, such as, e.g., APMV-8 goose/Delaware/1053/76 (Accession No. FJ215863). In certain embodiments, the non-NDV APMV is APMV-9, such as, e.g., APMV9/duck/New York/22/78 (Accession No. EU910942). In some embodiments, the non-NDV APMV is APMV-10, such as, e.g., APMV-10 penguin/Falkland Islands/324/2007 (Accession No. HM147142 or NC_025349). In certain embodiments, the non-NDV APMV is APMV-11, such as, e.g., APMV-11 common_snipe/France/100212/2010 (Accession No. JQ886184). In some embodiments, the non-NDV APMV is APMV-12, such as, e.g., APMV12/Wigeon/Italy/3920_1/05 (Accession No. KC333050). In some embodiments, the non-NDV APMV is APMV-14, such as, e.g., APMV-14 duck/Japan/110G0352/2011 (Accession No. KX258200). In certain embodiments, the non-NDV APMV is APMV-15, such as, e.g., APMV-15 calidris_fuscicollis/Brazil/RS-1177/2012 (Accession No. KX932454). In some embodiments, the non-NDV APMV is APMV-17, such as, e.g., APMV17/Antarctica/107/13 (Accession No. MK167211). In some embodiments, the non-NDV APMV is APMV-20, such as, e.g., APMV-20 Gull/Kazakhstan/2014 (Accession No. MF033136). In certain embodiments, the non-NDV APMV is APMV-21, such as, e.g., APMV21/pigeon/Taiwan/AHRI128/17 (Accession No. MK67743).

In a specific embodiment, the non-NDV APMV is APMV4/duck/Hongkong/D3/75 (Accession No. FJ177514). In another specific embodiment, the non-NDV APMV is APMV17/Antarctica/107/13 (Accession No. MK167211). In another specific embodiment, the non-NDV APMV is APMV9/duck/New York/22/78 (Accession No. EU910942). In another specific embodiment, the non-NDV is APMV7/dove/Tennessee/4/75 (Accession No. FJ231524). In another specific embodiment, the non-NDV APMV is APMV21/pigeon/Taiwan/AHRI128/17 (Accession No. MK67743). In another specific embodiment, the non-NDV APMV is APMV6/duck/HongKong/18/199/77 (Accession No. EU622637). In another specific embodiment, the non-NDV APMV is APMV11/common_snipe/France/100212/10 (Accession No. JQ886184). In another specific embodiment, the non-NDV APMV is APMV15/calidris_fuscicollis/Brazil/RS-1177/12 (Accession No. NC_034968). In another specific embodiment, the non-NDV APMV is APMV8/Goose/Delaware/1053/76 (Accession No. FJ215863). In another specific embodiment, the non-NDV APMV is APMV2/Chicken/California/Yucaipa/56 (Accession No. EU338414). In another specific embodiment, the non-NDV APMV is APMV3/Turkey/Wisconsin/68 (Accession No. EU782025). In another specific embodiment, the non-NDV APMV is APMV12/Wigeon/Italy/3920_1/05 (Accession No. KC333050). In another specific embodiment, the non-NDV APMV is APMV5/budgerigar/Japan/TI/75 (Accession No. LC168750). In another specific embodiment, the non-NDV APMV is APMV10/penguin/Falkland Islands/324/07 (Accession No. NC_025349).

In some embodiments, the non-NDV APMV is a member of the subfamily Avulavirinae from a different genus than NDV. In some embodiments, the non-NDV APMV is from a member of the subfamily Avulavirinae, but is not NDV. In some embodiments, the non-NDV APMV is a member of the subfamily Avulavirinae and the genus Metaavulavirus. In some embodiments, the non-NDV APMV is a member of the subfamily Avulavirinae and the genus paraavulavirus. In some embodiments, the non-NDV APMV is a member of the subfamily Avulavirinae and the genus orthoavulavirus but is not NDV.

In certain embodiments, a non-NDV APMV F protein is immunologically distinct from an NDV F protein. In certain embodiments, a variant of a non-NDV APMV F protein is immunologically distinct from an NDV F protein. In certain embodiments, a non-NDV APMV F protein or a variant thereof is immunologically distinct from an NDV F protein if antibodies directed to the NDV F protein do not cross-react with the non-NDV APMV F protein or variant thereof. In some embodiments, a non-NDV APMV F protein or a variant thereof is immunologically distinct from an NDV F protein if antibodies directed to the NDV F protein bind to the non-NDV APMV F protein or variant thereof with a 2-fold, 5-fold, 10-fold, 15-fold, 20-fold or lower affinity than to NDV F protein in an assay known to one of skill in the art or described herein. In certain embodiments, a non-NDV APMV F protein or a variant thereof is immunologically distinct from an NDV F protein if antibodies directed to the NDV F protein bind to the non-NDV APMV F protein or variant thereof with a 0.5 log, 1 log, 1.5 log, 2 log, 2.5 log, 3 log or lower affinity than to NDV F protein in an assay known to one of skill in the art or described herein. In certain embodiments, a non-NDV APMV F protein or a variant thereof is immunologically distinct from an NDV F protein if anti-NDV F antibodies do not substantially inhibit replication of NDV expressing the non-NDV APMV F protein or a variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a non-NDV APMV F protein or a variant thereof is immunologically distinct from an NDV F protein if anti-NDV F antibodies inhibit replication of NDV expressing the non-NDV APMV F protein or variant thereof in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In some embodiments, the non-NDV APMV F protein is an F protein from a different genus than NDV. In some embodiments, the non-NDV APMV F protein is an F protein from a member of the subfamily Avulavirinae from a different genus than NDV. In some embodiments, the non-NDV APMV F protein is an F protein from a member of the subfamily Avulavirinae, but is not NDV. In some embodiments, the non-NDV APMV F protein is an F protein from a member of the subfamily Avulavirinae and the genus metaavulavirus. In some embodiments, the non-NDV APMV F protein is an F protein from a member of the subfamily Avulavirinae and the genus paraavulavirus. In some embodiments, the non-NDV APMV F protein is an F protein from a member of the subfamily Avulavirinae and the genus orthoavulavirus but is not NDV.

In certain embodiments, a chimeric F protein is immunologically distinct from an NDV F protein. In certain embodiments, a chimeric F protein is immunologically distinct from an NDV F protein if antibodies directed to the NDV F protein do not cross-react with the chimeric F protein. In some embodiments, a chimeric F protein is immunologically distinct from an NDV F protein if antibodies directed to the NDV F protein bind to the chimeric F protein with a 2-fold, 5-fold, 10-fold, 15-fold, 20-fold or lower affinity than to NDV F protein in an assay known to one of skill in the art or described herein. In certain embodiments, a chimeric F protein is immunologically distinct from an NDV F protein if antibodies directed to the NDV F protein bind to the chimeric F protein with a 0.5 log, 1 log, 1.5 log, 2 log, 2.5 log, 3 log or lower affinity than to NDV F protein in an assay known to one of skill in the art or described herein. In certain embodiments, a chimeric F protein is immunologically distinct from an NDV F protein if anti-NDV F antibodies do not substantially inhibit replication of NDV expressing the non-NDV APMV F protein or a variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a chimeric F protein is immunologically distinct from an NDV F protein if anti-NDV F antibodies inhibit replication of NDV expressing the chimeric F protein in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs.

In specific embodiments, a non-NDV APMV F protein does not contain a multibasic cleavage site. In certain embodiments, a non-NDV APMV F protein is modified by, e.g., one or more amino acid substitutions so that the non-NDV APMV F protein no longer contains a multi-basic cleavage. In some embodiments, the original sequence of the cleavage site of the non-NDVAPMV F protein is modified by, e.g., one or more amino acid substitutions. For example, a leucine at the amino acid position of the non-NDV APMV F protein corresponding to amino acid position 289 of NDV F protein (as counted by the LaSota strain F protein) may be substituted for alanine to eliminate a multi-basic cleavage site.

In specific embodiments, a variant of a non-NDV APMV F protein does not contain a multibasic cleavage site. In certain embodiments, a variant of a non-NDV APMV F protein includes one or more amino acid substitutions so that the non-NDV APMV F protein no longer contains a multi-basic cleavage. In some embodiments, the original sequence of the cleavage site of the variant of the non-NDVAPMV F protein is modified by, e.g., one or more amino acid substitutions. For example, a variant of a non-NDV APMV F protein includes an amino acid substitution of alanine for leucine at the amino acid position of the non-NDV APMV F protein corresponding to amino acid position 289 of NDV F protein (as counted by the LaSota strain F protein).

In specific embodiments, a chimeric F protein does not contain a multibasic cleavage site. In certain embodiments, a chimeric F protein includes an amino acid substitution so that the ectodomain of the non-NDV APMV F protein no longer contains a multi-basic cleavage. In some embodiments, the original sequence of the cleavage site of the ectodomain of the non-NDVAPMV F protein is modified by, e.g., one or more amino acid substitutions. For example, a chimeric protein includes an amino acid substitution of alanine for leucine at the amino acid position of the ectodomain of the non-NDV APMV F protein corresponding to amino acid position 289 of NDV F protein (as counted by the LaSota strain F protein).

In specific embodiments, a variant of a non-NDV APMV F protein retains one or more functions of the non-NDV APMV F protein.

In certain embodiments, a variant of a non-NDV APMV F protein is at least 75%, at least 80%, or at least 85% identical to the non-NDV AMPV F protein. In some embodiments, a variant of a non-NDV APMV F protein is at least 90%, at least 95%, or at least 99% identical to the non-NDV APMV F protein. In certain embodiments, a variant of a non-NDV APMV F protein is 75% to 90%, 80% to 95% or 90% to 99.5% identical to the non-NDV AMPV F protein.

In certain embodiment, a variant of a non-NDV APMV F protein contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a non-NDV APMV F protein. In some embodiments, a variant of a non-NDV APMV F protein comprises the amino acid sequence of the non-NDV APMV F protein with 20, 15, 10, 9, 8, 7, 6, 5, 4, 3, 2 or 1 amino acid residues of the non-NDV APMV F protein substituted (e.g., conservatively substituted) with other amino acids. In certain embodiments, a variant of a non-NDV APMV F protein comprises the amino acid sequence of the non-NDV APMV F protein with up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In some embodiments, a variant of a non-NDV APMV F protein is a polypeptide encoded by nucleic acid sequence that can hybridize under high, moderate or typical stringency hybridization conditions to a nucleic acid sequence encoding the non-NDV APMV F protein. Hybridization conditions are known to one of skill in the art (see, e.g., U.S. Patent Application No. 2005/0048549 at, e.g., paragraphs 72 and 73).

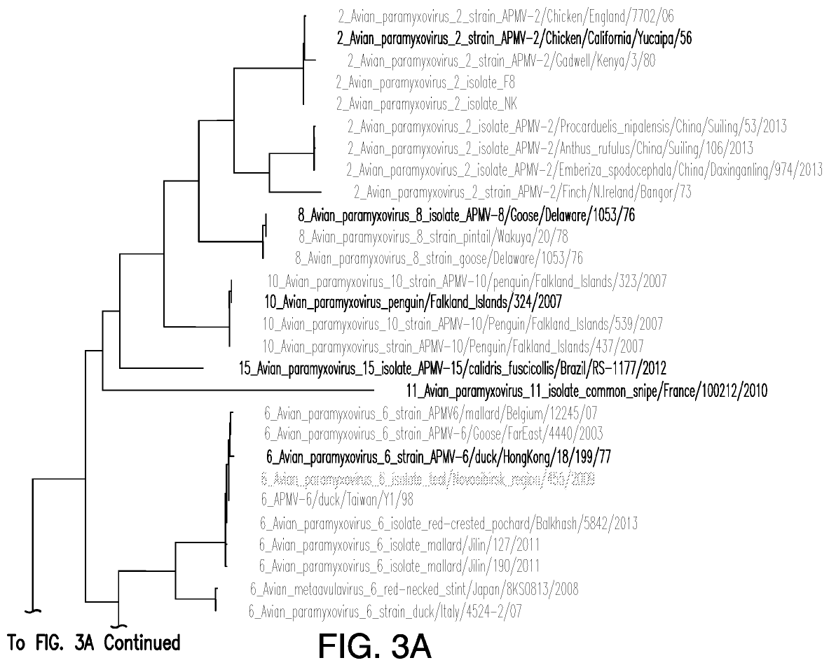

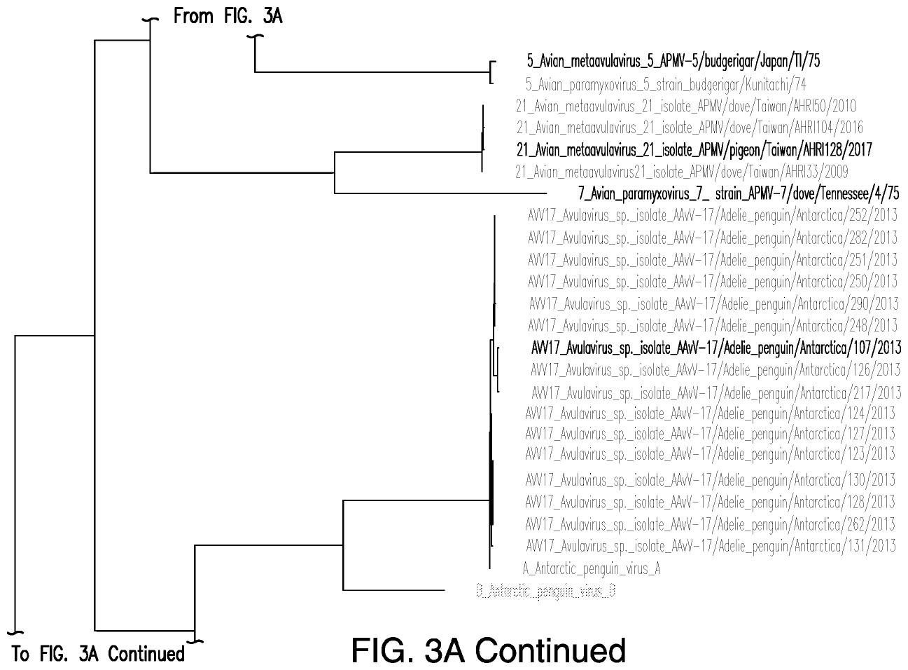

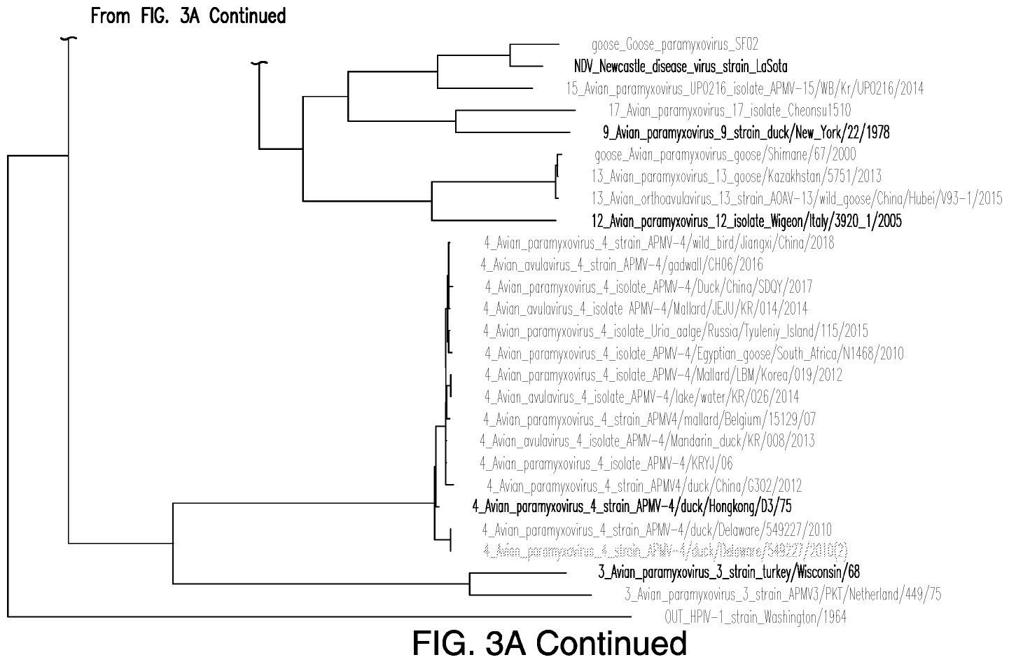

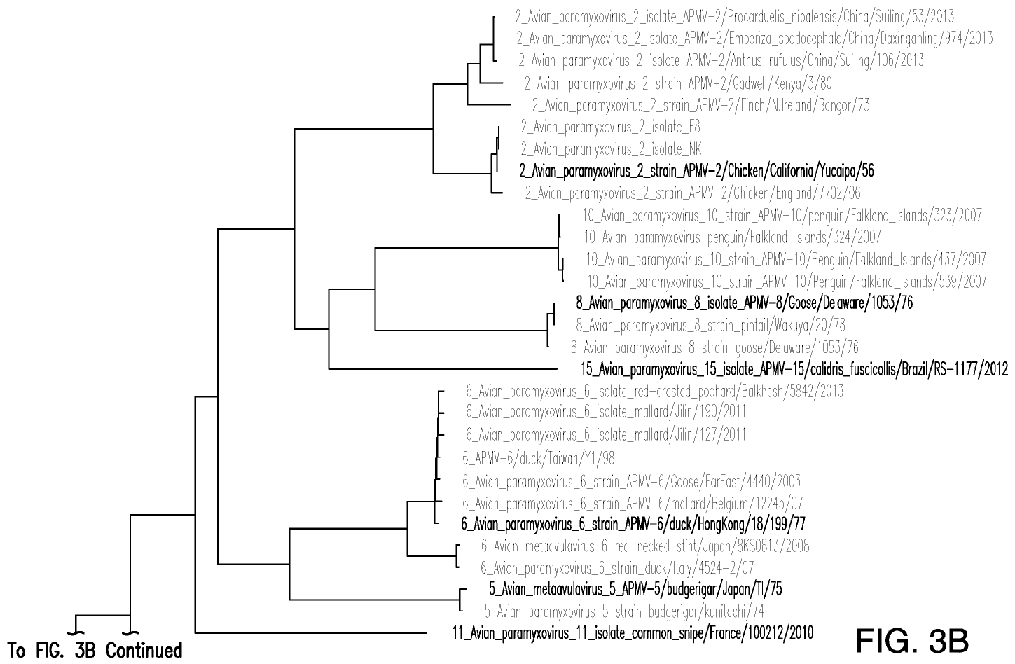

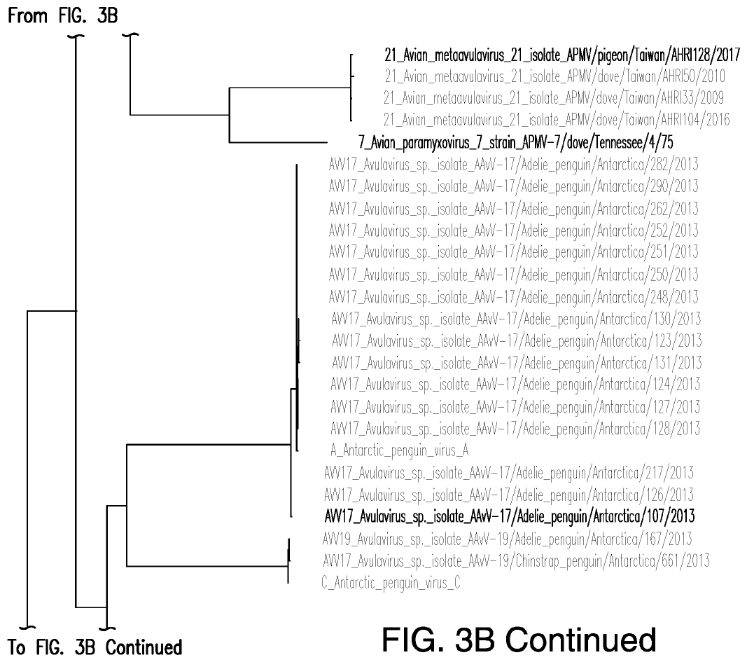

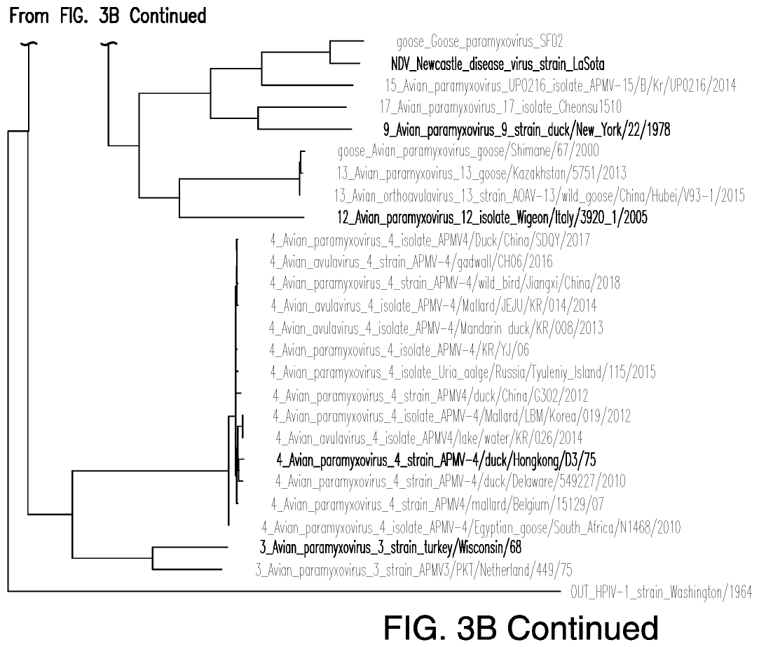

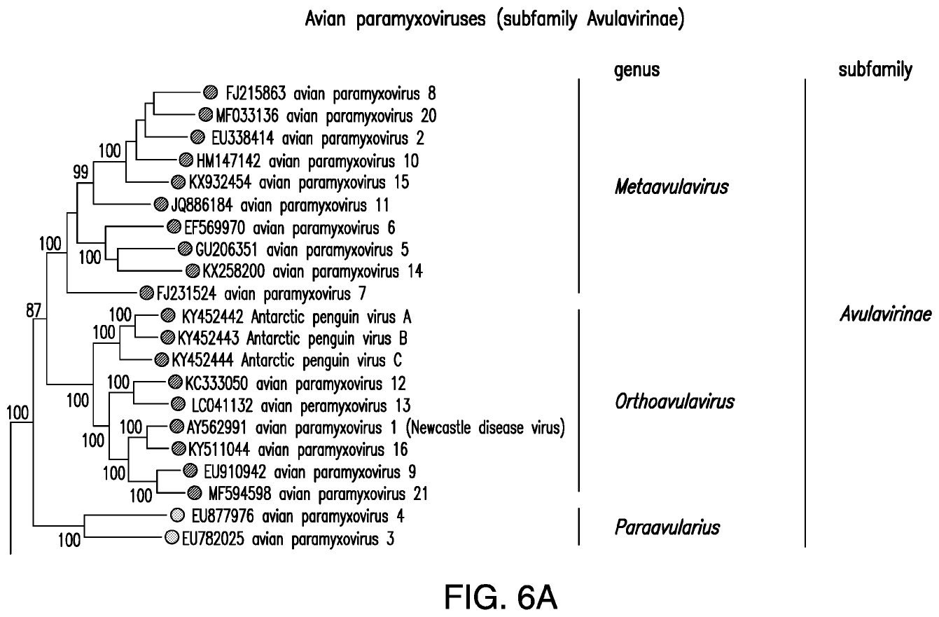

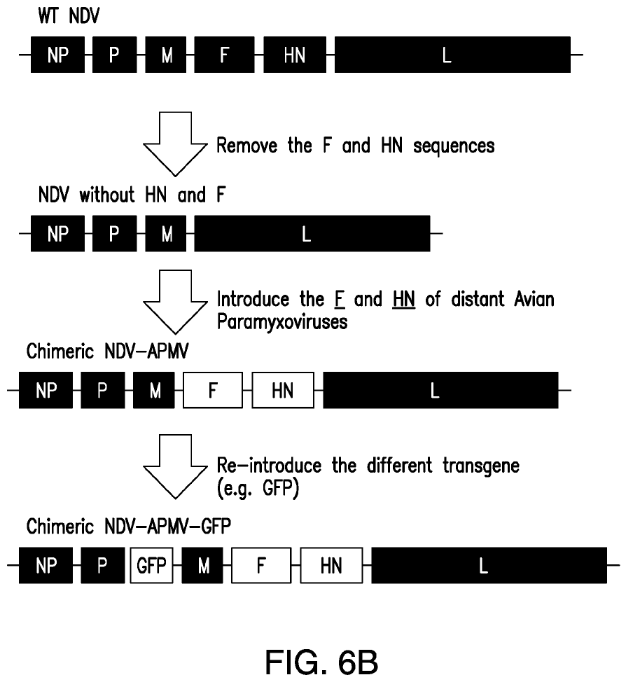

In a specific embodiment, a non-NDV APMV F protein is any non-NDV AMPV F protein that is immunologically distinct from an NDV F protein. In some embodiments, a non-NDV APMV F protein is the F protein of an APMV shown in FIG. 3A. In some embodiments, a non-NDV APMV F protein is the F protein of a member of a genus shown in FIG. 3A or FIG. 6A. In certain embodiments, a non-NDV APMV F protein is the F protein of AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, a non-NDV APMV F protein is the F protein of APMV-2, such as, e.g., Chicken/California/Yucaipa/56 (Accession No. EU338414). In certain embodiments, a non-NDV APMV F protein is the F protein of APMV-2 Yucaipa. In other embodiments, a non-NDV APMV F protein is not the F protein of APMV-2 Yucaipa. In certain embodiments, a non-NDV APMV F protein is the F protein of APMV-3, such as, e.g., APMV3/Turkey/Wisconsin/68 (Accession No. EU782025). In some embodiments, a non-NDV APMV F protein is the F protein of APMV-4, such as, e.g., aAPMV4/duck/Hongkong/D3/75 (Accession No. FJ177514), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, a non-NDV APMV F protein is the F protein of APMV-5, such as, e.g., APMV-5 budgerigar/Kunitachi/74 (Accession No. GU206351) or APMV5/budgerigar/Japan/TI/75 (Accession No. LC168750). In some embodiments, a non-NDV APMV F protein is the F protein of APMV-6, such as, e.g., APMV-6 Goose/FarEast/4440/2003 (Accession No. EF569970) or APMV6/duck/HongKong/18/199/77 (Accession No. EU622637). In certain embodiments, a non-NDV APMV F protein is the F protein of APMV-7, such as, e.g., APMV-7 dove/Tennessee/4/75 (Accession No. FJ231524). In some embodiments, a non-NDV APMV F protein is the F protein of APMV-8, such as, e.g., APMV-8 goose/Delaware/1053/76 (Accession No. FJ215863). In certain embodiments, a non-NDV APMV F protein is the F protein of APMV-9, such as, e.g., APMV9/duck/New York/22/78 (Accession No. EU910942). In some embodiments, a non-NDV APMV F protein is the F protein of APMV-10, such as, e.g., APMV-10 penguin/Falkland Islands/324/2007 (Accession No. HM147142 or NC_025349). In certain embodiments, a non-NDV APMV F protein is the F protein of APMV-11, such as, e.g., APMV-11 common_snipe/France/100212/2010 (Accession No. JQ886184). In some embodiments, a non-NDV APMV F protein is the F protein of APMV-12, such as, e.g., APMV12/Wigeon/Italy/3920_1/05 (Accession No. KC333050). In some embodiments, a non-NDV APMV F protein is the F protein of APMV-14, such as, e.g., APMV-14 duck/Japan/110G0352/2011 (Accession No. KX258200). In certain embodiments, a non-NDV APMV F protein is the F protein of APMV-15, such as, e.g., APMV-15 calidris_fuscicollis/Brazil/RS-1177/2012 (Accession No. KX932454). In some embodiments, a non-NDV APMV F protein is the F protein of APMV-17, such as, e.g., APMV17/Antarctica/107/13 (Accession No. MK167211). In some embodiments, a non-NDV APMV F protein is the F protein of APMV-20, such as, e.g., APMV-20 Gull/Kazakhstan/2014 (Accession No. MF033136). In certain embodiments, a non-NDV APMV F protein is the F protein of APMV-21, such as, e.g., APMV21/pigeon/Taiwan/AHRI128/17 (Accession No. MK67743).

In certain embodiments, a variant of a non-NDV APMV F protein is a variant of the F protein of AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, a variant of a non-NDV APMV F protein is a variant of the F protein of APMV-2, such as, e.g., Chicken/California/Yucaipa/56 (Accession No. EU338414). In certain embodiments, a variant of a non-NDV APMV F protein is a variant of the F protein of APMV-3, such as, e.g., APMV3/Turkey/Wisconsin/68 (Accession No. EU782025). In some embodiments, a variant of a non-NDV APMV F protein is a variant of the F protein of APMV-4, such as, e.g., APMV4/duck/Hongkong/D3/75 (Accession No. FJ177514), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, a variant of a non-NDV APMV F protein is a variant of the F protein of APMV-5, such as, e.g., APMV-5 budgerigar/Kunitachi/74 (Accession No. GU206351) or APMV5/budgerigar/Japan/TI/75 (Accession No. LC168750). In some embodiments, a variant of a non-NDV APMV F protein is a variant of the F protein of APMV-6, such as, e.g., APMV-6 Goose/FarEast/4440/2003 (Accession No. EF569970) or APMV6/duck/HongKong/18/199/77 (Accession No. EU622637). In certain embodiments, a variant of a non-NDV APMV F protein is a variant of the F protein of APMV-7, such as, e.g., APMV-7 dove/Tennessee/4/75 (Accession No. FJ231524). In some embodiments, a variant of a non-NDV APMV F protein is a variant of the F protein of APMV-8, such as, e.g., APMV-8 goose/Delaware/1053/76 (Accession No. FJ215863). In certain embodiments, a variant of a non-NDV APMV F protein is a variant of the F protein of APMV-9, such as, e.g., APMV9/duck/New York/22/78 (Accession No. EU910942). In some embodiments, a variant of a non-NDV APMV F protein is a variant of the F protein of APMV-10, such as, e.g., APMV-10 penguin/Falkland Islands/324/2007 (Accession No. HM147142 or NC_025349). In certain embodiments, a variant of a non-NDV APMV F protein is a variant of the F protein of APMV-11, such as, e.g., APMV-11 common_snipe/France/100212/2010 (Accession No. JQ886184). In some embodiments, a variant of a non-NDV APMV F protein is a variant of the F protein of APMV-12, such as, e.g., APMV12/Wigeon/Italy/3920_1/05 (Accession No. KC333050). In some embodiments, a variant of a non-NDV APMV F protein is a variant of the F protein of APMV-14, such as, e.g., APMV-14 duck/Japan/110G0352/2011 (Accession No. KX258200). In certain embodiments, a variant of a non-NDV APMV F protein is a variant of the F protein of APMV-15, such as, e.g., APMV-15 calidris_fuscicollis/Brazil/RS-1177/2012 (Accession No. KX932454). In some embodiments, a variant of a non-NDV APMV F protein is a variant of the F protein of APMV-17, such as, e.g., APMV17/Antarctica/107/13 (Accession No. MK167211). In some embodiments, a variant of a non-NDV APMV F protein is a variant of the F protein of APMV-20, such as, e.g., APMV-20 Gull/Kazakhstan/2014 (Accession No. MF033136). In certain embodiments, a variant of a non-NDV APMV F protein is a variant of the F protein of APMV-21, such as, e.g., APMV21/pigeon/Taiwan/AHRI128/17 (Accession No. MK67743).

In some embodiments, a non-NDV APMV F protein has less than 65% identity to an NDV F protein. In some embodiments, a non-NDV APMV F protein has less than 60% identity to an NDV F protein. In some embodiments, a non-NDV APMV F protein has less than 50% identity to an NDV F protein. In some embodiments, a non-NDV APMV F protein has less than 55% identity to an NDV F protein. In some embodiments, a non-NDV APMV F protein has less than 50% identity to an NDV F protein. In some embodiments, a non-NDV APMV F protein has less than 45% identity to an NDV F protein. In some embodiments, a non-NDV APMV F protein has less than 40% identity to an NDV F protein. In some embodiments, a non-NDV APMV F protein has less than 35% identity to an NDV F protein. In some embodiments, a non-NDV APMV F protein has at least 20% or at least 25% identity to an NDV F protein but less than 65%, less than 60%, less than 55%, less than 50%, or less than 45% identity. In some embodiments, the NDV F protein is the NDV LaSota F protein.

In certain embodiments, a non-NDV APMV HN protein is immunologically distinct from an NDV HN protein. In some embodiments, a variant of a non-NDV APMV HN protein is immunologically distinct from an NDV HN protein. In certain embodiments, a non-NDV APMV HN protein or a variant thereof is immunologically distinct from an NDV HN protein if antibodies directed to the NDV HN protein do not cross-react with the non-NDV APMV HN protein or variant thereof. In some embodiments, a non-NDV APMV HN protein or a variant thereof is immunologically distinct from an NDV HN protein if antibodies directed to the NDV HN protein bind to the variant with a 2-fold, 5-fold, 10-fold, 15-fold, 20-fold or lower affinity than to the non-NDV APMV HN protein or variant thereof in an assay known to one of skill in the art or described herein. In certain embodiments, a non-NDV APMV HN protein or a variant thereof is immunologically distinct from an NDV HN protein if antibodies directed to the NDV HN protein bind to the non-NDV APMV HN protein or variant thereof with a 0.5 log, 1 log, 1.5 log, 2 log, 2.5 log, 3 log or lower affinity than to NDV HN protein in an assay known to one of skill in the art or described herein. In certain embodiments, a non-NDV APMV HN protein or a variant thereof is immunologically distinct from an NDV HN protein if anti-NDV HN antibodies do not substantially inhibit replication of NDV expressing the non-NDV APMV HN protein or variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a non-NDV APMV HN protein or a variant thereof is immunologically distinct from an NDV HN protein if anti-NDV HN antibodies inhibit replication of NDV expressing the non-NDV APMV HN protein or variant thereof in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In some embodiments, the non-NDV APMV HN protein is an HN protein from a member of the subfamily Avulavirinae from a different genus than NDV. In some embodiments, the non-NDV APMV HN protein is an HN protein from a member of the subfamily Avulavirinae, but is not NDV. In some embodiments, the non-NDV APMV HN protein is an HN protein from a member of the subfamily Avulavirinae and the genus Metaavulavirus. In some embodiments, the non-NDV APMV HN protein is an HN protein from a member of the subfamily Avulavirinae and the genus paraavulavirus. In some embodiments, the non-NDV APMV HN protein is an HN protein from a member of the subfamily Avulavirinae and the genus orthoavulavirus but is not NDV.

In certain embodiments, a chimeric HN protein is immunologically distinct from an NDV HN protein. In certain embodiments, a chimeric HN protein is immunologically distinct from an NDV HN protein if antibodies directed to the NDV HN protein do not cross-react with the chimeric HN protein. In some embodiments, a chimeric HN protein is immunologically distinct from an NDV HN protein if antibodies directed to the NDV HN protein bind to the chimeric HN protein with a 2-fold, 5-fold, 10-fold, 15-fold, 20-fold or lower affinity than to NDV HN protein in an assay known to one of skill in the art or described herein. In certain embodiments, a chimeric HN protein is immunologically distinct from an NDV HN protein if antibodies directed to the NDV HN protein bind to the chimeric HN protein with a 0.5 log, 1 log, 1.5 log, 2 log, 2.5 log, 3 log or lower affinity than to NDV HN protein in an assay known to one of skill in the art or described herein. In certain embodiments, a chimeric HN protein is immunologically distinct from an NDV HN protein if anti-NDV HN antibodies do not substantially inhibit replication of NDV expressing the non-NDV APMV F protein or a variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a chimeric HN protein is immunologically distinct from an NDV HN protein if anti-NDV HN antibodies inhibit replication of NDV expressing the chimeric HN protein in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs.

In specific embodiments, a variant of a non-NDV APMV HN protein retains one or more functions of the non-NDV APMV HN protein.

In certain embodiments, a variant of a non-NDV APMV HN protein is at least 75%, at least 80%, or at least 85% identical to the non-NDV AMPV HN protein. In some embodiments, a variant of a non-NDV HN protein is at least 90%, at least 95%, or at least 99% identical to the non-NDV APMV HN protein. In certain embodiments, a variant of a non-NDV APMV HN protein is 75% to 90%, 80% to 95% or 90% to 99.5% identical to the non-NDV AMPV HN protein.

In certain embodiment, a variant of a non-NDV APMV HN protein contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a non-NDV APMV HN protein. In some embodiments, a variant of a non-NDV APMV HN protein comprises the amino acid sequence of the non-NDV APMV HN protein with 20, 15, 10, 9, 8, 7, 6, 5, 4, 3, 2 or 1 amino acid residues of the non-NDV APMV HN protein substituted (e.g., conservatively substituted) with other amino acids. In certain embodiments, a variant of a non-NDV APMV HN protein comprises the amino acid sequence of the non-NDV APMV HN protein with up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In some embodiments, a variant of a non-NDV APMV HN protein is a polypeptide encoded by nucleic acid sequence that can hybridize under high, moderate or typical stringency hybridization conditions to a nucleic acid sequence encoding the non-NDV APMV HN protein. Hybridization conditions are known to one of skill in the art (see, e.g., U.S. Patent Application No. 2005/0048549 at, e.g., paragraphs 72 and 73).

In a specific embodiment, a non-NDV APMV HN protein is any non-NDV AMPV HN protein that is immunologically distinct from an NDV HN protein. In some embodiments, a non-NDV APMV HN protein is the HN protein of an APMV shown in FIG. 3B. In some embodiments, a non-NDV APMV HN protein is the HN protein of a member of a genus shown in FIG. 3B or FIG. 6A. In certain embodiments, a non-NDV APMV HN protein is the HN protein of AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, a non-NDV APMV HN protein is the HN protein of APMV-2, such as, e.g., Chicken/California/Yucaipa/56 (Accession No. EU338414). In certain embodiments, a non-NDV APMV HN protein is the HN protein of APMV-2 Yucaipa. In other embodiments, a non-NDV APMV HN protein is not the HN protein of APMV-2 Yucaipa. In certain embodiments, a non-NDV APMV HN protein is the HN protein of APMV-3, such as, e.g., APMV3/Turkey/Wisconsin/68 (Accession No. EU782025). In some embodiments, a non-NDV APMV HN protein is the HN protein of APMV-4, such as, e.g., aAPMV4/duck/Hongkong/D3/75 (Accession No. FJ177514), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, a non-NDV APMV HN protein is the HN protein of APMV-5, such as, e.g., APMV-5 budgerigar/Kunitachi/74 (Accession No. GU206351) or APMV5/budgerigar/Japan/TI/75 (Accession No. LC168750). In some embodiments, a non-NDV APMV HN protein is the HN protein of APMV-6, such as, e.g., APMV-6 Goose/FarEast/4440/2003 (Accession No. EF569970) or APMV6/duck/HongKong/18/199/77 (Accession No. EU622637). In certain embodiments, a non-NDV APMV HN protein is the HN protein of APMV-7, such as, e.g., APMV-7 dove/Tennessee/4/75 (Accession No. FJ231524). In some embodiments, a non-NDV APMV HN protein is the HN protein of APMV-8, such as, e.g., APMV-8 goose/Delaware/1053/76 (Accession No. FJ215863). In certain embodiments, a non-NDV APMV HN protein is the HN protein of APMV-9, such as, e.g., APMV9/duck/New York/22/78 (Accession No. EU910942). In some embodiments, a non-NDV APMV HN protein is the HN protein of APMV-10, such as, e.g., APMV-10 penguin/Falkland Islands/324/2007 (Accession No. HM147142 or NC_025349). In certain embodiments, a non-NDV APMV HN protein is the HN protein of APMV-11, such as, e.g., APMV-11 common_snipe/France/100212/2010 (Accession No. JQ886184). In some embodiments, a non-NDV APMV HN protein is the HN protein of APMV-12, such as, e.g., APMV12/Wigeon/Italy/3920_1/05 (Accession No. KC333050). In some embodiments, a non-NDV APMV HN protein is the HN protein of APMV-14, such as, e.g., APMV-14 duck/Japan/110G0352/2011 (Accession No. KX258200). In certain embodiments, a non-NDV APMV HN protein is the HN protein of APMV-15, such as, e.g., APMV-15 calidris_fuscicollis/Brazil/RS-1177/2012 (Accession No. KX932454). In some embodiments, a non-NDV APMV HN protein is the HN protein of APMV-17, such as, e.g., APMV17/Antarctica/107/13 (Accession No. MK167211). In some embodiments, a non-NDV APMV HN protein is the HN protein of APMV-20, such as, e.g., APMV-20 Gull/Kazakhstan/2014 (Accession No. MF033136). In certain embodiments, a non-NDV APMV HN protein is the HN protein of APMV-21, such as, e.g., APMV21/pigeon/Taiwan/AHRI128/17 (Accession No. MK67743).

In certain embodiments, a variant of a non-NDV APMV HN protein is a variant of the HN protein of AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, a variant of a non-NDV APMV HN protein is a variant of the HN protein of APMV-2, such as, e.g., Chicken/California/Yucaipa/56 (Accession No. EU338414). In certain embodiments, a variant of a non-NDV APMV HN protein is a variant of the HN protein of APMV-3, such as, e.g., APMV3/Turkey/Wisconsin/68 (Accession No. EU782025). In some embodiments, a variant of a non-NDV APMV HN protein is a variant of the HN protein of APMV-4, such as, e.g., APMV4/duck/Hongkong/D3/75 (Accession No. FJ177514), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, a variant of a non-NDV APMV HN protein is a variant of the HN protein of APMV-5, such as, e.g., APMV-5 budgerigar/Kunitachi/74 (Accession No. GU206351) or APMV5/budgerigar/Japan/TI/75 (Accession No. LC168750). In some embodiments, a variant of a non-NDV APMV HN protein is a variant of the HN protein of APMV-6, such as, e.g., APMV-6 Goose/FarEast/4440/2003 (Accession No. EF569970) or APMV6/duck/HongKong/18/199/77 (Accession No. EU622637). In certain embodiments, a variant of a non-NDV APMV HN protein is a variant of the HN protein of APMV-7, such as, e.g., APMV-7 dove/Tennessee/4/75 (Accession No. FJ231524). In some embodiments, a variant of a non-NDV APMV HN protein is a variant of the HN protein of APMV-8, such as, e.g., APMV-8 goose/Delaware/1053/76 (Accession No. FJ215863). In certain embodiments, a variant of a non-NDV APMV HN protein is a variant of the HN protein of APMV-9, such as, e.g., APMV9/duck/New York/22/78 (Accession No. EU910942). In some embodiments, a variant of a non-NDV APMV HN protein is a variant of the HN protein of APMV-10, such as, e.g., APMV-10 penguin/Falkland Islands/324/2007 (Accession No. HM147142 or NC_025349). In certain embodiments, a variant of a non-NDV APMV HN protein is a variant of the HN protein of APMV-11, such as, e.g., APMV-11 common_snipe/France/100212/2010 (Accession No. JQ886184). In some embodiments, a variant of a non-NDV APMV HN protein is a variant of the HN protein of APMV-12, such as, e.g., APMV12/Wigeon/Italy/3920_1/05 (Accession No. KC333050). In some embodiments, a variant of a non-NDV APMV HN protein is a variant of the HN protein of APMV-14, such as, e.g., APMV-14 duck/Japan/110G0352/2011 (Accession No. KX258200). In certain embodiments, a variant of a non-NDV APMV HN protein is a variant of the HN protein of APMV-15, such as, e.g., APMV-15 calidris_fuscicollis/Brazil/RS-1177/2012 (Accession No. KX932454). In some embodiments, a variant of a non-NDV APMV HN protein is a variant of the HN protein of APMV-17, such as, e.g., APMV17/Antarctica/107/13 (Accession No. MK167211). In some embodiments, a variant of a non-NDV APMV HN protein is a variant of the HN protein of APMV-20, such as, e.g., APMV-20 Gull/Kazakhstan/2014 (Accession No. MF033136). In certain embodiments, a variant of a non-NDV APMV HN protein is a variant of the HN protein of APMV-21, such as, e.g., APMV21/pigeon/Taiwan/AHRI128/17 (Accession No. MK67743).

In some embodiments, a non-NDV APMV HN protein has less than 65% identity to an NDV HN protein. In some embodiments, a non-NDV APMV HN protein has less than 60% identity to an NDV HN protein. In some embodiments, a non-NDV APMV HN protein has less than 50% identity to an NDV HN protein. In some embodiments, a non-NDV APMV HN protein has less than 55% identity to an NDV HN protein. In some embodiments, a non-NDV APMV HN protein has less than 50% identity to an NDV HN protein. In some embodiments, a non-NDV APMV HN protein has less than 45% identity to an NDV HN protein. In some embodiments, a non-NDV APMV HN protein has less than 40% identity to an NDV HN protein. In some embodiments, a non-NDV APMV HN protein has less than 35% identity to an NDV HN protein. In some embodiments, a non-NDV APMV HN protein has at least 20% or at least 25% identity to an NDV HN protein but less than 65%, less than 60%, less than 55%, less than 50%, or less than 45% identity. In some embodiments, the NDV HN protein is the NDV LaSota HN protein.

In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:1. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:2. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:3. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:4. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:5. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:6. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:7. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:8. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:9. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:10. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:11. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:12. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:13. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of the coding sequence of the cDNA sequence set forth in SEQ ID NO:14. In specific embodiments, the NDV genome comprises the replaced NDV HN and F protein coding sequences as well as (1) a transcription unit encoding a NDV nucleocapsid (N) protein, (2) a transcription unit encoding a NDV phosphoprotein (P), (3) a transcription unit encoding a NDV matrix (M) protein, and (4) a transcription unit encoding a NDV large polymerase (L).

In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:1. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:2. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:3. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:4. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:5. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:6. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:7. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:8. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:9. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:10. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:11. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:12. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:13. In a specific embodiment, provided herein is a recombinant NDV comprising a packaged genome, wherein the packaged genome comprises a nucleotide sequence of a Newcastle disease virus genome in which the coding sequences of NDV HN protein and the NDV F protein have been replaced with an RNA sequence corresponding to the negative sense of a nucleotide sequence coding sequence that is at least 75%, at least 80%, at least 85%, at least 90%, at least 95% or at least 98% identical to the coding sequence of the cDNA sequence set forth in SEQ ID NO:14. In specific embodiments, the NDV genome comprises the replaced NDV HN and F protein coding sequences as well as (1) a transcription unit encoding a NDV nucleocapsid (N) protein, (2) a transcription unit encoding a NDV phosphoprotein (P), (3) a transcription unit encoding a NDV matrix (M) protein, and (4) a transcription unit encoding a NDV large polymerase (L).

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length. In a certain embodiment, the percent identity is determined over the entire length of an amino acid sequence or nucleotide sequence. In some embodiments, the length of sequence identity comparison may be over the full-length of the two sequences being compared (e.g., the full-length of a gene coding sequence, or a fragment thereof). In some embodiments, a fragment of a nucleotide sequence is at least 25, at least 50, at least 75, or at least 100 nucleotides. Similarly, “percent sequence identity” may be readily determined for amino acid sequences, over the full-length of a protein, or a fragment thereof. In some embodiments, a fragment of a protein comprises at least 20, at least 30, at least 40, at least 50 or more contiguous amino acids of the protein. In certain embodiments, a fragment of a protein comprises at least 75, at least 100, at least 125, at least 150 or more contiguous amino acids of the protein.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:1. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:2. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:3. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:4. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:5. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:6. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with a nucleotide sequence comprising the nucleotide sequence an RNA sequence corresponding to the negative sense of the cDNA sequence set forth in SEQ ID NO:7. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:8. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:9. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:10. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:11. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:12. In another specific embodiment, provided herein is a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:13. In another specific embodiment, provided herein a recombinant NDV comprising a packaged genome, wherein the packaged genome comprises a nucleotide sequence of a Newcastle disease virus genome in which the nucleotide sequences encoding the NDV HN protein and the NDV F protein have been replaced with an RNA sequence corresponding to the negative sense of the cDNA sequence set forth in SEQ ID NO:14. In specific embodiments, the NDV genome comprises the replaced NDV HN and F protein coding sequences as well as (1) a transcription unit encoding a NDV nucleocapsid (N) protein, (2) a transcription unit encoding a NDV phosphoprotein (P), (3) a transcription unit encoding a NDV matrix (M) protein, and (4) a transcription unit encoding a NDV large polymerase (L).

One skilled in the art will understand that the NDV genomic RNA sequence is an RNA sequence corresponding to the negative sense of a cDNA sequence encoding the NDV genome. Thus, any program that converts a nucleotide sequence to its reverse complement sequence may be utilized to convert a cDNA sequence encoding an NDV genome into the genomic RNA sequence (see, e.g., www.bioinformatics.org/sms/rev_comp.html, www.fr33.net/seqedit.php, and DNAStar). Accordingly, the nucleotide sequences provided in Table 1 and Table 3, infra, may be readily converted to the negative-sense RNA sequence of the NDV genome by one of skill in the art.

In some embodiments, the nucleotide sequence of a NDV genome is of an NDV of any strain known to one of skill in the art. See, e.g., Section 5.1.2 for exemplary strains. In a specific embodiments, the nucleotide sequence of a NDV genome is of the LaSota strain. In some embodiments, the nucleotide sequence of a NDV genome comprises an RNA sequence corresponding to the cDNA sequence set forth in SEQ ID NO:15. In certain embodiments, the nucleotide sequence of a NDV genome is of a lentogenic strain. In some embodiments, the nucleotide sequence of a NDV genome is of a mesogenic strain. In certain embodiments, the nucleotide sequence of a NDV genome is of a velogenic. The nucleotide sequence of a NDV genome may be a cDNA sequence or an RNA sequence (e.g., negative sense RNA or positive sense RNA).

In some embodiments, provided herein is a recombinant NDV comprising a packaged genome, wherein the packaged genome comprises a negative sense RNA sequence corresponding to the cDNA sequence of SEQ ID NO:44. In some embodiments, provided herein is a recombinant NDV comprising a packaged genome, wherein the packaged genome comprises a negative sense RNA sequence corresponding to the cDNA sequence of SEQ ID NO:44 without the GFP coding sequence. In some embodiments, provided herein is a recombinant NDV comprising a packaged genome, wherein the packaged genome comprises a negative sense RNA sequence corresponding to a nucleotide sequence that is at least 75%, at least 80%, at least 85%, at least 90%, at least 95%, at least 98%, or at least 99% identical to the cDNA sequence of SEQ ID NO:44. In some embodiments, provided herein is a recombinant NDV comprising a packaged genome, wherein the packaged genome comprises a negative sense RNA sequence corresponding to a nucleotide sequence that is at least 75%, at least 80%, at least 85%, at least 90%, at least 95%, at least 98%, or at least 99% identical to the cDNA sequence of SEQ ID NO:44 without the GFP coding sequence.

In some embodiments, provided herein is a recombinant NDV comprising a packaged genome, wherein the packaged genome comprises a negative sense RNA sequence corresponding to the cDNA sequence of SEQ ID NO:45. In some embodiments, provided herein is a recombinant NDV comprising a packaged genome, wherein the packaged genome comprises a negative sense RNA sequence corresponding to the cDNA sequence of SEQ ID NO:45 without the GFP coding sequence. In some embodiments, provided herein is a recombinant NDV comprising a packaged genome, wherein the packaged genome comprises a negative sense RNA sequence corresponding to a nucleotide sequence that is at least 75%, at least 80%, at least 85%, at least 90%, at least 95%, at least 98%, or at least 99% identical to the cDNA sequence of SEQ ID NO:45. In some embodiments, provided herein is a recombinant NDV comprising a packaged genome, wherein the packaged genome comprises a negative sense RNA sequence corresponding to a nucleotide sequence that is at least 75%, at least 80%, at least 85%, at least 90%, at least 95%, at least 98%, or at least 99% identical to the cDNA sequence of SEQ ID NO:45 without the GFP coding sequence.

In some embodiments, a nucleotide sequence described herein is codon optimized. See Section 5.1.4 for a description of codon optimization information and techniques.

In a specific embodiment, a recombinant NDV is one described in Section 6.

In certain embodiments, a packaged genome described herein does not comprise a heterologous sequence encoding a heterologous protein other than the non-NDV APMV HN protein or variant thereof. In certain embodiments, a packaged genome described herein does not comprise a heterologous sequence encoding a heterologous protein other than the non-NDV APMV F protein or variant thereof. In certain embodiments, a packaged genome described herein does not comprise a heterologous sequence encoding a heterologous protein other than the non-NDV APMV HN protein or variant thereof and non-NDV APMV F protein or variant thereof.

In certain embodiments, a packaged genome described herein further comprises a transgene comprising a nucleotide sequence encoding a heterologous sequence. In certain embodiments, a packaged genome described herein further comprises a transgene comprising a nucleotide sequence encoding an antigen. In some embodiments, a packaged genome described herein further comprises two or more transgenes, wherein each transgene comprises a nucleotide sequence encoding an antigen. See Section 5.1.3 for a description of transgenes that may be incorporated into a packaged genome described herein. In some embodiments, the antigen is a chimeric protein, such as described in Section 5.1.3, infra. In specific embodiments, a virion of a recombinant NDV described herein comprises an antigen encoded by a transgene described herein.

In some embodiments, a virion of a recombinant NDV described herein comprises a non-NDV APMV F protein or a variant thereof. In certain embodiments, a virion of a recombinant NDV described herein comprises a non-NDV APMV HN protein or a variant thereof. In specific embodiments, a virion of a recombinant NDV described herein comprises a non-NDV APMV F protein or a variant thereof and a non-NDV APMV HN protein or a variant thereof. In some embodiments, a virion of a recombinant NDV described herein comprises a chimeric F protein described herein. In certain embodiments, a virion of a recombinant NDV described herein comprises a chimeric HN protein described herein. In specific embodiments, a virion of a recombinant NDV described herein comprises a chimeric F protein described herein and a chimeric HN protein described herein.

In some embodiments, the presence of a non-NDV APMV F protein or variant thereof (e.g., APMV-4 F protein) and/or a non-NDV APMV HN protein (e.g., APMV-4 HN protein) in the virion of a recombinant NDV confers a functional benefit, such as increased interferon (Type 1 interferon) induction in cells infected with the virus relative to NDV without the non-NDV APMV F protein or variant thereof and/or non-NDV APMV HN protein (e.g., an NDV strain described in Section 6, infra). In some embodiments, the presence of an APMV-4 F protein and APMV-4 HN protein in the virion of a recombinant NDV confers a functional benefit, such as increased interferon (Type 1 interferon) induction in cells infected with the virus relative to NDV without the APMV-4 F protein and/or APMV-4 HN protein (e.g., an NDV strain described in Section 6, infra). In certain embodiments, interferon induction is assessed in vitro in an assay, such as described herein (e.g., in Section 6, infra) or known to one of skill in the art.

### 5.1.1 Recombinant Nucleic Acid Sequence

In one aspect, provided herein is a nucleic acid sequence comprising a nucleotide sequence of a Newcastle disease virus genome in which the nucleotide sequence encoding the NDV HN protein has been replaced with a nucleotide sequence encoding the HN protein of an avian paramyxovirus (APMV) other than NDV or a variant of the non-NDV-APMV HN protein, or the nucleotide sequence encoding the NDV F protein has been replaced with a nucleotide sequence encoding the F protein of an APMV other than NDV or a variant of the non-NDV-APMV F protein. In certain instances herein, the term “non-NDV APMV” is used to refer to an APMV other than NDV. In one embodiment, provided herein is a nucleic acid sequence comprising a nucleotide sequence of a Newcastle disease virus genome in which (1) the nucleotide sequence encoding the NDV HN protein has been replaced with a nucleotide sequence encoding a non-NDV APMV HN protein or a variant thereof, wherein NDV intergenic regions are before and after the non-NDV APMV HN protein coding sequence or variant HN protein coding sequence; or (2) the nucleotide sequence encoding the NDV F protein has been replaced with a nucleotide sequence encoding a non-NDV APMV F protein or a variant thereof, wherein NDV intergenic regions are before and after the non-NDV APMV F protein coding sequence or variant F protein coding sequence. In specific embodiments, the NDV intergenic regions before and after the non-NDV APMV HN protein coding sequence or variant non-NDV APMV HN protein coding sequence are NDV HN intergenic regions. In specific embodiments, the NDV intergenic regions before and after the non-NDV APMV F protein coding sequence or variant non-NDV APMV F protein coding sequence are NDV F intergenic regions. In another embodiment, provided herein is a nucleic acid sequence comprising a nucleotide sequence of a Newcastle disease virus genome in which (1) the transcription unit encoding the NDV HN protein has been replaced with a transcription unit comprising a nucleotide sequence encoding a non-NDV APMV HN protein or a variant thereof; or (2) the transcription unit encoding the NDV F protein has been replaced with a transcription unit comprising a nucleotide sequence encoding a non-NDV APMV F protein or a variant thereof. In specific embodiments, the non-NDV APMV F protein or variant thereof has one or more, or all of the functions of NDV F protein required for NDV to replicate in vitro, in vivo or both. In specific embodiments, the non-NDV APMV HN protein or variant thereof has one or more, or all of the functions of NDV HN protein required for NDV to replicate in vitro, in vivo or both. The NDV genome typically comprises the N gene, P gene, L gene, M gene, HN gene, and F gene.

In another embodiment, provided herein is a nucleic acid sequence comprising a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV APMV HN protein coding sequence; or (2) the nucleotide sequence encoding the NDV F protein has been replaced with a nucleotide sequence encoding a non-NDV APMV F protein, wherein NDV intergenic regions are before and after the non-NDV APMV F protein coding sequence. In specific embodiments, the NDV intergenic regions before and after the non-NDV APMV HN protein coding sequence are NDV HN intergenic regions. In specific embodiments, the NDV intergenic regions before and after the non-NDV APMV F protein coding sequence are NDV F intergenic regions. In another embodiment, provided herein is a nucleic acid sequence comprising a nucleotide sequence of a Newcastle disease virus genome in which (1) the transcription unit encoding the NDV HN protein has been replaced with a transcription unit comprising a nucleotide sequence encoding a non-NDV APMV HN protein; or (2) the transcription unit encoding the NDV F protein has been replaced with a transcription unit comprising a nucleotide sequence encoding a non-NDV APMV F protein. In specific embodiments, the non-NDV APMV F protein has one or more, or all of the functions of NDV F protein required for NDV to replicate in vitro, in vivo or both. In specific embodiments, the non-NDV APMV HN protein has one or more, or all of the functions of NDV HN protein required for NDV to replicate in vitro, in vivo or both.

In another embodiment, provided herein is a nucleic acid sequence comprising a nucleotide sequence of a Newcastle disease virus genome in which (1) the nucleotide sequence encoding the NDV HN protein has been replaced with a nucleotide sequence encoding a variant of a non-NDV APMV HN protein, wherein NDV intergenic regions are before and after the variant HN protein coding sequence; or (2) the nucleotide sequence encoding the NDV F protein has been replaced with a nucleotide sequence encoding a variant of a non-NDV APMV F protein, wherein NDV intergenic regions are before and after the variant F protein coding sequence. In specific embodiments, the NDV intergenic regions before and after the variant of the non-NDV APMV HN protein coding sequence are NDV HN intergenic regions. In specific embodiments, the NDV intergenic regions before and after the variant of the non-NDV APMV F protein coding sequence are NDV F intergenic regions. In another embodiment, provided herein is a nucleic acid sequence comprising a nucleotide sequence of a Newcastle disease virus genome in which (1) the transcription unit encoding the NDV HN protein has been replaced with a transcription unit comprising a nucleotide sequence encoding a variant of a non-NDV APMV HN protein; or (2) the transcription unit encoding the NDV F protein has been replaced with a transcription unit comprising a nucleotide sequence encoding a variant of a non-NDV APMV F protein. In specific embodiments, the variant of the non-NDV APMV F protein has one or more, or all of the functions of NDV F protein required for NDV to replicate in vitro, in vivo or both. In specific embodiments, the variant of the non-NDV APMV HN protein has one or more, or all of the functions of NDV HN protein required for NDV to replicate in vitro, in vivo or both.

In another aspect, provided herein is a nucleic acid sequence comprising a nucleotide sequence of a Newcastle disease virus genome in which the nucleotide sequence encoding the NDV HN protein has been replaced with a nucleotide sequence encoding the HN protein of an avian paramyxovirus (APMV) other than NDV or a variant of the non-NDV-APMV HN protein, and the nucleotide sequence encoding the NDV F protein has been replaced with a nucleotide sequence encoding the F protein of an APMV other than NDV or a variant of the non-NDV-APMV F protein. In one embodiment, provided herein is a nucleic acid sequence comprising a nucleotide sequence of a Newcastle disease virus genome in which (1) the nucleotide sequence encoding the NDV HN protein has been replaced with a nucleotide sequence encoding a non-NDV APMV HN protein or a variant thereof, and (2) the nucleotide sequence encoding the NDV F protein has been replaced with a nucleotide sequence encoding a non-NDV APMV F protein or a variant thereof, wherein NDV intergenic regions are before, in between and after the non-NDV APMV HN and F protein coding sequences or variant HN and F protein coding sequences. In specific embodiments, the NDV intergenic regions before and after the non-NDV APMV HN protein coding sequence or variant non-NDV APMV HN protein coding sequence are NDV HN intergenic regions. In specific embodiments, the NDV intergenic regions before and after the non-NDV APMV F protein coding sequence or variant non-NDV APMV F protein coding sequence are NDV F intergenic regions. In another embodiment, provided herein is a nucleic acid sequence comprising a nucleotide sequence of a Newcastle disease virus genome in which (1) the transcription unit encoding the NDV HN protein has been replaced with a transcription unit comprising a nucleotide sequence encoding a non-NDV APMV HN protein or a variant thereof; and (2) the transcription unit encoding the NDV F protein has been replaced with a transcription unit comprising a nucleotide sequence encoding a non-NDV APMV F protein or a variant thereof. In specific embodiments, the non-NDV APMV F protein or variant thereof has one or more, or all of the functions of NDV F protein required for NDV to replicate in vitro, in vivo or both. In specific embodiments, the non-NDV APMV HN protein or variant thereof has one or more, or all of the functions of NDV HN protein required for NDV to replicate in vitro, in vivo or both.

In another embodiment, provided herein is a nucleic acid sequence comprising a nucleotide sequence of a Newcastle disease virus genome in which (1) the nucleotide sequence encoding the NDV HN protein has been replaced with a nucleotide sequence encoding a non-NDV APMV HN protein; and (2) the nucleotide sequence encoding the NDV F protein has been replaced with a nucleotide sequence encoding a non-NDV APMV F protein, wherein NDV intergenic regions are before, in between and after the non-NDV APMV HN and F protein coding sequences. In specific embodiments, the NDV intergenic regions before and after the nucleotide sequence encoding the non-NDV APMV HN protein are NDV HN intergenic regions. In specific embodiments, the NDV intergenic regions before and after the nucleotide sequence encoding the non-NDV APMV F protein are NDV F intergenic regions. In another embodiment, provided herein is a nucleic acid sequence comprising a nucleotide sequence of a Newcastle disease virus genome in which (1) the transcription unit encoding the NDV HN protein has been replaced with a transcription unit comprising a nucleotide sequence encoding a non-NDV APMV HN protein; and (2) the transcription unit encoding the NDV F protein has been replaced with a transcription unit comprising a nucleotide sequence encoding a non-NDV APMV F protein. In specific embodiments, the non-NDV APMV HN and F proteins are found in nature in the same strain of APMV. For example, the non-NDV APMV HN and F proteins may both be found in nature in the same APMV-15 strain. In other embodiments, the non-NDV APMV HN and F proteins are found in nature in the different strains of APMV. In specific embodiments, the non-NDV AMPV F protein has one or more, or all of the functions of NDV F protein required for NDV to replicate in vitro, in vivo or both. In specific embodiments, the non-NDV AMPV HN protein has one or more, or all of the functions of NDV HN protein required for NDV to replicate in vitro, in vivo or both.

In another embodiment, provided herein is a nucleic acid sequence comprising a nucleotide sequence of a Newcastle disease virus genome in which (1) the nucleotide sequence encoding the NDV HN protein has been replaced with a nucleotide sequence encoding a variant of a non-NDV APMV HN protein; and (2) the nucleotide sequence encoding the NDV F protein has been replaced with a nucleotide sequence encoding a variant of a non-NDV APMV F protein, wherein NDV intergenic regions are before, in between and after the variant HN and F protein coding sequences. In specific embodiments, the NDV intergenic regions before and after the nucleotide sequence encoding the variant of the non-NDV APMV HN protein are NDV HN intergenic regions. In specific embodiments, the NDV intergenic regions before and after the nucleotide sequence encoding the variant of the non-NDV APMV F protein are NDV F intergenic regions. In another embodiment, provided herein is a nucleic acid sequence comprising a nucleotide sequence of a Newcastle disease virus genome in which (1) the transcription unit encoding the NDV HN protein has been replaced with a transcription unit comprising a nucleotide sequence encoding a variant of a non-NDV APMV HN protein; and (2) the transcription unit encoding the NDV F protein has been replaced with a transcription unit comprising a nucleotide sequence encoding a variant of a non-NDV APMV F protein. In specific embodiments, the variant HN and F proteins are derived from the same strain of APMV. For example, the variant HN and F proteins may both be derived from the same APMV-15 strain. In other embodiments, the variant HN and F proteins are derived from different strains of APMV. In specific embodiments, the variant of the non-NDV APMV F protein has one or more, or all of the functions of NDV F protein required for NDV to replicate in vitro, in vivo or both. In specific embodiments, the variant of the non-NDV APMV HN protein has one or more, or all of the functions of NDV HN protein required for NDV to replicate in vitro, in vivo or both.

In another aspect, provided herein is a nucleic acid sequence comprising a nucleotide sequence of a Newcastle disease virus genome in which the nucleotide sequence encoding the NDV HN protein has been replaced with a nucleotide sequence encoding a chimeric HN protein, or the nucleotide sequence encoding the NDV F protein has been replaced with a nucleotide sequence encoding a chimeric F protein. In one embodiment, provided herein is a nucleic acid sequence comprising a nucleotide sequence of a Newcastle disease virus genome in which (1) the nucleotide sequence encoding the NDV HN protein has been replaced with a nucleotide sequence encoding a chimeric HN protein, wherein NDV intergenic regions are before and after the chimeric HN protein coding sequence; or (2) the nucleotide sequence encoding the NDV F protein has been replaced with a nucleotide sequence encoding a chimeric F protein, wherein NDV intergenic regions are before and after the chimeric F protein coding sequence. In specific embodiments, the NDV intergenic regions before and after the nucleotide sequence encoding the chimeric HN protein are NDV HN intergenic regions. In specific embodiments, the NDV intergenic regions before and after the nucleotide sequence encoding the chimeric F protein are NDV F intergenic regions. In another embodiment, provided herein is a nucleic acid sequence comprising a nucleotide sequence of a Newcastle disease virus genome in which (1) the transcription unit encoding the NDV HN protein has been replaced with a transcription unit comprising a nucleotide sequence encoding a chimeric HN protein; or (2) the transcription unit encoding the NDV F protein has been replaced with a transcription unit comprising a nucleotide sequence encoding a chimeric F protein. In specific embodiments, the chimeric HN protein comprises a non-NDV APMV HN protein ectodomain and NDV HN protein transmembrane and cytoplasmic domains. In other words, the NDV HN protein transmembrane and cytoplasmic domains replace the non-NDV APMV HN protein transmembrane and cytoplasmic domains so that the chimeric HN protein does not include the non-NDV APMV HN protein transmembrane and cytoplasmic domains. The ectodomain, transmembrane and cytoplasmic domains of the non-NDV APMV HN protein and NDV HN protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the non-NDV APMV HN protein and NDV HN protein. In specific embodiments, the chimeric HN protein comprises an ectodomain of a variant of a non-NDV APMV HN protein and NDV HN protein transmembrane and cytoplasmic domains. In specific embodiments, the chimeric HN protein has one or more, or all of the functions of NDV HN required for NDV to replicate in vitro, in vivo or both. In specific embodiments, the chimeric F protein comprises a non-NDV APMV F protein ectodomain and NDV F protein transmembrane and cytoplasmic domains. In other words, the NDV F protein transmembrane and cytoplasmic domains replace the non-NDV APMV F protein transmembrane and cytoplasmic domains so that the chimeric F protein does not include the non-NDV APMV F protein transmembrane and cytoplasmic domains. The ectodomain, transmembrane and cytoplasmic domains of the non-NDV APMV F protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the non-NDV APMV F protein and NDV F protein. In specific embodiments, the chimeric F protein comprises an ectodomain of a variant of a non-NDV APMV F protein and NDV F protein transmembrane and cytoplasmic domains. In specific embodiments, the chimeric F protein has one or more, or all of the functions of NDV F protein required for NDV to replicate in vitro, in vivo or both.

In another aspect, provided herein is a nucleic acid sequence comprising a nucleotide sequence of a Newcastle disease virus genome in which the nucleotide sequence encoding the NDV HN protein has been replaced with a nucleotide sequence encoding a chimeric HN protein, and the nucleotide sequence encoding the NDV F protein has been replaced with a nucleotide sequence encoding a chimeric F protein. In one embodiment, provided herein is a nucleic acid sequence comprising a nucleotide sequence of a Newcastle disease virus genome in which (1) the nucleotide sequence encoding the NDV HN protein has been replaced with a nucleotide sequence encoding a chimeric HN protein, wherein NDV intergenic regions are before and after the chimeric HN protein coding sequence; and (2) the nucleotide sequence encoding the NDV F protein has been replaced with a nucleotide sequence encoding a chimeric F protein, wherein NDV intergenic regions are before and after the chimeric F protein coding sequence. In specific embodiments, the NDV intergenic regions before and after the nucleotide sequence encoding the chimeric HN protein are NDV HN intergenic regions. In specific embodiments, the NDV intergenic regions before and after the nucleotide sequence encoding the chimeric F protein are NDV F intergenic regions. In another embodiment, provided herein is a nucleic acid sequence comprising a nucleotide sequence of a Newcastle disease virus genome in which (1) the transcription unit encoding the NDV HN protein has been replaced with a transcription unit comprising a nucleotide sequence encoding a chimeric HN protein; and (2) the transcription unit encoding the NDV F protein has been replaced with a transcription unit comprising a nucleotide sequence encoding a chimeric F protein. In specific embodiments, the chimeric HN protein comprises a non-NDV APMV HN protein ectodomain and NDV HN protein transmembrane and cytoplasmic domains. In other words, the NDV HN protein transmembrane and cytoplasmic domains replace the non-NDV APMV HN protein transmembrane and cytoplasmic domains so that the chimeric HN protein does not include the non-NDV APMV HN protein transmembrane and cytoplasmic domains. The ectodomain, transmembrane and cytoplasmic domains of the non-NDV APMV HN protein and NDV HN protein may be determined using techniques known to one of skill in the art or described herein. In specific embodiments, the chimeric HN protein has one or more, or all of the functions of NDV HN required for NDV to replicate in vitro, in vivo or both. In specific embodiments, the chimeric HN protein comprises an ectodomain of a variant of a non-NDV APMV HN protein and NDV HN protein transmembrane and cytoplasmic domains. In specific embodiments, the chimeric F protein comprises a non-NDV APMV F protein ectodomain and NDV F protein transmembrane and cytoplasmic domains. In other words, the NDV F protein transmembrane and cytoplasmic domains replace the non-NDV APMV F protein transmembrane and cytoplasmic domains so that the chimeric F protein does not include the non-NDV APMV F protein transmembrane and cytoplasmic domains. The ectodomain, transmembrane and cytoplasmic domains of the non-NDV APMV F protein and NDV F protein may be determined using techniques known to one of skill in the art or described herein. In specific embodiments, the chimeric F protein comprises an ectodomain of a variant of a non-NDV APMV F protein and NDV F protein transmembrane and cytoplasmic domains. In specific embodiments, the chimeric F protein has one or more, or all of the functions of NDV F protein required for NDV to replicate in vitro, in vivo or both. In specific embodiments, the ectodomains of the non-NDV APMV HN and F proteins are found in nature in the same strain of APMV. For example, the ectodomains of the non-NDV APMV HN and F proteins may both be found in nature in the same APMV-15 strain. In other embodiments, the ectodomains of the non-NDV APMV HN and F proteins are found in nature in the different strains of APMV.

In another aspect, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV fusion (F) protein, (5) a transcription unit encoding a non-NDV APMV hemagglutinin-neuraminidase (HN) or a variant thereof, and (6) a transcription unit encoding a NDV large polymerase (L). In another aspect, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a non-NDV APMV fusion (F) protein or a variant thereof, (5) a transcription unit encoding a NDV hemagglutinin-neuraminidase (HN), and (6) a transcription unit encoding a NDV large polymerase (L). In specific embodiments, the non-NDV AMPV F protein or variant thereof has one or more, or all of the functions of NDV F protein required for NDV to replicate in vitro, in vivo or both. In specific embodiments, the non-NDV APMV HN protein or variant thereof has one or more, or all of the functions of NDV HN protein required for NDV to replicate in vitro, in vivo or both.

In another aspect, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a non-NDV APMV fusion (F) protein or a variant thereof, (5) a transcription unit encoding a non-NDV APMV hemagglutinin-neuraminidase (HN) or a variant thereof, and (6) a transcription unit encoding a NDV large polymerase (L). In specific embodiments, the non-NDV AMPV F protein or variant thereof has one or more, or all of the functions of NDV F protein required for NDV to replicate in vitro, in vivo or both. In specific embodiments, the non-NDV APMV HN protein or variant thereof has one or more, or all of the functions of NDV HN protein required for NDV to replicate in vitro, in vivo or both.

In one embodiment, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a non-NDV APMV fusion (F) protein, (5) a transcription unit encoding a non-NDV APMV hemagglutinin-neuraminidase (HN), and (6) a transcription unit encoding a NDV large polymerase (L). In specific embodiments, the non-NDV APMV HN and F proteins are found in nature in the same strain of APMV. For example, the non-NDV APMV HN and F proteins may both be found in nature in the same APMV-15 strain. In other embodiments, the non-NDV APMV HN and F proteins are found in nature in the different strains of APMV. In specific embodiments, the non-NDV APMV F protein has one or more, or all of the functions of NDV F protein required for NDV to replicate in vitro, in vivo or both. In specific embodiments, the non-NDV APMV HN protein has one or more, or all of the functions of NDV HN protein required for NDV to replicate in vitro, in vivo or both.

In another embodiment, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a variant of a non-NDV APMV fusion (F) protein, (5) a transcription unit encoding a variant of a non-NDV APMV hemagglutinin-neuraminidase (HN), and (6) a transcription unit encoding a NDV large polymerase (L). In specific embodiments, the variants of the non-NDV APMV HN and F proteins are derived from the same strain of APMV. For example, the variants of the non-NDV APMV HN and F proteins may both be derived from the same APMV-15 strain. In other embodiments, the variants of the non-NDV APMV HN and F proteins are derived from the different strains of APMV. In specific embodiments, the variant of the non-NDV APMV F protein has one or more, or all of the functions of NDV F protein required for NDV to replicate in vitro, in vivo or both. In specific embodiments, the variant of the non-NDV APMV HN protein has one or more, or all of the functions of NDV HN protein required for NDV to replicate in vitro, in vivo or both.

In another aspect, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV fusion (F) protein, (5) a transcription unit encoding a chimeric hemagglutinin-neuraminidase (HN), and (6) a transcription unit encoding a NDV large polymerase (L). In another aspect, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a chimeric fusion (F) protein, (5) a transcription unit encoding a NDV hemagglutinin-neuraminidase (HN), and (6) a transcription unit encoding a NDV large polymerase (L). In another aspect, provided herein is a nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a chimeric fusion (F) protein, (5) a transcription unit encoding a chimeric hemagglutinin-neuraminidase (HN), and (6) a transcription unit encoding a NDV large polymerase (L). In specific embodiments, the chimeric HN protein comprises a non-NDV APMV HN protein ectodomain and NDV HN protein transmembrane and cytoplasmic domains. In other words, the NDV HN protein transmembrane and cytoplasmic domains replace the non-NDV APMV HN protein transmembrane and cytoplasmic domains so that the chimeric HN protein does not include the non-NDV APMV HN protein transmembrane and cytoplasmic domains. The ectodomain, transmembrane and cytoplasmic domains of the non-NDV APMV HN protein and NDV HN protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the non-NDV APMV HN protein and NDV HN protein. In specific embodiments, the chimeric HN protein has one or more, or all of the functions of NDV HN required for NDV to replicate in vitro, in vivo or both. In specific embodiments, the chimeric F protein comprises a non-NDV APMV F protein ectodomain and NDV F protein transmembrane and cytoplasmic domains. In other words, the NDV F protein transmembrane and cytoplasmic domains replace the non-NDV APMV F protein transmembrane and cytoplasmic domains so that the chimeric F protein does not include the non-NDV APMV F protein transmembrane and cytoplasmic domains. The ectodomain, transmembrane and cytoplasmic domains of the non-NDV APMV F protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the non-NDV APMV F protein and NDV F protein. In specific embodiments, the chimeric F protein has one or more, or all of the functions of NDV F protein required for NDV to replicate in vitro, in vivo or both. In specific embodiments, the ectodomains of the non-NDV APMV HN and F proteins are found in nature in the same strain of APMV. For example, the ectodomains of the non-NDV APMV HN and F proteins may both be found in nature in the same APMV-15 strain. In other embodiments, the ectodomains of the non-NDV APMV HN and F proteins are found in nature in the different strains of APMV.

In specific embodiments, the non-NDV APMV is immunologically distinct from NDV. In certain embodiments, a non-NDV APMV is immunologically distinct from NDV if the non-NDV APMV and NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a non-NDV APMV is considered immunologically distinct from NDV if the non-NDV APMV and NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In a specific embodiment, a non-NDV APMV is considered immunologically distinct from NDV if NDV antiserum HI activity is significantly reduced against the non-NDV APMV in an HI assay, such as described below (e.g., in Example 3). In a specific embodiment, a non-NDV APMV is considered immunologically distinct from NDV if NDV antiserum HI activity is reduced by at least 2 fold, 3 fold, 4 fold, 5 fold, 10 fold, or more against the non-NDV APMV in an HI assay, such as described below (e.g., in Example 3), relative the NDV antiserum HI activity against NDV. In certain embodiments, the non-NDV APMV is AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, the non-NDV APMV is an APMV-2, such as, e.g., Chicken/California/Yucaipa/56 (Accession No. EU338414). In certain embodiments, the non-NDV APMV is APMV-3, such as, e.g., APMV3/Turkey/Wisconsin/68 (Accession No. EU782025). In some embodiments, the non-NDV APMV is APMV-4, such as, e.g., APMV4/duck/Hongkong/D3/75 (Accession No. FJ177514), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, the non-NDV APMV is an APMV-5, such as, e.g., APMV-5 budgerigar/Kunitachi/74 (Accession No. GU206351) or APMV5/budgerigar/Japan/TI/75 (Accession No. LC168750). In some embodiments, the non-NDV APMV is APMV-6, such as, e.g., APMV-6 Goose/FarEast/4440/2003 (Accession No. EF569970) or APMV6/duck/HongKong/18/199/77 (Accession No. EU622637). In certain embodiments, the non-NDV APMV is APMV-7, such as, e.g., APMV-7 dove/Tennessee/4/75 (Accession No. FJ231524). In some embodiments, the non-NDV APMV is APMV-8, such as, e.g., APMV-8 goose/Delaware/1053/76 (Accession No. FJ215863). In certain embodiments, the non-NDV APMV is APMV-9, such as, e.g., APMV9/duck/New York/22/78 (Accession No. EU910942). In some embodiments, the non-NDV APMV is APMV-10, such as, e.g., APMV-10 penguin/Falkland Islands/324/2007 (Accession No. HM147142 or NC_025349). In certain embodiments, the non-NDV APMV is APMV-11, such as, e.g., APMV-11 common_snipe/France/100212/2010 (Accession No. JQ886184). In some embodiments, the non-NDV APMV is APMV-12, such as, e.g., APMV12/Wigeon/Italy/3920_1/05 (Accession No. KC333050). In some embodiments, the non-NDV APMV is APMV-14, such as, e.g., APMV-14 duck/Japan/110G0352/2011 (Accession No. KX258200). In certain embodiments, the non-NDV APMV is APMV-15, such as, e.g., APMV-15 calidris_fuscicollis/Brazil/RS-1177/2012 (Accession No. KX932454). In some embodiments, the non-NDV APMV is APMV-17, such as, e.g., APMV17/Antarctica/107/13 (Accession No. MK167211). In some embodiments, the non-NDV APMV is APMV-20, such as, e.g., APMV-20 Gull/Kazakhstan/2014 (Accession No. MF033136). In certain embodiments, the non-NDV APMV is APMV-21, such as, e.g., APMV21/pigeon/Taiwan/AHRI128/17 (Accession No. MK67743).

In a specific embodiment, the non-NDV APMV is APMV4/duck/Hongkong/D3/75 (Accession No. FJ177514). In another specific embodiment, the non-NDV APMV is APMV17/Antarctica/107/13 (Accession No. MK167211). In another specific embodiment, the non-NDV APMV is APMV9/duck/New York/22/78 (Accession No. EU910942). In another specific embodiment, the non-NDV is APMV7/dove/Tennessee/4/75 (Accession No. FJ231524). In another specific embodiment, the non-NDV APMV is APMV21/pigeon/Taiwan/AHRI128/17 (Accession No. MK67743). In another specific embodiment, the non-NDV APMV is APMV6/duck/HongKong/18/199/77 (Accession No. EU622637). In another specific embodiment, the non-NDV APMV is APMV11/common_snipe/France/100212/10 (Accession No. JQ886184). In another specific embodiment, the non-NDV APMV is APMV15/calidris_fuscicollis/Brazil/RS-1177/12 (Accession No. NC_034968). In another specific embodiment, the non-NDV APMV is APMV8/Goose/Delaware/1053/76 (Accession No. FJ215863). In another specific embodiment, the non-NDV APMV is APMV2/Chicken/California/Yucaipa/56 (Accession No. EU338414). In another specific embodiment, the non-NDV APMV is APMV3/Turkey/Wisconsin/68 (Accession No. EU782025). In another specific embodiment, the non-NDV APMV is APMV12/Wigeon/Italy/3920_1/05 (Accession No. KC333050). In another specific embodiment, the non-NDV APMV is APMV5/budgerigar/Japan/TI/75 (Accession No. LC168750). In another specific embodiment, the non-NDV APMV is APMV10/penguin/Falkland Islands/324/07 (Accession No. NC_025349).

In some embodiments, the non-NDV APMV is a member of the subfamily Avulavirinae from a different genus than NDV. In some embodiments, the non-NDV APMV is from a member of the subfamily Avulavirinae, but is not NDV. In some embodiments, the non-NDV APMV is a member of the subfamily Avulavirinae and the genus Metaavulavirus. In some embodiments, the non-NDV APMV is a member of the subfamily Avulavirinae and the genus paraavulavirus. In some embodiments, the non-NDV APMV is a member of the subfamily Avulavirinae and the genus orthoavulavirus but is not NDV.

In certain embodiments, a non-NDV APMV F protein is immunologically distinct from an NDV F protein. In certain embodiments, a variant of a non-NDV APMV F protein is immunologically distinct from an NDV F protein. In certain embodiments, a non-NDV APMV F protein or a variant thereof is immunologically distinct from an NDV F protein if antibodies directed to the NDV F protein do not cross-react with the non-NDV APMV F protein or variant thereof. In some embodiments, a non-NDV APMV F protein or a variant thereof is immunologically distinct from an NDV F protein if antibodies directed to the NDV F protein bind to the non-NDV APMV F protein or variant thereof with a 2-fold, 5-fold, 10-fold, 15-fold, 20-fold or lower affinity than to NDV F protein in an assay known to one of skill in the art or described herein. In certain embodiments, a non-NDV APMV F protein or a variant thereof is immunologically distinct from an NDV F protein if antibodies directed to the NDV F protein bind to the non-NDV APMV F protein or variant thereof with a 0.5 log, 1 log, 1.5 log, 2 log, 2.5 log, 3 log or lower affinity than to NDV F protein in an assay known to one of skill in the art or described herein. In certain embodiments, a non-NDV APMV F protein or a variant thereof is immunologically distinct from an NDV F protein if anti-NDV F antibodies do not substantially inhibit replication of NDV expressing the non-NDV APMV F protein or a variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a non-NDV APMV F protein or a variant thereof is immunologically distinct from an NDV F protein if anti-NDV F antibodies inhibit replication of NDV expressing the non-NDV APMV F protein or variant thereof in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs.

In some embodiments, a non-NDV APMV F protein is an F protein from a member of the subfamily Avulavirinae, but not NDV. In some embodiments, a non-NDV APMV F protein is an F protein from a member of the subfamily Avulavirinae and the genus Metaavulavirus. In some embodiments, a non-NDV APMV F protein is an F protein from a member of the subfamily Avulavirinae and genus paraavulavirus. In some embodiments, a non-NDV APMV F protein is an F protein from a member of the subfamily Avulavirinae and the genus orthoavulavirus but is not NDV.

In certain embodiments, a chimeric F protein is immunologically distinct from an NDV F protein. In certain embodiments, a chimeric F protein is immunologically distinct from an NDV F protein if antibodies directed to the NDV F protein do not cross-react with the chimeric F protein. In some embodiments, a chimeric F protein is immunologically distinct from an NDV F protein if antibodies directed to the NDV F protein bind to the chimeric F protein with a 2-fold, 5-fold, 10-fold, 15-fold, 20-fold or lower affinity than to NDV F protein in an assay known to one of skill in the art or described herein. In certain embodiments, a chimeric F protein is immunologically distinct from an NDV F protein if antibodies directed to the NDV F protein bind to the chimeric F protein with a 0.5 log, 1 log, 1.5 log, 2 log, 2.5 log, 3 log or lower affinity than to NDV F protein in an assay known to one of skill in the art or described herein. In certain embodiments, a chimeric F protein is immunologically distinct from an NDV F protein if anti-NDV F antibodies do not substantially inhibit replication of NDV expressing the non-NDV APMV F protein or a variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a chimeric F protein is immunologically distinct from an NDV F protein if anti-NDV F antibodies inhibit replication of NDV expressing the chimeric F protein in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs.

In specific embodiments, a non-NDV APMV F protein does not contain a multibasic cleavage site. In certain embodiments, a non-NDV APMV F protein is modified by, e.g., one or more amino acid substitutions so that the non-NDV APMV F protein no longer contains a multi-basic cleavage. In some embodiments, the original sequence of the cleavage site of the non-NDV APMV F protein is modified by, e.g., one or more amino acid substitutions. For example, a leucine at the amino acid position of the non-NDV APMV F protein corresponding to amino acid position 289 of NDV F protein (as counted by the LaSota strain F protein) may be substituted for alanine to eliminate a multi-basic cleavage site.

In specific embodiments, a variant of a non-NDV APMV F protein does not contain a multibasic cleavage site. In certain embodiments, a variant of a non-NDV APMV F protein includes one or more amino acid substitutions so that the non-NDV APMV F protein no longer contains a multi-basic cleavage. In some embodiments, the original sequence of the cleavage site of the variant of the non-NDV APMV F protein is modified by, e.g., one or more amino acid substitutions. For example, a variant of a non-NDV APMV F protein includes an amino acid substitution of alanine for leucine at the amino acid position of the non-NDV APMV F protein corresponding to amino acid position 289 of NDV F protein (as counted by the LaSota strain F protein).

In specific embodiments, a chimeric F protein does not contain a multibasic cleavage site. In certain embodiments, a chimeric F protein includes one or more amino acid substitutions so that the ectodomain of the non-NDV APMV F protein no longer contains a multi-basic cleavage. In some embodiments, the original sequence of the cleavage site of the ectodomain of the non-NDV APMV F protein is modified by, e.g., one or more amino acid substitutions. For example, a chimeric protein includes an amino acid substitution of alanine for leucine at the amino acid position of the ectodomain of the non-NDV APMV F protein corresponding to amino acid position 289 of NDV F protein (as counted by the LaSota strain F protein).

In specific embodiments, a variant of a non-NDV APMV F protein retains one or more functions of the non-NDV APMV F protein.

In certain embodiments, a variant of a non-NDV APMV F protein is at least 75%, at least 80%, or at least 85% identical to the non-NDV AMPV F protein. In some embodiments, a variant of a non-NDV APMV F protein is at least 90%, at least 95%, or at least 99% identical to the non-NDV APMV F protein. In certain embodiments, a variant of a non-NDV APMV F protein is 75% to 90%, 80% to 95% or 90% to 99.5% identical to the non-NDV AMPV F protein.

In certain embodiment, a variant of a non-NDV APMV F protein contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a non-NDV APMV F protein. In some embodiments, a variant of a non-NDV APMV F protein comprises the amino acid sequence of the non-NDV APMV F protein with 20, 15, 10, 9, 8, 7, 6, 5, 4, 3, 2 or 1 amino acid residues of the non-NDV APMV F protein substituted (e.g., conservatively substituted) with other amino acids. In certain embodiments, a variant of a non-NDV APMV F protein comprises the amino acid sequence of the non-NDV APMV F protein with up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In some embodiments, a variant of a non-NDV APMV F protein is a polypeptide encoded by nucleic acid sequence that can hybridize under high, moderate or typical stringency hybridization conditions to a nucleic acid sequence encoding the non-NDV APMV F protein. Hybridization conditions are known to one of skill in the art (see, e.g., U.S. Patent Application No. 2005/0048549 at, e.g., paragraphs 72 and 73).

In some embodiments, a non-NDV APMV F protein is the F protein of an APMV shown in FIG. 3A. In some embodiments, a non-NDV APMV F protein is the F protein of a member of a genus shown in FIG. 3A or FIG. 6A. In certain embodiments, a non-NDV APMV F protein is the F protein of AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, a non-NDV APMV F protein is the F protein of APMV-2, such as, e.g., Chicken/California/Yucaipa/56 (Accession No. EU338414). In certain embodiments, a non-NDV APMV F protein is the F protein of APMV-2 Yucaipa. In other embodiments, non-NDV APMV F protein is not the F protein of APMV-2 Yucaipa. In certain embodiments, a non-NDV APMV F protein is the F protein of APMV-3, such as, e.g., APMV3/Turkey/Wisconsin/68 (Accession No. EU782025). In some embodiments, a non-NDV APMV F protein is the F protein of APMV-4, such as, e.g., APMV4/duck/Hongkong/D3/75 (Accession No. FJ177514),), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, a non-NDV APMV F protein is the F protein of APMV-5, such as, e.g., APMV-5 budgerigar/Kunitachi/74 (Accession No. GU206351) or APMV5/budgerigar/Japan/TI/75 (Accession No. LC168750). In some embodiments, a non-NDV APMV F protein is the F protein of APMV-6, such as, e.g., APMV-6 Goose/FarEast/4440/2003 (Accession No. EF569970) or APMV6/duck/HongKong/18/199/77 (Accession No. EU622637). In certain embodiments, a non-NDV APMV F protein is the F protein of APMV-7, such as, e.g., APMV-7 dove/Tennessee/4/75 (Accession No. FJ231524). In some embodiments, a non-NDV APMV F protein is the F protein of APMV-8, such as, e.g., APMV-8 goose/Delaware/1053/76 (Accession No. FJ215863). In certain embodiments, a non-NDV APMV F protein is the F protein of APMV-9, such as, e.g., APMV9/duck/New York/22/78 (Accession No. EU910942). In some embodiments, a non-NDV APMV F protein is the F protein of APMV-10, such as, e.g., APMV-10 penguin/Falkland Islands/324/2007 (Accession No. HM147142 or NC_025349). In certain embodiments, a non-NDV APMV F protein is the F protein of APMV-11, such as, e.g., APMV-11 common_snipe/France/100212/2010 (Accession No. JQ886184). In some embodiments, a non-NDV APMV F protein is the F protein of APMV-12, such as, e.g., APMV12/Wigeon/Italy/3920_1/05 (Accession No. KC333050). In some embodiments, a non-NDV APMV F protein is the F protein of APMV-14, such as, e.g., APMV-14 duck/Japan/110G0352/2011 (Accession No. KX258200). In certain embodiments, a non-NDV APMV F protein is the F protein of APMV-15, such as, e.g., APMV-15 calidris_fuscicollis/Brazil/RS-1177/2012 (Accession No. KX932454). In some embodiments, a non-NDV APMV F protein is the F protein of APMV-17, such as, e.g., APMV17/Antarctica/107/13 (Accession No. MK167211). In some embodiments, a non-NDV APMV F protein is the F protein of APMV-20, such as, e.g., APMV-20 Gull/Kazakhstan/2014 (Accession No. MF033136). In certain embodiments, a non-NDV APMV F protein is the F protein of APMV-21, such as, e.g., APMV21/pigeon/Taiwan/AHRI128/17 (Accession No. MK67743).

In certain embodiments, a variant of a non-NDV APMV F protein is a variant of the F protein of AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, a variant of a non-NDV APMV F protein is a variant of the F protein of APMV-2, such as, e.g., Chicken/California/Yucaipa/56 (Accession No. EU338414). In certain embodiments, a variant of a non-NDV APMV F protein is a variant of the F protein of APMV-3, such as, e.g., APMV3/Turkey/Wisconsin/68 (Accession No. EU782025). In some embodiments, a variant of a non-NDV APMV F protein is a variant of the F protein of APMV-4, such as, e.g., APMV4/duck/Hongkong/D3/75 (Accession No. FJ177514),), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, a variant of a non-NDV APMV F protein is a variant of the F protein of APMV-5, such as, e.g., APMV-5 budgerigar/Kunitachi/74 (Accession No. GU206351) or APMV5/budgerigar/Japan/TI/75 (Accession No. LC168750). In some embodiments, a variant of a non-NDV APMV F protein is a variant of the F protein of APMV-6, such as, e.g., APMV-6 Goose/FarEast/4440/2003 (Accession No. EF569970) or APMV6/duck/HongKong/18/199/77 (Accession No. EU622637). In certain embodiments, a variant of a non-NDV APMV F protein is a variant of the F protein of APMV-7, such as, e.g., APMV-7 dove/Tennessee/4/75 (Accession No. FJ231524). In some embodiments, a variant of a non-NDV APMV F protein is a variant of the F protein of APMV-8, such as, e.g., APMV-8 goose/Delaware/1053/76 (Accession No. FJ215863). In certain embodiments, a variant of a non-NDV APMV F protein is a variant of the F protein of APMV-9, such as, e.g., APMV9/duck/New York/22/78 (Accession No. EU910942). In some embodiments, a variant of a non-NDV APMV F protein is a variant of the F protein of APMV-10, such as, e.g., APMV-10 penguin/Falkland Islands/324/2007 (Accession No. HM147142 or NC_025349). In certain embodiments, a variant of a non-NDV APMV F protein is a variant of the F protein of APMV-11, such as, e.g., APMV-11 common_snipe/France/100212/2010 (Accession No. JQ886184). In some embodiments, a variant of a non-NDV APMV F protein is a variant of the F protein of APMV-12, such as, e.g., APMV12/Wigeon/Italy/3920_1/05 (Accession No. KC333050). In some embodiments, a variant of a non-NDV APMV F protein is a variant of the F protein of APMV-14, such as, e.g., APMV-14 duck/Japan/110G0352/2011 (Accession No. KX258200). In certain embodiments, a variant of a non-NDV APMV F protein is a variant of the F protein of APMV-15, such as, e.g., APMV-15 calidris_fuscicollis/Brazil/RS-1177/2012 (Accession No. KX932454). In some embodiments, a variant of a non-NDV APMV F protein is a variant of the F protein of APMV-17, such as, e.g., APMV17/Antarctica/107/13 (Accession No. MK167211). In some embodiments, a variant of a non-NDV APMV F protein is a variant of the F protein of APMV-20, such as, e.g., APMV-20 Gull/Kazakhstan/2014 (Accession No. MF033136). In certain embodiments, a variant of a non-NDV APMV F protein is a variant of the F protein of APMV-21, such as, e.g., APMV21/pigeon/Taiwan/AHRI128/17 (Accession No. MK67743).

In some embodiments, a non-NDV APMV F protein has less than 65% identity to an NDV F protein. In some embodiments, a non-NDV APMV F protein has less than 60% identity to an NDV F protein. In some embodiments, a non-NDV APMV F protein has less than 50% identity to an NDV F protein. In some embodiments, a non-NDV APMV F protein has less than 55% identity to an NDV F protein. In some embodiments, a non-NDV APMV F protein has less than 50% identity to an NDV F protein. In some embodiments, a non-NDV APMV F protein has less than 45% identity to an NDV F protein. In some embodiments, a non-NDV APMV F protein has less than 40% identity to an NDV F protein. In some embodiments, a non-NDV APMV F protein has less than 35% identity to an NDV F protein. In some embodiments, a non-NDV APMV F protein has at least 20% or at least 25% identity to an NDV F protein but less than 65%, less than 60%, less than 55%, less than 50%, or less than 45% identity. In some embodiments, the NDV F protein is the NDV LaSota HN protein.

In certain embodiments, a non-NDV APMV HN protein or a variant thereof is immunologically distinct from an NDV HN protein. In certain embodiments, a non-NDV APMV HN protein or a variant thereof is immunologically distinct from an NDV HN protein if antibodies directed to the NDV HN protein do not cross-react with the non-NDV APMV HN protein or variant thereof. In some embodiments, a non-NDV APMV HN protein or a variant thereof is immunologically distinct from an NDV HN protein if antibodies directed to the NDV HN protein bind to the variant with a 2-fold, 5-fold, 10-fold, 15-fold, 20-fold or lower affinity than to the non-NDV APMV HN protein or variant thereof in an assay known to one of skill in the art or described herein. In certain embodiments, a non-NDV APMV HN protein or a variant thereof is immunologically distinct from an NDV HN protein if antibodies directed to the NDV HN protein bind to the non-NDV APMV HN protein or variant thereof with a 0.5 log, 1 log, 1.5 log, 2 log, 2.5 log, 3 log or lower affinity than to NDV HN protein in an assay known to one of skill in the art or described herein. In certain embodiments, a non-NDV APMV HN protein or a variant thereof is immunologically distinct from an NDV HN protein if anti-NDV HN antibodies do not substantially inhibit replication of NDV expressing the non-NDV APMV HN protein or variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a non-NDV APMV HN protein or a variant thereof is immunologically distinct from an NDV HN protein if anti-NDV HN antibodies inhibit replication of NDV expressing the non-NDV APMV HN protein or variant thereof in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs.

In some embodiments, a non-NDV APMV HN protein is an HN protein from a member of the subfamily Avulavirinae, but not NDV. In some embodiments, a non-NDV APMV HN protein is an HN protein from a member of the subfamily Avulavirinae and the genus Metaavulavirus. In some embodiments, a non-NDV APMV HN protein is an HN protein from a member of the subfamily Avulavirinae and genus paraavulavirus. In some embodiments, a non-NDV APMV HN protein is an HN protein from a member of the subfamily Avulavirinae and the genus orthoavulavirus but is not NDV.

In certain embodiments, a chimeric HN protein is immunologically distinct from an NDV HN protein. In certain embodiments, a chimeric HN protein is immunologically distinct from an NDV HN protein if antibodies directed to the NDV HN protein do not cross-react with the chimeric HN protein. In some embodiments, a chimeric HN protein is immunologically distinct from an NDV HN protein if antibodies directed to the NDV HN protein bind to the chimeric HN protein with a 2-fold, 5-fold, 10-fold, 15-fold, 20-fold or lower affinity than to NDV HN protein in an assay known to one of skill in the art or described herein. In certain embodiments, a chimeric HN protein is immunologically distinct from an NDV HN protein if antibodies directed to the NDV HN protein bind to the chimeric HN protein with a 0.5 log, 1 log, 1.5 log, 2 log, 2.5 log, 3 log or lower affinity than to NDV HN protein in an assay known to one of skill in the art or described herein. In certain embodiments, a chimeric HN protein is immunologically distinct from an NDV HN protein if anti-NDV HN antibodies do not substantially inhibit replication of NDV expressing the non-NDV APMV F protein or a variant thereof as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a chimeric HN protein is immunologically distinct from an NDV HN protein if anti-NDV HN antibodies inhibit replication of NDV expressing the chimeric HN protein in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein by less than about 0.5 logs, less than about 1 log, less than about 1.5 logs, or less than about 2 logs.

In specific embodiments, a variant of a non-NDV APMV HN protein retains one or more functions of the non-NDV APMV HN protein.

In certain embodiments, a variant of a non-NDV APMV HN protein is at least 75%, at least 80%, or at least 85% identical to the non-NDV AMPV HN protein. In some embodiments, a variant of a non-NDV HN protein is at least 90%, at least 95%, or at least 99% identical to the non-NDV APMV HN protein. In certain embodiments, a variant of a non-NDV APMV HN protein is 75% to 90%, 80% to 95% or 90% to 99.5% identical to the non-NDV AMPV HN protein.

In certain embodiment, a variant of a non-NDV APMV HN protein contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a non-NDV APMV HN protein. In some embodiments, a variant of a non-NDV APMV HN protein comprises the amino acid sequence of the non-NDV APMV HN protein with 20, 15, 10, 9, 8, 7, 6, 5, 4, 3, 2 or 1 amino acid residues of the non-NDV APMV HN protein substituted (e.g., conservatively substituted) with other amino acids. In certain embodiments, a variant of a non-NDV APMV HN protein comprises the amino acid sequence of the non-NDV APMV HN protein with up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In some embodiments, a variant of a non-NDV APMV HN protein is a polypeptide encoded by nucleic acid sequence that can hybridize under high, moderate or typical stringency hybridization conditions to a nucleic acid sequence encoding the non-NDV APMV HN protein. Hybridization conditions are known to one of skill in the art (see, e.g., U.S. Patent Application No. 2005/0048549 at, e.g., paragraphs 72 and 73).

In some embodiments, a non-NDV APMV HN protein is the HN protein of an APMV shown in FIG. 3B. In some embodiments, a non-NDV APMV HN protein is the F protein of a member of a genus shown in FIG. 3B or FIG. 6A. In certain embodiments, a non-NDV APMV HN protein is the HN protein of AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, a non-NDV APMV HN protein is the HN protein of APMV-2, such as, e.g., Chicken/California/Yucaipa/56 (Accession No. EU338414). In certain embodiments, a non-NDV APMV HN protein is the HN protein of APMV-2 Yucaipa. In other embodiments, a non-NDV APMV HN protein is not the HN protein of APMV-2 Yucaipa. In certain embodiments, a non-NDV APMV HN protein is the HN protein of APMV-3, such as, e.g., APMV3/Turkey/Wisconsin/68 (Accession No. EU782025). In some embodiments, a non-NDV APMV HN protein is the HN protein of APMV-4, such as, e.g., a APMV4/duck/Hongkong/D3/75 (Accession No. FJ177514), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, a non-NDV APMV HN protein is the HN protein of APMV-5, such as, e.g., APMV-5 budgerigar/Kunitachi/74 (Accession No. GU206351) or APMV5/budgerigar/Japan/TI/75 (Accession No. LC168750). In some embodiments, a non-NDV APMV HN protein is the HN protein of APMV-6, such as, e.g., APMV-6 Goose/FarEast/4440/2003 (Accession No. EF569970) or APMV6/duck/HongKong/18/199/77 (Accession No. EU622637). In certain embodiments, a non-NDV APMV HN protein is the HN protein of APMV-7, such as, e.g., APMV-7 dove/Tennessee/4/75 (Accession No. FJ231524). In some embodiments, a non-NDV APMV HN protein is the HN protein of APMV-8, such as, e.g., APMV-8 goose/Delaware/1053/76 (Accession No. FJ215863). In certain embodiments, a non-NDV APMV HN protein is the HN protein of APMV-9, such as, e.g., APMV9/duck/New York/22/78 (Accession No. EU910942). In some embodiments, a non-NDV APMV HN protein is the HN protein of APMV-10, such as, e.g., APMV-10 penguin/Falkland Islands/324/2007 (Accession No. HM147142 or NC_025349). In certain embodiments, a non-NDV APMV HN protein is the HN protein of APMV-11, such as, e.g., APMV-11 common_snipe/France/100212/2010 (Accession No. JQ886184). In some embodiments, a non-NDV APMV HN protein is the HN protein of APMV-12, such as, e.g., APMV12/Wigeon/Italy/3920_1/05 (Accession No. KC333050). In some embodiments, a non-NDV APMV HN protein is the HN protein of APMV-14, such as, e.g., APMV-14 duck/Japan/110G0352/2011 (Accession No. KX258200). In certain embodiments, a non-NDV APMV HN protein is the HN protein of APMV-15, such as, e.g., APMV-15 calidris_fuscicollis/Brazil/RS-1177/2012 (Accession No. KX932454). In some embodiments, a non-NDV APMV HN protein is the HN protein of APMV-17, such as, e.g., APMV17/Antarctica/107/13 (Accession No. MK167211). In some embodiments, a non-NDV APMV HN protein is the HN protein of APMV-20, such as, e.g., APMV-20 Gull/Kazakhstan/2014 (Accession No. MF033136). In certain embodiments, a non-NDV APMV HN protein is the HN protein of APMV-21, such as, e.g., APMV21/pigeon/Taiwan/AHRI128/17 (Accession No. MK67743).

In certain embodiments, a variant of a non-NDV APMV HN protein is a variant of the HN protein of AMPV-2, AMPV-3, AMPV-4, AMPV-5, AMPV-6, AMPV-7, AMPV-8, AMPV-9, AMPV-10, AMPV-11, AMPV-12, AMPV-13, AMPV-14, AMPV-15, AMPV-16, AMPV-17, AMPV-18, AMPV-19, AMPV-20, or AMPV-21. In some embodiments, a variant of a non-NDV APMV HN protein is a variant of the HN protein of APMV-2, such as, e.g., Chicken/California/Yucaipa/56 (Accession No. EU338414). In certain embodiments, a variant of a non-NDV APMV HN protein is a variant of the HN protein of APMV-3, such as, e.g., APMV3/Turkey/Wisconsin/68 (Accession No. EU782025). In some embodiments, a variant of a non-NDV APMV HN protein is a variant of the HN protein of APMV-4, such as, e.g., APMV4/duck/Hongkong/D3/75 (Accession No. FJ177514),), APMV4/Duck/China/G302/2012 (GenBank No. KC439346.1), APMV4/mallard/Belgium/15129/07 (GenBank No. JN571485), APMV4/Uriah_aalge/Russia/Tyuleniy_Island/115/2015 (GenBank No. KU601399.1) APMV-4/Egyptian goose/South Africa/N1468/2010 (GenBank No. JX133079.1), or APMV4/duck/Delaware/549227/2010 (GenBank No. JX987283.1). In certain embodiments, a variant of a non-NDV APMV HN protein is a variant of the HN protein of APMV-5, such as, e.g., APMV-5 budgerigar/Kunitachi/74 (Accession No. GU206351) or APMV5/budgerigar/Japan/TI/75 (Accession No. LC168750). In some embodiments, a variant of a non-NDV APMV HN protein is a variant of the HN protein of APMV-6, such as, e.g., APMV-6 Goose/FarEast/4440/2003 (Accession No. EF569970) or APMV6/duck/HongKong/18/199/77 (Accession No. EU622637). In certain embodiments, a variant of a non-NDV APMV HN protein is a variant of the HN protein of APMV-7, such as, e.g., APMV-7 dove/Tennessee/4/75 (Accession No. FJ231524). In some embodiments, a variant of a non-NDV APMV HN protein is a variant of the HN protein of APMV-8, such as, e.g., APMV-8 goose/Delaware/1053/76 (Accession No. FJ215863). In certain embodiments, a variant of a non-NDV APMV HN protein is a variant of the HN protein of APMV-9, such as, e.g., APMV9/duck/New York/22/78 (Accession No. EU910942). In some embodiments, a variant of a non-NDV APMV HN protein is a variant of the HN protein of APMV-10, such as, e.g., APMV-10 penguin/Falkland Islands/324/2007 (Accession No. HM147142 or NC_025349). In certain embodiments, a variant of a non-NDV APMV HN protein is a variant of the HN protein of APMV-11, such as, e.g., APMV-11 common_snipe/France/100212/2010 (Accession No. JQ886184). In some embodiments, a variant of a non-NDV APMV HN protein is a variant of the HN protein of APMV-12, such as, e.g., APMV12/Wigeon/Italy/3920_1/05 (Accession No. KC333050). In some embodiments, a variant of a non-NDV APMV HN protein is a variant of the HN protein of APMV-14, such as, e.g., APMV-14 duck/Japan/110G0352/2011 (Accession No. KX258200). In certain embodiments, a variant of a non-NDV APMV HN protein is a variant of the HN protein of APMV-15, such as, e.g., APMV-15 calidris_fuscicollis/Brazil/RS-1177/2012 (Accession No. KX932454). In some embodiments, a variant of a non-NDV APMV HN protein is a variant of the HN protein of APMV-17, such as, e.g., APMV17/Antarctica/107/13 (Accession No. MK167211). In some embodiments, a variant of a non-NDV APMV HN protein is a variant of the HN protein of APMV-20, such as, e.g., APMV-20 Gull/Kazakhstan/2014 (Accession No. MF033136). In certain embodiments, a variant of a non-NDV APMV HN protein is a variant of the HN protein of APMV-21, such as, e.g., APMV21/pigeon/Taiwan/AHRI128/17 (Accession No. MK67743).

In some embodiments, a non-NDV APMV HN protein has less than 65% identity to an NDV HN protein. In some embodiments, a non-NDV APMV HN protein has less than 60% identity to an NDV HN protein. In some embodiments, a non-NDV APMV HN protein has less than 50% identity to an NDV HN protein. In some embodiments, a non-NDV APMV HN protein has less than 55% identity to an NDV HN protein. In some embodiments, a non-NDV APMV HN protein has less than 50% identity to an NDV HN protein. In some embodiments, a non-NDV APMV HN protein has less than 45% identity to an NDV HN protein. In some embodiments, a non-NDV APMV HN protein has less than 40% identity to an NDV HN protein. In some embodiments, a non-NDV APMV HN protein has less than 35% identity to an NDV HN protein. In some embodiments, a non-NDV APMV HN protein has at least 20% or at least 25% identity to an NDV HN protein but less than 65%, less than 60%, less than 55%, less than 50%, or less than 45% identity. In some embodiments, the NDV HN protein is the NDV LaSota HN protein.

In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:1. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:2. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:3. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:4. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:5. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:6. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:7. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:8. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:9. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:10. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:11. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:12. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:13. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising the coding sequence set forth in SEQ ID NO:14. In specific embodiments, the NDV genome comprises the replaced NDV HN and F protein coding sequences as well as (1) a transcription unit encoding a NDV nucleocapsid (N) protein, (2) a transcription unit encoding a NDV phosphoprotein (P), (3) a transcription unit encoding a NDV matrix (M) protein, and (4) a transcription unit encoding a NDV large polymerase (L).

In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:1. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:2. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:3. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:4. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:5. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:6. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:7. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:8. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:9. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:10. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:11. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:12. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:13. In a specific embodiment, provided herein is a nucleic acid sequence comprising a nucleotide sequence of a Newcastle disease virus genome in which the coding sequences of NDV HN protein and the NDV F protein have been replaced with a nucleotide sequence comprising a coding sequence that is at least 75%, at least 80%, at least 85%, at least 90%, at least 95% or at least 98% identical to the coding sequence set forth in SEQ ID NO:14. In specific embodiments, the NDV genome comprises the replaced NDV HN and F protein coding sequences as well as (1) a transcription unit encoding a NDV nucleocapsid (N) protein, (2) a transcription unit encoding a NDV phosphoprotein (P), (3) a transcription unit encoding a NDV matrix (M) protein, and (4) a transcription unit encoding a NDV large polymerase (L).

In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:1. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:2. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:3. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:4. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:5. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:6. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:7. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:8. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:9. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:10. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:11. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:12. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:13. In another specific embodiment, provided herein is a nucleic acid sequence comprising a nucleotide sequence of a Newcastle disease virus genome in which the nucleotide sequences encoding the NDV HN protein and the NDV F protein have been replaced with a nucleotide sequence comprising the nucleotide sequence set forth in SEQ ID NO:14. In specific embodiments, the NDV genome comprises the replaced NDV HN and F protein coding sequences as well as (1) a transcription unit encoding a NDV nucleocapsid (N) protein, (2) a transcription unit encoding a NDV phosphoprotein (P), (3) a transcription unit encoding a NDV matrix (M) protein, and (4) a transcription unit encoding a NDV large polymerase (L).

One skilled in the art will understand that the NDV genomic RNA sequence is an RNA sequence corresponding to the negative sense of a cDNA sequence encoding the NDV genome. Thus, any program that converts a nucleotide sequence to its reverse complement sequence may be utilized to convert a cDNA sequence encoding an NDV genome into the genomic RNA sequence (see, e.g., www.bioinformatics.org/sms/rev_comp.html, www.fr33.net/seqedit.php, and DNAStar). Accordingly, the nucleotide sequences provided in Tables 1 and 3, infra, may be readily converted to the negative-sense RNA sequence of the NDV genome by one of skill in the art. In some embodiments, the nucleotide sequence of a NDV genome is of an NDV of any strain known to one of skill in the art. See, e.g., Section 5.1.2 for exemplary strains. In a specific embodiments, the nucleotide sequence of a NDV genome is of the LaSota strain. In certain embodiments, the nucleotide sequence of a NDV genome is of a lentogenic strain. In some embodiments, the nucleotide sequence of a NDV genome is of a mesogenic strain. In certain embodiments, the nucleotide sequence of a NDV genome is of a velogenic. The nucleotide sequence of a NDV genome may be a cDNA sequence or an RNA sequence (e.g., negative sense RNA or positive sense RNA).

In some embodiments, provided herein is a nucleic acid sequence comprising a nucleotide sequence of SEQ ID NO:44. In some embodiments, provided herein is a nucleic acid sequence comprising a nucleotide sequence of SEQ ID NO:44 without the GFP coding sequence. In some embodiments, provided herein is a nucleic acid sequence comprising a nucleotide sequence that is at least 75%, at least 80%, at least 85%, at least 90%, at least 95%, at least 98%, or at least 99% identical to the nucleotide sequence of SEQ ID NO:44. In some embodiments, provided herein is a nucleic acid sequence comprising a nucleotide sequence that is at least 75%, at least 80%, at least 85%, at least 90%, at least 95%, at least 98%, or at least 99% identical to the nucleotide sequence of SEQ ID NO:44 without the GFP coding sequence. In some embodiment, provided herein is a nucleic acid sequence comprising the nucleotide sequence of SEQ ID NO:44 without the GFP coding sequence and a transgene encoding a heterologous sequence, such as an antigen.

In some embodiments, provided herein is a nucleic acid sequence comprising a nucleotide sequence of SEQ ID NO:45. In some embodiments, provided herein is a nucleic acid sequence comprising a nucleotide sequence of SEQ ID NO:45 without the GFP coding sequence. In some embodiments, provided herein is a nucleic acid sequence comprising a nucleotide sequence that is at least 75%, at least 80%, at least 85%, at least 90%, at least 95%, at least 98%, or at least 99% identical to the nucleotide sequence of SEQ ID NO:45. In some embodiments, provided herein is a nucleic acid sequence comprising a nucleotide sequence that is at least 75%, at least 80%, at least 85%, at least 90%, at least 95%, at least 98%, or at least 99% identical to the nucleotide sequence of SEQ ID NO:45 without the GFP coding sequence. In some embodiment, provided herein is a nucleic acid sequence comprising the nucleotide sequence of SEQ ID NO:45 without the GFP coding sequence and a transgene encoding a heterologous sequence, such as an antigen.

In some embodiments, a nucleic acid sequence or nucleotide sequence described herein is codon optimized. See Section 5.1.4 for a description of codon optimization information and techniques.

In certain embodiments, a nucleic acid sequence described herein does not comprise a heterologous sequence encoding a heterologous protein other than the non-NDV APMV HN protein or variant thereof. In certain embodiments, a nucleic acid sequence described herein does not comprise a heterologous sequence encoding a heterologous protein other than the non-NDV APMV F protein or variant thereof. In certain embodiments, a nucleic acid sequence described herein does not comprise a heterologous sequence encoding a heterologous protein other than the non-NDV APMV HN protein or variant thereof and non-NDV APMV F protein or variant thereof.

In certain embodiments, a nucleic acid sequence described herein further comprises a transgene comprising a nucleotide sequence encoding a heterologous sequence (e.g., a heterologous protein). In certain embodiments, a nucleic acid sequence described herein further comprises a transgene comprising a nucleotide sequence encoding an antigen. See Section 5.1.3 for a description of transgenes that may be incorporated into a nucleic acid sequence described herein.

In specific embodiments, a nucleic acid sequence described herein is used in the production of a recombinant NDV described herein. In specific embodiments, a nucleic acid sequence described herein is part of a recombinant NDV described herein.

In specific embodiments, a nucleic acid sequence or nucleotide sequence described herein is a recombinant nucleic acid sequence or recombinant nucleotide sequence. In certain embodiments, a nucleotide sequence or nucleic acid sequence described herein may be a DNA molecule (e.g., cDNA), an RNA molecule, or a combination of a DNA and RNA molecule. In some embodiments, a nucleotide sequence or nucleic acid sequence described herein may comprise analogs of DNA or RNA molecules. Such analogs can be generated using, for example, nucleotide analogs, which include, but are not limited to, inosine, methylcytosine, pseudouridine, or tritylated bases. Such analogs can also comprise DNA or RNA molecules comprising modified backbones that lend beneficial attributes to the molecules such as, for example, nuclease resistance or an increased ability to cross cellular membranes. The nucleic acid or nucleotide sequences can be single-stranded, double-stranded, may contain both single-stranded and double-stranded portions, and may contain triple-stranded portions. In a specific embodiment, a nucleotide sequence or nucleic acid sequence described herein is a negative sense single-stranded RNA. In another specific embodiment, a nucleotide sequence or nucleic acid sequence described herein is a positive sense single-stranded RNA. In another specific embodiment, a nucleotide sequence or nucleic acid sequence described herein is a cDNA.

In specific embodiments, a nucleic acid sequence is isolated. In certain embodiments, an “isolated” nucleic acid sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences in which the nucleic acid sequence is separated from cellular components of the cells from which it is isolated or recombinantly produced. Thus, nucleic acid sequence that is substantially free of cellular material includes preparations of nucleic acid sequence having less than about 30%, 20%, 10%, or 5% (by dry weight) of other nucleic acids. The term “substantially free of culture medium” includes preparations of nucleic acid sequence in which the culture medium represents less than about 50%, 20%, 10%, or 5% of the volume of the preparation. The term “substantially free of chemical precursors or other chemicals” includes preparations in which the nucleic acid sequence is separated from chemical precursors or other chemicals which are involved in the synthesis of the nucleic acid sequence. In specific embodiments, such preparations of the nucleic acid sequence have less than about 50%, 30%, 20%, 10%, 5% (by dry weight) of chemical precursors or compounds other than the nucleic acid sequence of interest.

### 5.1.2 NDV Backbone

Any NDV type or strain may be serve as the “backbone” into which the nucleotide sequence encoding the NDV F protein and/or the nucleotide sequence encoding the NDV HN protein are replaced with a non-NDV APMV F protein coding sequence or a variant thereof and/or a non-NDV HN coding sequence or variant thereof, respectively. In addition, any NDV type or strain may be serve as the “backbone” in which the nucleotide sequence encoding the NDV F protein and/or the nucleotide sequence encoding the NDV HN protein are replaced with a chimeric F protein coding sequence and/or a chimeric HN coding sequence, respectively. For example the NDV may be a naturally-occurring strain, a variant, a mutant, a mutagenized virus, and/or a genetically engineered virus. In a specific embodiment, the NDV backbone is a lentogenic NDV. In another specific embodiment, the NDV backbone is strain LaSota. Other examples of NDV strains which may be used as the NDV backbone include the NDV Fuller, the NDV Ulster strain or the NDV Hitchner B1 strain. In some embodiments, a lentogenic strain other than NDV Hitchner B1 strain is used as the backbone. In a specific embodiment, the NDV backbone is a naturally-occurring strain. In certain embodiments, the NDV backbone is a lytic strain. In other embodiments, the NDV backbone is a non-lytic strain. In certain embodiments, the NDV backbone is lentogenic strain. In some embodiments, the NDV backbone is a mesogenic strain. In other embodiments, the NDV backbone is a velogenic strain. Specific examples of NDV strains include, but are not limited to, the 73-T strain, NDV HUJ strain, Ulster strain (see, e.g., GenBank No. U25837), Fuller strain, MTH-68 strain, Italien strain (see, e.g., GenBank No. EU293914), Hickman strain (see, e.g., Genbank No. AF309418), PV701 strain, Hitchner B1 strain (see, e.g., GenBank No. AF309418 or NC_002617), La Sota strain (see, e.g., GenBank Nos. AY845400, AF07761.1 and JF950510.1 and GI No. 56799463), YG97 strain (see, e.g., GenBank Nos. AY351959 or AY390310), MET95 strain (see, e.g., GenBank No. AY143159), Roakin strain (see, e.g., GenBank No. AF124443), and F48E9 strain (see, e.g., GenBank Nos. AF163440 and U25837). In a specific embodiment, the NDV backbone is the Hitchner B1 strain. In another embodiment, the NDV backbone is a B1 strain as identified by GenBank No. AF309418 or NC_002617. In another specific embodiment, the NDV backbone is the La Sota strain. In a specific embodiment, the nucleotide sequence of the La Sota genome comprises an RNA sequence corresponding to the negative sense of the cDNA sequence set forth in SEQ ID NO:15. In another embodiment, the NDV backbone is a LaSota strain as identified by GenBank Nos. AY845400, AF07761.1 or JF950510.1.

One skilled in the art will understand that the NDV genomic RNA sequence is an RNA sequence corresponding to the negative sense of a cDNA sequence encoding the NDV genome. Thus, any program that converts a nucleotide sequence to its reverse complement sequence may be utilized to convert a cDNA sequence encoding an NDV genome into the genomic RNA sequence (see, e.g., www.bioinformatics.org/sms/rev_comp.html, www.fr33.net/seqedit.php, and DNAStar).

In specific embodiments, the NDV backbone is not pathogenic in birds as assessed by a technique known to one of skill. In certain specific embodiments, the NDV backbone is not pathogenic as assessed by intracranial injection of 1-day-old chicks with the virus, and disease development and death as scored for 8 days. In some embodiments, the NDV backbone has an intracranial pathogenicity index of less than 0.7, less than 0.6, less than 0.5, less than 0.4, less than 0.3, less than 0.2 or less than 0.1. In certain embodiments, the NDV backbone has an intracranial pathogenicity index of zero. See, e.g., OIE Terrestrial Manual 2012, Chapter 2.3.14, entitled “Newcastle Disease (Infection With Newcastle Disease Virus) for a description of this assay, which is found at the following website www.oie.int/fileadmin/Home/eng/Health_standards/tahm/2.03.14_NEWCASTLE_DIS.pdf, which is incorporated herein by reference in its entirety.

In certain embodiments, the NDV backbone is a mesogenic strain that has been genetically engineered so as not be a considered pathogenic in birds as assessed by techniques known to one skilled in the art. In certain embodiments, the NDV backbone is a velogenic strain that has been genetically engineered so as not be a considered pathogenic in birds as assessed by techniques known to one skilled in the art.

In preferred embodiments, the NDV backbone is non-pathogenic in humans or bovine. In preferred embodiments, the NDV backbone is non-pathogenic in humans, bovines and avians. In certain embodiments, the NDV backbone is attenuated such that the NDV remains, at least partially, infectious and can replicate in vivo, but only generate low titers resulting in subclinical levels of infection that are non-pathogenic (see, e.g., Khattar et al., 2009, J. Virol. 83:7779-7782). Such attenuated NDVs may be especially suited for embodiments wherein the virus is administered to a subject in order to act as an immunogen, e.g., a live vaccine. The viruses may be attenuated by any method known in the art. In a specific embodiment, the NDV genome comprises sequences necessary for infection and replication of the attenuated virus such that progeny is produced and the infection level is subclinical.

### 5.1.3 Transgenes

In a specific embodiment, a transgene comprising a nucleotide sequence encoding an antigen is incorporated into the nucleic acid sequence described herein (e.g., Section 5.1.1 or Section 6), which comprises a nucleotide sequence of a NDV genome in which the NDV F protein coding sequence and/or NDV HN protein coding sequence have been replaced as described herein. The transgene may inserted into a nucleotide sequence of a NDV genome of any NDV type or strain (e.g., NDV LaSota strain) in which the NDV F protein coding sequence and/or NDV HN protein coding sequence have been replaced as described herein. One of skill in the art would be able to use the sequence information of the antigen to produce a transgene for incorporation into the nucleotide sequence of a NDV genome of any NDV type or strain in which the NDV F protein coding sequence and/or NDV HN protein coding sequence have been replaced as described herein. Given the degeneracy of the nucleic acid code, there are a number of different nucleic acid sequences that may encode the same antigen. In a specific embodiment, a transgene comprising a nucleotide sequence encoding an antigen is codon optimized. In a specific embodiment, the coding sequence of an antigen is codon optimized. See, e.g., Section 5.1.4, infra, for a discussion regarding codon optimization. The transgene comprising a nucleotide sequence encoding an antigen may be incorporated between any two NDV transcription units (e.g., between the NDV P and M transcription units, or between the HN and L transcription units). A transgene described herein, which is incorporated into the genome of a NDV, results in the expression of an antigen encoded by the transgene by a cell(s) infected with a recombinant NDV described herein.

In another embodiment, described herein are transgenes comprising a nucleotide sequence encoding a chimeric antigen, wherein the chimeric antigen comprises an antigen ectodomain and NDV F protein transmembrane and cytoplasmic domains. In other words, the NDV F protein transmembrane and cytoplasmic domains replace the antigen's transmembrane and cytoplasmic domains so that the chimeric protein does not include the antigen transmembrane and cytoplasmic domains. In certain embodiments, one, two or more amino acid residues of the transmembrane domain of the antigen but less than 10 amino acid residues of the transmembrane domain of the antigen are part of the chimeric antigen. The ectodomain, transmembrane and cytoplasmic domains of the antigen and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the antigen and NDV F protein. See, e.g., Park et al., 2006, PNAS May 23, 2006 103 (21) 8203-8208, International Patent Application No. WO2007/064802, and U.S. Pat. No. 9,387,242 B2 for methods for producing chimeric antigens. In a specific embodiment, described herein are transgenes comprising a nucleotide sequence encoding a chimeric antigen, wherein the chimeric antigen comprises an ectodomain of a class I protein antigen and NDV F protein transmembrane and cytoplasmic domains.

In another embodiment, described herein are transgenes comprising a nucleotide sequence encoding a chimeric antigen, wherein the chimeric antigen comprises an antigen ectodomain and NDV HN protein transmembrane and cytoplasmic domains. In other words, the NDV HN protein transmembrane and cytoplasmic domains replace the antigen's transmembrane and cytoplasmic domains so that the chimeric protein does not include the antigen transmembrane and cytoplasmic domains. In certain embodiments, one, two or more amino acid residues of the transmembrane domain of the antigen but less than 10 amino acid residues of the transmembrane domain of the antigen are part of the chimeric antigen. The ectodomain, transmembrane and cytoplasmic domains of the antigen and NDV HN protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the antigen and NDV HN protein. See, e.g., Park et al., 2006, PNAS May 23, 2006 103 (21) 8203-8208, International Patent Application No. WO2007/064802, and U.S. Pat. No. 9,387,242 B2 for methods for producing chimeric antigens. In a specific embodiment, described herein are transgenes comprising a nucleotide sequence encoding a chimeric antigen, wherein the chimeric antigen comprises an ectodomain of a class II protein antigen and NDV HN protein transmembrane and cytoplasmic domains.

In certain embodiments, a transgene comprises a nucleotide sequence encoding a chimeric antigen, wherein the chimeric antigen comprises a SARS-CoV-2 spike protein ectodomain or fragment thereof (e.g., a fragment comprising the receptor binding domain) and NDV F protein transmembrane and cytoplasmic domains. In some embodiments, a transgene comprises a nucleotide sequence encoding a chimeric antigen, wherein the chimeric antigen comprises an hMPV F protein ectodomain or fragment thereof and NDV F protein transmembrane and cytoplasmic domains. In certain embodiments, a transgene comprises a nucleotide sequence encoding a chimeric antigen, wherein the chimeric antigen comprises an RSV F protein ectodomain or a fragment thereof and NDV F protein transmembrane and cytoplasmic domains.

The transgene may inserted into a nucleotide sequence of a NDV genome of any NDV type or strain (e.g., NDV LaSota strain) in which the NDV F protein coding sequence and/or NDV HN protein coding sequence have been replaced as described herein. One of skill in the art would be able to use the sequence information of the chimeric antigen to produce a transgene for incorporation into the nucleotide sequence of a NDV genome of any NDV type or strain in which the NDV F protein coding sequence and/or NDV HN protein coding sequence have been replaced as described herein. Given the degeneracy of the nucleic acid code, there are a number of different nucleic acid sequences that may encode the same chimeric antigen. In a specific embodiment, a transgene comprising a nucleotide sequence encoding a chimeric antigen is codon optimized. In a specific embodiment, described herein is a transgene comprising a nucleotide sequence encoding a chimeric antigen, wherein the chimeric antigen comprises an antigen ectodomain and NDV F protein transmembrane and cytoplasmic domains, and wherein the ectodomain of the antigen is encoded by a codon optimized nucleic acid sequence. See, e.g., Section 5.1.4, infra, for a discussion regarding codon optimization. The transgene encoding a nucleotide sequence encoding chimeric antigen may be incorporated between any two NDV transcription units (e.g., between the NDV P and M transcription units, or between the HN and L transcription units).

In certain embodiments, a transgene comprising a nucleotide sequence encoding an antigen or a chimeric antigen comprises NDV regulatory signals (e.g., gene end, intergenic, and gene start sequences) and Kozak sequences. In some embodiments, a transgene comprising a nucleotide sequence encoding an antigen or a chimeric antigen comprises NDV regulatory signals (e.g., gene end, intergenic, and gene start sequences), Kozak sequences and restriction sites to facilitate cloning. In certain embodiments, a transgene encoding an antigen or a chimeric antigen comprises NDV regulatory signals (gene end, intergenic and gene start sequences), Kozak sequences, restriction sites to facilitate cloning, and additional nucleotides in the non-coding region to ensure compliance with the rule of six. In a preferred embodiment, the transgene complies with the rule of six.

In certain embodiments, an antigen is an infectious disease antigen. Infectious diseases include those diseases caused by viruses, bacteria, fungi, and protozoa. In some embodiments, an antigen is an antigen of a pathogen. In certain embodiments, an antigen is a viral, bacterial, fungal or protozoa antigen. The antigen may be a fragment of a protein expressed by a virus, bacteria, fungus, protozoa or other pathogen. In a specific embodiment, an antigen is viral antigen. The viral antigen may be a severe acute respiratory syndrome coronavirus 2 (SARS-CoV-2) antigen, human metapneumovirus antigen, respiratory syncytial virus antigen, an Ebola antigen, Lassa virus antigen, Nipah virus antigen, or Middle East respiratory syndrome coronavirus (MERS-CoV) antigen. In some embodiments, the viral antigen is a surface glycoprotein. The viral antigen may be a fragment of a surface glycoprotein or envelope protein. In some embodiments, an antigen used herein has at least 75%, at least 80%, at least 85%, at least 90%, at least 95% or at least 98% identity to an antigen found in nature. For example, an antigen may have at least 75%, at least 80%, at least 85%, at least 90%, at least 95% or at least 98% identity to a naturally occurring a viral antigen (e.g., a SARS-CoV-2 antigen, a RSV antigen, an Ebola virus antigen, a MERS-CoV antigen, a hMPV antigen, Lassa virus antigen or Nipah virus antigen). In certain embodiment, an antigen is an antigen from or derived from a pathogen (e.g., virus, bacteria, etc.) that causes a pandemic or epidemic.

In one embodiment, the viral antigen is a SARS-CoV-2 antigen. In another embodiment, the viral antigen is a SARS-CoV-2 nucleocapsid protein or a fragment thereof. As used herein, the terms “SARS-CoV-2 nucleocapsid” refers to a SARS-CoV-2 nucleocapsid known to those of skill in the art. In certain embodiments, the nucleocapsid protein comprises the amino acid or nucleic acid sequence found at GenBank Accession No. MT081068.1, MT081066.1 or MN908947.3. See also, e.g., GenBank Accession Nos. MN908947.3, MT447160, MT44636, MT446360, MT444593, MT444529, MT370887, and MT334558 for examples of amino acid sequences of SARS-CoV-2 nucleocapsid protein and nucleotide sequences encoding SARS-CoV-2 nucleocapsid protein.

In another embodiment, the viral antigen is a SARS-CoV-2 spike protein or a fragment thereof. In some embodiments, the fragment of the SARS-CoV-2 spike protein comprises (or consists of) the receptor binding domain of the protein. In certain embodiments, the fragment of the SARS-CoV-2 spike protein comprises (or consists of) the S1 or S2 domain of the protein. In some embodiments, the fragment of the SARS-CoV-2 spike protein comprises (or consists of) the ectodomain of the protein. As used herein, the terms “SARS-CoV-2 spike protein” and “spike protein of SARS-CoV-2” refer to a SARS-CoV-2 spike protein known to those of skill in the art. See, e.g., GenBank Accession Nos. MN908947.3, MT447160, MT44636, MT446360, MT444593, MT444529, MT370887, and MT334558 for examples of amino acid sequences of SARS-CoV-2 spike protein and nucleotide sequences encoding SARS-CoV-2 spike protein. In certain embodiments, the spike protein comprises the amino acid or nucleic acid sequence found at GenBank Accession No. MN908947.3. In certain embodiments, the spike protein comprises the amino acid or nucleic acid sequence of a variant of SARS-CoV-2. In some embodiments, the spike protein comprises the amino acid or nucleic acid sequence of B.1.1.7. In specific embodiments, the spike protein comprises the amino acid or nucleic acid sequence of 20I/501Y.V1 (BEI Reference isolate NR-54000). In some embodiments, the spike protein comprises the amino acid or nucleic acid sequence of P.1. In specific embodiments, the spike protein comprises the amino acid or nucleic acid sequence of 20J/501Y.V3 (BEI Reference isolate NR-54982). In some embodiments, the spike protein comprises the amino acid or nucleic acid sequence of B.1.351. In specific embodiments, the spike protein comprises the amino acid or nucleic acid sequence of 20H/501.V2 (BEI Reference isolate NR-54009). In some embodiments, the spike protein comprises the amino acid or nucleic acid sequence of B.1.4271. In specific embodiments, the spike protein comprises the amino acid or nucleic acid sequence of 20C/S:452R. In some embodiments, the spike protein comprises the amino acid or nucleic acid sequence of B.1.429. In specific embodiments, the spike protein comprises the amino acid or nucleic acid sequence of 20C/S:452R. A typical spike protein comprises domains known to those of skill in the art including an S1 domain, a receptor binding domain, an S2 domain, a transmembrane domain and a cytoplasmic domain. See, e.g., Wrapp et al., 2020, Science 367: 1260-1263 for a description of SARS-CoV-2 spike protein (in particular, the structure of such protein). The spike protein may be characterized has having a signal peptide (e.g., a signal peptide of 1-14 amino acid residues of the amino acid sequence of GenBank Accession No. MN908947.3), a receptor binding domain (e.g., a receptor binding domain of 319-541 amino acid residues of GenBank Accession No. MN908947.3), an ectodomain (e.g., an ectodomain of 15-1213 amino acid residues of GenBank Accession No. MN908947.3), and a transmembrane and endodomain (e.g., a transmembrane and endodomain of 1214-1273 amino acid residues of GenBank Accession No. MN908947.3). In certain embodiments, the viral antigen is a fragment of a SARS-CoV-2 spike protein. The fragment may comprise the receptor binding domain of the SARS-CoV-2 spike protein. The fragment may comprise the S1 domain, S2 domain or the ectodomain of the SARS-CoV-2 spike protein. The terms “SARS-CoV-2 spike protein” encompass SARS-CoV-2 spike polypeptides that are modified by post-translational processing such as signal peptide cleavage, disulfide bond formation, glycosylation (e.g., N-linked glycosylation), protease cleavage and lipid modification (e.g. S-palmitoylation). In some embodiments, the SARS-CoV-2 spike protein includes a signal sequence. In other embodiments, SARS-CoV-2 spike protein does not include a signal sequence. The signal sequence can be the naturally occurring signal peptide sequence or a variant thereof. In some embodiments, the signal peptide is an SARS-CoV-2 spike protein signal peptide. In some embodiments, the signal peptide is heterologous to an SARS-CoV-2 spike protein signal peptide.

In some embodiments, provided herein is a SARS-CoV-2 antigen comprises a derivative SARS-CoV-2 spike protein ectodomain and NDV F protein transmembrane and cytoplasmic domains, wherein the derivative comprises a SARS-CoV-2 spike protein ectodomain in which: (1) amino acid residues corresponding to amino acid residues 817, 892, 899, 942, 986, and 987 of SARS-CoV-2 spike protein found at GenBank Accession No. MN908947.3 are substituted with prolines, and (2) amino acid residues corresponding to amino acid residues 682 to 685 are substituted such that the polybasic cleavage site is inactivated. In specific embodiments, a polybasic cleavage site is inactivated if the site cannot be cleaved by, e.g., furin. In a specific embodiment, amino acid residues corresponding to amino acid residues 682 to 685 of the polybasic cleavage site of the SARS-CoV-2 spike protein found at GenBank Accession No. MN908947.3 are substituted with a single alanine. In certain embodiments, the NDV F protein transmembrane and cytoplasmic domains are fused to the derivative of the SARS-CoV-2 spike protein ectodomain through a linker sequence (e.g., GGGGS (SEQ ID NO:46)). In some embodiments, the linker is a glycine (G) linker or glycine and serine (GS) linker. For example, the linker may comprise the sequence of (GGGGS)ₙ, wherein n is 1, 2, 3, 4, 5 or more (SEQ ID NO:47). In another example, the linker may comprise (G)ₙ, wherein n is 2, 3, 4, 5, 6, 7, 8 or more. In a specific embodiment, the linker comprises the sequence GGGGS (SEQ ID NO:46). In some embodiments, the NDV F protein transmembrane and cytoplasmic domains are fused directly to the derivative of the SARS-CoV-2 spike protein ectodomain. In a specific embodiment, the NDV F protein and chimeric F protein is incorporated into the NDV virion.

In one embodiment, the viral antigen is a human metapneumovirus antigen. In another embodiment, the viral antigen is a human metapneumovirus G protein or a fragment thereof. “Human Metapneumovirus G protein” and “hMPV G protein” refer to any Human Metapneumovirus G protein known to those of skill in the art. In another embodiment, the viral antigen is a human metapneumovirus F protein or a fragment thereof “Human Metapneumovirus F protein” and “hMPV F protein” refer to any Human Metapneumovirus F protein known to those of skill in the art. The hMPV F protein is synthesized as a F0 inactive precursor. The F0 inactive precursor requires cleavage during intracellular maturation. The hMPV F is cleaved to form F1 and F2. The hMPV F protein exists in two conformations, prefusion and post-fusion. GenBank™ accession number AY145301.1 and KJ627437.1, provide exemplary nucleic acid sequences encoding hMPV F protein. GenBank™ accession numbers AAN52915.1, AHV79975.1, AGJ74035.1, and AGZ48845.1 provide exemplary hMPV F protein amino acid sequences. The terms “hMPV F protein” and “human metapneumovirus F protein” encompass hMPV F polypeptides that are modified by post-translational processing such as signal peptide cleavage, disulfide bond formation, glycosylation (e.g., N-linked glycosylation), protease cleavage and lipid modification (e.g. S-palmitoylation). In some embodiments, the hMPV F protein includes a signal sequence. In other embodiments, hMPV F protein does not include a signal sequence. The signal sequence can be the naturally occurring signal peptide sequence or a variant thereof. The hMPV F protein signal sequence is typically 18 amino acids in length. In some embodiments, the signal peptide is an hMPV F protein signal peptide. In some embodiments, the signal peptide is heterologous to an hMPV F protein signal peptide.

In one embodiment, the viral antigen is a RSV G protein or a fragment thereof. “RSV G protein” and “respiratory syncytial virus G protein” refer to any respiratory syncytial G protein known to those of skill in the art. In one embodiment, the viral antigen is a RSV F protein or a fragment thereof. “RSV F protein” and “respiratory syncytial virus F protein” refer to any respiratory syncytial F protein known to those of skill in the art. The RSV F protein typically exists as a homotrimer. The RSV F protein is synthesized as a F0 inactive precursor which is heavily N-glycosylated. The F0 inactive precursor requires cleavage during intracellular maturation by a furin-like proteases. The RSV F contains two furin sites, and cleavage by furin-like proteases leads to three polypeptides: F2, p27 and F1, with the latter containing a hydrophobic fusion peptide at its N terminus. The RSV F protein exists in two conformations, prefusion and post-fusion. The RSV F protein may be human RSV F protein or bovine F protein. GenBank™ accession numbers KJ155694.1, KU950686.1, KJ672481.1, KP119747, and AF035006.1 provide exemplary nucleic acid sequences encoding human RSV F protein. GenBank™ accession numbers AHL84194.1, AMT79817.1, AHX57603.1, AIY70220.1 and AAC14902.1 provide exemplary human RSV F protein amino acid sequences. GenBank™ accession numbers AF295543.1, AF092942.1, and Y17970.1 provide exemplary nucleic acid sequences encoding bovine RSV F protein. GenBank™ accession numbers AAL49399.1, NP_048055.1, AAC96308.1, and CAA76980.1 provide exemplary bovine RSV F protein amino acid sequences. The terms “RSV F protein” and “respiratory syncytial virus F protein” encompass RSV F polypeptides that are modified by post-translational processing such as signal peptide cleavage, disulfide bond formation, glycosylation (e.g., N-linked glycosylation), protease cleavage and lipid modification (e.g. S-palmitoylation). In some embodiments, the RSV F protein includes a signal sequence. In other embodiments, RSV F protein does not include a signal sequence. The signal sequence can be the naturally occurring signal peptide sequence or a variant thereof. The RSV F protein signal sequence is typically 25 amino acids in length. In some embodiments, the signal peptide is an RSV F protein signal peptide. In some embodiments, the signal peptide is heterologous to an RSV F protein signal peptide.

In one embodiment, an antigen is an Ebola virus antigen (e.g., Ebola virus glycoprotein GP or a fragment thereof, or Ebola virus nucleocapsid or a fragment thereof). In another embodiment, an antigen is a Lassa virus antigen (e.g., a Lassa virus envelope glycoprotein GP1 or a fragment thereof, or a Lassa virus envelope glycoprotein GP2 or a fragment thereof). In another embodiment, an antigen is Nipah virus antigen (e.g., Nipah virus F or a fragment thereof, or a Nipah virus G protein or a fragment thereof). In another embodiment, an antigen is a MERS-CoV antigen (e.g., a MERS-CoV spike protein or a fragment thereof, or nucleocapsid protein or a fragment thereof).

In certain embodiments, a fragment of a protein comprises at least 8, at least 10, at least 12, at least 15 or more contiguous amino acids of the protein. In some embodiments, a fragment of a protein comprises at least 20, at least 30, at least 40, at least 50 or more contiguous amino acids of the protein. In certain embodiments, a fragment of a protein comprises at least 75, at least 100, at least 125, at least 150 or more contiguous amino acids of the protein. In some embodiments, a fragment of a protein comprises at least 175, at least 200, at least 250, at least 300, at least 350 or more contiguous amino acids of the protein.

In some embodiments, an antigen is a cancer or tumor antigen or tumor antigen (e.g., tumor-associated antigens and tumor-specific antigens). Antigens that are characteristic of tumor antigens can be derived from the cell surface, cytoplasm, nucleus, organelles and the like of cells of tumor tissue. Examples include antigens characteristic of tumor proteins, including proteins encoded by mutated oncogenes, viral proteins associated with tumors, and glycoproteins. Tumors include, but are not limited to, those derived from the types of cancer: lip, nasopharynx, pharynx and oral cavity, esophagus, stomach, colon, rectum, liver, gall bladder, pancreas, larynx, lung and bronchus, melanoma of skin, breast, cervix, uterine, ovary, bladder, kidney, uterus, brain and other parts of the nervous system, thyroid, prostate, testes, Hodgkin's disease, non-Hodgkin's lymphoma, multiple myeloma and leukemia. In some embodiments, the cancer antigen or tumor antigen is HER2, EGFR, VEGF, CD33, CD20, ErbB2, prostate specific membrane antigen (PSMA), APO-1, or MUC-1.

### 5.1.4 Codon Optimization

Any codon optimization technique known to one of skill in the art may be used to codon optimize a nucleic acid sequence or nucleotide sequence described herein. Methods of codon optimization are known in the art, e.g., the OptimumGene™ (GenScript®) protocol and Genewiz® protocol, which are incorporated by reference herein in its entirety. See also U.S. Pat. No. 8,326,547 for methods for codon optimization, which is incorporated herein by reference in its entirety.

As an exemplary method for codon optimization, each codon in the open frame of the nucleic acid sequence or nucleotide sequence described herein) is replaced by the codon most frequently used in mammalian proteins. This may be done using a web-based program (www.encorbio.com/protocols/Codon.htm) that uses the Codon Usage Database, maintained by the Department of Plant Gene Research in Kazusa, Japan. The nucleic acid sequence or nucleotide sequence optimized for mammalian expression may be inspected for: (1) the presence of stretches of 5×A or more that may act as transcription terminators; (2) the presence of restriction sites that may interfere with subcloning; (3) compliance with the rule of six. Following inspection, (1) stretches of 5×A or more that may act as transcription terminators may be replaced by synonymous mutations; (2) restriction sites that may interfere with subcloning may be replaced by synonymous mutations; (3) NDV regulatory signals (gene end, intergenic and gene start sequences), and Kozak sequences for optimal protein expression may be added; and (4) nucleotides may be added in the non-coding region to ensure compliance with the rule of six. Synonymous mutations are typically nucleotide changes that do not change the amino acid encoded. For example, in the case of a stretch of 6 As (AAAAAA), which sequence encodes Lys-Lys, a synonymous sequence would be AAGAAG, which sequence also encodes Lys-Lys.

### 5.2 Construction of NDVS

The recombinant NDVs described herein (see, e.g., Sections 5.1 and 6) can be generated using the reverse genetics technique. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in U.S. Pat. No. 6,146,642 issued Nov. 14, 2000; in European Patent Publication EP 0702085A1, published Feb. 20, 1996; in U.S. patent application Ser. No. 09/152,845; in International Patent Publications PCT WO97/12032 published Apr. 3, 1997; WO96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

The helper-free plasmid technology can also be utilized to engineer a NDV described herein. Briefly, a complete cDNA of a NDV (e.g., the Hitchner B1 strain or LaSota strain) is constructed, inserted into a plasmid vector and engineered to contain a unique restriction site between two transcription units (e.g., the NDV P and M genes; or the NDV HN and L genes). A nucleotide sequence encoding a heterologous amino acid sequence (e.g., a transgene or other sequence described herein such as, e.g., a nucleotide sequence encoding a SARS-CoV-2 spike protein, a nucleotide sequence encoding an RSV F protein, a chimeric F protein, hMPV F protein) may be inserted into the viral genome at the unique restriction site. Alternatively, a nucleotide sequence encoding a heterologous amino acid sequence (e.g., a transgene or other sequence described herein such as, e.g., a nucleotide sequence encoding a SARS-CoV-2 spike protein, a nucleotide sequence encoding an RSV F protein, a chimeric F protein, hMPV F protein) may be engineered into a NDV transcription unit so long as the insertion does not affect the ability of the virus to infect and replicate. The single segment is positioned between a T7 promoter and the hepatitis delta virus ribozyme to produce an exact negative or positive transcript from the T7 polymerase. The plasmid vector and expression vectors comprising the necessary viral proteins are transfected into cells leading to production of recombinant viral particles (see, e.g., International Publication No. WO 01/04333; U.S. Pat. Nos. 7,442,379, 6,146,642, 6,649,372, 6,544,785 and 7,384,774; Swayne et al. (2003). Avian Dis. 47:1047-1050; and Swayne et al. (2001). J. Virol. 11868-11873, each of which is incorporated by reference in its entirety).

Bicistronic techniques to produce multiple proteins from a single mRNA are known to one of skill in the art. Bicistronic techniques allow the engineering of coding sequences of multiple proteins into a single mRNA through the use of IRES sequences. IRES sequences direct the internal recruitment of ribosomes to the RNA molecule and allow downstream translation in a cap independent manner. Briefly, a coding region of one protein is inserted downstream of the ORF of a second protein. The insertion is flanked by an IRES and any untranslated signal sequences necessary for proper expression and/or function. The insertion must not disrupt the open reading frame, polyadenylation or transcriptional promoters of the second protein (see, e.g., Garcia-Sastre et al., 1994, J. Virol. 68:6254-6261 and Garcia-Sastre et al., 1994 Dev. Biol. Stand. 82:237-246, each of which are incorporated by reference herein in their entirety).

Methods for cloning recombinant NDV to encode a transgene and express a heterologous protein encoded by the transgene (e.g., a transgene encoding a SARS-CoV-2 spike protein, an RSV F protein, a chimeric F protein, hMPV F protein) are known to one skilled in the art, such as, e.g., insertion of the transgene into a restriction site that has been engineered into the NDV genome, inclusion an appropriate signals in the transgene for recognition by the NDV RNA-dependent-RNA polymerase (e.g., sequences upstream of the open reading frame of the transgene that allow for the NDV polymerase to recognize the end of the previous gene and the beginning of the transgene, which may be, e.g., spaced by a single nucleotide intergenic sequence), inclusion of a valid Kozak sequence (e.g., to improve eukaryotic ribosomal translation); incorporation of a transgene that satisfies the “rule of six” for NDV cloning; and inclusion of silent mutations to remove extraneous gene end and/or gene start sequences within the transgene. Regarding the rule of six, one skilled in the art will understand that efficient replication of NDV (and more generally, most members of the paramyxoviridae family) is dependent on the genome length being a multiple of six, known as the “rule of six” (see, e.g., Calain, P. & Roux, L. The rule of six, a basic feature of efficient replication of Sendai virus defective interfering RNA. J. Virol. 67, 4822-4830 (1993)). Thus, when constructing a recombinant NDV described herein, care should be taken to satisfy the “Rule of Six” for NDV cloning. Methods known to one skilled in the art to satisfy the Rule of Six for NDV cloning may be used, such as, e.g., addition of nucleotides downstream of the transgene. See, e.g., Ayllon et al., Rescue of Recombinant Newcastle Disease Virus from cDNA. J. Vis. Exp. (80), e50830, doi:10.3791/50830 (2013) for a discussion of methods for cloning and rescuing of NDV (e.g., recombinant NDV), which is incorporated by reference herein in its entirety.

In a specific embodiment, an NDV described herein (see, e.g., Sections 5.1 and 6) may be generated according to a method described in Section 6, infra.

### 5.3 Propagation of NDVS

The recombinant NDVs described herein (e.g., Sections 5.1 and 6) can be propagated in any substrate that allows the virus to grow to titers that permit the uses of the viruses described herein. In one embodiment, the substrate allows the recombinant NDVs described herein to grow to titers comparable to those determined for the corresponding wild-type viruses.

The recombinant NDVs described herein (e.g., Sections 5.1 and 6) may be grown in cells (e.g., avian cells, chicken cells, etc.) that are susceptible to infection by the viruses, embryonated eggs (e.g., chicken eggs or quail eggs) or animals (e.g., birds). Such methods are well-known to those skilled in the art. In a specific embodiment, the recombinant NDVs described herein may be propagated in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). In another specific embodiment, the recombinant NDVs described herein may be propagated in cell lines, e.g., cancer cell lines such as HeLa cells, MCF7 cells, THP-1 cells, U87 cells, DU145 cells, Lncap cells, and T47D cells. In certain embodiments, the cells or cell lines (e.g., cancer cells or cancer cell lines) are obtained, derived, or obtained and derived from a human(s). In another embodiment, the recombinant NDVs described herein are propagated in interferon deficient systems or interferon (IFN) deficient substrates, such as, e.g., IFN deficient cells (e.g., IFN deficient cell lines) or IFN deficient embyronated eggs. In another embodiment, the recombinant NDVs described herein are propagated in chicken cells or embryonated chicken eggs. Representative chicken cells include, but are not limited to, chicken embryo fibroblasts and chicken embryo kidney cells. In a specific embodiment, the recombinant NDVs described herein are propagated in Vero cells. In another specific embodiment, the recombinant NDVs described herein are propagated in chicken eggs or quail eggs. In certain embodiments, a recombinant NDV virus described herein is first propagated in embryonated eggs and then propagated in cells (e.g., a cell line).

The recombinant NDVs described herein may be propagated in embryonated eggs, e.g., from 6 to 14 days old, 6 to 12 days old, 6 to 10 days old, 6 to 9 days old, 6 to 8 days old, 8 to 10 day old, or 10 to 12 days old. In a specific embodiment, 10 day old embryonated chicken eggs are used to propagate the recombinant NDVs described herein. Young or immature embryonated eggs can be used to propagate the recombinant NDVs described herein. Immature embryonated eggs encompass eggs which are less than ten day old eggs, e.g., eggs 6 to 9 days old or 6 to 8 days old that are IFN-deficient. Immature embryonated eggs also encompass eggs which artificially mimic immature eggs up to, but less than ten day old, as a result of alterations to the growth conditions, e.g., changes in incubation temperatures; treating with drugs; or any other alteration which results in an egg with a retarded development, such that the IFN system is not fully developed as compared with ten to twelve day old eggs. The recombinant NDVs described herein can be propagated in different locations of the embryonated egg, e.g., the allantoic cavity. For a detailed discussion on the growth and propagation viruses, see, e.g., U.S. Pat. Nos. 6,852,522 and 7,494,808, both of which are hereby incorporated by reference in their entireties.

For virus isolation, the recombinant NDVs described herein can be removed from embryonated eggs or cell culture and separated from cellular components, typically by well-known clarification procedures, e.g., such as centrifugation, depth filtration, and microfiltration, and may be further purified as desired using procedures well known to those skilled in the art, e.g., tangential flow filtration (TFF), density gradient centrifugation, differential extraction, or chromatography.

In a specific embodiment, virus isolation from allantoic fluid of an infected egg (e.g., a chicken egg) begins with harvesting allantoic fluid, which is clarified using a filtration system to remove cells and other large debris, specifically, comprising a membrane having a net positive charge such that there is a measurable reduction in host cell DNA. The clarified bulk is subsequently processed by tangential flow filtration. The concentrated clarified bulk is then diafiltered against four diavolumes of high salt buffer, followed by four diavolumes of low salt formulation buffer and subsequently concentrated approximately 10-fold. Accordingly, residual egg proteins, e.g., primarily ovalbumin, and residual DNA are reduced to acceptable levels, and the buffer is exchanged to a buffer compatible with formulation of the recombinant NDV for a composition to be administered to a subject. The resulting product is then sterile filtered through a filter, e.g., a 0.2 μm filter, dispensed into appropriate sterile storage containers, frozen, and stored at −70 degrees Celsius.

In a specific embodiment, a recombinant NDV described herein (see, e.g., Sections 5.1 and 6) is propagated, isolated, and/or purified according to a method described in Section 6. In a specific embodiment, a recombinant NDV described herein (see, e.g., Sections 5.1 and 6) is either propagated, isolated, or purified, or any two or all of the foregoing.

In a specific embodiment, provided herein is a cell (e.g., a cell line) or embryonated egg (e.g., a chicken embryonated egg) comprising a recombinant NDV described herein. In some embodiments, the cell is in vitro or ex vivo. The cell may be a primary cell or cell line. The cell may be a mammalian (e.g., human) cell or cell line. In some embodiments, the cell is a cell or cell line recited herein. In some embodiments, the embryonated egg is an IFN-deficient substrate. In some embodiments, the embryonated egg is one described herein. In another specific embodiment, provided herein is a method for propagating a recombinant NDV described herein, the method comprising culturing a cell (e.g., a cell line) or embryonated egg (e.g., a chicken embryonated egg) infected with the recombinant NDV. In some embodiments, the method may further comprise isolating or purifying the recombinant NDV from the cell or embryonated egg. In a specific embodiment, provided herein is a method for propagating a recombinant NDV described herein, the method comprising (a) culturing a cell (e.g., a cell line) or embyronated egg infected with a recombinant NDV described herein; and (b) isolating the recombinant NDV from the cell or embyronated egg. The cell or embyronated egg may be one described herein or known to one of skill in the art. In some embodiments, the cell or embyronated egg is IFN deficient.

In a specific embodiment, provided herein is a method for producing a pharmaceutical composition (e.g., an immunogenic composition) comprising a recombinant NDV described herein, the method comprising (a) propagating a recombinant NDV described herein a cell (e.g., a cell line) or embyronated egg; and (b) isolating the recombinant NDV from the cell or embyronated egg. The method may further comprise adding the recombinant NDV to a container along with a pharmaceutically acceptable carrier.

### 5.4 Compositions and Routes of Administration

Provided herein are compositions comprising a recombinant NDV described herein (e.g., Section 5.1 or 6). In a specific embodiment, the compositions are pharmaceutical compositions, such as immunogenic compositions (e.g., vaccine compositions). In a specific embodiment, provided herein are immunogenic compositions comprising a recombinant NDV described herein (e.g., Section 5.1 or 6). The compositions may be used in methods of inducing an immune response to an antigen, such as described herein (e.g., in Section 5.1.3). The compositions may be used in methods for immunizing against an antigen (e.g., an antigen described herein (e.g., in Section 5.1.3)). The compositions may be used in methods for immunizing against a disease associated with an antigen (e.g., an antigen described herein (e.g., in Section 5.1.3)). The compositions may be used in methods for preventing a disease with which an antigen, such as an antigen described herein, is associated.

In one embodiments, a pharmaceutical composition (e.g., immunogenic composition) comprises a recombinant NDV described herein (e.g., Section 5.1 or 6), in an admixture with a pharmaceutically acceptable carrier. The composition may comprise 10⁴ to 10¹² PFU of a recombinant NDV described herein. In some embodiments, the pharmaceutical composition further comprises one or more additional prophylactic or therapeutic agents, such as described in Section 5.5.2, infra. In a specific embodiment, a pharmaceutical composition comprises an effective amount of a recombinant NDV described herein (e.g., Section 5.1 or 6), and optionally one or more additional prophylactic or therapeutic agents, in a pharmaceutically acceptable carrier. In certain embodiments, a pharmaceutical composition described herein comprises two recombinant NDV described herein, wherein the two recombinant NDV described herein are immunologically distinct from each other. In some embodiments, the recombinant NDV (e.g., Section 5.1 or 6) is the only active ingredient included in the pharmaceutical composition. In specific embodiments, two or more recombinant NDV are included in the pharmaceutical composition. In a particular embodiment, the pharmaceutical composition is an immunogenic composition.

In a specific embodiment, the recombinant NDV included in a pharmaceutical composition described herein is a live virus. In particular, embodiment, the recombinant NDV included in a pharmaceutical composition described herein is an attenuated live virus. In some embodiments, the recombinant NDV included in a pharmaceutical composition described herein is inactivated. Techniques known to one of skill in the art may be used to inactivate recombinant NDV.

The pharmaceutical compositions provided herein can be in any form that allows for the composition to be administered to a subject. In a specific embodiment, the pharmaceutical compositions are suitable for veterinary administration, human administration, or both. As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E.W. Martin. The formulation should suit the mode of administration.

In a specific embodiment, the pharmaceutical compositions are formulated to be suitable for the intended route of administration to a subject. For example, the pharmaceutical composition may be formulated to be suitable for parenteral, intravenous, intra-arterial, intrapleural, inhalation, intranasal, intraperitoneal, oral, intradermal, colorectal, intraperitoneal, intracranial, and intratumoral administration. In one embodiment, the pharmaceutical composition may be formulated for intravenous, intra-arterial, oral, intraperitoneal, intranasal, intratracheal, intrapleural, intracranial, subcutaneous, intramuscular, topical, pulmonary, or intratumoral administration. In a specific embodiment, the pharmaceutical composition may be formulated for intranasal administration.

In a specific embodiment, the pharmaceutical composition comprising a recombinant NDV described herein (see, e.g., Section 5.1 or 6) is formulated to be suitable for intranasal administration to the subject (e.g., human subject).

### 5.5 Uses of a Recombinant NDV

In another aspect, provided herein are methods for inducing an immune response in a subject (e.g., a human subject), the methods comprising administering the subject (e.g., a human subject) a recombinant NDV described herein or a composition thereof. In one embodiment, provided herein is a method for inducing an immune response in a subject (e.g., a human subject), the method comprising administering the subject (e.g., a human subject) an effective amount of a recombinant NDV described herein. See, e.g., Section 5.1 and 6 for recombinant NDV. In a specific embodiment, the immune response induced is an immune response to an antigen (e.g., an infectious disease antigen, or cancer or tumor antigen). The recombinant NDV may be administered to a subject by any route of administration. In another specific embodiment, the recombinant NDV is administered to a subject intranasally. In some embodiments, the recombinant NDV is administered to a subject intramuscularly.

In some embodiments, provided herein are methods for inducing antibodies in a subject. In some embodiments, provided herein are methods for inducing antibodies in a subject, comprising administering to the subject a recombinant NDV described herein, or a composition described herein. In certain embodiments, the subject is a non-human subject (e.g., a mouse, guinea pig, dog, cat, rabbit, monkey, chimpanzee, etc.) In other embodiments, the subject is human. The antibodies produced may be isolated and cloned as well as recombinantly engineered to, e.g., improve one or more of the properties of the antibody. In some embodiments, the antibodies induced bind to an antigen expressed by the recombinant NDV.

In another aspect, provided herein are methods for immunizing against a disease associated with an antigen (e.g., an infectious disease antigen, or cancer or tumor antigen), the methods comprising administering to a subject (e.g., a human subject) a recombinant NDV described herein or a composition thereof, wherein the recombinant NDV comprises a packaged genome comprising a transgene, wherein the transgene comprises a nucleotide sequence encoding an antigen associated with the disease (e.g., an infectious disease antigen, or cancer or tumor antigen). In one embodiment, provided herein is a method for immunizing against a disease associated with an antigen (e.g., an infectious disease antigen, or cancer or tumor antigen) in a subject (e.g., a human subject), the method comprising administering the subject (e.g., a human subject) an effective amount of a recombinant NDV described herein, wherein the recombinant NDV comprises a packaged genome comprising a transgene encoding an antigen associated with the disease (e.g., an infectious disease antigen, or cancer or tumor antigen). See, e.g., Section 5.1 and 6 for recombinant NDV. In a specific embodiment, the antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly.

In a specific embodiment, provided herein are methods for immunizing against a SARS-CoV-2 disease (e.g., COVID-19) comprising administering to a subject (e.g., a human subject) a recombinant NDV described herein or a composition thereof, wherein the recombinant NDV comprises a packaged genome comprising a transgene, and wherein the transgene comprises a nucleotide sequence encoding SARS-CoV-2 antigen (e.g., SARS-CoV-2 spike protein or a fragment thereof, such as a fragment comprising the receptor binding domain). In a specific embodiment, the SARS-CoV-2 antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly.

In a specific embodiment, provided herein are methods for immunizing against Ebola virus disease comprising administering to a subject (e.g., a human subject) a recombinant NDV described herein or a composition thereof, wherein the recombinant NDV comprises a packaged genome comprising a transgene, and wherein the transgene comprises a nucleotide sequence encoding an Ebola virus disease antigen. In a specific embodiment, the Ebola virus antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly.

In a specific embodiment, provided herein are methods for immunizing against Nipah virus disease comprising administering to a subject (e.g., a human subject) a recombinant NDV described herein or a composition thereof, wherein the recombinant NDV comprises a packaged genome comprising a transgene, and wherein the transgene comprises a nucleotide sequence encoding a Nipah virus disease antigen. In a specific embodiment, the Nipah virus antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly.

In a specific embodiment, provided herein are methods for immunizing against MERS-CoV disease comprising administering to a subject (e.g., a human subject) a recombinant NDV described herein or a composition thereof, wherein the recombinant NDV comprises a packaged genome comprising a transgene, and wherein the transgene comprises a nucleotide sequence encoding a MERS-CoV disease antigen. In a specific embodiment, the MERS-CoV antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly.

In a specific embodiment, provided herein are methods for immunizing against Lassa virus disease comprising administering to a subject (e.g., a human subject) a recombinant NDV described herein or a composition thereof, wherein the recombinant NDV comprises a packaged genome comprising a transgene, and wherein the transgene comprises a nucleotide sequence encoding a Lassa virus disease antigen. In a specific embodiment, the Lassa virus antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly.

In another aspect, provided herein are methods for immunizing a subject (e.g., a human subject) against an infectious disease, comprising administering to the subject a first recombinant NDV or a composition thereof and administering to the subject a second recombinant NDV or a composition thereof, wherein the first and second recombinant NDV are immunologically distinct from each other. In one embodiment, provided herein are methods for sequentially immunizing a subject (e.g., a human subject) against an infectious disease, comprising administering to the subject a first recombinant NDV or a composition thereof and administering to the subject a second recombinant NDV or a composition thereof, wherein the first and second recombinant NDV are immunologically distinct from each other. In certain embodiments, the first and second recombinant NDV may be administered 2 weeks, 3 weeks, 4 weeks, 6 weeks, 1 month, 3 months, 6 months, 9 months or 1 year apart. In some embodiments, the first and second recombinant NDV may be administered 2 to 4 weeks, 4 to 6 weeks, 1 to 3 months, 3 to 6 months, 3 to 9 months, 6 months to 1 year, or 1 to 2 years apart. The first and second recombinant NDVs or compositions thereof may be administered by the same route of administration or different routes of administration. In a specific embodiment, the first and second recombinant NDV are immunologically distinct from each other due to the replacement of the NDV F protein and/or HN protein with different a non-NDV APMV F protein and/or a different non-NDV APMV HN protein from each other. For example, the first recombinant NDV may comprise the F and HN proteins of APMV-15 and the second recombinant NDV may comprise the F and HN proteins from APMV-21. In certain embodiments, the first recombinant NDV is immunologically distinct from the second recombinant NDV if the first recombinant NDV and second recombinant NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a first recombinant NDV is considered immunologically distinct from a second recombinant NDV if the first recombinant NDV and the second recombinant NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In some embodiments, the method further comprises administering to the subject a third recombinant NDV or a composition thereof, wherein the third recombinant NDV is immunologically distinct from the first recombinant NDV, second recombinant NDV, or both the first and second recombinant NDVs. In certain embodiments, the method further comprises administering to the subject a third recombinant NDV or a composition thereof and a fourth recombinant NDV or a composition thereof, wherein the third recombinant NDV and fourth recombinant NDV are immunologically distinct from each other and third and fourth recombinant NDVs are immunologically distinct from the first recombinant NDV, second recombinant NDV, or both the first and second recombinant NDVs. In a specific embodiment, a recombinant NDV is considered immunologically distinct from another recombinant NDV if the recombinant NDV induces antibodies that inhibit the replication of another recombinant NDV in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs.

In one embodiment, provided herein are methods for sequentially immunizing a subject (e.g., a human subject) against an infectious disease, comprising administering to the subject a first recombinant NDV, administering to the subject a second recombinant NDV, and administering the subject a third recombinant NDV, wherein the first recombinant NDV, the second recombinant NDV and the third recombinant NDV are immunologically distinct from each other. In certain embodiments, the first and second recombinant NDV may be administered 2 weeks, 3 weeks, 4 weeks, 6 weeks, 1 month, 3 months, 6 months, 9 months or 1 year apart. In some embodiments, the first and second recombinant NDV may be administered 2 to 4 weeks, 4 to 6 weeks, 1 to 3 months, 3 to 6 months, 3 to 9 months, 6 months to 1 year, or 1 to 2 years apart. In a specific embodiment, the first, second and third recombinant NDV are immunologically distinct from each other due to the replacement of the NDV F protein and/or HN protein with different a non-NDV APMV F protein and/or a different non-NDV APMV HN protein from each other. For example, the first recombinant NDV may comprise the F and HN proteins of APMV-15, the second recombinant NDV may comprise the F and HN proteins from APMV-21, and the third recombinant NDV may comprise the F and HN proteins of APMV-10.

In a specific embodiment, two or more recombinant NDV described herein that are immunologically distinct from each other may be used to immunize a subject (e.g., human) against an infectious disease. In another specific embodiment, two or more recombinant NDV described herein that are immunologically distinct from each other may be used to immunize a subject (e.g., human) against cancer. In specific embodiments, the use of two or more recombinant NDVs having the NDV F protein and/or NDV HN protein replaced with a different non-NDV APMV F protein or variant thereof and/or a different non-NDV APMV HN protein or a variant thereof from each other permits multiple administrations of an antigen(s) to a subject (e.g., a human) in order to induce a robust immune response against the antigen(s).

In another aspect, provided herein are methods for inducing an immune response to an infectious disease antigen in a subject (e.g., a human subject), comprising administering to the subject a first recombinant NDV or a composition thereof and administering to the subject a second recombinant NDV or a composition thereof, wherein the first and second recombinant NDV are immunologically distinct from each other. In certain embodiments, the first and second recombinant NDV may be administered 2 weeks, 3 weeks, 4 weeks, 6 weeks, 1 month, 3 months, 6 months, 9 months or 1 year apart. In some embodiments, the first and second recombinant NDV may be administered 2 to 4 weeks, 4 to 6 weeks, 1 to 3 months, 3 to 6 months, 3 to 9 months, 6 months to 1 year, or 1 to 2 years apart. The first and second recombinant NDVs or compositions thereof may be administered by the same route of administration or different routes of administration. In some embodiments, the antigen expressed by the first recombinant NDV and the antigen expressed by the second recombinant NDV are from or derived from different pathogens. In other embodiments, the antigen expressed by the first recombinant NDV and the antigen expressed by the second recombinant NDV are from or derived from the same pathogen. The antigens expressed by the first and second recombinant NDVs may be identical or the antigen expressed by the second recombinant NDV may a variant thereof. For example, the antigen expressed by the first recombinant NDV may be a SARS-CoV-2 spike protein or a fragment thereof (e.g., a fragment comprising the receptor binding domain) from one strain and the antigen expressed by the second recombinant NDV may be a SARS-CoV-2 spike protein or a fragment thereof (e.g., a fragment comprising the receptor binding domain) from a variant strain of SARS-CoV-2. In a specific embodiment, the first and second recombinant NDV are immunologically distinct from each other due to the replacement of the NDV F protein and/or HN protein with different a non-NDV APMV F protein and/or a different non-NDV APMV HN protein from each other. For example, the first recombinant NDV may comprise the F and HN proteins of APMV-15 and the second recombinant NDV may comprise the F and HN proteins from APMV-21. In certain embodiments, the first recombinant NDV is immunologically distinct from the second recombinant NDV if the first recombinant NDV and second recombinant NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a first recombinant NDV is considered immunologically distinct from a second recombinant NDV if the first recombinant NDV and the second recombinant NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In some embodiments, the method further comprises administering to the subject a third recombinant NDV or a composition thereof, wherein the third recombinant NDV is immunologically distinct from the first recombinant NDV, second recombinant NDV, or both the first and second recombinant NDVs. In certain embodiments, the method further comprises administering to the subject a third recombinant NDV or a composition thereof and a fourth recombinant NDV or a composition thereof, wherein the third recombinant NDV and fourth recombinant NDV are immunologically distinct from each other and third and fourth recombinant NDVs are immunologically distinct from the first recombinant NDV, second recombinant NDV, or both the first and second recombinant NDVs.

In another aspect, provided herein are methods for immunizing a subject (e.g., a human subject) against cancer, comprising administering to the subject a first recombinant NDV or a composition thereof and administering to the subject a second recombinant NDV or a composition thereof, wherein the first and second recombinant NDV are immunologically distinct from each other. In one embodiment, provided herein are methods for sequentially immunizing a subject (e.g., a human subject) against cancer, comprising administering to the subject a first recombinant NDV or a composition thereof and administering to the subject a second recombinant NDV or a composition thereof, wherein the first and second recombinant NDV are immunologically distinct from each other. In certain embodiments, the first and second recombinant NDV may be administered 2 weeks, 3 weeks, 4 weeks, 6 weeks, 1 month, 3 months, 6 months, 9 months or 1 year apart. In some embodiments, the first and second recombinant NDV may be administered 2 to 4 weeks, 4 to 6 weeks, 1 to 3 months, 3 to 6 months, 3 to 9 months, 6 months to 1 year, or 1 to 2 years apart. The first and second recombinant NDVs or compositions thereof may be administered by the same route of administration or different routes of administration. In a specific embodiment, the first and second recombinant NDV are immunologically distinct from each other due to the replacement of the NDV F protein and/or HN protein with different a non-NDV APMV F protein and/or a different non-NDV APMV HN protein from each other. For example, the first recombinant NDV may comprise the F and HN proteins of APMV-15 and the second recombinant NDV may comprise the F and HN proteins from APMV-21. In certain embodiments, the first recombinant NDV is immunologically distinct from the second recombinant NDV if the first recombinant NDV and second recombinant NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a first recombinant NDV is considered immunologically distinct from a second recombinant NDV if the first recombinant NDV and the second recombinant NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In some embodiments, the method further comprises administering to the subject a third recombinant NDV or a composition thereof, wherein the third recombinant NDV is immunologically distinct from the first recombinant NDV, the second recombinant NDV, or both the first and second recombinant NDVs. In certain embodiments, the method further comprises administering to the subject a third recombinant NDV or a composition thereof and a fourth recombinant NDV or a composition thereof, wherein the third recombinant NDV and fourth recombinant NDV are immunologically distinct from each other and third and fourth recombinant NDVs are immunologically distinct from the first recombinant NDV, the second recombinant NDV, or both the first and second recombinant NDVs.

In another aspect, provided herein are methods for inducing an immune response to a cancer or tumor antigen in a subject (e.g., a human subject), comprising administering to the subject a first recombinant NDV or a composition thereof and administering to the subject a second recombinant NDV or a composition thereof, wherein the first and second recombinant NDV are immunologically distinct from each other. In certain embodiments, the first and second recombinant NDV may be administered 2 weeks, 3 weeks, 4 weeks, 6 weeks, 1 month, 3 months, 6 months, 9 months or 1 year apart. In some embodiments, the first and second recombinant NDV may be administered 2 to 4 weeks, 4 to 6 weeks, 1 to 3 months, 3 to 6 months, 3 to 9 months, 6 months to 1 year, or 1 to 2 years apart. In some embodiments, the cancer or tumor antigen expressed by the first recombinant NDV and the cancer or tumor antigen expressed by the second recombinant NDV are different. In certain embodiments, the cancer or tumor antigen expressed by the first recombinant NDV and the cancer or tumor antigen expressed by the second recombinant NDV are from or derived from the same type of cancer or tumor. The cancer or tumor antigen expressed by the first and second recombinant NDVs may be identical or the cancer or tumor antigen expressed by the second recombinant NDV may a variant thereof. In a specific embodiment, the first and second recombinant NDV are immunologically distinct from each other due to the replacement of the NDV F protein and/or HN protein with different a non-NDV APMV F protein and/or a different non-NDV APMV HN protein from each other. For example, the first recombinant NDV may comprise the F and HN proteins of APMV-15 and the second recombinant NDV may comprise the F and HN proteins from APMV-21. In certain embodiments, the first recombinant NDV is immunologically distinct from the second recombinant NDV if the first recombinant NDV and second recombinant NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a first recombinant NDV is considered immunologically distinct from a second recombinant NDV if the first recombinant NDV and the second recombinant NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In some embodiments, the method further comprises administering to the subject a third recombinant NDV or a composition thereof, wherein the third recombinant NDV is immunologically distinct from the first recombinant NDV, the second recombinant NDV, or both the first and second recombinant NDVs. In certain embodiments, the method further comprises administering to the subject a third recombinant NDV or a composition thereof and a fourth recombinant NDV or a composition thereof, wherein the third recombinant NDV and fourth recombinant NDV are immunologically distinct from each other and third and fourth recombinant NDVs are immunologically distinct from the first recombinant NDV, the second recombinant NDV, or both the first and second recombinant NDVs.

In another aspect, provided herein are methods for the prevention of an infectious disease, the methods comprising administering to a subject (e.g., a human subject) the recombinant NDV described herein or a composition thereof, wherein the recombinant NDV comprises a packaged genome comprising a transgene encoding an antigen associated with the infectious disease. In one embodiment, provided herein is a method for the prevention of an infectious disease, the method comprising administering the subject (e.g., a human subject) an effective amount of a recombinant NDV described herein, wherein the recombinant NDV comprises a packaged genome comprising a transgene encoding an antigen associated with the infectious disease. See, e.g., Section 5.1 and 6 for recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. In certain embodiments, the first and second recombinant NDV may be administered 2 weeks, 3 weeks, 4 weeks, 6 weeks, 1 month, 3 months, 6 months, 9 months or 1 year apart. In some embodiments, the first and second recombinant NDV may be administered 2 to 4 weeks, 4 to 6 weeks, 1 to 3 months, 3 to 6 months, 3 to 9 months, 6 months to 1 year, or 1 to 2 years apart. The first and second recombinant NDVs or compositions thereof may be administered by the same route of administration or different routes of administration. In a specific embodiment, the first and second recombinant NDV are immunologically distinct from each other due to the replacement of the NDV F protein and/or HN protein with different a non-NDV APMV F protein and/or a different non-NDV APMV HN protein from each other. For example, the first recombinant NDV may comprise the F and HN proteins of APMV-15 and the second recombinant NDV may comprise the F and HN proteins from APMV-21. In certain embodiments, the first recombinant NDV is immunologically distinct from the second recombinant NDV if the first recombinant NDV and second recombinant NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a first recombinant NDV is considered immunologically distinct from a second recombinant NDV if the first recombinant NDV and the second recombinant NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In some embodiments, the method further comprises administering to the subject a third recombinant NDV or a composition thereof, wherein the third recombinant NDV is immunologically distinct from the first recombinant NDV, the second recombinant NDV, or both the first and second recombinant NDVs. In certain embodiments, the method further comprises administering to the subject a third recombinant NDV or a composition thereof and a fourth recombinant NDV or a composition thereof, wherein the third recombinant NDV and fourth recombinant NDV are immunologically distinct from each other and third and fourth recombinant NDVs are immunologically distinct from the first recombinant NDV, the second recombinant NDV, or both the first and second recombinant NDVs.

In a specific embodiment, provided herein are methods for the prevention of RSV disease comprising administering to a subject (e.g., a human subject) a recombinant NDV described herein or a composition thereof, wherein the recombinant NDV comprises a packaged genome comprising a transgene, and wherein the transgene comprises a nucleotide sequence encoding a RSV antigen. In a specific embodiment, the RSV antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. The second recombinant NDV may comprise transgene comprising a nucleotide sequence encoding the same RSV antigen or a different RSV antigen.

In a specific embodiment, provided herein are methods for preventing human metapneumovirus disease comprising administering to a subject (e.g., a human subject) a recombinant NDV described herein or a composition thereof, wherein the recombinant NDV comprises a packaged genome comprising a transgene, and wherein the transgene comprises a nucleotide sequence encoding a human metapneumovirus antigen. In a specific embodiment, the human metapneumovirus antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. The second recombinant NDV may comprise transgene comprising a nucleotide sequence encoding the same hMPV antigen or a different hMPV antigen.

In a specific embodiment, provided herein are methods for preventing COVID-19 comprising administering to a subject (e.g., a human subject) a recombinant NDV described herein or a composition thereof, wherein the recombinant NDV comprises a packaged genome comprising a transgene, and wherein the transgene comprises a nucleotide sequence encoding a SARS-CoV-2 antigen (e.g., SARS-CoV-2 spike protein or a fragment thereof, such a fragment comprising the receptor binding domain). In a specific embodiment, the SARS-CoV-2 antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. The second recombinant NDV may comprise transgene comprising a nucleotide sequence encoding the same SARS-CoV-2 antigen or a different SARS-CoV-2 antigen.

In a specific embodiment, provided herein are methods for preventing Ebola virus disease comprising administering to a subject (e.g., a human subject) a recombinant NDV described herein or a composition thereof, wherein the recombinant NDV comprises a packaged genome comprising a transgene, and wherein the transgene comprises a Ebola virus disease antigen. In a specific embodiment, the Ebola virus antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. The second recombinant NDV may comprise transgene comprising a nucleotide sequence encoding the same Ebola virus antigen or a different Ebola antigen.

In a specific embodiment, provided herein are methods for preventing Nipah virus disease comprising administering to a subject (e.g., a human subject) a recombinant NDV described herein or a composition thereof, wherein the recombinant NDV comprises a packaged genome comprising a transgene, and wherein the transgene comprises a Nipah virus disease antigen. In a specific embodiment, the Nipah virus antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. The second recombinant NDV may comprise transgene comprising a nucleotide sequence encoding the same Nipah antigen or a different Nipah antigen.

In a specific embodiment, provided herein are methods for preventing MERS-CoV disease comprising administering to a subject (e.g., a human subject) a recombinant NDV described herein or a composition thereof, wherein the recombinant NDV comprises a packaged genome comprising a transgene, and wherein the transgene comprises a MERS-CoV disease antigen. In a specific embodiment, the MERS-CoV antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. The second recombinant NDV may comprise transgene comprising a nucleotide sequence encoding the same MERS-CoV antigen or a different MERS-CoV antigen.

In a specific embodiment, provided herein are methods for preventing Lassa virus disease comprising administering to a subject (e.g., a human subject) a recombinant NDV described herein or a composition thereof, wherein the recombinant NDV comprises a packaged genome comprising a transgene, and wherein the transgene comprises a Lassa virus disease antigen. In a specific embodiment, the Lassa virus antigen is expressed by cells infected with the recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. The second recombinant NDV may comprise transgene comprising a nucleotide sequence encoding the same Lassa virus antigen or a different Lassa virus antigen.

In a specific embodiment, a first recombinant NDV is considered immunologically distinct from a second recombinant NDV if the first recombinant NDV and the second recombinant NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs.

In another aspect, provided herein are methods for treating cancer, the methods comprising administering to a subject (e.g., a human subject) a recombinant NDV described herein or a composition thereof. See, e.g., Sections 5.1 and 6 for recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. In certain embodiments, the first and second recombinant NDV may be administered 2 weeks, 3 weeks, 4 weeks, 6 weeks, 1 month, 3 months, 6 months, 9 months or 1 year apart. In some embodiments, the first and second recombinant NDV may be administered 2 to 4 weeks, 4 to 6 weeks, 1 to 3 months, 3 to 6 months, 3 to 9 months, 6 months to 1 year, or 1 to 2 years apart. The first and second recombinant NDVs or compositions thereof may be administered by the same route of administration or different routes of administration. In a specific embodiment, the first and second recombinant NDV are immunologically distinct from each other due to the replacement of the NDV F protein and/or HN protein with different a non-NDV APMV F protein and/or a different non-NDV APMV HN protein from each other. For example, the first recombinant NDV may comprise the F and HN proteins of APMV-15 and the second recombinant NDV may comprise the F and HN proteins from APMV-21. In certain embodiments, the first recombinant NDV is immunologically distinct from the second recombinant NDV if the first recombinant NDV and second recombinant NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a first recombinant NDV is considered immunologically distinct from a second recombinant NDV if the first recombinant NDV and the second recombinant NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In some embodiments, the method further comprises administering to the subject a third recombinant NDV or a composition thereof, wherein the third recombinant NDV is immunologically distinct from the first recombinant NDV, the second recombinant NDV, or both the first and second recombinant NDVs. In certain embodiments, the method further comprises administering to the subject a third recombinant NDV or a composition thereof and a fourth recombinant NDV or a composition thereof, wherein the third recombinant NDV and fourth recombinant NDV are immunologically distinct from each other and third and fourth recombinant NDVs are immunologically distinct from the first recombinant NDV, the second recombinant NDV, or both the first and second recombinant NDVs.

In another aspect, provided herein are methods for treating cancer, the methods comprising administering to a subject (e.g., a human subject) a recombinant NDV described herein or a composition thereof, wherein the recombinant NDV comprises a packaged genome comprising a transgene, wherein the transgene comprises a nucleotide sequence encoding a cancer or tumor antigen. In one embodiment, provided herein is a method for treating cancer, the method comprising administering the subject (e.g., a human subject) an effective amount of a recombinant NDV described herein, wherein the recombinant NDV comprises a packaged genome comprising a transgene, and wherein the transgene comprises a nucleotide sequence encoding a cancer or tumor antigen associated with the cancer. See, e.g., Sections 5.1 and 6 for recombinant NDV. The recombinant NDV may be administered to a subject by any route of administration. In another specific embodiment, the recombinant NDV is administered to a subject intranasally. In some embodiments, the recombinant NDV is administered to a subject intramuscularly. In some embodiments, the method further comprise administering to the subject a second recombinant NDV or a composition thereof, wherein the second recombinant NDV is immunologically distinct than the first recombinant NDV. In certain embodiments, the first and second recombinant NDV may be administered 2 weeks, 3 weeks, 4 weeks, 6 weeks, 1 month, 3 months, 6 months, 9 months or 1 year apart. In some embodiments, the first and second recombinant NDV may be administered 2 to 4 weeks, 4 to 6 weeks, 1 to 3 months, 3 to 6 months, 3 to 9 months, 6 months to 1 year, or 1 to 2 years apart. The cancer or tumor antigen expressed by the first recombinant NDV may be the same or different than the cancer or tumor antigen expressed by the second recombinant NDV. The first and second recombinant NDVs or compositions thereof may be administered by the same route of administration or different routes of administration. In a specific embodiment, the first and second recombinant NDV are immunologically distinct from each other due to the replacement of the NDV F protein and/or HN protein with different a non-NDV APMV F protein and/or a different non-NDV APMV HN protein from each other. For example, the first recombinant NDV may comprise the F and HN proteins of APMV-15 and the second recombinant NDV may comprise the F and HN proteins from APMV-21. In certain embodiments, the first recombinant NDV is immunologically distinct from the second recombinant NDV if the first recombinant NDV and second recombinant NDV do not induce antibodies that substantially inhibit replication of the other as assessed by a virus neutralization assay, such as described in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein. In a specific embodiment, a first recombinant NDV is considered immunologically distinct from a second recombinant NDV if the first recombinant NDV and the second recombinant NDV induce antibodies that inhibit the replication of each other in a virus neutralization assay, such as described, e.g., in Chumbe et al., 2017, Virology Journal 14: 232 and Reynolds et al., 1999, Avian Dis. 143:564-71, Sun et al., 2020, EBioMedicine 62: 103132, or Sun et al., 2020, Vaccines 8: 771, or described herein, by less than about 0.5 logs, less than about 1 log, less than about 1.5 logs, or less than about 2 logs. In some embodiments, the method further comprises administering to the subject a third recombinant NDV or a composition thereof, wherein the third recombinant NDV is immunologically distinct from the first recombinant NDV, the second recombinant NDV, or both the first and second recombinant NDVs. In certain embodiments, the method further comprises administering to the subject a third recombinant NDV or a composition thereof and a fourth recombinant NDV or a composition thereof, wherein the third recombinant NDV and fourth recombinant NDV are immunologically distinct from each other and third and fourth recombinant NDVs are immunologically distinct from the first recombinant NDV, the second recombinant NDV, or both the first and second recombinant NDVs.

The recombinant NDV described herein may be administered to a subject in combination with one or more other therapies. The recombinant NDV and one or more other therapies may be administered by the same or different routes of administration to the subject. In a specific embodiment, the recombinant NDV is administered to a subject intranasally. See, e.g., Sections 5.1, and 6, infra for information regarding recombinant NDV, Section 5.5.2 for information regarding other therapies, and Section 5.4, infra, for information regarding compositions and routes of administration.

The recombinant NDV and one or more additional therapies may be administered concurrently or sequentially to the subject. In certain embodiments, the recombinant NDV and one or more additional therapies are administered in the same composition. In other embodiments, the recombinant NDV and one or more additional therapies are administered in different compositions. The recombinant NDV and one or more other therapies may be administered by the same or different routes of administration to the subject. Any route known to one of skill in the art or described herein may be used to administer the recombinant NDV and one or more other therapies. In a specific embodiment, the recombinant NDV is administered intranasally and the one or more other therapies is administered intravenously.

In some embodiments, a recombinant NDV described herein or a composition thereof, or a combination therapy described herein is administered to a subject that has previously been vaccinated or administered NDV composition (e.g., a vaccine). In certain embodiments, a recombinant NDV described herein or a composition thereof, or a combination therapy described herein is administered to a subject that has previously been vaccinated or administered an APMV-based composition (e.g. a vaccine). In some embodiments, a recombinant NDV described herein or a composition thereof, or a combination therapy described herein is administered to a subject that has previously been vaccinated or administered NDV composition (e.g., a vaccine) and an APMV-based composition (e.g. a vaccine). In specific embodiments, the APMV-based composition is a non-NDV APMV.

In some embodiments, a recombinant NDV described herein or a composition thereof, or a combination therapy described herein is administered to a patient to prevent the onset of one, two or more symptoms of an infectious disease (such a patient may be at risk of developing an infection). In a specific embodiment, the administration of a recombinant NDV described herein or a composition thereof, or a combination therapy described herein to a subject prevents the onset or development of one, two or more symptoms of infectious disease, reduces the severity of one, two or more symptoms of infectious disease, or prevents the onset or development of one, two or more symptoms of infectious disease and reduces the severity of one, two or more symptoms of infectious disease.

In some embodiments, a recombinant NDV described herein or a composition thereof, or a combination therapy described herein is administered to a patient to prevent the onset of one, two or more symptoms of RSV disease. In a specific embodiment, the administration of a recombinant NDV described herein or a composition thereof, or a combination therapy described herein to a subject prevents the onset or development of one, two or more symptoms of RSV disease, reduces the severity of one, two or more symptoms of RSV disease, or prevents the onset or development of one, two or more symptoms of RSV disease and reduces the severity of one, two or more symptoms of RSV disease. Symptoms of RSV disease include congested or runny nose, cough, fever, sore throat, headache, wheezing, rapid or shallow breathing or difficulty breathing, bluish color the skin due to lack of oxygen, lack of appetite, lethargy and irritability. In another specific embodiment, the administration of a recombinant NDV described herein or a composition thereof, or a combination therapy described herein to a subject prevents otitis media caused by a RSV infection. In another specific embodiment, the administration of a recombinant NDV described herein or a composition thereof, or a combination therapy described herein to a subject prevents bronchiolitis caused by a RSV infection. In another specific embodiment, the administration of a recombinant NDV described herein or a composition thereof, or a combination therapy described herein to a subject prevents pneumonia caused by a RSV infection.

In some embodiments, a recombinant NDV described herein or a composition thereof, or a combination therapy described herein is administered to a patient to prevent the onset of one, two or more symptoms of Ebola virus disease. In a specific embodiment, the administration of a recombinant NDV described herein or a composition thereof, or a combination therapy described herein to a subject prevents the onset or development of one, two or more symptoms of Ebola virus disease, reduces the severity of one, two or more symptoms of Ebola virus disease, or prevents the onset or development of one, two or more symptoms of Ebola virus disease and reduces the severity of one, two or more symptoms of Ebola virus disease. Symptoms of Ebola virus disease include fever, aches and pains (e.g., a severe headache, muscle and joint pain, and abdominal (stomach) pain), weakness and fatigue, gastrointestinal symptoms (e.g., diarrhea and vomiting), abdominal (stomach) pain, and unexplained hemorrhaging, bleeding or bruising.

In some embodiments, a recombinant NDV described herein or a composition thereof, or a combination therapy described herein is administered to a patient to prevent the onset of one, two or more symptoms of an hMPV disease (e.g., such a patient is at risk of developing an hMPV infection). In a specific embodiment, the administration of a recombinant NDV described herein or a composition thereof, or a combination therapy described herein to a subject prevents the onset or development of one, two or more symptoms of hMPV disease, reduces the severity of one, two or more symptoms of hMPV disease, or prevents the onset or development of one, two or more symptoms of hMPV disease and reduces the severity of one, two or more symptoms of hMPV disease. Symptoms of hMPV disease include nasal congestion, runny nose, fever, cough, sore throat, wheezing, difficulty breathing, lack of appetite, lethargy, and irritability. In another specific embodiment, the administration of a recombinant NDV described herein or a composition thereof, or a combination therapy described herein to a subject prevents bronchiolitis caused by an hMPV infection. In another specific embodiment, the administration of a recombinant NDV described herein or a composition thereof, or a combination therapy described herein to a subject prevents pneumonia caused by an hMPV infection.

In some embodiments, a recombinant NDV described herein or a composition thereof, or a combination therapy described herein is administered to a patient to prevent the onset of one, two or more symptoms of Lassa virus disease. In a specific embodiment, the administration of a recombinant NDV described herein or a composition thereof, or a combination therapy described herein to a subject prevents the onset or development of one, two or more symptoms of Lassa virus disease, reduces the severity of one, two or more symptoms of Lassa virus disease, or prevents the onset or development of one, two or more symptoms of Lassa virus disease and reduces the severity of one, two or more symptoms of Lassa virus disease. Symptoms of Lassa virus disease include light fever, general malaise and weakness, headache, hemorrhaging, respiratory distress, repeated vomiting, facial swelling, pain in the chest, back, and abdomen, shock, hearing loss, tremors, and encephalitis.

In some embodiments, a recombinant NDV described herein or a composition thereof, or a combination therapy described herein is administered to a patient to prevent the onset of one, two or more symptoms of MERS-CoV disease. In a specific embodiment, the administration of a recombinant NDV described herein or a composition thereof, or a combination therapy described herein to a subject prevents the onset or development of one, two or more symptoms of MERS-CoV disease, reduces the severity of one, two or more symptoms of MERS-CoV disease, or prevents the onset or development of one, two or more symptoms of MERS-CoV disease and reduces the severity of one, two or more symptoms of MERS-CoV disease. Symptoms of MERS-CoV disease include fever, cough, and shortness of breath.

In some embodiments, a recombinant NDV described herein or a composition thereof, or a combination therapy described herein is administered to a patient to prevent the onset of one, two or more symptoms of Nipah virus disease. In a specific embodiment, the administration of a recombinant NDV described herein or a composition thereof, or a combination therapy described herein to a subject prevents the onset or development of one, two or more symptoms of Nipah virus disease, reduces the severity of one, two or more symptoms of Nipah virus disease, or prevents the onset or development of one, two or more symptoms of Nipah virus disease and reduces the severity of one, two or more symptoms of Nipah virus disease. Symptoms of Nipah virus disease include disorientation, drowsiness, confusion, seizures, coma, and brain swelling (encephalitis).

In some embodiments, a recombinant NDV described herein or a composition thereof, or a combination therapy described herein is administered to a patient to prevent the onset of one, two or more symptoms of COVID-19. In a specific embodiment, the administration of a recombinant NDV described herein or a composition thereof, or a combination therapy described herein to a subject prevents the onset or development of one, two or more symptoms of COVID-19, reduces the severity of one, two or more symptoms of COVID-19, or prevents the onset or development of one, two or more symptoms of COVID-19 and reduces the severity of one, two or more symptoms of COVID-19. Symptoms of COVID-19 include congested or runny nose, cough, fever, sore throat, headache, wheezing, rapid or shallow breathing or difficulty breathing, bluish color the skin due to lack of oxygen, chills, muscle pain, loss of taste and/or smell, nausea, vomiting, and diarrhea. In another specific embodiment, the administration of a recombinant NDV described herein or a composition thereof, or a combination therapy described herein to a subject prevents pneumonia caused by a SARS-CoV-2 infection.

In a specific embodiment, the administration of a recombinant NDV described herein or a composition thereof, or a combination therapy described herein to a subject prevents the spread of an infection. In another specific embodiment, the administration of a recombinant NDV described herein or a composition thereof, or a combination therapy described herein to a subject prevents hospitalization. In another specific embodiment, the administration of a recombinant NDV described herein or a composition thereof, or a combination therapy described herein to a subject prevents recurring infections.

In some embodiments, a recombinant NDV described herein or a composition thereof, or a combination therapy described herein is administered to a subject suffering from an infectious disease. In other embodiments, an NDV (e.g., a recombinant NDV) described herein or a composition thereof, or a combination therapy described herein is administered to a subject predisposed or susceptible to an infectious disease. In some embodiments, a recombinant NDV described herein or a composition thereof, or a combination therapy described herein is administered to a subject diagnosed as having an infectious disease. In some embodiments, an NDV (e.g., a recombinant NDV) or a composition thereof, or a combination therapy described herein is administered to a subject seronegative for antibodies to a pathogen (e.g., antibodies to a SARS-CoV-2 antigen, RSV antigen, human metapneumovirus antigen, Nipah virus antigen, MERS-CoV antigen, Lassa virus antigen or Ebola virus antigen). In some embodiments, an NDV (e.g., a recombinant NDV) or a composition thereof, or a combination therapy described herein is administered to a subject seropositive for antibodies to a pathogen (e.g., antibodies to a SARS-CoV-2 antigen, RSV antigen, human metapneumovirus antigen, Nipah virus antigen, MERS-CoV antigen, Lassa virus antigen or Ebola virus antigen). In certain embodiments, the subject is assessed for antibodies prior to administration of a recombinant NDV described herein or a composition thereof, or a combination therapy described herein. In other embodiments, the subject is not assessed for antibodies prior to administration of a recombinant NDV described herein or a composition thereof, or a combination therapy described herein.

In a specific embodiment, a method of treating cancer described herein may result in a beneficial effect for a subject, such as the reduction, decrease, attenuation, diminishment, stabilization, remission, suppression, inhibition or arrest of the development or progression of cancer, or a symptom thereof. In certain embodiments, a method of treating cancer described herein results in at least one, two or more of the following effects: (i) the reduction or amelioration of the severity of cancer and/or a symptom associated therewith; (ii) the reduction in the duration of a symptom associated with cancer; (iii) the prevention in the recurrence of a symptom associated with cancer; (iv) the regression of cancer and/or a symptom associated therewith; (v) the reduction in hospitalization of a subject; (vi) the reduction in hospitalization length; (vii) the increase in the survival of a subject; (viii) the inhibition of the progression of cancer and/or a symptom associated therewith; (ix) the enhancement or improvement of the therapeutic effect of another therapy; (x) a reduction or elimination in the cancer cell population; (xi) a reduction in the growth of a tumor or neoplasm; (xii) a decrease in tumor size; (xiii) a reduction in the formation of a tumor; (xiv) eradication, removal, or control of primary, regional and/or metastatic cancer; (xv) a decrease in the number or size of metastases; (xvi) a reduction in mortality; (xvii) an increase in cancer-free survival rate of patients; (xviii) an increase in relapse-free survival; (xix) an increase in the number of patients in remission; (xx) a decrease in hospitalization rate; (xxi) the size of the tumor is maintained and does not increase in size or increases the size of the tumor by less than 5% or 10% after administration of a therapy as measured by conventional methods available to one of skill in the art, such as MRI, X-ray, CT Scan and PET scan; (xxii) the prevention of the development or onset of cancer and/or a symptom associated therewith; (xxiii) an increase in the length of remission in patients; (xxiv) the reduction in the number of symptoms associated with cancer; (xxv) an increase in symptom-free survival of cancer patients; (xxvi) limitation of or reduction in metastasis; (xxvii) overall survival; (xxviii) progression-free survival (as assessed, e.g., by RECIST v1.1.); (xxix) overall response rate; and/or (xxx) an increase in response duration. In some embodiments, the treatment/therapy that a subject receives does not cure cancer, but prevents the progression or worsening of the disease. In certain embodiments, a method of treating cancer described herein does not prevent the onset/development of cancer, but may prevent the onset of cancer symptoms. Any method known to the skilled artisan may be utilized to evaluate the treatment/therapy that a subject receives. In a specific embodiment, the efficacy of a treatment/therapy is evaluated according to the Response Evaluation Criteria In Solid Tumors (“RECIST”) published rules. In a specific embodiment, the efficacy of a treatment/therapy is evaluated according to the RECIST rules published in February 2000 (also referred to as “RECIST 1”) (see, e.g., Therasse et al., 2000, Journal of National Cancer Institute, 92(3):205-216, which is incorporated by reference herein in its entirety). In a specific embodiment, the efficacy of a treatment/therapy is evaluated according to the RECIST rules published in January 2009 (also referred to as “RECIST 1.1”) (see, e.g., Eisenhauer et al., 2009, European Journal of Cancer, 45:228-247, which is incorporated by reference herein in its entirety). In a specific embodiment, the efficacy of a treatment/therapy is evaluated according to the RECIST rules utilized by the skilled artisan at the time of the evaluation. In a specific embodiment, the efficacy is evaluated according to the immune related RECIST (“irRECIST”) published rules (see, e.g., Bohnsack et al., 2014, ESMO Abstract 4958, which is incorporated by reference herein in its entirety).

In some embodiments, the administration of a recombinant NDV described herein or a composition thereof, or a combination therapy described herein to a subject increases infiltration of one, two or all of the following cell types into a tumor: (i) T-cells, (ii) natural killer (NK) cells, and (iii) dendritic cells. In certain embodiments, the administration of a recombinant NDV described herein or a composition thereof, or a combination therapy described herein to a subject described herein increases lymphocyte infiltration into a tumor. In a specific embodiment, the administration of a recombinant NDV described herein or a composition thereof, or a combination therapy described herein to a subject increases T cell infiltration (CD4+ T cell infiltration and/or CD8+ T cell infiltration) into a tumor. In certain embodiments, the administration of a recombinant NDV described herein or a composition thereof, or a combination therapy to a subject increases cytokine production in a tumor (e.g., increases INFγ, IL-2, and/or TNF production).

In another specific embodiment, the administration of a recombinant NDV described herein or a composition thereof, or a combination therapy described herein induces antibodies to an antigen (e.g., an infectious disease antigen, or cancer or tumor antigen). In another specific embodiment, the administration of a recombinant NDV described herein or a composition thereof, or a combination therapy described herein induces both mucosal and systemic antibodies to an antigen (e.g., an infectious disease antigen, or cancer or tumor antigen), such as, e.g., neutralizing antibodies. In another specific embodiment, the administration of a recombinant NDV described herein or a composition thereof, or a combination therapy described herein to a subject induces neutralizing antibody to an antigen (e.g., an infectious disease antigen, or cancer or tumor antigen).

In some embodiments, a recombinant NDV described herein or a composition thereof, or a combination therapy described herein is administered to a subject suffering cancer. In other embodiments, an NDV (e.g., a recombinant NDV) described herein or a composition thereof, or a combination therapy described herein is administered to a subject predisposed or susceptible to cancer. In some embodiments, a recombinant NDV described herein or a composition thereof, or a combination therapy described herein is administered to a subject diagnosed as having cancer.

In specific embodiments, a recombinant NDV described herein or a composition thereof, or a combination therapy described herein is administered to a human.

### 5.5.1 Dosage and Frequency

The amount of a recombinant NDV or a composition thereof, which will be effective in the prevention of disease, immunization against a pathogen, or in treating cancer will depend on the route of administration, the general health of the subject, etc. Suitable dosage ranges of a recombinant NDV for administration are generally about 10⁴ to about 10¹², and can be administered to a subject once, twice, three, four or more times with intervals as often as needed. In certain embodiments, dosages similar to those currently being used in clinical trials for NDV are administered to a subject.

In certain embodiments, a recombinant NDV or a composition thereof is administered to a subject as a single dose followed by a second dose 1 to 6 weeks, 1 to 5 weeks, 1 to 4 weeks, 1 to 3 weeks, 1 to 2 weeks, 6 to 12 weeks, 3 to 6 months, 6 to 9 months, 6 to 12 months, or 6 to 9 months later. In accordance with these embodiments, booster inoculations may be administered to the subject at 3 to 6 month or 6 to 12 month intervals following the second inoculation.

In certain embodiments, administration of the same recombinant NDV or a composition thereof may be repeated and the administrations may be separated by at least 1 day, 2 days, 3 days, 5 days, 6 says, 7 days, 10 days, 14 days, 15 days, 21 days, 28 days, 30 days, 45 days, 2 months, 75 days, 3 months, or at least 6 months. In other embodiments, administration of the same recombinant NDV or a composition thereof may be repeated and the administrations may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months. In some embodiments, a first recombinant NDV or a composition thereof is administered to a subject followed by the administration of a second recombinant NDV or a composition thereof. In some embodiments, the first and second recombinant NDV are different from each other. For example, the first recombinant NDV may comprise nucleotide sequences encoding the F and HN proteins of a first type of non-NDV APMV (e.g. APMV-12) and the second recombinant NDV may comprise nucleotide sequences encoding the F and HN proteins of a second type of non-NDV APMV (e.g., APMV-10). In a specific embodiment, the first and second recombinant NDV are immunologically distinct from each other. In certain embodiments, the first and second recombinant NDVs or compositions thereof may be separated by at least 1 day, 2 days, 3 days, 5 days, 6 days, 7 days, 10 days, 14 days, 15 days, 21 days, 28 days, 30 days, 45 days, 2 months, 75 days, 3 months, or at least 6 months. In other embodiments, the first and second recombinant NDVs or compositions thereof may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months.

In certain embodiments, a recombinant NDV or composition thereof is administered to a subject in combination with one or more additional therapies, such as a therapy described in Section 5.5.2, infra. The dosage of the other one or more additional therapies will depend upon various factors including, e.g., the therapy, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. Recommended doses for approved therapies can be found in the Physician's Desk Reference.

In certain embodiments, a recombinant NDV or composition thereof is administered to a subject concurrently with the administration of one or more additional therapies. In certain embodiments, the recombinant NDV and or composition thereof and one or more additional therapies are administered to the subject within 1 minute, 2 minutes, 5 minutes, 10 minutes, 15 minutes, 20 minutes, 30 minutes, 45 minutes, 60 minutes, 1.5 hours, 2 hours, 3 hours, 4 hours, 5 hours, or 6 hours of each other. In certain embodiments, the recombinant NDV and or composition thereof and one or more additional therapies are administered to the subject within 12 hours, 1 day, 2 days, 3 days, 4 days, 5 days, 6 days, 7 days, 2 weeks, 3 weeks, 4 weeks, 5 weeks, 6 weeks, 7 weeks, 8 weeks or 12 weeks of each other. In certain embodiments, the recombinant NDV and or composition thereof and one or more additional therapies are administered to the subject within 3-6 months, 6-9 months, 6-12 months, or 3 months, 4 months, 6 months, 9 months, or 12 months of each other.

In certain embodiments, a first pharmaceutical composition is administered to a subject as a priming dose and after a certain period (e.g., 1 month, 2 months, 3 months, 4 months, 5 months, 6 months, or 1-6 months) a booster dose of a second pharmaceutical composition is administered. In some embodiments, the second pharmaceutical composition comprises the same recombinant NDV as the first pharmaceutical composition. In other embodiments, the second pharmaceutical composition comprises a recombinant NDV that is immunologically distinct than the recombinant NDV of the first pharmaceutical composition. In specific embodiments, the second pharmaceutical composition comprises the same recombinant NDV as the first pharmaceutical composition with the exception that the F protein and/or HN protein, are from a different non-NDV APMV F protein or variant thereof and/or a different non-NDV APMV HN protein or a variant thereof.

### 5.5.2 Additional Therapies

Additional therapies that can be used in a combination with a recombinant NDV described herein or a composition thereof include, but are not limited to, acetaminophen, a chemotherapeutic, a checkpoint inhibitor, an immunotherapy, ibuprofen, throat lozenges, cough suppressants, inhalers, antibiotics and oxygen. In a specific embodiment, the additional therapy is a second recombinant NDV described herein.

### 5.6 Biological Assays

In a specific embodiment, a biological assay known to one of skill in the art to characterize a recombinant NDV described herein, or an antigen. In specific embodiments, a microneutralization assay known to one of skill in the art or described herein is used to assess for antibodies that bind to a recombinant NDV described herein. In some embodiments, the ability of anti-NDV F antibodies to bind to a non-NDV APMV F protein or a variant thereof may be assessed by any method know to one of skill in the art (e.g., an immunoassay). In certain embodiments, the ability of anti-NDV HN antibodies to bind to a non-NDV APMV HN protein or a variant thereof may be assessed by any method know to one of skill in the art (e.g., an immunoassay). In some embodiments, a hemagglutinin inhibition assay, which is known to one of skill in the art or described herein, may be used may be used to assess whether two recombinant NDVs described herein, or an NDV and non-NDV APMV are immunologically distinct.

### 5.6.1 In Vitro Viral Assays

Viral assays include those that indirectly measure viral replication (as determined, e.g., by plaque formation) or the production of viral proteins (as determined, e.g., by western blot analysis) or viral RNAs (as determined, e.g., by RT-PCR or northern blot analysis) in cultured cells in vitro using methods which are well known in the art.

Growth of the recombinant NDVs described herein can be assessed by any method known in the art or described herein (e.g., in cell culture (e.g., cultures of chicken embryonic kidney cells or cultures of chicken embryonic fibroblasts (CEF)). Viral titer may be determined by inoculating serial dilutions of a recombinant NDV described herein into cell cultures (e.g., CEF, MDCK, EFK-2 cells, Vero cells, primary human umbilical vein endothelial cells (HUVEC), H292 human epithelial cell line or HeLa cells), chick embryos, or live animals (e.g., avians). After incubation of the virus for a specified time, the virus is isolated using standard methods. Physical quantitation of the virus titer can be performed using PCR applied to viral supernatants (Quinn & Trevor, 1997; Morgan et al., 1990), hemagglutination assays, tissue culture infectious doses (TCID50) or egg infectious doses (EID50).

Incorporation of nucleotide sequences encoding a heterologous peptide or protein (e.g., a transgene into the genome of a recombinant NDV described herein can be assessed by any method known in the art or described herein (e.g., in cell culture, an animal model or viral culture in embryonated eggs)). For example, viral particles from cell culture of the allantoic fluid of embryonated eggs can be purified by centrifugation through a sucrose cushion and subsequently analyzed for protein expression by Western blotting using methods well known in the art. Other immunoassays, such as ELISA may be used to detect protein expression.

Immunofluorescence-based approaches may also be used to detect virus and assess viral growth. Such approaches are well known to those of skill in the art, e.g., fluorescence microscopy and flow cytometry. Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

### 5.6.2 Interferon Assays

IFN induction and release by a recombinant NDV described herein may be determined using techniques known to one of skill in the art. For example, the amount of IFN induced in cells following infection with a recombinant NDV described herein may be determined using an immunoassay (e.g., an ELISA or Western blot assay) to measure IFN expression or to measure the expression of a protein whose expression is induced by IFN. Alternatively, the amount of IFN induced may be measured at the RNA level by assays, such as Northern blots and quantitative RT-PCR, known to one of skill in the art. In specific embodiments, the amount of IFN released may be measured using an ELISPOT assay. Further, the induction and release of cytokines and/or interferon-stimulated genes may be determined by, e.g., an immunoassay or ELISPOT assay at the protein level and/or quantitative RT-PCR or northern blots at the RNA level.

### 5.6.3 Activation Marker Assays and Immune Cell Infiltration Assay

Techniques for assessing the expression of T cell marker, B cell marker, activation marker, co-stimulatory molecule, ligand, or inhibitory molecule by immune cells are known to one of skill in the art. For example, the expression of T cell marker, B cell marker, an activation marker, co-stimulatory molecule, ligand, or inhibitory molecule by an immune cell can be assessed by flow cytometry.

### 5.6.4 Toxicity Studies

In some embodiments, the recombinant NDVs described herein or compositions thereof, or combination therapies described herein are tested for cytotoxicity in mammalian, preferably human, cell lines. In certain embodiments, cytotoxicity is assessed in one or more of the following non-limiting examples of cell lines: U937, a human monocyte cell line; primary peripheral blood mononuclear cells (PBMC); Huh7, a human hepatoblastoma cell line; HL60 cells, HT1080, HEK 293T and 293H, MLPC cells, human embryonic kidney cell lines; human melanoma cell lines, such as SkMel2, SkMel-119 and SkMel-197; THP-1, monocytic cells; a HeLa cell line; and neuroblastoma cells lines, such as MC-IXC, SK-N-MC, SK-N-MC, SK-N-DZ, SH-SY5Y, and BE(2)-C. In some embodiments, the ToxLite assay is used to assess cytotoxicity.

Many assays well-known in the art can be used to assess viability of cells or cell lines following infection with a recombinant NDV described herein or composition thereof, and, thus, determine the cytotoxicity of the recombinant NDV or composition thereof. For example, cell proliferation can be assayed by measuring Bromodeoxyuridine (BrdU) incorporation, (³H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc.). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability. In preferred embodiments, a recombinant NDV described herein or composition thereof does not kill healthy (i.e., non-cancerous) cells.

In specific embodiments, cell viability may be measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes.

The recombinant NDVs described herein or compositions thereof, or combination therapies can be tested for in vivo toxicity in animal models. For example, animal models, known in the art to test the effects of compounds on RSV infection or hMPV infection can also be used to determine the in vivo toxicity of the recombinant NDVs described herein or compositions thereof, or combination therapies. For example, animals are administered a range of pfu of a recombinant NDV described herein, and subsequently, the animals are monitored over time for various parameters, such as one, two or more of the following: lethality, weight loss or failure to gain weight, and levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and regimen in addition to dosages.

The toxicity, efficacy or both of a recombinant NDV described herein or a composition thereof, or a combination therapy described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from the cell culture assays and animal studies can be used in formulating a range of dosage of the therapies for use in subjects. The dosage of such agents lies preferably within a range of circulating concentrations that include the ED50 with little or no toxicity. The dosage may vary within this range depending upon the dosage form employed and the route of administration utilized. For any therapy described herein, the therapeutically effective dose can be estimated initially from cell culture assays.

### 5.6.5 Biological Activity Assays

The recombinant NDVs described herein or compositions thereof, or combination therapies described herein can be tested for biological activity using animal models for inhibiting an infectious disease or cancer, antibody response to the recombinant NDVs, etc. Such animal model systems include, but are not limited to, rats, mice, hamsters, cotton rats, chicken, cows, monkeys (e.g., African green monkey), pigs, dogs, rabbits, etc.

In a specific embodiment, the recombinant NDVs described herein or compositions thereof, or combination therapies described herein may be tested using animal models for the ability to induce a certain geometric mean titer of antibody(ies) that binds to the antigen. In another specific embodiment, the recombinant NDVs described herein or compositions thereof, or combination therapies described herein may be tested using animal models for the ability to induce antibodies that have neutralizing activity against an antigen in a microneutralization assay. In some embodiments, the recombinant NDVs described herein or compositions thereof, or combination therapies described herein may be tested using animal models for the ability to induce a certain geometric mean titer of antibody(ies) that binds to the antigen (e.g., SARS-CoV-2 antigen, Ebola virus antigen, MERS-CoV antigen, Lassa virus antigen, RSV antigen, or human metapneumovirus antigen) and neutralizes the virus associated with the antigen in a microneutralization assay. In a specific embodiment, the recombinant NDVs described herein or compositions thereof, or combination therapies described herein may be tested using animal models for the ability to induce a certain fold increase in levels of antibody(ies) that binds to antigen post-immunization with a recombinant NDV described herein or a composition thereof relative to the levels of such antibody pre-immunization. For example, a 3 fold, 4 fold, 5 fold, 6 fold, 7 fold, 8 fold or greater increase in levels of antibody(ies) that binds antigen post-immunization with a recombinant NDV described herein or a composition thereof relative to the levels of such antibody(ies) pre-immunization.

### 5.6.6 Expression of Transgene

Assays for testing the expression of a non-NDV APMV F protein, a non-NDV APMV HN protein, a chimeric F protein, a chimeric HN protein, an antigen (including a chimeric antigen) in cells infected with a recombinant NDV described herein may be conducted using any assay known in the art, such as, e.g., western blot, immunofluorescence, and ELISA, or any assay described herein.

In a specific aspect, ELISA is utilized to detect expression of a non-NDV APMV F protein, a non-NDV APMV HN protein, a chimeric F protein, a chimeric HN protein, an antigen (including a chimeric antigen) in cells infected with a recombinant NDV described herein.

In one embodiment, a non-NDV APMV F protein, a non-NDV APMV HN protein, a chimeric F protein, a chimeric HN protein, an antigen (including a chimeric antigen) encoded by a packaged genome of a recombinant NDV described herein is assayed for proper folding by testing its ability to bind specifically to an antibody using any assay for antibody-antigen interaction known in the art. In another embodiment, encoded by a packaged genome of a recombinant NDV described herein is assayed for proper folding by determination of the structure or conformation of a non-NDV APMV F protein, a non-NDV APMV HN protein, a chimeric F protein, a chimeric HN protein, an antigen (including a chimeric antigen) using any method known in the art such as, e.g., NMR, X-ray crystallographic methods, or secondary structure prediction methods, e.g., circular dichroism. Additional assays assessing the conformation and antigenicity of a non-NDV APMV F protein, a non-NDV APMV HN protein, a chimeric F protein, a chimeric HN protein, an antigen (including a chimeric antigen) may include, e.g., immunofluorescence microscopy, flow cytometry, western blot, and ELISA may be used. In vivo immunization in animal models, such as cotton rats or mice, may also be used to assess the antigenicity of a non-NDV APMV F protein, a non-NDV APMV HN protein, a chimeric F protein, a chimeric HN protein, an antigen (including a chimeric antigen).

Assays for testing the functionality of a non-NDV APMV F protein, a non-NDV APMV HN protein, a chimeric F protein, a chimeric HN protein, an antigen (including a chimeric antigen) in cells infected with a recombinant NDV described herein may be conducted using any assay known in the art. For example, the receptor binding and neuraminidase activities of the HN protein may be assessed. The fusion of the virus to host cell may also be assessed.

### 5.7 Kits

In one aspect, provided herein is a pharmaceutical pack or kit comprising one or more containers filled with one or more of the ingredients of a composition (e.g., a pharmaceutical compositions) described herein. In a specific embodiment, provided herein is a pharmaceutical pack or kit comprising a container, wherein the container comprises a recombinant NDV described herein, or a pharmaceutical composition comprising the recombinant NDV. In certain embodiments, the pharmaceutical pack or kit further comprises a second recombinant NDV, or a pharmaceutical composition comprising the second recombinant NDV. In specific embodiments, that is second recombinant NDV is immunologically distinct from the first recombinant NDV. In some embodiments, provided herein is pharmaceutical pack or kit comprising the pNDV-F-HNless acceptor plasmid described in Section 6. In certain embodiments, the pack or kit further comprises a nucleic acid sequence comprising a nucleotide of any one of SEQ ID NOS:1-14. In certain embodiments, provided herein is a pharmaceutical pack or kit comprising a nucleic acid sequence comprising a nucleotide sequence of any one of SEQ ID NOS:1-14, or a nucleotide sequence that is at least 80%, at least 85%, at least 90%, at least 95%, or at least 98% identical to the nucleotide sequence of any one of SEQ ID NOS:1-14. Optionally associated with such container(s) can be a notice in the form prescribed by a governmental agency regulating the manufacture, use or sale of pharmaceuticals or biological products, which notice reflects approval by the agency of manufacture, use or sale for human administration.

In some embodiments, provided herein is pharmaceutical pack or kit comprising a nucleic acid sequence comprising a nucleotide sequence that is at least 80%, at least 85%, at least 90%, at least 95%, or at least 98% identical to the nucleotide sequence of any one of SEQ ID NOS:1-14. In some embodiments, the pack or kit further comprises a nucleic acid sequence comprising (or consisting of): (1) a transcription unit encoding a NDV nucleocapsid (N) protein, (2) a transcription unit encoding a NDV phosphoprotein (P), (3) a transcription unit encoding a NDV matrix (M) protein, and (4) a transcription unit encoding a NDV large polymerase (L). In some embodiments, the NDV is of the LaSota strain.

In some embodiments, provided herein is pharmaceutical pack or kit comprising a nucleic acid sequence comprising SEQ ID NO:44 or 45. In some embodiments, provided herein is pharmaceutical pack or kit comprising a nucleic acid sequence comprising SEQ ID NO:44 or 45 without the GFP coding sequence. In some embodiments, the pack or kit further comprises a nucleic acid sequence comprising a nucleotide sequence of any one of SEQ ID NOS:1-14. In some embodiments, the pack or kit further comprises a nucleic acid sequence comprising a nucleotide sequence that is at least 80%, at least 85%, at least 90%, at least 95%, or at least 98% identical to the nucleotide sequence of any one of SEQ ID NOS:1-14. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.8 Sequences

The sequences disclosed in this section may be used to produce the recombinant NDV described herein.

TABLE 1
Synthetic sequences for the generation of chimeric NDV-APMV
rescue plasmids and NDV LaSota Sequence

| | | SEQ |
|---|---|---|
| Description | Sequence | ID NO. |
| APMV4/duck/ | atttacagttagtttacctgtctatcaagttagaaaaaacacgggtagaagattctggatcccg | 1 |
| Hongkong/ | gttggcgccctccaggtgcaagATGAGGCTATCAAACACAATCTTG | |
| D3/75 | ACCTTGATTCTCATCATACTTACCGGCTATTTGATAGGT | |
| (lower cases | GTCCACTCCACCGATGTGAATGAGAAACCAAAGTCCG | |
| correspond | AAGGGATTAGGGGTGATCTTACACCAGGTGCGGGTATT | |
| to NDV | TTCGTAACTCAAGTCCGACAGCTCCAGATCTACCAACA | |
| derived | GTCTGGGTACCATGATCTTGTCATCAGATTGTTACCTCT | |
| sequences | TCTACCAACAGAGCTTAATGATTGTCAAAGGGAAGTTG | |
| and upper | TCACAGAGTACAATAACACTGTATCACAGCTGTTGCAG | |
| cases | CCTATCAAAACCAACCTGGATACTTTGTTGGCAGATGG | |
| correspond | TAGCACAAGGGATGTTGATATACAGCCGCGATTCATTG | |
| to APMV F | GGGCAATAATAGCCACAGGTGCCCTGGCTGTAGCAAC | |
| and HN | GGTAGCTGAGGTAACTGCAGCTCAAGCACTATCTCAGT | |
| coding | CAAAAACGAATGCTCAAAATATTCTCAAGTTGAGAGA | |
| sequences) | TAGTATTCAGGCCACCAACCAAGCAGTTTTTGAAATTT | |
| | CACAGGGACTCGAAGCAACTGCAACCGTGCTATCAAA | |
| | ACTGCAAACTGAGCTCAATGAGAATATCATCCCAAGTC | |
| | TGAACAACTTGTCCTGTGCTGCCATGGGGAATCGCCTT | |
| | GGTGTATCACTCTCACTCTATTTGACCTTAATGACCACT | |
| | CTATTTGGGGACCAGATCACAAACCCAGTGCTGACGCC | |
| | AATCTCTTACAGCACCCTATCGGCAATGGCGGGTGGTC | |
| | ACATTGGTCCAGTGATGAGTAAGATATTAGCCGGATCT | |
| | GTCACAAGTCAGTTGGGGGCAGAACAACTGATTGCTA | |
| | GTGGCTTAATACAGTCACAGGTAGTAGGTTATGATTCC | |
| | CAGTATCAGCTGTTGGTTATCAGGGTCAACCTTGTACG | |
| | GATTCAGGAAGTCCAGAATACTAGGGTTGTATCACTAA | |
| | GAACACTAGCAGTCAATAGGGATGGTGGACTTTACAG | |
| | AGCCCAGGTGCCACCCGAGGTAGTTGAGCGATCTGGC | |
| | ATTGCAGAGCGGTTTTATGCAGATGATTGTGTTCTAAC | |
| | TACAACTGATTACATCTGCTCATCGATCCGATCTTCTCG | |
| | GCTTAATCCAGAGTTAGTCAAGTGTCTCAGTGGGGCAC | |
| | TTGATTCATGCACATTTGAGAGGGAAAGTGCATTACTG | |
| | TCAACTCCCTTCTTTGTATACAACAAGGCAGTCGTCGC | |
| | AAATTGTAAAGCAGCGACATGTAGATGTAATAAACCG | |
| | CCATCTATCATTGCCCAATACTCTGCATCAGCTCTAGT | |
| | AACCATCACCACCGACACTTGTGCTGACCTTGAAATTG | |
| | AGGGTTATCGTTTCAACATACAGACTGAATCCAACTCA | |
| | TGGGTTGCACCAAACTTCACGGTCTCAACCTCACAAAT | |
| | AGTATCGGTTGATCCAATAGACATATCCTCTGACATTG | |
| | CCAAAATTAACAATTCTATCGAGGCTGCGCGAGAGCA | |
| | GCTGGAACTGAGCAACCAGATCCTTTCCCGAATCAACC | |
| | CACGGATTGTGAACGACGAATCACTAATAGCTATTATC | |
| | GTGACAATTGTTGTGCTTAGTCTCCTTGTAATTGGTCTT | |
| | ATTATTGTTCTCGGTGTGATGTACAAGAATCTTAAGAA | |
| | AGTCCAACGAGCTCAAGCTGCTATGATGATGCAGCAA | |
| | ATGAGCTCATCACAGCCTGTGACCACCAAATTGGGGAC | |
| | ACCCTTCTGAtgaacacagatgaggaacgaaggtttccctaatagtaatttgtgtga | |
| | aagttctggtagtctgtcagttcagagagttaagaaaaaactaccggttgtagatgaccaaa | |
| | ggacgatatacgggtagaacggtaagagaggccgcccctcaattgcgagccaggcttca | |
| | caacctccgttctaccgcttcaccgacaacagtcctcaatcATGCAGGGCGACA | |
| | TGGAGGGTAGCCGTGATAACCTCACAGTAGATGATGA | |
| | ATTAAAGACAACATGGAGGTTAGCTTATAGAGTTGTAT | |
| | CCCTCCTATTGATGGTGAGTGCCTTGATAATCTCTATA | |
| | GTAATCCTGACGAGAGATAACAGCCAAAGCATAATCA | |
| | CGGCGATCAACCAGTCGTATGACGCAGACTCAAAGTG | |
| | GCAAACAGGGATAGAAGGGAAAATCACCTCAATCATG | |
| | ACTGATACGCTCGATACCAGGAATGCAGCTCTTCTCCA | |
| | CATTCCACTCCAGCTCAATACACTTGAGGCAAACCTGT | |
| | TGTCCGCCCTCGGAGGTTACACGGGAATTGGCCCCGGA | |
| | GATCTAGAGCACTGTCGTTATCCGGTTCATGACTCCGC | |
| | TTACCTGCATGGAGTCAATCGATTACTCATCAATCAAA | |
| | CAGCTGACTACACAGCAGAAGGCCCCCTGGATCATGT | |
| | GAACTTCATTCCGGCACCAGTTACGACTACTGGATGCA | |
| | CAAGGATCCCATCCTTTTCTGTATCATCATCCATTTGGT | |
| | GCTATACACACAATGTGATTGAAACAGGTTGCAATGAC | |
| | CACTCAGGTAGTAATCAATATATCAGTATGGGGGTGAT | |
| | TAAGAGGGCTGGCAACGGCTTACCTTACTTCTCAACAG | |
| | TCGTGAGTAAGTATCTGACCGATGGGTTGAATAGAAA | |
| | AAGCTGTTCCGTAGCTGCGGGATCCGGGCATTGTTACC | |
| | TCCTTTGTAGCCTAGTGTCAGAGCCCGAACCTGATGAC | |
| | TATGTGTCACCAGATCCCACACCGATGAGGTTAGGGGT | |
| | GCTAACAAGGGATGGGTCTTACACTGAACAGGTGGTA | |
| | CCCGAAAGAATATTTAAGAACATATGGAGCGCAAACT | |
| | ACCCTGGGGTAGGGTCAGGTGCTATAGCAGGAAATAA | |
| | GGTGTTATTCCCATTTTACGGCGGAGTGAAGAATGGAT | |
| | CAACCCCTGAGGTGATGAATAGGGGAAGATATTACTA | |
| | CATCCAGGATCCAAATGACTATTGCCCTGACCCGCTGC | |
| | AAGATCAGATCTTAAGGGCAGAACAATCGTATTATCCT | |
| | ACTCGATTTGGTAGGAGGATGGTAATGCAGGGAGTCCT | |
| | AACATGTCCAGTATCCAACAATTCAACAATAGCCAGCC | |
| | AATGCCAATCTTACTATTTCAACAACTCATTAGGATTC | |
| | ATCGGGGCGGAATCTAGGATCTATTACCTCAATGGTAA | |
| | CATTTACCTTTATCAAAGAAGCTCGAGCTGGTGGCCTC | |
| | ACCCCCAAATTTACCTACTTGATTCCAGGATTGCAAGT | |
| | CCGGGTACGCAGAACATTGACTCAGGCGTTAACCTCAA | |
| | GATGTTAAATGTTACTGTCATTACACGACCATCATCTG | |
| | GCTTTTGTAATAGTCAGTCAAGATGCCCTAATGACTGC | |
| | TTATTCGGGGTTTATTCAGATGTCTGGCCTCTTAGCCTT | |
| | ACCTCAGACAGCATATTTGCATTTACAATGTACTTACA | |
| | AGGGAAGACGACACGTATTGACCCAGCTTGGGCGCTA | |
| | TTCTCCAATCATGTAATTGGGCATGAGGCTCGTTTGTTC | |
| | AACAAGGAGGTTAGTGCTGCTTATTCTACCACCACTTG | |
| | TTTTTCGGACACCATCCAAAACCAGGTGTATTGTCTGA | |
| | GTATACTTGAAGTCAGAAGTGAGCTCTTGGGGGCATTC | |
| | AAGATAGTGCCATTCCTCTATCGTGTCTTAtagttgagtcaatta | |
| | taaaggagttggaaagatggcattgtatcacctatcttctgcgacatcaagaatca | |
| APMV17/ | atttacagttagtttacctgtctatcaagttagaaaaaacacgggtagaagattctggatcccg | 2 |
| Antarctica/ | gttggcgccctccaggtgcaagATGCAATTGTACTCAGTCCTGGCT | |
| 107/13 | CTGGCCTTACTGGCAGTTCAAGCGGAGCTCGGTATAAT | |
| (lower cases | CCCCTCCCTGAGCAACACACACTCAATAGATGCAGGAT | |
| correspond | TTGTGTTCCAGTCTGAGCGAGCAGTCAATATCTACACC | |
| to NDV | AATTCATTAACCGGGAGTGTGGTAGTTAAACTGCTCCC | |
| derived | TAACTTACCAGATCACCTCAAAAGCTGCCACCTTGATG | |
| sequences | TCCTAAGCAGTTACAACCGCACCCTTACATCTATCTTC | |
| and upper | CAACCGETAGGTGAGAGTATAAAGCATATATGGGGTA | |
| cases | ACACTACAGGAGGGTCAGCCGCAGGAGGAATCCAGTC | |
| correspond | TCGGATAGTGGGTGCCATACTTGGAGGTGTAGCCTTAG | |
| to APMV F | GTGTAGCAACATCAGCACAGATTACAGCAGGAGTTGC | |
| and HN | CTTAGCACAATCCAGGCAGAATGCAGAGAATATCTTA | |
| coding | AAGTTGAAACAGTCTATTGCTGCAACAAATGATGCTGT | |
| sequences) | GCAAGAGGTTATTGCAGGACAACGAGAGCTTGTGATT | |
| | GCAATAGGTAAAATGCAGGATTACATCAATCAAGCGC | |
| | TTAACAGCACTATCCAGCAAATCGACTGTGTTACTGCT | |
| | GCAAATCGGCTCGGAGTGGAACTTAGCTTGTATCTGAC | |
| | TCAGCTTACCACCGCCTTCAGTAATCAAATCCAGAACC | |
| | CTGCACTCACTCCGTTGTCTATTCAGGCGTTATATAACC | |
| | TAGCTGGAGGTAATTTGGATAGGTTCCTCAATCGCATT | |
| | GGAGCTACCACATCTAATCTACAGTCCATCATATCAAG | |
| | TGGGCTAATTCAGGGGCAACCTATTGGCTACGACTCTG | |
| | AGAAGCAGCTATTGATCCTGTCTGTATCTGTACCAAGC | |
| | ATAAATGCAGTGGATAATTTGCGCATGGCGCAGCTGAC | |
| | CCCCATAGTGGTGTCCACTAGCCAGGGGCTGGGAGCTG | |
| | TAGTTATCCCCAAATATATCATTGCAATCGCAGACTTA | |
| | ATAGAGGAGTTTGTGGCTGATGACTGTATCTTCACAAC | |
| | ATCTGATGCATATTGTACCAGCCTTACCACACTACCTC | |
| | TCAGCAATTCACTGCAGCAATGCATCAGGGGCAATGTG | |
| | TCAGCATGCTCGTACTCGCTGGTGAGGGGAGTACTATC | |
| | GACCAAGTTCATCACCCTTGATGGCTCTGTTATAGCCA | |
| | ACTGCCAAGCAGTGACATGTAGATGCATTGATCCCCCC | |
| | AAAATCATATCACAATTTGCCGGGAAGCCGCTCACCAT | |
| | TATCAATAGCAAGATATGCAACATTATCAATATTGAGC | |
| | AAGTTACGCTAAGGCTGTCTGGTCATTTCATGTCACAA | |
| | TATGGTGCTAATCTTAGTATCAGCGAAGGGCAAATTGT | |
| | GGTTACTGGACCCCTGGACATTAGCAATGAGCTTGGTC | |
| | GAGTCAACCAAAGTATCACGAATGCGCAAGCATCCAT | |
| | AGATAAGAGTAACCAGATTCTGGAAGGTGTCAATGTC | |
| | AGGCTGATACAGGTGCCAGCCTTAGCAACGTCACTTGC | |
| | TCTGGCAATTGCAGGGACTGTATTGGGCGCGCTGGCAA | |
| | TAATTGGGATCTTAGTGTTATGGGCAGCTAATAAGAAG | |
| | CAGAGCAAAAAGATGGAGTGGCTGCTCGCATCAAAGG | |
| | CATCAAGGATGTGAtgaacacagatgaggaacgaaggtttccctaatagtaat | |
| | ttgtgtgaaagttctggtagtctgtcagttcagagagttaagaaaaaactaccggttgtagatg | |
| | accaaaggacgatatacgggtagaacggtaagagaggccgcccctcaattgcgagccag | |
| | gcttcacaacctccgttctaccgottcaccgacaacagtcctcaatcATGCCATCCA | |
| | TCATGACATCCACCTCATCGCAAAGTCGGGAGCATCTT | |
| | GCATCACGGGATGATGACGATGACAGCAAGTGTACCT | |
| | GGCGGCTGGTGTTCAGGGTGTCGGCCATAGCCCTACTT | |
| | CTTACCATTCTTGGTCTCTCGATTGCAACATTCTGTAAA | |
| | ATACCTTCAAAGGATTTTGAACCCATAATAGAAGAAGC | |
| | TGTACATGAGATCACTTCGATATTGACACCGCTAGGTG | |
| | CAGGGATTACTGCAATCTTGGATTACTGCCAAAAGATA | |
| | TACCGCCAGAGTGTGCTAGAGACTCCCCTGCAGTTGTC | |
| | AGCCATGCAAACTAGTATACTTCAGAGCCTCAGTGCGT | |
| | TGTCTTATCAAATTAGTCTGGAGGCCAACGGGAGTAAT | |
| | TGCGGCGCACCCATTCATGATGAGGCCTTTGCAGGAGG | |
| | TATTGAGACACCTCTTTTCTCTGGGAAATTCACTAATG | |
| | GTAAGCAATTCAGGGTAAGTAAGTATATAGAACACCTT | |
| | AACTTCATACCGGCACCTACCACTGGGAGAGGCTGCAC | |
| | CAGAATACCATCGTTCTCTCTGTCCACTAGTCACGGAT | |
| | GCTACACACACAATGTGATCTTGGACGGATGTGCAGAT | |
| | CATGGCGCGTCCCACCAGTATATATCAATCGGAACTCT | |
| | AAGAGTGTCCCCATCTGGGAGAATATACTTCTCAACCC | |
| | TCCGGAGTGTCAACTTGGATGATGGGGTGAACCGGAA | |
| | GTCATGCAGCATCGCAGCTACCCGCTATGGGTGTGACC | |
| | TGTTATGCTCAGTGGTAACAGAAACGGAAAGGAGCGA | |
| | CTATGCGTCAAACCCACCTACCCGGATGATACACGGAA | |
| | GGTTGGATTTCGGGGGTTCTTATAGCGAGACTGATATC | |
| | AATAGTCAGGTGCTCTTCTCTGACTGGGCAGCTAACTA | |
| | CCCAGGTGTGGGGTCAGGAGTTGCAGTAGATGATAGG | |
| | ATTCTGTTCCCGATATATGGGGGACTCAGAGCGGGAAC | |
| | CCCTTCCTATAACAGGAACTATGGTTCATATGCCATCT | |
| | ATCAGAGAAGCGGTGATGTGTGTCCTGACAACAATGCT | |
| | ACTCAAGTAAGAAATGCAAAAGCATCTTACATTGTGCC | |
| | TCTATTCTCCAATCGATTGATACAACAGGCCATCCTAT | |
| | CTATCAAGTTAGATCCCGGTCTAGGGAAGGATACTACA | |
| | CTTCACATTTCATCTAATAATGTGACATTGATGGGTGC | |
| | AGAGGCTAGACTAGTAGCCATTGATGGCCAGGTGTAC | |
| | ATGTATCAAAGAGGCAGCTCATGGTTCCCAGCTGCAGT | |
| | CCTGTACCCCATACACAGAAAGAATGGCACATTCGCAT | |
| | TTGGGAGACCATACATATATGATAACTTTACGAGGCCT | |
| | GGCACAGGCTTCTGTTCTGCAGCTAGCAGATGCCCAAA | |
| | CACATGTATTACAGGGGTTTACACGGATGCTTTCCCAA | |
| | TTGTCTTTTCTGCGGACAAAAAGCCGATAGGGGTGTTC | |
| | GGGACTTATCTCAACCATCGTAGTGATCGTCAAAATCC | |
| | TAGATCTGCTGTGTTCTTTGATGTCTCCATGAGCAATGC | |
| | AACTAATGTCTCCACACCCCCCGTAGGTGCTGCATACA | |
| | CTACATCCACATGTTTCAAAATGACCTCAACCGGACGG | |
| | CGGTACTGCATATCAATAGCAGAAATTAGGAACACCA | |
| | TTTTCGGGGAATACAGAATTGTGCCTCTCCTTGTTGAA | |
| | ATAGAACAGGTGtagttgagtcaattataaaggagttggaaagatggcattgtat | |
| | cacctatcttctgcgacatcaagaatca | |
| APMV9/duck/ | atttacagttagtttacctgtctatcaagttagaaaaaacacgggtagaagattctggatcccg | 3 |
| New York/ | gttggcgccctccaggtgcaagATGGGGTACTTCCACCTATTACTT | |
| 22/78 | ATACTAACAGCGATTGCCATATCTGCGCACCTCTGCTA | |
| (lower cases | TACCACGACATTGGATGGTAGAAAACTGCTTGGTGCAG | |
| correspond | GCATAGTGATAACAGAAGAGAAGCAAGTTAGGGTGTA | |
| to NDV | CACAGCTGCGCAATCAGGAACAATTGTCTTAAGGTCTT | |
| derived | TCCGTGTGGTCTCCTTAGACAGATACTCGTGCATGGAA | |
| sequences | TCCACTATTGAGTCATATAACAAGACTGTATATAACAT | |
| and upper | ACTTGCACCTCTGGGCGATGCAATCCGCCGAATACAGG | |
| cases | CAAGTGGTGTATCGGTTGAGCGTATCCGAGAGGGCCG | |
| correspond | CATATTTGGTGCCATCCTTGGGGGAGTTGCCTTAGGTG | |
| to APMV F | TAGCCACCGCAGCACAGATAACAGCTGCAATTGCTTTG | |
| and HN | ATTCAGGCTAACGAGAACGCAAAAAACATCCTGCGTA | |
| coding | TTAAAGACAGTATAACTAAGACCAACGAGGCAGTGAG | |
| sequences) | AGATGTAACTAATGGCGTGTCGCAGTTAACTATCGCTG | |
| | TAGGTAAATTACAGGACTTCGTCAATAAGGAATTCAAT | |
| | AAGACAACTGAGGCCATTAATTGTGTACAGGCAGCTC | |
| | AACAATTAGGTGTGGAGCTAAGCCTCTATCTGACCGAG | |
| | ATCACTACAGTCTTCGGACCTCAGATAACCTCTCCTGC | |
| | TTTAAGCAAATTGACTATCCAAGCGCTGTATAATTTGG | |
| | CGGGCGTAAGCTTGGATGTACTACTGGGAAGGCTCGG | |
| | AGCAGACAATTCACAGTTATCATCTTTGGTTAGTAGTG | |
| | GTCTTATTACCGGACAGCCCATTCTCTACGACTCGGAA | |
| | TCTCAAATATTGGCACTGCAAGTGTCACTACCCTCCAT | |
| | TAGTGACTTAAGGGGAGTGAGAGCGACATACTTAGAC | |
| | ACGTTGGCTGTCAACACTGCAGCAGGACTTGCATCTGC | |
| | TATGATTCCAAAGGTAGTAATCCAATCTAATAATATAG | |
| | TTGAAGAATTAGATACTACAGCATGTATAGCAGCAGA | |
| | AGCTGACTTATACTGTACGAGGATTACTACATTCCCCA | |
| | TTGCGTCGGCTGTATCAGCCTGCATTCTTGGGGATGTA | |
| | TCGCAATGCCTTTATTCAAAGACTAATGGCGTCTTAAC | |
| | CACTCCATATGTAGCAGTAAAGGGGAAAATTGTAGCC | |
| | AATTGTAAGCATGTCACATGTAGGTGTGTAGATCCTAC | |
| | ATCCATCATATCTCAAAATTACGGTGAAGCAGCGACTC | |
| | TTATCGATGATCAGCTATGCAAGGTAATCAACTTAGAT | |
| | GGTGTGTCCATACAGCTGAGCGGCACATTTGAATCGAC | |
| | TTATGTGCGCAACGTCTCGATAAGTGCAAACAAGGTCA | |
| | TTGTCTCAAGCAGTATAGATATATCTAATGAGCTGGAG | |
| | AATGTTAACAGCTCTTTAAGTTCGGCTCTGGAAAAACT | |
| | GGATGAAAGTGACGCTGCGCTAAGCAAAGTAAATGTT | |
| | CACTTAACTAGCACCTCAGCTATGGCCACATACATTGT | |
| | TCTAACTGTAATTGCTCTTATCTTGGGGTTTGTCGGCCT | |
| | AGGATTGGGTTGCTTTGCTATGATAAAAGTAAAGTCTC | |
| | AAGCAAAGACACTACTATGGCTTGGTGCACATGCTGAC | |
| | CGATCATATATACTCCAGAGTAAGCCGGCTCAATCGTC | |
| | CACAtgaacacagatgaggaacgaaggtttccctaatagtaatttgtgtgaaagttctggt | |
| | agtctgtcagttcagagagttaagaaaaaactaccggttgtagatgaccaaaggacgatata | |
| | cgggtagaacggtaagagaggccgcccctcaattgcgagccaggcttcacaacctccgtt | |
| | ctaccgcttcaccgacaacagtcctcaatcATGGAATCAGGAATCAGCC | |
| | AGGCATCTCTTGTCAATGACAACATAGAATTAAGGAAT | |
| | ACGTGGCGCACGGCCTTCCGTGTGGTCTCCTTATTACT | |
| | CGGCTTCACCAGCTTGGTGCTCACTGCTTGCGCTTTAC | |
| | ACTTCGCTTTGAATGCCGCTACCCCTGCGGATCTCTCTA | |
| | GTATCCCAGTCGCTGTTGACCAAAGTCATCATGAAATT | |
| | CTACAAACCTTGAGTCTGATGAGCGACATTGGCAATAA | |
| | GATTTACAAGCAGGTAGCACTAGATAGTCCAGTGGCG | |
| | CTGCTCAACACTGAATCAACCTTAATGAGCGCAATTAC | |
| | ATCACTATCTTATCAGATTAACAATGCAGCGAATAACT | |
| | CAGGTTGTGGCGCCCCTGTGCATGATAAGGATTTTATC | |
| | AATGGAGTGGCAAAGGAATTATTTGTAGGGTCTCAATA | |
| | CAATGCCTCGAACTATCGACCCTCCAGGTTCCTTGAGC | |
| | ATCTAAATTTCATCCCCGCCCCTACTACGGGAAAAGGT | |
| | TGCACCAGAATTCCGTCCTTTGATCTAGCTGCAACACA | |
| | TTGGTGTTATACTCACAATGTGATTCTTAATGGTTGTAA | |
| | TGATCATGCTCAATCTTATCAATACATATCCCTCGGGA | |
| | TACTCAAGGTGTCAGCCACGGGAAACGTGTTCTTATCT | |
| | ACTCTCAGATCTATCAACCTGGATGATGATGAAAACCG | |
| | GAAATCATGTAGCATATCAGCAACGCCACTAGGGTGT | |
| | GACTTACTTTGTGCTAAAGTCACTGAGAGAGAAGAGG | |
| | CAGATTACAATTCAGATGCAGCGACGAGATTAGTTCAT | |
| | GGCAGGTTAGGTTTTGATGGGGTATACCATGAGCAGGC | |
| | CCTGCCTGTAGAATCATTGTTCAGTGACTGGGTTGCAA | |
| | ACTATCCGTCAGTCGGCGGAGGCAGTTACTTTGATAAT | |
| | AGGGTATGGTTTGGCGTGTATGGGGGGATCAGACCTG | |
| | GCTCTCAGACTGATCTGCTCCAGTCTGAGAAGTACGCG | |
| | ATATATCGTAGGTACAATAATACCTGCCCTGATAATAA | |
| | TCCCACCCAGATTGAGCGGGCCAAATCATCTTATCGTC | |
| | CGCAGCGGTTTGGCCAGCGGCTTGTACAACAAGCAATT | |
| | CTATCAATTAGAGTGGAGCCATCTTTGGGTAATGATCC | |
| | TAAACTATCTGTGTTAGATAATACAGTCGTGTTGATGG | |
| | GGGCGGAAGCAAGGATAATGACATTTGGCCACGTGGC | |
| | ATTAATGTATCAAAGAGGGTCATCATATTTTCCTTCTG | |
| | CACTATTATACCCTCTCAGTTTAACAAATGGTAGTGCA | |
| | GCAGCATCCAAGCCTTTCATATTCGAGCAATATACAAG | |
| | GCCAGGTAGCCCACCTTGTCAGGCCACTGCAAGATGTC | |
| | CAAATTCATGTGTTACTGGTGTCTACACAGACGCATAC | |
| | CCGTTATTTTGGTCTGAAGATCATAAAGTGAATGGTGT | |
| | ATATGGTATGATGTTAGATGACATCACATCACGGTTAA | |
| | ACCCGGTAGCAGCTATATTTGATAGGTATGGTAGGAGT | |
| | AGAGTGACTAGGGTTAGCAGTAGCAGCACGAAGGCAG | |
| | CTTACACTACAAATACATGCTTTAAGGTTGTCAAAACA | |
| | AAGAGAGTATACTGCTTGAGCATTGCCGAGATAGAGA | |
| | ATACACTGTTTGGAGAATTCAGAATAACCCCTTTACTC | |
| | TCCGAGATAATATTTGACCCAAACCTTGAACCCTCAGA | |
| | CACGAGCCGTAACtagttgagtcaattataaaggagttggaaagatggcattgt | |
| | atcacctatcttctgcgacatcaagaatca | |
| APMV7/Dove/ | atttacagttagtttacctgtctatcaagttagaaaaaacacgggtagaagattctggatcccg | 4 |
| Tennessee/ | gttggcgccctccaggtgcaagATGAGAGTACGACCTTTAATAATA | |
| 4/75 | ATCCTGGTGCTTTTAGTGTTGCTGTGGTTAAATATTCTA | |
| (lower cases | CCCGTAATTGGCTTAGACAATTCAAAGATTGCACAAGC | |
| correspond | AGGTATTATCAGTGCACAAGAATATGCAGTTAATGTGT | |
| to NDV | ATTCACAGAGTAATGAGGCTTACATTGCACTGCGCACT | |
| derived | GTGCCATATATACCTCCACACAATCTCTCTTGTTTCCAG | |
| sequences | GATTTAATCAACACATACAATACAACGATTCAAAACAT | |
| and upper | ATTCTCACCAATTCAGGATCAAATCACATCTATAACAT | |
| cases | CGGCGTCAACGCTCCCCTCATCAAGATTTGCAGGATTA | |
| correspond | GTAGTCGGTGCAATCGCTCTCGGAGTAGCGACATCTGC | |
| to APMV F | ACAAATAACTGCAGCCGTGGCACTCACAAAGGCACAG | |
| and HN | CAGAACGCTCAAGAAATAATACGATTACGTGATTCTAT | |
| coding | CCAAAATACTATCAATGCTGTGAATGACATAACAGTAG | |
| sequences) | GGTTAAGTTCAATAGGAGTAGCACTAAGCAAGGTCCA | |
| | AAACTACTTGAATGATGTGATAAACCCTGCTCTGCAGA | |
| | ACCTGAGCTGCCAGGTTTCTGCATTAAACTTAGGGATC | |
| | CAATTAAATCTTTATTTAACCGAAATTACAACTATCTTT | |
| | GGACCGCAAATTACAAATCCATCATTGACCCCATTGTC | |
| | AATTCAGGCATTATACACCCTAGCAGGAGATAACCTGA | |
| | TGCAATTTCTTACCAGGTATGGCTATGGAGAGACAAGT | |
| | GTTAGCAGTATTCTCGAGTCAGGACTAATATCAGCACA | |
| | AATTGTATCTTTTGATAAACAGACAGGCATTGCAATAT | |
| | TGTATGTCACATTACCATCAATTGCGACTCTTTCCGGTT | |
| | CTAGAGTTACCAAATTGATGTCAGTTAGTGTCCAAACT | |
| | GGAGTTGGAGAGGGTTCTGCTATTGTACCATCATACGT | |
| | TATTCAGCAGGGAACAGTAATAGAAGAATTTATTCCTG | |
| | ACAGTTGCATCTTCACAAGATCAGATGTTTATTGTACT | |
| | CAATTGTACAGTAAATTATTGCCTGATAGCATATTGCA | |
| | ATGCCTCCAGGGATCAATGGCAGATTGCCAATTTACTC | |
| | GCTCATTGGGTTCATTTGCAAACAGATTCATGACCGTT | |
| | GCAGGTGGGGTGATAGCAAATTGTCAGACAGTCCTGT | |
| | GCCGATGCTATAATCCAGTTATGATTATTCCCCAGAAC | |
| | AATGGAATTGCTGTCACTCTGATAGATGGTAGTTTATG | |
| | TAAAGAACTTGAATTGGAGGGGATAAGACTAACAATG | |
| | GCAGACCCAGTATTTGCTTCATACTCTCGTGATCTGATT | |
| | ATAAATGGGAATCAATTTGCTCCGTCTGATGCTTTAGA | |
| | CATTAGTAGCGAATTAGGTCAACTGAATAACTCAATTA | |
| | GCTCAGCAACTGATAATTTACAGAAGGCACAGGAATC | |
| | ATTGAATAAGAGTATCATTCCAGCTGCGACTTCCAGCT | |
| | GGTTAATTATATTACTATTTGTATTAGTATCAATCTCAT | |
| | TAGTGATAGGATGTATCTCCATTTATTTTATATATAAAC | |
| | ATTCAACCACAAATAGATCACGAAATCTCTCAAGTGAC | |
| | ATCATCAGTAATCCTTATATACAGAAAGCTAATtgaacaca | |
| | gatgaggaacgaaggtttccctaatagtaatttgtgtgaaagttctggtagtctgtcagttcag | |
| | agagttaagaaaaaactaccggttgtagatgaccaaaggacgatatacgggtagaacggt | |
| | aagagaggccgcccctcaattgcgagccaggcttcacaacctccgttctaccgcttcaccg | |
| | acaacagtcctcaatcATGGAGTCAATCGGGAAAGGAACCTGG | |
| | AGAACTGTGTATAGAGTCCTTACGATTCTATTAGATGT | |
| | AGTGATCATTATTCTCTCTGTGATTGCTCTGATTTCATT | |
| | GGGTCTGAAGCCAGGTGAGAGGATCATCAATGAAGTC | |
| | AATGGATCTATCCATAATCAACTTGTTCCCTTATCGGG | |
| | GATTACTTCCGATATTCAGGCAAAAGTCAGCAGCATAT | |
| | ATCGGAGCAACTTGCTAAGTATCCCACTACAACTTGAT | |
| | CAAATCAACCAGGCAATATCATCATCTGCTAGGCAAAT | |
| | TGCTGATACAATCAACTCGTTTCTCGCTCTGAATGGCA | |
| | GTGGAACTTTTATTTATACAAATTCACCTGAGTTTGCA | |
| | AATGGTTTCAATAGAGCAATGTTCCCAACCCTAAATCA | |
| | AAGCTTAAATATGCTAACACCTGGTAATCTAATTGAAT | |
| | TTACTAATTTTATTCCAACTCCAACAACAAAATCAGGA | |
| | TGTATCAGAATACCATCATTTTCAATGTCATCAAGTCA | |
| | CTGGTGTTATACCCATAATATCATTGCTAGTGGATGTC | |
| | AGGATCATTCAACCAGTAGTGAATACATATCGATGGG | |
| | GGTTGTTGAAGTGACTGATCAGGCTTACCCGAACTTTC | |
| | GGACAACTCTTTCTATTACATTAGCTGATAATCTAAAC | |
| | AGAAAGTCATGTAGCATTGCAGCAACTGGGTTCGGGT | |
| | GTGATATATTATGTAGTGTTGTCACTGAGACAGAAAAT | |
| | GATGATTATCAATCACCAGAACCGACTCAGATGATCTA | |
| | TGGAAGATTATTTTTTAATGGCACATATTCAGAGATGT | |
| | CATTGAATGTGAACCAAATGTTCGCAGATTGGGTTGCA | |
| | AATTATCCAGCAGTTGGATCAGGAGTAGAGTTAGCAG | |
| | ATTTTGTCATTTTCCCACTCTATGGAGGTGTTAAAATCA | |
| | CTTCAACCCTAGGAGCATCTTTAAGCCAGTATTACTAT | |
| | ATTCCCAAGGTGCCCACAGTCAATTGCTCTGAGACAGA | |
| | TGCACAACAAATAGAGAAGGCAAAAGCATCCTATTCA | |
| | CCACCTAAAGTGGCTCCAAATATCTGGGCTCAGGCAGT | |
| | CGTTAGGTGCAATAAATCTGTTAATCTTGCAAATTCAT | |
| | GTGAAATTCTGACATTTAACACTAGCACTATGATGATG | |
| | GGTGCTGAGGGAAGACTCTTGATGATAGGAAAGAATG | |
| | TATACTTTTATCAACGATCTAGTTCGTATTGGCCAGTG | |
| | GGAATTATATATAAATTAGATCTACAAGAATTGACAAC | |
| | ATTTTCATCAAATCAATTGCTGTCAACAATACCAATTC | |
| | CATTTGAGAAATTCCCTAGACCTGCATCTACTGCTGGT | |
| | GTATGTTCAAAACCAAATGTGTGTCCTGCAGTATGCCA | |
| | GACTGGTGTTTATCAAGATCTCTGGGTACTATATGATC | |
| | TTGGCAAATTAGAAAATACCACAGCAGTAGGATTGTAT | |
| | CTAAACTCAGCAGTAGGCCGAATGAACCCTTTTATTGG | |
| | GATTGCAAATACGCTATCTTGGTATAATACAACTAGAT | |
| | TATTCGCACAGGGTACTCCAGCATCATATTCAACAACG | |
| | ACCTGCTTCAAAAATACTAAGATTGACACGGCATACTG | |
| | CTTATCAATATTAGAATTAAGTGATTCTTTGTTAGGATC | |
| | ATGGAGAATTACACCATTATTGTACAATATCACTTTAA | |
| | GTATTATGAGCtagttgagtcaattataaaggagttggaaagatggcattgtatca | |
| | cctatcttctgcgacatcaagaatca | |
| APMV21/ | atttacagttagtttacctgtctatcaagttagaaaaaacacgggtagaagattctggatcccg | 5 |
| pigeon/Taiwan/ | gttggcgccctccaggtgcaagATGACAAAGAGGATCAATCTAAC | |
| AHRI128/17 | GTTGTCCTTATATATACTGGTCACAATCTCAGTGTGTCT | |
| (lower cases | CCCAACAACTCGATGTCTAGACAATAGCAAATTGGCTC | |
| correspond | GAGCCGGGATAATAAGTTCAGCCGAATATGCAGTGAG | |
| to NDV | TGTGTATGCTCAGACAAATGAAGCATACATAGCACTTA | |
| derived | GAACCATTCCATATTTACCAGCCAATCCGAATAATTGC | |
| sequences | TTCACACCAACTATAACCACATATAACACTACAATACA | |
| and upper | ATCTATTTTCTCACCTATTGTCTCACAGATTAATGCAAT | |
| cases | TACTTCGAGCACCTCGACCCAACAAGAGAGACTCTTCG | |
| correspond | GAGTGATCATAGGTACTGTTGCTTTGGGAGTGGCCACT | |
| to APMV F | GCAGCACAAGTGACTGCTGCAGTTGCGCTAACACAAG | |
| and HN | CTCAAAGCAATGCAAAAGCAATCCTACAACTCAAATC | |
| coding | ATCAATACAAAATACTATTGCTGCTGTGTCAGAAGTGA | |
| sequences) | AAGACGGGTTAAGCACAATTGGGATTGCTCTAGGAAA | |
| | AATCCAAGTTTATGTCAATGAAGTGATAAATCCCCAAC | |
| | TTGCTAACCTGACTTGCCAAACAGCTGCTGCGAATTTA | |
| | GGAGTCCAACTTAGTCTATATCTAACAGAATTGACAAC | |
| | TGTGTTTGGGCCCCAAATCACAAACCCTGCATTATCAC | |
| | CACTGACTATTCAAGCACTTTATAATCTGGCAGGATCG | |
| | AACTTAGATACATTCTTTGAGAAATATGGTTATAAGCA | |
| | AGCAACTGCAACAAGTGTGCTAGAGGCTGGACTAGTA | |
| | ACTGGCCAGATTGTATCTTTTGATCCAGCTACAGGTAT | |
| | TGGAATTATTCGAGTTTCCCTTCCTAGCATAGCAACAC | |
| | TATCTTCTGCTCGTGTCACCAAACTTGAAACTGTGAGT | |
| | GTTAGTACCTCAACAGGAGAAGCTGTAGCCATTGTCCC | |
| | GTCATTCATAATACAGCAAGGGACAGTTATAGAAGAA | |
| | TTCATAATTGACGGCTGTATAAGAACAAGTGCTGATAT | |
| | ATATTGCACTCGGCTGTTTACAAAAATACTGCCTGATA | |
| | GCGTACTGAATTGCTTGCAAGGATTAGTTAATGAGTGT | |
| | CAATTTACCCGTGGCTTAGGGACCTATGCAAATAGGTT | |
| | TGTAACAATCAATGGTGGAATTGTTGCAAATTGTCAAA | |
| | CATTACTCTGCCGCTGCTATAGCCCGTCATATATTATTA | |
| | CGCAAAATTCCAATATAGCAGTCACCTTAATCGACTCA | |
| | AGTACCTGTCGTGACTTAGACTTGGACGGCATAAGATT | |
| | AGCTTTAGGAAATACTGAATTCTCAGAGTATGCCAAAA | |
| | ATCTAACAATAGCAGAGTCCCAATTCGCACCCTCTGAT | |
| | GCATTGGATATCAGCAGTGAAATAGGGAAACTAAATG | |
| | CTACGATATCAAGAGTGGAAGACTACCTCAACCAAGC | |
| | AACAAAAGATGTCACTGCTATATCAATTAATAAGTCAG | |
| | CAGCAGACATAATTCTGATTGTGACATTAATACTTACC | |
| | ATCCTTCTAATAATTACTGTCATAGTCATAGTTGTTATC | |
| | ATCATAAAACAAAGGAGGGTAATTACACACAAAACGA | |
| | CAAATGAGGATATGATTTCGAATCCATACGTTACAAAT | |
| | GCCAAGTGAtgaacacagatgaggaacgaaggtttccctaatagtaatttgtgtgaa | |
| | agttctggtagtctgtcagttcagagagttaagaaaaaactaccggttgtagatgaccaaag | |
| | gacgatatacgggtagaacggtaagagaggccgcccctcaattgcgagccaggcttcac | |
| | aacctccgttctaccgcttcaccgacaacagtcctcaatcATGGACTCACATAT | |
| | CCCAGCCAAGAACACATGGCGGACAGTATATCGCGTG | |
| | GTGACCATACTGTTAGATATCGTGATAATTGTCCTTGC | |
| | CATCATATCTCTTGTGTCTTTAGGCTTAAAACCTGGTGA | |
| | GAAGATCTTGACAGGGGTAAATGATTCAGTGCATGCA | |
| | GAGTTAGGCATGATGAGACCAGCATTATTAGATATAG | |
| | ATAGTAAGGTGAGTACAATTTACAGGTACAATCTCATC | |
| | AACCTGCCTCTACAGTTGGATGATATCCAGACAGCCAT | |
| | AGTGTCTTCTTCAAGGCAATTAGCTGACACTATAAATA | |
| | GCTTCTTAGCAATCAATGGTTCATCTGCAGTGCTATAC | |
| | ACAACAGGTCCCGAGTTCTCAAACGGATTTAATAAGG | |
| | AGCTTTATCCCAACTTTAATCAGACTCGAGATTCTATA | |
| | TCAATTGGGCAGCTAGTTGAGTTCACAAACTTCATACC | |
| | AACACCAACAACTAAGCCGGGGTGCATAAGAATACCC | |
| | ACATTTGCTGCAGGACAATCTCATTGGTGTTATAGTCA | |
| | CAATATAATTGCATCCGGATGTCAAGACCATTCAACAA | |
| | GTAGTCAGTACATAGCCATGGGAGTAATCATCATTAAT | |
| | CAACAACAATCACCTGACTTCAGAACAACAACTTCTAT | |
| | CACCCTGTCGGATAATAAGAATAGAAAATCATGTAGT | |
| | GTCGGAGTGTCGGAATACGGATGTGATCTTTTGTGCAG | |
| | TGTGGTAACAGAAACAGAGAATGAAGATTACAAGTCA | |
| | GAACCCCCGACAGACATGATATACGGGAGGTTATTCTT | |
| | TAACGGGACATACAGTGAAGTTGATCTCCCTGTGTCTA | |
| | CACTGTTCTCTGAATGGGTTGCGAATTACCCAGGAGTA | |
| | GGGTCAGGGGTTGTGTATCGCAGGAAGATGTATTTCCC | |
| | TATTTATGGAGGAATTAAGATTTCCTCTAATTTGGGAA | |
| | ACTACTTGTCTCACTTTTACTATATACCACAAGTTCCCA | |
| | CTGTCAATTGTACAGACAGTGATGAGATACAGATTACC | |
| | AATGCAAAGGCGTCATACTCTCCTCCTAAAGTTGCTCC | |
| | GAATTTATGGGGGCAAGCATTGCTTGCTTGTAATATCA | |
| | GCGTTAATTTGCCGAGTTCCTGTAGATTACTAGTCTTCA | |
| | ATACGTCATCAATGATGATGGGTGCTGAAGGTAGAAT | |
| | ATATAACATCAATGAGCAGTATTATTTCTACCAAAGAT | |
| | CAAGCAGTTATTGGCCAGTGGGCCTCATCTATAGGCTG | |
| | GAAATGACAAGCCTTGACAGCATGACTGATTCAGGTAT | |
| | TATCAACACTACTCCCATCCCCTTTGAGAAATTCCCAC | |
| | GGCCAGCATCACAGGCTGGTGTGTGCTCAATTCCAAGC | |
| | GTTTGTCCTCGAGTCTGTCAAACTGGCGTTTATCAGGA | |
| | CATCTGGGTGTTATCACAACCAAGTGTTGAAATGAATA | |
| | CTACAGCAATAGGTATCTATCTTAACTCAGCTGTCGGT | |
| | AGGACAAATCCGAAAATTGGCGTTGCAAATACCTTAA | |
| | GCTGGATAGACGCTGTTCAATTGTTTCAACCAACTACT | |
| | CCTGCTAGTTACTCCACAACTACTTGTTTTAAGAATAC | |
| | AGCAAGAGATATATCTTATTGTCTGTCAATCCTGGAAC | |
| | TAAGCGATTCACTACTTGGTTCTTGGAGGATAGCTCCA | |
| | TTGCTGTATAATCTTACACTGGTTCCTAATTCAtagttgagtc | |
| | aattataaaggagttggaaagatggcattgtatcacctatcttctgcgacatcaagaatca | |
| APMV6/duck/ | atttacagttagtttacctgtctatcaagttagaaaaaacacgggtagaagattctggatcccg | 6 |
| HongKong/ | gttggcgccctccaggtgcaagATGGGAGCCCGACTGGGGCCCTTT | |
| 18/199/77 | ACAATGGCACCCGGCCGGTATGTGATTATTTTCAACCT | |
| (lower cases | CATCCTTCTCCACAAGGTTGTGTCACTAGACAATTCAA | |
| correspond | GATTACTACAGCAGGGGATTATGAGTGCAACCGAAAG | |
| to NDV | AGAAATCAAAGTGTACACAAACTCCATAACTGGAAGC | |
| derived | ATTGCTGTGAGATTGATTCCCAACCTACCTCAAGAAGT | |
| sequences | GCTTAAATGTTCTGCTGGGCAGATCAAATCATACAATG | |
| and upper | ACACCCTTAATCGAATTTTCACACCTATCAAGGCGAAT | |
| cases | CTTGAGAGGTTACTGGCTACACCGAGTATGCTTGAACA | |
| correspond | CAACCAGAACCCTGCCCCAGAACCTCGCCTGATTGGAG | |
| to APMV F | CAATTATAGGCACAGCAGCACTGGGGCTGGCAACAGC | |
| and HN | AGCTCAGGTTACAGCTGCACTCGCCCTTAACCAGGCCC | |
| coding | AGGATAATGCTAAGGCCATCTTAAACCTCAAAGAGTCC | |
| sequences) | ATAACAAAAACAAATGAAGCTGTGCTTGAGCTTAAGG | |
| | ATGCAACAGGGCAAATTGCGATAGCGCTAGATAAGAC | |
| | TCAAAGATTCATAAATGACAATATCTTACCGGCAATCA | |
| | ATAATCTGACATGTGAAGTAGCAGGTGCTAAAGTAGG | |
| | TGTGGAACTATCATTATACTTGACCGAGTTAAGCACTG | |
| | TGTTTGGGTCGCAGATAACCAATCCAGCACTCTCCACT | |
| | CTATCCATTCAAGCCCTCATGTCACTCTGCGGTAATGA | |
| | TTTTAATTACCTCCTGAACCTAATGGGGGCCAAACACT | |
| | CCGATCTGGGTGCACTTTATGAGGCAAACTTAATCAAT | |
| | GGCAGAATCATTCAATATGACCAAGCAAGCCAAATCA | |
| | TGGTTATCCAGGTCTCCGTGCCTAGCATATCATCGATTT | |
| | CGGGGTTGCGACTGACAGAATTGTTTACTCTGAGCATT | |
| | GAAACACCTGTCGGTGAGGGCAAGGCAGTGGTACCTC | |
| | AGTTTGTTGTAGAATCTGGCCAGCTTCTTGAAGAGATC | |
| | GACACCCAGGCATGCACACTCACTGACACCACCGCTTA | |
| | CTGTACTATAGTTAGAACAAAACCATTGCCAGAACTAG | |
| | TCGCACAATGTCTCCGAGGGGATGAGTCTAGATGCCAA | |
| | TATACGACTGGAATCGGTATGCTTGAATCTCGATTTGG | |
| | GGTATTTGATGGACTTGTTATTGCTAATTGTAAGGCCA | |
| | CCATCTGCCGATGTCTAGCCCCTGAGATGATAATAACT | |
| | CAAAACAAGGGACTCCCCCTTACAGTCATATCACAAG | |
| | AAACTTGCAAGAGAATCCTGATAGATGGGGTTACTCTG | |
| | CAGATAGAAGCTCAAGTTAGCGGATCGTATTCCAGGA | |
| | ATATAACGGTCGGGAACAGCCAAATTGCCCCATCTGG | |
| | ACCCCTTGACATCTCAAGCGAACTCGGAAAGGTCAACC | |
| | AGAGTCTATCTAATGTCGAGGATCTTATTGACCAGAGC | |
| | AATCAGCTCTTGAATAGGGTGAATCCAAACATAGTAA | |
| | ACAACACCGCAATTATAGTCACAATAGTATTGCTAGTT | |
| | ATCCTGGTATTATGGTGTTTGGCCCTAACGATTAGTAT | |
| | CTTGTATGTATCAAAACATGCTGTGCGAATGATAAAGA | |
| | CAGTTCCGAATCCGTATGTAATGCAAGCAAAGTCGCCG | |
| | GGAAGTGCCACACAGTTCtgaacacagatgaggaacgaaggtttccctaa | |
| | tagtaatttgtgtgaaagttctggtagtctgtcagttcagagagttaagaaaaaactaccggtt | |
| | gtagatgaccaaaggacgatatacgggtagaacggtaagagaggccgcccctcaattgc | |
| | gagccaggcttcacaacctccgttctaccgcttcaccgacaacagtcctcaatcATGGC | |
| | TTCCTCAGGCGATATGAGACAGAGTCAGGCAACTCTAT | |
| | ATGAGGGTGACCCTAACAGCAAAAGGACATGGAGGAC | |
| | TGTGTACCGGGTTGTCACCATATTGCTAGATATAACCG | |
| | TCCTTTGTGTTGGCATAGTGGCAATAGTTAGGATGTCA | |
| | ACCATTACAACAAAAGATATTGATAACAGTATCTCATC | |
| | ATCTATTACATCCCTGAGTGCCGATTACCAGCCAATAT | |
| | GGTCAGATACCCATCAGAAAGTTAACAGTATTTTCAAG | |
| | GAAGTTGGAATCACTATCCCTGTCACACTCGACAAGAT | |
| | GCAAGTAGAAATGGGAACAGCGGTTAACATAATCACT | |
| | GATGCTGTAAGACAACTACAAGGAGTCAATGGGTCAG | |
| | CAGGATTTAGCATTACCAATTCCCCAGAGTATAGTGGA | |
| | GGGATAGACACACTGATATACCCTCTTAATTCACTTAA | |
| | TGGAAAGGCTCTAGCTGTATCAGACTTACTAGAACACC | |
| | CGAGCTTCATACCGACGCCTACCACCTCTCACGGTTGT | |
| | ACCCGCATTCCTACATTCCACCTAGGGTACCGTCATTG | |
| | GTGTTATAGTCACAACACGATAGAGTCTGGTTGTCACG | |
| | ATGCAGGAGAAAGCATTATGTACGTATCCATGGGTGC | |
| | GGTAGGGGTCGGCCATCGCGGGAAACCTGTGTTTACG | |
| | ACAAGTGCAGCGACAATCCTAGATGATGGAAGGAACA | |
| | GGAAAAGTTGTAGCATCATAGCAAACCCTAATGGGTG | |
| | TGATGTCTTATGCAGCTTGGTTAAGCAGACAGAAAATG | |
| | AAGGCTACGCTGACCCTACACCGACCCCAATGATCCAC | |
| | GGTAGGCTCCACTTCAATGGCACATACACTGAGTCTGA | |
| | ACTTGACCCTGGCCTATTTAATAACCATTGGGTCGCTC | |
| | AATATCCAGCAGTTGGTAGCGGTGTCGTCAGCCACAGA | |
| | AAACTATTTTTCCCGCTCTACGGAGGGATATCACCGAA | |
| | GTCAAAACTGTTCAATGAGCTCAAGTCATTTGCTTACT | |
| | TTACTCATAATGCTGAATTGAAATGTGAGAACCTGACA | |
| | GAGAGACAGAAGGAAGACCTTTATAACGCATATAGGC | |
| | CTGGGAAAATAGCAGGATCTCTCTGGGCTCAAGGGGTT | |
| | GTAACATGTAATCTGACCAATTTAGCTGATTGCAAAGT | |
| | TGCAATTGCGAACACGAGCACCATGATGATGGCTGCC | |
| | GAGGGGAGGTTACAGCTTGTGCAAGATAAGATTGTCTT | |
| | CTACCAAAGATCCTCATCATGGTGGCCAGTCCTAATAT | |
| | ATTATGATATCCCTATTAGTGACCTTATCAGTGCCGAT | |
| | CATTTAGGGATAGTGAACTGGACTCCGTATCCACAGTC | |
| | TAAGTTTCCGAGGCCCACCTGGACAAAGGGCGTATGTG | |
| | AGAAACCGGCGATATGCCCCGCTGTATGTGTAACGGGT | |
| | GTTTACCAAGATGTTTGGGTAGTTAGTATAGGGTCACA | |
| | GAGCAATGAGACTGTTGTGGTTGGCGGGTACTTAGATG | |
| | CTGCAGCAGCCCGTCAGGATCCATGGATTGCAGCAGCT | |
| | AACCAGTACAACTGGCTGGTTAGGCGTCGCCTCTTTAC | |
| | ATCCCAAACTAAAGCAGCATACTCATCAACCACTTGCT | |
| | TCAGAAACACGAAGCAGGATAGAGTGTTCTGCCTGAC | |
| | TATAATGGAAGTCACAGACAACCTACTCGGAGACTGG | |
| | AGGATCGCCCCGCTGTTGTATGAAGTTACTGTGGCTGA | |
| | TAAGCAGCAGGGCAATCGCAATTACGTGCCTATGGGG | |
| | AGGGTGGGGACAGATAAGTTCCAATATTATACCCCAG | |
| | GTGACAGATATACTCCTCAGCATtagttgagtcaattataaaggagttg | |
| | gaaagatggcattgtatcacctatcttctgcgacatcaagaatca | |
| APMV11/ | atttacagttagtttacctgtctatcaagttagaaaaaacacgggtagaagattctggatcccg | 7 |
| common_snipe/ | gttggcgccctccaggtgcaagATGGGCACATGCCTAAACAACCGT | |
| France/100212/ | CTGTCGACTATTCCCTCTATCAAAACTGTACAATGTATT | |
| 10 | TTGATTATCTTATCTTACATAATACCATACTCTGCAACA | |
| (lower cases | GACAACCCAATTGCCGACCGGTCATTACTGCGCGCCGG | |
| correspond | AATTGTACCCATATATTCTAAGAGTCTCAGCGTATACA | |
| to NDV | CTAATTCAATCTCAGGTTATCTGACAGTGCGCATGTTA | |
| derived | CCCCCTCTTCCAAAGAATCTAACTGAATGTAGTCAAGA | |
| sequences | AGTTGTTAGTAACTATAATAAAACAATTACAAGAATGT | |
| and upper | TTCAACCTATATCTGATAATCTAATGCGAATTCAAGAG | |
| cases | GGGACAGATAGTGGGACAAAAAGATTTGTGGGTGCCG | |
| correspond | TGATAGGATCAGTTGCTCTCGGTGTTGCTACTTCTGCCC | |
| to APMV F | AAATTACTGCTGCTTTGGCGATGGTACAGGCTCAGGAT | |
| and HN | AATGCAAAAGCCATCTGGAAACTCAAGGAAGCGATTT | |
| coding | CTTCAACAAATCAGGCTGTATTAGAGTTAAAGGAGGG | |
| sequences) | CGTAAATACATTGGGAGTCGCAGTAGACAAGATCCAA | |
| | GGATATATAAATAATGAAATACTCCCCTCATTGTCAGA | |
| | GCTAGAGTGTCGAGTTAATGCAAATAAGTTGGCCTCTC | |
| | AGTTAAATCTATATTTGATTGAGTTAACCACTATATTTG | |
| | GGGATCAGATAACGAATCCAGCATTAACACCCTTAAG | |
| | CCTTCAGGCATTGTATACTCTTGCAGGAGACACAATGG | |
| | GAAGCTTCTTACAATATATTGGTGCACAGGATAACGAA | |
| | ATTGAGTCACTATATGATAGTGGATTAATTAATGGGCA | |
| | GATTGTGTCATATGATGCATCAATCCAGACCATAATTA | |
| | TTAAGGTATCCATTCCATCCATATCATCTCTCTCACGAT | |
| | TTTCTATTATGAGGCTGGCAACAGTTAGCTCATCAGTT | |
| | GGAGGTTTTGAGAAAACTCCCTTGGTGCCTGAGTACTT | |
| | GCTCATAAGTGACAATCACATTGAAGAGTTCAGTATTG | |
| | TTGATTGCAAAGAGTCATCAGATATTTTTTATTGCCCTC | |
| | AAATTTTGTCAATGCCAATATCAACTGCCACTGTAGAA | |
| | TGCCTCAAGGGTAGAATTGATCAGTGCATATATACATC | |
| | ACAGCTAACTATACTTTCTCATCGCATAGTAACATACA | |
| | ATGGTGTCGTTGTTGCCAATTGTTTTGCAGAATTATGTA | |
| | GATGTACTAATCCTAGTTACATAATTAGACAAGACCGG | |
| | GATGTTGCTGTCACAGTAATTGATAAAGATTTGTGTAA | |
| | ACGAGTTCAGATAGGAGATATAGAACTAATTGTACAA | |
| | GCATCTATTGCTAATGAATACAAGGTTAACTTTACTGT | |
| | ATCAGAGGACCAACTTGCGCCCTCTACCCCTATCGATA | |
| | TCAGTAATGAATTAAATTCATTGAATCAAACTTTAGAT | |
| | AAGGTAGGACAATTGATCAATACGAGTAACCAAATTC | |
| | TGGCATCACTGAACCCAAAATTGGTGAACAATACATCT | |
| | ATTATTGTATTAATTGTAATGGGAGTGGTGCTAATTTT | |
| | ATGGCTGTTGGCTCTTACCATCTACTCGATATATGCTGC | |
| | AAGAAATCTAAACTCTATAGGACGACTAGCTAAATCTG | |
| | CATATGCATCTTATGTAGCTGATAAAAATGTATATAAA | |
| | AACGAATCAACTAGCTCTAGCAGTATTTGAtgaacacagatga | |
| | ggaacgaaggtttccctaatagtaatttgtgtgaaagttctggtagtctgtcagttcagagagt | |
| | taagaaaaaactaccggttgtagatgaccaaaggacgatatacgggtagaacggtaagag | |
| | aggccgcccctcaattgcgagccaggcttcacaacctccgttctaccgcttcaccgacaac | |
| | agtcctcaatcATGGACCGATCTAGGAGCCTTGATTACTTAG | |
| | CTGATTCACCTGAAATCAAAAATACATGGCGACAGTTC | |
| | TTCAGAGTGGTCCTTATTATACTCCAAATTACCATGCT | |
| | GTGTATCAGCATTTCTGCACTGGCTATTACAATTCAAG | |
| | TTCGTGATCAACATCTTCCTTCTCTAATAAAAGAGAAT | |
| | CCTAAAACCACCTCATCACTTATATCAAGTGAACTCAA | |
| | CCCGCTGCTATCCTATCTTCCTGGTATAAATAGAGAAG | |
| | TTCAGTTGAATATACCTATTCAATTAGATAAAATTCAA | |
| | CAATCTGCGACCTCAGAAATCAATCGGCTTACTGCTGC | |
| | TATTAATCAGATGGCATTTGGCACTCTTTCACCTGGAC | |
| | TCCTGCTAAAGAACTCAAAGGATTATGTAGGAGGTATT | |
| | AATAAGCCGCTTATTCCTTCTGACAAACTCAATTGGAC | |
| | AAATGCAACAATATCAGGATTTATTGAACACCCTAGTT | |
| | TCATACCCGGACCAACCACCAAAAAGGGGTGTACACG | |
| | AATACCATCTTTCCATTTGGCCGAGTCTCATTGGTGCTA | |
| | TACACATAATACTATAGCTTCAGGGTGTGAAGACCATG | |
| | GTGTGTCTTCTATGTATATCTCTGGTGGGATTTTATATA | |
| | AGGGCTCTAATAAAGAGCCTTCTCTTCTAACGACAGTA | |
| | AGTATCTTGTTGGCAGACGAGCTTAATAGGAAAAGCTG | |
| | CAGTATAATTGCTTCTTCTTATGGATGTGATGTCCTCTG | |
| | TTCTCTAGTCACAGAAAGCGAGAGTCAAGATTATAAGT | |
| | CAGTTAATCCGACACCCATGGTACATGGTCGGTTGTTT | |
| | TTTAATGGATCATATTCTGAACAGGAACTTGATCCCAG | |
| | GATTTTTGGAGATTTGTGGACTGCGAACTACCCTGGAG | |
| | TCGGATCAGGGATACTTTTAAAAGATAGACTTGTGTTC | |
| | CCAATTTATGGAGGTCTAGATGAGACAAAGCTGAATCT | |
| | AACATCTTATCTTAACCATCCCTTGTACACAAAAAATG | |
| | AATGGGTGTCATGTAATAAATCATATGACGAGGTTGTA | |
| | CAGACATTAAGAGCTGCATATCGGCCTTCTTGGTTCGC | |
| | TGGGAGAGTTGTTACTCAAGGAGTGATGGTCTGCCATT | |
| | ATGATAGAGAATTGCTAGGGAGGTGTCTCATAGCACG | |
| | CTTTAATACATCTACAGTAATGATGGGAGCAGAAAGTA | |
| | GATTAGTGATGCAAGGTGACTCACTCCTTCTATACCAA | |
| | CGATCAAGTTCATGGTGGCCAGTTGGAATTGTTTATTT | |
| | AGTTCCGGAATCCATCATCTCAATTAATGAGACAAATT | |
| | CGGTATTCGACTTATCGCCTATCCCATTGTCCAAATTCC | |
| | CGAGACCTACTAATAAGAAAGGATATTGTGAACGACC | |
| | TGCTGTTTGTCCTGCAGTTTGTGTGACCGGTGTGTATCA | |
| | AGATCTTTGGCCGTTATCACCATTAGCTATTGAGAACA | |
| | GGACAGCCACCAACCCTACTTTTGCAGGTGCATTCCTT | |
| | AATGCATTTACAACAAGAACAGCTCCTTACTTTGGAGT | |
| | AGCAGGGCCAAACAAGTGGGCTCGGTCAGTACAGTTA | |
| | TTTACTGACCAGACTCCAGCATCATATTCAACAACTAC | |
| | TTGCTTTAAGGACACCATAACTACACAAACTTATTGCT | |
| | TAATTATCATTGAACTACAAGAGAATCTTTTGGGTACC | |
| | TGGAAAATTGTACCCCTTTTGGTTAAAGTATCTTTAGTT | |
| | TACTCGtagttgagtcaattataaaggagttggaaagatggcattgtatcacctatcttct | |
| | gcgacatcaagaatca | |
| APMV15/ | atttacagttagtttacctgtctatcaagttagaaaaaacacgggtagaagattctggatcccg | 8 |
| calidris_ | gttggcgccctccaggtgcaagATGTATGTACCAGGTGTAATTCTA | |
| fuscicollis/ | GCTCTCTTGATGATCAATCCCTGTCTAACTCTAGACAA | |
| Brazil/RS- | TTCTAAGCTAGCACCCGTGGGTATTATTAGTGCTGCGG | |
| 1177/12 | AGCATGAGTTGGCTATATATACGAATACACTCTCTGGC | |
| (lower cases | TCTATTGCTGTCAGATTCCTGCCCAATTTGCCAGCTAAT | |
| correspond | CTTACACATTGTCAGAAGACAATCTTAGATAACTACAA | |
| to NDV | TGTAACTGTGACACGCATTTTGAAGCCGATTGCGGATA | |
| derived | ATCTGAATATACTCAAGCATGGATTAGAAGTTCCAAAA | |
| sequences | GAGAGATTAGTAGGTGCTATCATAGGCACTGTTGCTCT | |
| and upper | TGGTGTGGCCACATCAGCTCAAATCACAGCTGCAGTTG | |
| cases | CAGTTGCCCAAGCTCAGCAGAATGCAAAAGATATCTG | |
| correspond | GAAGCTTAAAAATGCAATCCTTAGTACTAATGAGGCTG | |
| to APMV F | TACTAGAATTAAAGACAGGCTTGCAACAAACTGCCATC | |
| and HN | GCACTAGACAAAATACAGGATTATATCAATAATGAGA | |
| coding | TTATACCAACAGTTAATAATTTAACCTGTGAAGTGATG | |
| sequences) | GCAAACAGACTTGGTGTATATTTGTCCTTGTATTTGAC | |
| | GGAGCTAACCACAGTATTTGGGAATCAAATAACCAAT | |
| | CCTGCCCTCAGCACAATTTCATATCAAGGACTGACGAA | |
| | TTTGTGTGGGAATAATATTGGAGCACTGACAAAATTAA | |
| | TAGGGTTAAAAGATGATAATGTAGAATCAATATATGA | |
| | AGCGGGATTAATAACTGGTCAAGTAGTTGACTATGACC | |
| | CTGCAAGTCAAATCTTAATCATCCAGGTTAGTTATCCA | |
| | AGTATATCAAGATTGAGTGATATAAGAGCTACTGAGTT | |
| | AATCACTGTTGGTGTGACAACTCCTTTTGGTGAAGGAA | |
| | GGGCAATTGTCCCGAAGTATGTAGCACAGAGCACTGT | |
| | ATTAATTGAGGAGTTGGACATCTCATCTTGTAAATTCA | |
| | GTTCAACTACATTATACTGTACTCAGATTAATACTCGC | |
| | CCGTTACCTCCGAGAGTATCAAGCTGCCTTAAAGGTGA | |
| | TTATGAGAATTGTCAGTTTACAACAGAAGTGGGGGTGC | |
| | TTGCATCTAGGTATGCATCTATAGGGAAGGGAGTAGTA | |
| | GTCAATTGCAGATCAATTATATGTAAATGTTTAGAGCC | |
| | TCCTAGAATTATACCTCAGAATAGCTTGGCATCTATAA | |
| | CAGTCATAGATAGCAAGATCTGTAAGAAGCTCCAATTA | |
| | CCTGATGTTATATTGCGTCTAGATGGTAACCTCGAATC | |
| | TCAGTATTTCACTAATATATCAATCAATGGTGGGCAAG | |
| | TGACCCCTTCTGGGCCACTTGATATTAGCAGTGAGATA | |
| | GGGAACATCAATCAAACTGTAAATCGAGTTGAGGATTT | |
| | GATTCATGAATCTGAAAGCTGGCTGTCTCGAGTCAATC | |
| | CCAAGCTAATATCAAACACAGCAATCATTGTTCTCTGT | |
| | GTCTTATCATCGCTGTGTGTGCTTTGGCTTATATTAATC | |
| | ACTGCATTCATGGCTAAATTACTAAGTAATGTTAAAAA | |
| | GATAGAAAGGAAGGTAGCGGTATCTTCCCTAATAGGT | |
| | AATCCTTATGTTTATACCAATCCTGGCTATTCAGGTTCT | |
| | AAGAGCGCATGAtgaacacagatgaggaacgaaggtttccctaatagtaatttgt | |
| | gtgaaagttctggtagtctgtcagttcagagagttaagaaaaaactaccggttgtagatgacc | |
| | aaaggacgatatacgggtagaacggtaagagaggccgcccctcaattgcgagccaggct | |
| | tcacaacctccgttctaccgcttcaccgacaacagtcctcaatcATGAATTCAAG | |
| | TTACTCTCAAGATAATTTATATACAAATCAAACTGCTG | |
| | CTCAACCTAGAGGAACTTGGAGAGTTCTTTATAGGGCC | |
| | GTATCATTGATATTCCAGATATTAATCTTCTCACTAGTA | |
| | CTGACAAATGTCATCCAATATTCAAATCTCCATAGCCC | |
| | CTCTGTGTCAGAGATTTCTGCAGCCACTACAACTGAGA | |
| | CCATTGATGGGTTAAAACCACACTTAGAAACTCCACTT | |
| | AACCAGATAAATGACATCTTTCGCTTAACTGCCCTTGA | |
| | CTTGCCCATACAGATGAACACGATGACTCGAGAAATC | |
| | ACAAGTCAACTCAATATCCTGACCAGTGGAATTAATGA | |
| | GCTTGTTACCTCGAATAATTCGGGAAGACTTCTCCAGA | |
| | CTACAGACCCTGCATATACAGGTGGTATTGGAGTCTTT | |
| | GTGCTCAATAATTACTTAGATTATCCACCGAATCTGCA | |
| | GAACATGTCATTATTAGAGCAGCCTAATTTTGTACCCG | |
| | GTTCTACCACCACTGGGGGTTGTACACGGATTCCCACT | |
| | TTCCATTTGTCATCAACTCATTGGTGTTATTCTCATAAC | |
| | ATCATTGAGAAAGGCTGCCATGATGCAGGACACTCGA | |
| | GTATGTACATATCTATTGGTGTGGTCCAAGTATCCTCA | |
| | CGCGGTGTTCCAGTGTTTCTGACGACTCAGAGTGTAAT | |
| | TGTTGACGACGAAACCAACCGAAAATCCTGTAGCATTG | |
| | TGTCAACTGAATATGGTTGTGATATATTATGCAGCATA | |
| | GTTACTGAGCGTGAGTCAGATGACTATAAATCAGATCC | |
| | ACCTACTAGAATGCTACATGGTAGACTCTTGTTCAACG | |
| | GCTCTTATGTAGAAGCTGCTGTCAAATTCACCAATGAC | |
| | ATCAATAAATTCTCCGCAAATTACCCGGGAGTCGGTTC | |
| | AGGAATCCTCCTAGGCAATAAAATTCTATTCCCTCTGT | |
| | ATGGAGGCATAAAGCAGAGTACAGATTTGTTTAATTAC | |
| | TTACACAACAGGACTGCTCAAGTATCAAACAATAAAA | |
| | CAGTATGTAGCACCGGTTATGATAAAAAGAAACTAGA | |
| | AGCTGCATATCGACCGCCACTAATTGGAGGGAGATTTT | |
| | GGGCAATCGGGATAGTTATATGTAAGTTCAGCATAAAT | |
| | TCACTTGGAGATTGCAGATACAAAATATATGACAGCA | |
| | GCGTAGTCATGATGGGTTCAGAAAATCGCCTCATGAAG | |
| | GTAGGCAATCAGGTATTCTTGTATCAGAGATCCAGCTC | |
| | ATGGTGGCCTATTGGATTGACCTACATACTCAATAGCA | |
| | CTGACTTGCTTAACACCGATTCTGACATAGTCAGCAGT | |
| | ATAATCCCCATATATCATACAAAATTCCCGCGCCCTAC | |
| | TTATGATAGGAATGCATGCACTAGACCAAACGTTTGTC | |
| | CTGCCACATGCATAGAAGGTGTTTATGCAGATATTTGG | |
| | CCACTTAATAATCCGGCAGAACCGAGTAAAATTATATG | |
| | GGTCAGTCATTATCTCAATTCAGAAGTAGGGAGAGAAT | |
| | TCCCTGCTATCGGTGTTGCCAACCAATATGAATGGGTA | |
| | AAAGAATTTCGTCCACTTCCACCCACAACAGGTGCAGC | |
| | GTATGCAACTACTTCTTGTTTTAAGAATACAATAAGCA | |
| | ACCGCATCTTCTGTGTTAGTGTAGCTGAATTCAAGGAC | |
| | AATTTATTTGGGCAATTCAGAATCGTACCGCTTCTATA | |
| | TGAGATTAAAGTAATCAACtagttgagtcaattataaaggagttggaaag | |
| | atggcattgtatcacctatcttctgcgacatcaagaatca | |
| APMV8/Goose/ | atttacagttagtttacctgtctatcaagttagaaaaaacacgggtagaagattctggatcccg | 9 |
| Delaware/ | gttggcgccctccaggtgcaagATGGGTAAAATATCAATATATCTA | |
| 1053/76 | ATTAATAGCGTGCTATTATTGCTGGTATATCCTGTGAA | |
| (lower cases | TTCGATTGACAATACACTCGTTGCCCCAATCGGAGTCG | |
| correspond | CCAGCGCAAATGAATGGCAGCTTGCTGCATATACAAC | |
| to NDV | ATCACTTTCAGGGACAATTGCCGTGCGATTCCTACCTG | |
| derived | TGCTCCCGGATAATATGACTACCTGTCTTAGAGAAACA | |
| sequences | ATAACTACATATAATAATACTGTCAACAACATCTTAGG | |
| and upper | CCCACTCAAATCCAATCTGGATGCACTGCTCTCATCTG | |
| cases | AGACTTATCCCCAGACAAGATTAATTGGGGCAGTTATA | |
| correspond | GGTTCAATTGCTCTTGGTGTTGCAACATCGGCTCAAAT | |
| to APMV F | CACTGCTGCAGTCGCTCTCAAGCAAGCACAAGATAATG | |
| and HN | CAAGAAACATACTGGCACTCAAAGAGGCACTGTCCAA | |
| coding | AACTAATGAGGCGGTCAAGGAGCTTAGCAGTGGATTG | |
| sequences) | CAACAAACAGCTATTGCACTTGGTAAGATACAGAGCTT | |
| | TGTGAATGAGGAAATTCTGCCATCTATCAACCAACTGA | |
| | GCTGCGAGGTGACAGCCAATAAACTTGGGGTGTATTTA | |
| | TCTCTGTATCTCACAGAACTGACCACTATATTCGGTGC | |
| | ACAGTTGACTAACCCTGCATTGACTTCATTATCATATC | |
| | AAGCGCTGTACAACCTGTGTGGTGGCAACATGGCAAT | |
| | GCTTACTCAGAAGATTGGAATTAAACAGCAAGACGTT | |
| | AATTCGCTATATGAAGCCGGACTAATCACAGGACAAG | |
| | TCATTGGTTATGACTCTCAGTACCAGCTGCTGGTCATC | |
| | CAGGTCAATTATCCAAGCATTTCTGAGGTAACTGGTGT | |
| | GCGTGCGACAGAATTAGTCACTGTTAGTGTAACAACAG | |
| | ACAAGGGTGAAGGGAAAGCAATTGTACCCCAATTTGT | |
| | AGCTGAAAGTCGGGTGACTATTGAGGAGCTTGATGTA | |
| | GCATCTTGTAAATTCAGCAGCACAACCCTATACTGCAG | |
| | GCAGGTCAACACAAGGGCACTTCCCCCGCTAGTGGCTA | |
| | GCTGTCTCCGAGGTAACTATGATGATTGTCAATATACC | |
| | ACAGAGATTGGAGCATTATCATCCCGGTATATAACACT | |
| | AGATGGAGGGGTCTTAGTCAATTGTAAGTCAATTGTTT | |
| | GTAGGTGCCTTAATCCAAGTAAGATCATCTCTCAAAAT | |
| | ACAAATGCTGCAGTAACATATGTTGATGCTACAATATG | |
| | CAAAACAATTCAATTGGATGACATACAACTCCAGTTGG | |
| | AAGGGTCACTATCATCAGTTTATGCAAGGAACATCTCA | |
| | ATTGAGATCAGTCAGGTGACTACCTCCGGTTCTTTGGA | |
| | TATCAGCAGTGAGATAGGGAACATCAATAATACGGTG | |
| | AATCGTGTGGAGGATTTAATCCACCAATCGGAGGAAT | |
| | GGCTGGCAAAAGTTAACCCACACATTGTTAATAATACT | |
| | ACACTAATTGTACTCTGTGTGTTAAGTGCGCTTGCTGT | |
| | GATCTGGCTGGCAGTATTAACGGCTATTATAATATACT | |
| | TGAGAACAAAGTTGAAGACTATATCGGCATTGGCTGTA | |
| | ACCAATACAATACAGTCTAATCCCTATGTTAACCAAAC | |
| | GAAACGTGAATCTAAGTTTtgaacacagatgaggaacgaaggtttccct | |
| | aatagtaatttgtgtgaaagttctggtagtctgtcagttcagagagttaagaaaaaactaccg | |
| | gttgtagatgaccaaaggacgatatacgggtagaacggtaagagaggccgcccctcaatt | |
| | gcgagccaggcttcacaacctccgttctaccgcttcaccgacaacagtcctcaatcATG | |
| | AGTAACATTGCATCCAGTTTAGAAAATATTGTGGAGCA | |
| | GGATAGTCGAAAAACAACTTGGAGGGCCATCTTTAGA | |
| | TGGTCCGTTCTTCTTATTACAACAGGATGCTTAGCCTTA | |
| | TCCATTGTTAGCATAGTTCAAATTGGGAATTTGAAAAT | |
| | TCCTTCTGTAGGGGATCTGGCGGACGAGGTGGTAACAC | |
| | CTTTGAAAACCACTCTGTCTGATACACTCAGGAATCCA | |
| | ATTAACCAGATAAATGACATATTCAGGATTGTTGCCCT | |
| | TGATATTCCATTGCAAGTAACTAGTATCCAAAAAGACC | |
| | TCGCAAGTCAATTTAGCATGTTGATAGATAGTTTAAAT | |
| | GCTATCAAATTGGGCAACGGGACCAACCTTATCATACC | |
| | TACATCAGATAAGGAGTATGCAGGAGGAATTGGAAAC | |
| | CCTGTCTTTACTGTCGATGCTGGAGGTTCTATAGGATTC | |
| | AAGCAATTTAGCTTAATAGAACATCCGAGCTTTATTGC | |
| | TGGACCTACAACGACCCGAGGCTGTACAAGAATACCC | |
| | ACTTTTCACATGTCAGAAAGTCATTGGTGCTACTCACA | |
| | CAACATCATCGCTGCTGGCTGTCAAGATGCCAGTGCAT | |
| | CTAGTATGTATATCTCAATGGGGGTTCTCCATGTGTCTT | |
| | CATCTGGCACTCCTATCTTTCTTACTACTGCAAGTGAAC | |
| | TGATAGACGATGGAGTTAATCGTAAGTCATGCAGTATT | |
| | GTAGCAACCCAATTCGGCTGTGACATTTTGTGCAGTAT | |
| | TGTCATAGAGAAGGAGGGAGATGATTATTGGTCTGAT | |
| | ACTCCGACTCCAATGCGCCACGGCCGTTTTTCATTCAA | |
| | TGGGAGTTTTGTAGAAACCGAACTACCCGTGTCCAGTA | |
| | TGTTCTCGTCATTCTCTGCCAACTACCCTGCTGTGGGAT | |
| | CAGGCGAAATTGTAAAAGATAGAATATTATTCCCAATT | |
| | TACGGAGGTATAAAGCAGACTTCACCAGAGTTTACCG | |
| | AATTAGTGAAATATGGACTCTTTGTGTCAACACCTACA | |
| | ACTGTATGTCAGAGTAGCTGGACTTATGACCAGGTAAA | |
| | AGCAGCGTATAGGCCAGATTACATATCAGGCCGGTTCT | |
| | GGGCACAAGTGATACTCAGCTGCGCTCTTGATGCAGTC | |
| | GACTTATCAAGTTGTATTGTAAAGATTATGAATAGCAG | |
| | CACAGTGATGATGGCAGCAGAAGGAAGGATAATAAAG | |
| | ATAGGGATTGATTACTTTTACTATCAGCGGTCATCTTCT | |
| | TGGTGGCCATTGGCATTTGTTACAAAACTAGACCCGCA | |
| | AGAGTTAGCAGACACAAACTCGATATGGCTGACCAAT | |
| | TCCATACCAATCCCACAATCAAAGTTCCCTCGGCCTTC | |
| | ATATTCAGAAAATTATTGCACAAAGCCAGCAGTTTGCC | |
| | CTGCTACTTGTGTCACTGGTGTATACTCTGATATTTGGC | |
| | CCTTGACCTCATCTTCATCACTCCCGAGCATAATTTGG | |
| | ATCGGCCAGTACCTTGATGCCCCTGTTGGAAGGACTTA | |
| | TCCCAGATTTGGAATTGCAAATCAATCACACTGGTACC | |
| | TTCAAGAAGATATTCTACCCACCTCCACTGCAAGTGCG | |
| | TATTCAACCACTACATGTTTTAAGAATACTGCCAGGAA | |
| | TAGAGTGTTCTGCGTCACCATTGCTGAATTTGCAGATG | |
| | GGTTGTTTGGAGAGTACAGGATAACACCTCAGTTGTAT | |
| | GAATTAGTGAGAAATAATtagttgagtcaattataaaggagttggaaagat | |
| | ggcattgtatcacctatcttctgcgacatcaagaatca | |
| APMV2/ | atttacagttagtttacctgtctatcaagttagaaaaaacacgggtagaagattctggatcccg | 10 |
| Chicken/ | gttggcgccctccaggtgcaagATGAATCAAGCACTCGTGATTTTG | |
| California/ | TTGGTATCTTTCCAGCTCGGCGTTGCCTTAGATAACTCA | |
| Yucaipa/56 | GTGTTGGCTCCAATAGGAGTAGCTAGCGCACAGGAGT | |
| (lower cases | GGCAACTGGCGGCATATACAACGACCCTCACAGGGAC | |
| correspond | CATCGCAGTGAGATTTATCCCGGTCCTGCCTGGGAACC | |
| to NDV | TATCAACATGTGCACAGGAGACGCTGCAGGAATATAA | |
| derived | TAGAACTGTGACTAATATCTTAGGCCCGTTGAGAGAGA | |
| sequences | ACTTGGATGCTCTCCTATCTGACTTCGATAAACCTGCA | |
| and upper | TCGAGGTTCGTGGGCGCCATCATTGGGTCGGTGGCCTT | |
| cases | GGGGGTAGCAACAGCTGCACAAATCACAGCCGCCGTG | |
| correspond | GCTCTCAATCAAGCACAAGAGAATGCCCGGAATATAT | |
| to APMV F | GGCGTCTCAAGGAATCGATAAAGAAAACCAATGCGGC | |
| and HN | TGTGTTGGAATTGAAGGATGGACTTGCAACGACTGCTA | |
| coding | TAGCTTTGGACAAAGTGCAAAAGTTTATCAATGATGAT | |
| sequences) | ATTATACCACAGATTAAGGACATTGACTGCCAGGTAGT | |
| | TGCAAATAAATTAGGCGTCTACCTCTCCTTATACTTAA | |
| | CAGAGCTTACAACTGTATTTGGTTCTCAGATCACTAAT | |
| | CCTGCATTATCAACGCTCTCTTACCAGGCGCTGTACAG | |
| | CTTATGTGGAGGGGATATGGGAAAGCTAACTGAGCTG | |
| | ATCGGTGTCAATGCAAAGGATGTGGGATCCCTCTACGA | |
| | GGCTAACCTCATAACCGGCCAAATCGTTGGATATGACC | |
| | CTGAACTACAGATAATCCTCATACAAGTATCTTACCCA | |
| | AGTGTGTCTGAAGTGACAGGAGTCCGGGCTACTGAGTT | |
| | AGTCACTGTCAGTGTCACTACACCAAAAGGAGAAGGG | |
| | CAGGCAATTGTTCCGAGATATGTGGCACAGAGTAGAG | |
| | TGCTGACAGAGGAGTTGGATGTCTCGACTTGTAGGTTT | |
| | AGCAAAACAACTCTTTATTGTAGGTCGATTCTCACACG | |
| | GCCCCTACCAACTTTGATCGCCAGCTGCCTGTCAGGGA | |
| | AGTACGACGATTGTCAGTACACAACAGAGATAGGAGC | |
| | GCTATCTTCGAGATTCATCACAGTCAATGGTGGAGTCC | |
| | TTGCAAACTGCAGAGCAATTGTGTGTAAGTGTGTCTCA | |
| | CCCCCGCATATAATACCACAAAACGACATTGGCTCCGT | |
| | AACAGTTATTGACTCAAGTATATGCAAGGAAGTTGTCT | |
| | TAGAGAGTGTGCAGCTTAGGTTAGAAGGAAAGCTGTC | |
| | ATCCCAATACTTCTCCAACGTGACAATTGACCTTTCCC | |
| | AAATCACAACGTCAGGGTCGCTGGATATAAGCAGTGA | |
| | AATTGGTAGCATTAACAACACAGTTAATCGGGTCGACG | |
| | AGTTAATCAAGGAATCCAACGAGTGGCTGAACGCTGT | |
| | GAACCCCCGCCTTGTGAACAATACGAGCATCATAGTCC | |
| | TCTGTGTCCTTGCCGCCCTGATTATTGTCTGGCTAATAG | |
| | CGCTGACAGTATGCTTCTGTTACTCCGCAAGATACTCA | |
| | GCTAAGTCAAAACAGATGAGGGGCGCTATGACAGGGA | |
| | TCGATAATCCATATGTAATACAGAGTGCAACTAAGATG | |
| | tgaacacagatgaggaacgaaggtttccctaatagtaatttgtgtgaaagttctggtagtctgt | |
| | cagttcagagagttaagaaaaaactaccggttgtagatgaccaaaggacgatatacgggta | |
| | gaacggtaagagaggccgcccctcaattgcgagccaggcttcacaacctccgttctaccg | |
| | cttcaccgacaacagtcctcaatcATGGATTTCCCATCTAGGGAGAA | |
| | CCTGGCAGCAGGTGACATATCGGGGCGGAAGACTTGG | |
| | AGATTACTGTTCCGGATCCTCACATTGAGCATAGGTGT | |
| | GGTCTGTCTTGCCATCAATATTGCCACAATTGCAAAAT | |
| | TGGATCACCTGGATAACATGGCTTCGAACACATGGACA | |
| | ACAACTGAGGCTGACCGTGTGATATCTAGCATCACGAC | |
| | TCCGCTCAAAGTCCCTGTCAACCAGATTAATGACATGT | |
| | TTCGGATTGTAGCGCTTGACCTACCTCTGCAGATGACA | |
| | TCATTACAGAAAGAAATAACATCCCAAGTCGGGTTCTT | |
| | GGCTGAAAGTATCAACAATGTTTTATCCAAGAATGGAT | |
| | CTGCAGGCCTGGTTCTTGTTAATGACCCTGAATATGCA | |
| | GGGGGGATCGCTGTCAGCTTGTACCAAGGAGATGCAT | |
| | CTGCAGGCCTAAATTTCCAGCCCATTTCTTTAATAGAA | |
| | CATCCAAGTTTTGTCCCTGGTCCTACTACTGCTAAGGG | |
| | CTGTATAAGGATCCCGACCTTCCATATGGGCCCTTCAC | |
| | ATTGGTGTTACTCACATAACATCATTGCATCAGGTTGC | |
| | CAGGATGCGAGCCACTCCAGTATGTATATCTCTCTGGG | |
| | GGTGCTGAAAGCATCGCAGACCGGGTCGCCTATCTTCT | |
| | TGACAACGGCCAGCCATCTCGTGGATGACAACATCAA | |
| | CCGGAAGTCATGCAGCATCGTAGCCTCAAAATACGGTT | |
| | GTGATATCCTATGCAGTATTGTGATTGAAACAGAGAAT | |
| | GAGGATTATAGGTCTGATCCGGCTACTAGCATGATTAT | |
| | AGGTAGGCTGTTCTTCAACGGGTCATACACAGAGAGC | |
| | AAGATTAACACAGGGTCCATCTTCAGTCTATTCTCTGC | |
| | TAACTACCCTGCGGTGGGGTCGGGTATTGTAGTCGGGG | |
| | ATGAAGCCGCATTCCCAATATATGGTGGGGTCAAGCA | |
| | GAACACATGGTTGTTCAACCAGCTCAAGGATTTTGGTT | |
| | ACTTCACCCATAATGATGTGTACAAGTGCAATCGGACT | |
| | GATATACAGCAAACTATCCTGGATGCATACAGGCCACC | |
| | TAAAATCTCAGGAAGGTTATGGGTACAAGGCATCCTAT | |
| | TGTGCCCAGTTTCACTGAGACCTGATCCTGGCTGTCGC | |
| | TTAAAGGTGTTCAATACCAGCAATGTGATGATGGGGGC | |
| | AGAAGCGAGGTTGATCCAAGTAGGCTCAACCGTGTAT | |
| | CTATACCAACGCTCATCCTCATGGTGGGTGGTAGGACT | |
| | GACTTACAAATTAGATGTGTCAGAAATAACTTCACAGA | |
| | CAGGTAACACACTCAACCATGTAGACCCCATTGCCCAT | |
| | ACAAAGTTCCCAAGACCATCTTTCAGGCGAGATGCGTG | |
| | TGCGAGGCCAAACATATGCCCTGCTGTCTGTGTCTCCG | |
| | GAGTTTATCAGGACATTTGGCCGATCAGTACAGCCACC | |
| | AATAACAGCAACATTGTGTGGGTTGGACAGTACTTAGA | |
| | AGCATTCTATTCCAGGAAAGACCCAAGAATAGGGATA | |
| | GCAACCCAGTATGAGTGGAAAGTCACCAACCAGCTGT | |
| | TCAATTCGAATACTGAGGGAGGGTACTCAACCACAAC | |
| | ATGCTTCCGGAACACCAAACGGGACAAGGCATATTGT | |
| | GTAGTGATATCAGAGTACGCTGATGGGGTGTTCGGATC | |
| | ATACAGGATCGTTCCTCAGCTTATAGAGATTAGAACAA | |
| | CCACCGGTAAATCTGAGtagttgagtcaattataaaggagttggaaagatg | |
| | gcattgtatcacctatcttctgcgacatcaagaatca | |
| APMV3/Turkey/ | atttacagttagtttacctgtctatcaagttagaaaaaacacgggtagaagattctggatcccg | 11 |
| Wisconsin/68 | gttggcgccctccaggtgcaagATGGCCTCCCCAATGGTCCCACTA | |
| (lower cases | CTCATCATAACGGTAGTACCCGCACTCATTTCAAGTCA | |
| correspond | ATCAGCTAATATTGATAAGCTCATTCAAGCAGGGATTA | |
| to NDV | TCATGGGCTCAGGGAAGGAACTCCACATTTATCAAGA | |
| derived | ATCTGGCTCTCTTGATTTGTATCTTAGACTATTGCCAGT | |
| sequences | TATCCCTTCAAATCTTTCTCATTGCCAGAGTGAAGTAA | |
| and upper | TAACACAATATAACTCGACTGTAACGAGACTATTATCA | |
| cases | CCAATTGCAAAAAATCTAAACCATTTGCTACAACCGAG | |
| correspond | ACCGTCTGGCAGGTTATTTGGCGCTGTAATTGGATCGA | |
| to APMV F | TTGCCTTAGGGGTAGCTACATCCGCACAGATTTCAGCT | |
| and HN | GCTATAGCATTGGTCCGTGCTCAACAGAATGCAAACGA | |
| coding | TATCCTCGCTCTTAAAGCTGCAATACAATCTAGTAATG | |
| sequences) | AGGCAATAAAACAACTTACTTATGGCCAAGAAAAGCA | |
| | ACTACTAGCAATATCAAAAATACAAAAAGCCGTAAAT | |
| | GAACAAGTAATCCCTGCATTGACTGCACTTGACTGTGC | |
| | AGTTCTTGGAAATAAACTAGCTGCACAACTGAACCTCT | |
| | ACCTCATTGAAATGACGACTATTTTTGGTGACCAAATA | |
| | AATAACCCAGTCCTAACTCCAATACCACTCAGTTATCT | |
| | CCTGCGGTTGACAGGCTCTGAGTTAAATGATGTATTAT | |
| | TACAACAGACTCGATCCTCTTTGAGCCTAATCCACCTT | |
| | GTCTCTAAAGGCTTATTAAGTGGTCAGATTATAGGATA | |
| | TGACCCTTCAGTACAAGGCATCATTATCAGAATAGGAC | |
| | TGATCAGGACTCAAAGAATAGATCGGTCACTAGTTTTC | |
| | CaACCTTACGTATTACCAATTACTATTAGTTCTAACATA | |
| | GCCACACCAATTATACCCGACTGTGTGGTCAAGAAGG | |
| | GAGTAATAATTGAGGGAATGCTTAAGAGTAATTGTATA | |
| | GAATTGGAACGAGATATAATTTGCAAGACTATCAACA | |
| | CATACCAAATAACTAAGGAAACTAGAGCATGCTTACA | |
| | AGGTAATATAACAATGTGTAAGTACCAGCAGTCCAGG | |
| | ACACAGTTGAGCACCCCCTTTATTACATATAATGGAGT | |
| | TGTAATTGCAAATTGTGATTTGGTATCATGCCGATGCA | |
| | TAAGACCCCCTATGATTATCACACAAGTAAAAGGTTAC | |
| | CCTCTGACAATTATAAATAGGAATTTATGTACCGAGTT | |
| | GTCGGTGGATAATTTAATTTTAAATATTGAAACAAACC | |
| | ATAACTTTTCATTAAACCCTACTATTATAGATTCACAAT | |
| | CCCGGCTTATAGCTACTAGTCCATTAGAAATAGATGCC | |
| | CTTATTCAAGATGCGCAACATCACGCGGCTGCGGCCCT | |
| | TCTTAAAGTAGAAGAAAGCAATGCTCACTTATTAAGAG | |
| | TTACAGGGCTGGGCTCATCAAGTTGGCACATCATACTT | |
| | ATATTAACATTGCTTGTATGCACCATAGCATGGCTCAT | |
| | TGGTTTATCTATTTATGTCTGCCGCATTAAAAATGATG | |
| | ACTCGACCGACAAAGAACCTACAACCCAATCATCGAA | |
| | CCGaGGCATTGGGGTTGGATCTATACAATATATGACAT | |
| | GAtgaacacagatgaggaacgaaggtttccctaatagtaatttgtgtgaaagttctggtagt | |
| | ctgtcagttcagagagttaagaaaaaactaccggttgtagatgaccaaaggacgatatacg | |
| | ggtagaacggtaagagaggccgcccctcaattgcgagccaggcttcacaacctccgttct | |
| | accgcttcaccgacaacagtcctcaatcATGGAGCCGACAGGATCAAA | |
| | AGTTGACATTGTCCCTTCCCAAGGTACCAAGAGAACAT | |
| | GTCGAACCTTTTATCGCCTCTTAATTCTTATTTTGAATC | |
| | TTATTATAATTATATTAACAATTATCAGTATTTATGTCT | |
| | CTATCTCAACAGATCAACACAAATTGTGCAATAATGAG | |
| | GCTGACTCACTTTTACACTCAATAGTAGAACCCATAAC | |
| | AGTCCCCCTAGGAACAGACTCGGATGTTGAGGATGAA | |
| | TTACGTGAGATTCGACGTGATACAGGCATAAATATTCC | |
| | TATCCAAATTGACAACACAGAGAACATCATATTAACTA | |
| | CATTAGCAAGTATCAACTCTAACATTGCACGCCTTCAT | |
| | AACGCCACCGATGAAAGCCCAACATGCCTGTCACCAG | |
| | TTAATGATCCCAGGTTTATAGCAGGGATTAATAAGATA | |
| | ACCAAAGGGTCGATGATATATAGGAATTTCAGCAATTT | |
| | GATAGAACATGTTAACTTTATACCATCTCCAACGACAT | |
| | TATCAGGCTGTACAAGAATTCCATCTTTTTCACTATCTA | |
| | AAACACATTGGTGTTACTCGCATAATGTAATATCTACT | |
| | GGTTGTCAAGACCATGCTGCGAGTTCACAGTATATTTC | |
| | CATAGGAATAGTAGATACAGGATTGAATAATGAGCCC | |
| | TATTTGCGTACAATGTCTTCACGCTTGCTAAATGATGG | |
| | CCTAAATAGAAAGAGCTGCTCTGTCACAGCCGGCGCTG | |
| | GTGTCTGTTGGCTATTGTGTAGTGTTGTAACAGAAAGT | |
| | GAATCAGCTGACTACAGATCAAGAGCCCCCACTGCAA | |
| | TGATTCTCGGAAGGTTCAATTTTTATGGTGATTACACT | |
| | GAATCCCCTGTTCCTGCATCTTTGTTCAGCGGTCGTTTC | |
| | ACTGCTAATTACCCTGGAGTTGGCTCAGGAACCCAATT | |
| | AAATGGGACCCTTTATTTTCCAATATATGGGGGTGTTG | |
| | TTAACGACTCTGATATTGAGTTATCGAACCGAGGGAAG | |
| | TCATTCAGACCTAGGAACCCTACAAACCCATGTCCAGA | |
| | TCCTGAGGTGACCCAAAGTCAGAGGGCTCAGGCAAGT | |
| | TACTATCCGACAAGGTTTGGCAGGCTGCTCATACAACA | |
| | AGCAATACTAGCTTGTCGTATTAGTGACACTACATGCA | |
| | CTGATTATTATCTTCTATACTTTGATAATAATCAAGTCA | |
| | TGATGGGTGCAGAAGCCCGAATTTATTATTTAAACAAT | |
| | CAGATGTACTTATATCAAAGATCTTCGAGTTGGTGGCC | |
| | GCATCCGCTTTTTTACAGATTCTCACTGCCTCATTGTGA | |
| | ACCTATGTCTGTCTGTATGATCACCGATACACACTTAA | |
| | TATTGACATATGCTACCTCACGCCCTGGCACTTCAATTT | |
| | GTACAGGGGCCTCGCGATGTCCTAATAACTGTGTTGAT | |
| | GGTGTCTATACAGACGTTTGGCCCTTGACTGAGGGTAC | |
| | AACACAAGATCCAGATTCCTACTACACAGTATTCCTCA | |
| | ACAGTCCCAACCGCAGGATCAGTCCTACAATTAGCATT | |
| | TACAGCTACAACCAGAAGATTAGCTCTCGTCTGGCTGT | |
| | AGGAAGTGAAATAGGAGCTGCTTACACGACCAGTACA | |
| | TGTTTTAGCAGGACAGACACTGGGGCACTATACTGCAT | |
| | CACTATAATAGAAGCTGTAAACACAATCTTTGGACAAT | |
| | ACCGAATAGTACCGATCCTTGTTCAACTAATTAGTGACt | |
| | agttgagtcaattataaaggagttggaaagatggcattgtatcacctatcttctgcgacatcaa | |
| | gaatca | |
| APMV12/ | atttacagttagtttacctgtctatcaagttagaaaaaacacgggtagaagattctggatcccg | 12 |
| Wigeon/Italy/ | gttggcgccctccaggtgcaagATGGCCATCCCAGTGCCCTCTTCG | |
| 3920_1/05 | ACCGCTCTGATGATCTTCAACATTCTAGTGTCCCTCGCC | |
| (lower cases | CCCGCCTCCGCTCTGGATGGCAGACTGTTGTTAGGAGC | |
| correspond | AGGTATCGTACCTACGGGAGACAGACAGGTAAATGTG | |
| to NDV | TATACTTCATCTCAAACCGGTATAATTGCCTTAAAATT | |
| derived | GCTGCCCAACCTCCCAAAGGATAAGGAGAATTGCGCT | |
| sequences | GAGGTGTCTATCAGATCCTACAACGAGACTCTGACCCG | |
| and upper | CATCCTCACCCCTCTCGCTCAATCCATGGCAGCCATAA | |
| cases | GGGGTAATTCAACAGTATCAACTCGTGGAAGAGAGCC | |
| correspond | AAGACTAGTGGGTGCCATCATAGGAGGCGTAGCTCTA | |
| to APMV F | GGTGTAGCTACGGCAGCACAGATCACAGCGGCAACGG | |
| and HN | CCCTTATCCAAGCCAATCAAAATGCAGAGAACATTGCA | |
| coding | AGACTTGCCAAAGGTCTAGCAGCTACCAATGAGGCAG | |
| sequences) | TGACGGATTTAACGAAAGGAGTGGGCTCTCTTGCTATT | |
| | GGGGTTGGAAAGTTACAGGATTATGTAAATGAGCAAT | |
| | TTAATAGGACGGGAGAGGCAATCGAATGTTTGACGAT | |
| | CGAATCTAGAGTAGGTGTCCAGCTCAGTCTCTATCTAA | |
| | CAGAGGTTATTGGAGTCTTCGGTGATCAGATCACATCT | |
| | CCAGCATTATCTGATATCAGTATTCAGGCATTATACAA | |
| | TCTGGCTGGAGGGAACTTGAACGTCTTGCTGCAGAAGA | |
| | TGGGTATTGAAGGGACACAGCTAGGCTCCTTAATCAAC | |
| | AGCGGATTGATAAAAGGCAGACCAATCATGTATGATG | |
| | ATGGTAACAAAATTTTAGGTATCCAAGTAACTCTTCCA | |
| | TCAGTGGGTAGGATCAATGGCGCACGAGCAACTCTACT | |
| | TGAGGCAATTGCGGTGGCTACTCCTAAAGGGAATGCTA | |
| | GCCCATTAATACCTAGAGCTGTTATCTCAGTGGGATCG | |
| | CTAGTGGAAGAATTAGATATGACTCCATGCGTGCTGAC | |
| | TCCAACAGACATCTTTTGCACCAGGATCTTGTCTTATCC | |
| | ATTAAGTGATTCTCTCACCACTTGTCTCAAAGGGAATC | |
| | TTTCGTCTTGCGTCTTCTCACGTACGGAAGGGGCATTA | |
| | TCGACACCTTATGTTTCTGTGCATGGTAAGATTGTTGCC | |
| | AATTGTAAGTCTGTGGTTTGCCGATGTGTGGAGCCACA | |
| | ACAAATCATATCCCAAAACTATGGGGAGGCCCTTAGCC | |
| | TGATAGATGAGTCCCTATGTAGGATCTTAGAACTAAAC | |
| | GGAGTGATCCTTAAGATGGACGGACAGTTCACATCAG | |
| | AATACACAAAAAACATAACTATAGATCCTGTGCAGGT | |
| | CATAATATCTGGACCGATCGATATATCTTCTGAGCTTT | |
| | CGCAGGTCAACCAATCACTAGATAGCGCACTGGAAAA | |
| | TATAAAAGAGAGCAATTCATACCTGTCAAAAGTGAAT | |
| | GTGAAGCTGATCAGCTCCTCGGCCATGATCACGTACAT | |
| | TGTGATAACTGTGATTTGCCTGATTTTGACTTTCGTAGC | |
| | GTTAGTCCTTGGGATATACTCATATACAAAAATCAGGT | |
| | CTCAACAGAAGACTCTGATATGGATGGGTAATAACATT | |
| | GCGAGGTCAAAAGAGGGGAACCGGTTTtgaacacagatgagga | |
| | acgaaggtttccctaatagtaatttgtgtgaaagttctggtagtctgtcagttcagagagttaa | |
| | gaaaaaactaccggttgtagatgaccaaaggacgatatacgggtagaacggtaagagag | |
| | gccgcccctcaattgcgagccaggcttcacaacctccgttctaccgcttcaccgacaacag | |
| | tcctcaatcATGGAGAGTGCAACCAGCCAAGTGTCCTTTGA | |
| | AAATGACAAAACCTCTGATCGTCGGACTTGGCGAGCA | |
| | GTATTTAGAGTACTGATGATAATACTCGCTCTTAGTAG | |
| | CCTATGTGTAACTGTAGCAGCTCTTATATACTCAGCGA | |
| | AGGCCGCAATCCCTGGGAACATCGATGCATCTGAACA | |
| | AAGGATATTATCATCCGTTGAGGCCGTTCAGGTGCCCG | |
| | TATCAAGGTTAGAAGACACCAGTCAGAAGATATACCG | |
| | CCAGGTCATTCTCGAGGCGCCGGTAACTCAGCTCAACA | |
| | TGGAGACGAATATTCTAAATGCTATTACATCCCTTTCA | |
| | TATCAAATTGATGCTTCAGCCAACTCTTCTGGTTGCGG | |
| | TGCCCCTGTCCATGACTCTGACTTCACAGGGGGTGTCG | |
| | GTCGAGAGCTACTTCAAGAGGCAGAAGTTAATCTGAC | |
| | CATAATCAGGCCCTCCAAATTCTTGGAGCACTTAAACT | |
| | TCATACCAGCCCCGACAACAGGGAATGGCTGCACAAG | |
| | GATACCATCGTTTGATCTAGGCCAAACTCATTGGTGCT | |
| | ACACACACAACGTCGTGCTCAATGGCTGCAGAGACCGa | |
| | GGCCACTCTTTTCAATATGTTGCACTAGGCATACTCAG | |
| | GACATCAGCTACAGGGTCAGTATTCTTATCAACACTCC | |
| | GATCTGTAAATTTAGACGACGACCGTAACAGAAAGTC | |
| | ATGTAGTGTAAGTGCAACCCCGATAGGCTGCGAGATG | |
| | CTCTGTTCTCTTGTCACAGAGACTGAAGAAGGAGATTA | |
| | TGATAGCATCGACCCGACCCCTATGGTGCATGGCAGGT | |
| | TAGGATTTGATGGCAAATATAGGGAAGTGGACCTTAG | |
| | CGAAAAGGAGATATTCGCTGACTGGCGCGCCAATTATC | |
| | CTGCTGTTGGCGGTGGCGCTTTTTTTGGTAATCGTGTAT | |
| | GGTTCCCTGTTTATGGAGGTCTGAAGGAAGGAACCCAA | |
| | AGTGAGAGAGATGCAGAGAAAGGTTATGCAATATATA | |
| | AACGCTTCAATAACACTTGCCCTGACGATAATACAACT | |
| | CAAATCGCGAATGCTAAAGCATCATATCGGCCATCTCG | |
| | ATTTGGCGGACGATTTATCCAACAGGGTATCCTCTCTT | |
| | TTAAAGTTGAAGGGAACTTAGGATCAGATCCGATCCTC | |
| | AGCCTGACTGACAACTCAATCACATTGATGGGTGCCGA | |
| | GGCACGTGTGATGAATATTGAGAATAAACTATACCTCT | |
| | ATCAGAGAGGTACTTCTTGGTTTCCATCTGCCTTAGTAT | |
| | ACCCCTTGGATGTAGCTAATACAGCTGTAAAAGTGCGG | |
| | GCGCCATACATTTTTGACAAATTCACTAGGCCCGGAGG | |
| | ACATCCATGCAGCGCCAGTTCACGGTGCCCTAACGTAT | |
| | GCGTCACAGGGGTTTATACGGATGCCTATCCACTTGTA | |
| | TTTTCAAGGAGTCATGACATTGTGGCAGTCTACGGTAT | |
| | GCAGTTGGCGGCGGGCACTGCACGACTTGATCCTCAGG | |
| | CAGCAATATGGTATGGGAACGAGATGAGTACACCTAC | |
| | TAAAGTAAGTAGCTCAACTACTAAAGCTGCCTATACTA | |
| | CTTCCACATGTTTTAAGGTGACAAAAACTAAGAGAATC | |
| | TACTGTATAAGTATAGCAGAAATAGGGAACACACTCTT | |
| | TGGCGAGTTTAGGATAGTGCCACTATTAATCGAAGTAC | |
| | AAAAGACTCCTCTCACTAGGAGAAGCGAGCTCCGGCA | |
| | ACAAATGCCCCAACCTCCCATCGATTTGGTTATTGACA | |
| | ATCCGTTCTGTGCGCCCTCTGGTAACTTGAGCAGAAAG | |
| | AATGCCATTGACGAGTATGCCAATTCATGGCCAtagttgagt | |
| | caattataaaggagttggaaagatggcattgtatcacctatcttctgcgacatcaagaatca | |
| APMV5/ | atttacagttagtttacctgtctatcaagttagaaaaaacacgggtagaagattctggatcccg | 13 |
| budgerigar/ | gttggcgccctccaggtgcaagATGTTCCAACTTCCTTTGACCATTC | |
| Japan/TI/75 | TTCTTAGCATTCTTAGTGTTCACCAGTCGCTTTGTCTAG | |
| (lower cases | ACAACAGTAAGCTCATTCATGCAGGAATCATGAGTACT | |
| correspond | ACTGAGAGAGAAGTTAATGTTTATGCACAATCTATTAC | |
| to NDV | TGGGTCAATAGTGGTGAGGTTGATTCCAAATATCCCAA | |
| derived | GTAACCATAAATCTTGTGCAACTAGCCAAATCAAACTA | |
| sequences | TACAATGACACGTTAACAAGATTGTTGACCCCAATTAG | |
| and upper | AGCTAATCTAGAAGGACTTATTAGTGCTGTTTCTCAGG | |
| cases | ACCAATCGCAGAATTCTGGGAtaAGAgAGccGCGTTTTGT | |
| correspond | AGGCGCAGTAATTGGAGCAGCTGCCCTCGGCTTGGCA | |
| to APMV F | ACCGCTGCACAGGTGACTGCCACTGTAGCGTTAAATCA | |
| and HN | AGCGCAAGAAAACGCTCGGAATATCCTAAAGCTTAAA | |
| coding | AACTCGATTCAGAAGACAAACGAGGCGGTGATGGAAC | |
| sequences) | TTAAAGATGCTGTGGGCCAAACAGCAGTAGCTATTGAC | |
| | AAAACTCAGGCCTTCATAAATAATCAAATCTTGCCTGC | |
| | AATTTCAAATCTCTCATGTGAGGTCCTAGGGAATAAAA | |
| | TTGGGGTCCAATTATCTTTGTACCTTACTGAATTAACA | |
| | ACAGTATTCGGCAATCAACTGACAAACCCAGCCCTTAC | |
| | CACACTGTCATTACAAGCCTTGTACAATCTTTGTGGAG | |
| | ATGACTTCAATTACTTAATCAACCTATTAAATGCAAAA | |
| | AATCGTAACTTAGCCTCACTTTATGAAGCAAACCTAAT | |
| | TCAGGGGAGAATCACTCAATATGACTCAATGAATCAGT | |
| | TATTAATTATTCAGGTACAAATACCAAGCATCTCCATA | |
| | GTGTCAGGAATGAGGGTCACAGAATTATTCACACTTAG | |
| | TGTTGATACACCTATAGGAGAGGGAAAGGCCCTAGTA | |
| | CCAAAATATGTCCTGTCCTCAGGGAGAATAATGGAAG | |
| | AGGTTGACCTAAGCAGTTGCGCTATAACATCAACATCA | |
| | GTTTTCTGTTCCTCTATCATCTCTAGACCCCTTCCACTT | |
| | GAAACAATAAATTGCCTGAATGGGAATGTTACACAGT | |
| | GTCAATTTACCGCCAACACAGGAACCCTTGAATCGAGA | |
| | TACGCTGTTATAGGAGGCTTGGTGATTGCTAACTGTAA | |
| | GGCTATAGTATGCAGGTGCCTAAATCCACCAGGTGTCA | |
| | TTGCGCAAAATCTTGGCTTACCAATTACAATCATCTCA | |
| | TCCAATACTTGTCAGCGAATTAATTTAGAACAAATCAC | |
| | TTTGTCTCTTGGGAACAGCATATTATCTACATACAGTG | |
| | CCAATTTATCCCAAGTTGAGATGAATTTAGCTCCATCA | |
| | AATCCTCTGGATATCTCAGTTGAATTGAATCGAGTCAA | |
| | CACCAGTCTCTCTAAAGTGGAATCTCTAATAAAAGAAA | |
| | GCAATAGTATCCTGGACTCAGTTAACCCTCAAATTTTA | |
| | AATGTCAAGACAGTAATTATCCTGGCCTTCATAATAGG | |
| | ACTCATTGTTGTGTGGTGTTTCATATTGACATGTCTAAT | |
| | AATTAGAGGATTTATGCTTCTTGTAAAACAACAAAAGT | |
| | TTAAAGGACTCTCTGTTCAGAATAATCCGTATGTTTCT | |
| | AACAATTCTCATtgaacacagatgaggaacgaaggtttccctaatagtaatttgt | |
| | gtgaaagttctggtagtctgtcagttcagagagttaagaaaaaactaccggttgtagatgacc | |
| | aaaggacgatatacgggtagaacggtaagagaggccgcccctcaattgcgagccaggct | |
| | tcacaacctccgttctaccgcttcaccgacaacagtcctcaatcATGGACAAATC | |
| | ATATTACATAGAGCCTGAAGATCAAAGAGGTAACTCTC | |
| | GAACATGGAGACTATTATTTAGGTTGATTGTATTAACG | |
| | TTGCTCTGTCTGATCGCATGTATCTTAGTAAGTCAATTG | |
| | TTCTACCCTTGGCTCCCCCAAGTCTTGTCCACTCTGATC | |
| | AGCCTAAATAACTCAATTATCACAAGCAGCAATGGTCT | |
| | CAAAAAGGAAATCCTGAACCAGAACATAAAAGAGGAC | |
| | CTCATATATAGAGAAGTTGCTATAAATATACCTTTAAC | |
| | ATTAGATAGGGTTACTGTTGAGGTAGGGACTGCAGTAA | |
| | ACCAGATTACTGATGCACTCAGGCAACTCCAGTCAGTT | |
| | AATGGATCTGCTGCATTCGCCTCATCAAACTCTCCTGA | |
| | TTATAGTGGGGGAATAGAACACCTGATTTTCCAAAGGA | |
| | ATACGCTTATTAATCGCTCAGTGAGTGTCTCAGATTTA | |
| | ATAGAACACCCCAGTTTCATACCAACTCCTACTACACA | |
| | GCATGGTTGTACCAGAATCCCCACATTCCACCTAGGAA | |
| | CTCGCCACTGGTGCTATAGTCACAATATAATAGGTCAG | |
| | GGATGTGCTGATTCTAGAGCTAGTGTGATGTATATTTC | |
| | AATGGGAGCACTGGGTGTCAGTTCATTGGGAACCCCG | |
| | ACCTTCACAACATCTGCTGCATCAATATTATCTGATAG | |
| | CCTCAATCGGAAGAGTTGCAGTATAGTAGCAACAACT | |
| | GAGGGTTGTGACGTACTCTGCAGTATAGTTACACAAAC | |
| | AGAAGACCAAGATTATGCTGATCACACTCCTACTCCAA | |
| | TGATACATGGTAGATTATGGTTTAATGGCACATACACA | |
| | GAGAGATCCTTATCCCAGAGTTTATTCCTTGGAACATG | |
| | GGCTGCGCAATATCCGGCTGTAGGATCTGGTATAATGA | |
| | CACCTGGGCGAGTTATATTCCCTTTCTATGGAGGTGTG | |
| | ATCCCTAACTCTCCTCTCTTCTTGGATCTCGAAAGATTC | |
| | GCTTTATTCACACATAATGGAGACTTAGAATGCATGAA | |
| | CTTAACACAATATCAGAAAGAAGCAATTTACTCTGCAT | |
| | ATAAGCCTCCCAAGATTAGAGGATCACTGTGGGCACA | |
| | AGGCTTCATAGTATGTTCAGTAGGAGACATGGGGAATT | |
| | GCTCTCTTAAAGTGATCAATACAAGCACAGTTATGATG | |
| | GGTGCAGAAGGTCGGCTACAATTAGTTGGGGACTCCGT | |
| | TATGTACTATCAGAGATCATCATCCTGGTGGCCTGTAG | |
| | GAATTCTTTATCGGTTGAGTCTTGTAGACATCATCGCC | |
| | GGAGATATACAGGTCGTCATAAACAGTGAACCACTCC | |
| | CTCTGAGCAAGTTCCCaCGGCCAACCTGGACTCCAGGA | |
| | GTGTGTCAAAAACCAAATGTATGCCCTGCAGTTTGTGT | |
| | AACTGGGGTCTATCAAGACCTTTGGGCAATTTCCGCAG | |
| | GGGAGACACTATCTGAAATGACATTCTTTGGAGGATAT | |
| | TTAGAGGCATCCACCCAACGAAAAGATCCATGGATAG | |
| | GCGTTGCTAATCAATATAGTTGGTTCATGAGAAGAAGA | |
| | TTATTCAAGACAAGCACTGAAGCTGCATATTCGTCATC | |
| | AACGTGTTTTAGGAACACTAGACTGGATCGAAATTTCT | |
| | GCCTATTAGTCTTTGAATTAACTGATAACTTACTTGGA | |
| | GACTGGAGAATTGTCCCCCTCTTATTTGAATTAACCAT | |
| | CGTAtagttgagtcaattataaaggagttggaaagatggcattgtatcacctatcttctgcg | |
| | acatcaagaatca | |
| APMV10/ | atttacagttagtttacctgtctatcaagttagaaaaaacacgggtagaagattctggatcccg | 14 |
| penguin/ | gttggcgccctccaggtgcaagATGACTCGTACTCGGTTGCTCTTT | |
| Falkland | CTCCTCACTTGTTACATTCCAGGTGCTGTCTCACTTGAC | |
| Islands/324 | AACTCTATATTAGCTCCAGCTGGGATAATAAGCGCTTC | |
| /07 | GGAGAGACAGATTGCTATATATACTCAAACTCTGCAGG | |
| (lower cases | GAACTATTGCACTTCGATTTATACCTGTACTTCCGCAG | |
| correspond | AACTTATCATCATGTGCTAAAGACACCCTGGAATCCTA | |
| to NDV | TAACTCCACTGTTTCAAATCTTTTATTGCCTATTGCTGA | |
| derived | GAACCTAAATGCCTTGTTAAAAGACGCCGATAAGCCAT | |
| sequences | CCCAACGAATTATTGGGGCTATCATAGGATCAGTAGCG | |
| and upper | CTAGGCGTAGCAACAACTGCACAAGTGACTGCAGCCC | |
| cases | TTGCAATGACGCAGGCACAACAAAATGCACGAAATAT | |
| correspond | ATGGAAGTTGAAAGAGTCTATCAAAAATACAAATCAA | |
| to APMV F | GCAGTATTGGAACTAAAAGACGGGCTACAACAATCTG | |
| and HN | CTATTGCACTTGACAAGGTCCAGTCCTTCATCAACTCG | |
| coding | GAAATATTACCTCAAATAAATCAGTTAGGATGCGAGGT | |
| sequences) | TGCAGCAAACAAATTGGGAATATTTCTATCTCTCTACC | |
| | TAACTGAAATCACTACAGTGTTCAAAAATCAAATCACG | |
| | AATCCCGCTCTTTCCACATTATCATACCAAGCCCTTTAT | |
| | AATTTGTGTGGGGGCAATATGGCTGCTTTAACAAAACA | |
| | AATAGGAATTAAAGATACAGAAATTAACTCATTATATG | |
| | AAGCAGAATTGATCACAGGACAAGTTATAGGGTATGA | |
| | TTCAGCAGACCAGATACTGTTGATTCAAGTATCATATC | |
| | CAAGTGTTTCAAGGGTCCAAGGGGTTAGGGCAGTAGA | |
| | ACTCTTGACAGTCAGTGTGGCAACACCAAAAGGTGAG | |
| | GGGAAAGCAATTGCTCCAAGCTTTATAGCTCAGAGCA | |
| | ATATAATTGCTGAAGAGTTAGATACACAACCATGTAAG | |
| | TTTAGTAAGACAACACTCTACTGTAGACAAGTTAACAC | |
| | TAGGACATTACCAGTTAGGGTAGCAAACTGCCTTAAAG | |
| | GCAAATATAATGACTGCCAATATACCACAGAAATAGG | |
| | TGCATTGGCATCACGATATGTCACGATTACAAATGGGG | |
| | TTGTTGCCAACTGCAGATCTATCATCTGTAGGTGCCTG | |
| | GACCCGGAGGGAATAGTTGCCCAAAATTCTGACGCAG | |
| | CAATCACTGTTATTGATAGGTCCACTTGCAAGTTGATC | |
| | CAGTTAGGTGATATTACCCTCAGATTAGAAGGCAAATT | |
| | ATCCTCATCATACTCAAAGAATATAACCATTGATATAT | |
| | CTCAAGTAACTACATCTGGTTCTTTAGATATAAGTAGC | |
| | GAATTAGGCTCTATTAATAATACTATAACCAAAGTAGA | |
| | AGATTTGATAAGTAAGTCCAATGATTGGTTGAGTAAAG | |
| | TAAATCCTACCCTAATATCGAATGACACTATCATTGCC | |
| | CTCTGTGTGATTGCTGGTATTGTCGTTATCTGGTTAGTT | |
| | ATAATCACAATACTATCGTATTATATACTCATAAAACT | |
| | TAAAAATGTAGCATTGCTTTCAACCATGCCAAAGAAAG | |
| | ATCTAAACCCGTATGTTAACAACACTAAATTTTGAtgaac | |
| | acagatgaggaacgaaggtttccctaatagtaatttgtgtgaaagttctggtagtctgtcagtt | |
| | cagagagttaagaaaaaactaccggttgtagatgaccaaaggacgatatacgggtagaac | |
| | ggtaagagaggccgcccctcaattgcgagccaggcttcacaacctccgttctaccgcttca | |
| | ccgacaacagtcctcaatcATGGACTCATCACAAATGAATATTTT | |
| | GGATGCTATGGATAGGGAAAGTAGTAAAAGGACATGG | |
| | AGGGGAGTATTCCGAGTTACTACTATAATCATGGTTGT | |
| | CACATGTGTCGTCCTATCTGCAATCACACTATCGAAGG | |
| | TTGCCCATCCTCAGGGGTTCGACACCAATGAGCTGGGT | |
| | AATGGCATCGTGGATCGAGTCAGCGATAAAATAACTG | |
| | AGGCGTTAACAGTGCCAAACAATCAAATAGGTGAAAT | |
| | ATTCAAAATTGTGGCACTGGACTTGCATGTTCTGGTCA | |
| | GCTCATCGCAACAGGCAATTGCAGGACAAATTGGCAT | |
| | GCTTGCTGAGAGTATCAACAGTATATTAAGCCAAAATG | |
| | GATCTGCATCGACCATTCTATCATCCAGCCCAGAATAT | |
| | GCAGGGGGTATAGGTGTCCCTCTCTTTAGCAATAAGTT | |
| | AACAAATGGAACTGTAATAAAACCCATAACCTTAATTG | |
| | AACACCCTAGTTTCATCCCGGGCCCAACTACTATAGGA | |
| | GGTTGCACTCGGATTCCCACATTCCATATGGCCTCGTC | |
| | TCATTGGTGTTACTCTCATAATATCATAGAGAAAGGTT | |
| | GTAAGGATAGTGGGATATCATCCATGTATATATCATTA | |
| | GGAGTATTACAAGTATTGAAGAAAGGAACTCCTGTATT | |
| | TTTGGTAACTGCAAGCGCTGTACTATCTGATGATAGGA | |
| | ACCGGAAATCGTGCAGTATTATAAGTTCAAGGTTTGGG | |
| | TGTGAGATACTATGCAGCCTTGTAACAGAAGCAGAGTC | |
| | TGACGATTACAAATCTGATACGCCAACTGGAATGGTGC | |
| | ACGGCAGATTATATTTCAATGGGACATACAGAGAAGG | |
| | GCTGGTAGATACAGAGACTATATTCCGAGACTTTTCTG | |
| | CTAATTATCCCGGGGTCGGATCAGGGGAAATTGTAGA | |
| | GGGGCACATACATTTTCCCATATACGGAGGAGTGAAG | |
| | CAGAATACTGGCCTATACAACAGTCTTACCCCTTATTG | |
| | GCTCGATGCAAAGAACAAATATGACTATTGCAAGTTGC | |
| | CATATACAAATCAAACAATCCAGAACTCGTATAAACCC | |
| | CCATTCATCCACGGAAGATTTTGGGCACAAGGAATACT | |
| | ATCATGTGAATTAGATCTATTCAATTTGGGGAATTGCA | |
| | ATCTCAAGATAATCCGAAGTGATAAAGTCATGATGGG | |
| | AGCAGAAAGTCGGTTAATGCTTGTGGGGTCAAAATTGC | |
| | TAATGTATCAGAGGGCATCGTCCTGGTGGCCGCTGGGG | |
| | ATTACACAGGAGATAGATATAGCTGAGCTACACTCAA | |
| | GTAATACTACCATATTAAGAGAAGTTAAACCCATACTG | |
| | TCATCGAAGTTCCCaCGGCCGTCCTATCAGCCGAATTAT | |
| | TGCACGAAGCCAAGTGTATGTCCTGCAGTGTGTGTCAC | |
| | AGGAGTATACACTGACATGTGGCCTATTTCAATCACTG | |
| | GCAACATATCTGATTATGCTTGGATTAGCCATTATTTG | |
| | GATGCACCAACATCAAGACAACAGCCGAGGATTGGGA | |
| | TTGCAAACCAATATTTCTGGATCCATCAGACGACTATA | |
| | TTTCCAACCAATACTCAGAGCTCCTACTCCACTACCAC | |
| | ATGCTTTAGGAATCAGGTGAGAAGTAGGATGTTTTGTT | |
| | TATCCATTGCAGAGTTTGCAGATGGAGTGTTTGGGGAA | |
| | TTTAGGATCGTGCCCTTATTATATGAGTTGAGAGTAtagtt | |
| | gagtcaattataaaggagttggaaagatggcattgtatcacctatcttctgcgacatcaagaa | |
| | tca | |
| cDNA of | accaaacagagaatccgtgagttacgataaaaggcgaaggagcaattgaagtcgcacgg | 15 |
| genomic | gtagaaggtgtgaatctcgagtgcgagcccgaagcacaaactcgagaaagccttctgcca | |
| sequence of | acatgtcttccgtatttgatgagtacgaacagctcctcgcggctcagactcgccccaatgga | |
| NDV strain | gctcatggagggggagaaaaagggagtaccttaaaagtagacgtcccggtattcactctta | |
| LaSota | acagtgatgacccagaagatagatggagctttgtggtattctgcctccggattgctgttagcg | |
| | aagatgccaacaaaccactcaggcaaggtgctctcatatctcttttatgctcccactcacagg | |
| | taatgaggaaccatgttgccCttgcagggaaacagaatgaagccacattggccgtgcttga | |
| | gattgatggctttgccaacggcacgccccagttcaacaataggagtggagtgtctgaagag | |
| | agagcacagagatttgcgatgatagcaggatctctccctcgggcatgcagcaacggaacc | |
| | ccgttcgtcacagccggggcCgaagatgatgcaccagaagacatcaccgataccctgga | |
| | gaggatcctctctatccaggctcaagtatgggtcacagtagcaaaagccatgactgcgtatg | |
| | agactgcagatgagtcggaaacaaggcgaatcaataagtatatgcagcaaggcagggtc | |
| | caaaagaaatacatcctctaccccgtatgcaggagcacaatccaactcacgatcagacagt | |
| | ctcttgcagtccgcatctttttggttagcgagctcaagagaggccgcaacacggcaggtggt | |
| | acctctacttattataacctggtaggggacgtagactcatacatcaggaataccgggcttact | |
| | gcattcttcttgacactcaagtacggaatcaacaccaagacatcagcccttgcacttagtagc | |
| | ctctcaggcgacatccagaagatgaagcagctcatgcgtttgtatcggatgaaaggagata | |
| | atgcgccgtacatgacattacttggtgatagtgaccagatgagctttgcgcctgccgagtat | |
| | gcacaactttactcctttgccatgggtatggcatcagtcctagataaaggtactgggaaatac | |
| | caatttgccagggactttatgagcacatcattctggagacttggagtagagtacgctcaggct | |
| | cagggaagtagcattaacgaggatatggctgccgagctaaagctaaccccagcagcaaG | |
| | gaGgggcctggcagctgctgcccaacgggtctccgaGgaGaccagcagcataGaca | |
| | tgcctactcaacaagtcggagtcctcactgggcttagcgagggggggtcccaagctctaca | |
| | aggcggatcgaatagatcgcaagggcaaccagaagccggggatggggagacccaattc | |
| | ctggatctgatgagagcggtagcaaatagcatgagggaggcgccaaactctgcacaggg | |
| | cactccccaatcggggcctcccccaactcctgggccatcccaagataacgacaccgactg | |
| | ggggtattgatggacaaaacccagcctgcttccacaaaaacatcccaatgccctcacccgt | |
| | agtcgacccctcgatttgcggctctatatgaccacaccctcaaacaaacatccccctetttcc | |
| | tccctccccctgctgtacaactAcgTacgccctagataccacaggcacaatgoggctcac | |
| | taacaatcaaaacagagccgagggaattagaaaaaagtacgggtagaagagggatattca | |
| | gagatcagggcaagtctcccgagtctctgctctctcctctacctgatagaccaggacaaaca | |
| | tggccacctttacagatgcagagatcgacgagctatttgagacaagtggaactgtcattgac | |
| | aacataattacagcccagggtaaaccagcagagactgttggaaggagtgcaatcccacaa | |
| | ggcaagaccaaggtgctgagcgcagcatgggagaagcatgggagcatccagccaccg | |
| | gccagtcaagacaaccccgatcgacaggacagatctgacaaacaaccatccacacccga | |
| | gcaaacgaccccgcatgacagcccgccggccacatccgccgaccagccccccacccag | |
| | gccacagacgaagccgtcgacacacagCtcaggaccggagcaagcaactctctgctgtt | |
| | gatgcttgacaagctcagcaataaatcgtccaatgctaaaaagggcccatggtcgagcccc | |
| | caagaggggaatcaccaacgtccgactcaacagcaggggagtcaacccagtcgcggaa | |
| | acagtcaggaaagaccgcagaaccaagtcaaggccgcccctggaaaccagggcacag | |
| | acgtgaacacagcatatcatggacaatgggaggagtcacaactatcagctggtgcaaccc | |
| | ctcatgctctccgatcaaggcagagccaagacaatacccttgtatctgcggatcatgtccag | |
| | ccacctgtagactttgtgcaagcgatgatgtctatgatggaggcgatatcacagagagtaag | |
| | taaggttgactatcagctagatcttgtcttgaaacagacatcctccatccctatgatgcggtcc | |
| | gaaatccaacagctgaaaacatctgttgcagtcatggaagccaacttgggaatgatgaaga | |
| | ttctggatcccggttgtgccaacatttcatctctgagtgatctacgggcagttgcccgatctca | |
| | cccggttttagtttcaggccctggagacccctctccctatgtgacacaaggaggcgaaatg | |
| | gcacttaataaactttcgcaaccagtgccacatccatctgaattgattaaacccgccactgca | |
| | tgcgggcctgatataggagtggaaaaggacactgtccgtgcattgatcatgtcacgcccaa | |
| | tgcacccgagttcttcagccaagctcctaagcaagttagatgcagccgggtcgatcgagga | |
| | aatcaggaaaatcaagcgccttgctctaaatggctaattactactgccacacgtagcgggtc | |
| | cctgtccactcggcatcacacggaatctgcaccgagttcccccccgcGgacccaaggtcc | |
| | aactctccaagcggcaatcctctctcgcttcctcagccccactgaatgAtcgcgtaaccgta | |
| | attaatctagctacatttaagattaagaaaaaatacgggtagaattggagtgccccaattgtg | |
| | ccaagatggactcatctaggacaattgggctgtactttgattctgcccattcttctagcaacct | |
| | gttagcatttccgatcgtcctacaagAcacaggagatgggaagaagcaaatcgccccgca | |
| | atataggatccagcgccttgacttgtggactgatagtaaggaggactcagtattcatcacca | |
| | cctatggattcatctttcaagttgggaatgaagaagccacCgtcggcatgatcgatgataaa | |
| | cccaagcgcgagttactttccgctgcgatgctctgcctaggaagcgtcccaaataccggag | |
| | accttattgagctggcaagggcctgtctcactatgatagtcacatgcaagaagagtgcaact | |
| | aatactgagagaatggttttctcagtagtgcaggcaccccaagtgctgcaaagctgtagggt | |
| | tgtggcaaacaaatactcatcagtgaatgcagtcaagcacgtgaaagcgccagagaagatt | |
| | cccgggagtggaaccctagaatacaaggtgaactttgtctccttgactgtggtaccgaaga | |
| | Gggatgtctacaagatcccagctgcagtattgaaggtttctggctcgagtctgtacaatcttg | |
| | cgctcaatgtcactattaatgtggaggtagacccgaggagtcctttggttaaatctCtgtcta | |
| | agtctgacagcggatactatgctaacctcttcttgcatattggacttatgaccacTgtagata | |
| | ggaaggggaagaaagtgacatttgacaagctggaaaagaaaataaggagccttgatctat | |
| | ctgtcgggctcagtgatgtgctcgggccttccgtgttggtaaaagcaagaggtgcacggac | |
| | taagcttttggcacctttcttctctagcagtgggacagcctgctatcccatagcaaatgcttctc | |
| | ctcaggtggccaagatactctggagtcaaaccgcgtgcctgcggagcgttaaaatcattatc | |
| | caagcaggtacccaacgcgctgtcgcagtgaccgccgaccacgaggttacctctactaag | |
| | ctggagaaggggcacacccttgccaaatacaatccttttaagaaataagctgcgtctctgag | |
| | attgcgctccgcccactcacccagatcatcatgacacaaaaaactaatctgtcttgattattta | |
| | cagttagtttacctgtctatcaagttagaaaaaacacgggtagaagattctggatcccggttg | |
| | gcgccctccaggtgcaagatgggctccagaccttctaccaagaacccagcacctatgatg | |
| | ctgactatccgggttgcgctggtactgagttgcatctgtccggcaaactccattgatggcag | |
| | gcctcttgcagctgcaggaattgtggttacaggagacaaagccgtcaacatatacacctcat | |
| | cccagacaggatcaatcatagttaagctcctcccgaatctgcccaaggataaggaggcatg | |
| | tgcgaaagcccccttggatgcatacaacaggacattgaccactttgctcaccccccttggtg | |
| | actctatccgtaggatacaagagtctgtgactacatctggaggggggagacaggggcgcc | |
| | ttataggcgccattattggcggtgtggctcttggggttgcaactgccgcacaaataacagcg | |
| | gccgcagctctgatacaagccaaacaaaatgctgccaacatcctccgacttaaagagagc | |
| | attgccgcaaccaatgaggctgtgcatgaggtcactgacggattatcgcaactagcagtgg | |
| | cagttgggaagatgcagcagtttgttaatgaccaatttaataaaacagctcaggaattagact | |
| | gcatcaaaattgcacagcaagttggtgtagagctcaacctgtacctaaccgaattgactaca | |
| | gtattcggaccacaaatcacttcacctgctttaaacaagctgactattcaggcactttacaatc | |
| | tagctggtggaaatatggattacttattgactaagttaggtgtagggaacaatcaactcagct | |
| | cattaatcggtagcggcttaatcaccggtaaccctattctatacgactcacagactcaactctt | |
| | gggtatacaggtaactctaccttcagtcgggaacctaaataatatgcgtgccacctacttgga | |
| | aaccttatccgtaagcacaaccaggggatttgcctcggcacttgtcccAaaagtggtgaca | |
| | caggtcggttctgtgatagaagaacttgacacctcatactgtatagaaactgacttagatttat | |
| | attgtacaagaatagtaacgttccctatgtcccctggtatttattcctgcttgagcggcaatacg | |
| | tcggcctgtatgtactcaaagaccgaaggcgcacttactacaccatacatgactatcaaagg | |
| | ttcagtcatcgccaactgcaagatgacaacatgtagatgtgtaaaccccccgggtatcatat | |
| | cgcaaaactatggagaagccgtgtctctaatagataaacaatcatgcaatgttttatccttagg | |
| | cgggataactttaaggctcagtggggaattcgatgtaacttatcagaagaatatctcaataca | |
| | agattctcaagtaataataacaggcaatcttgatatctcaactgagcttgggaatgtcaacaa | |
| | ctcgatcagtaatgctttgaataagttagaggaaagcaacagaaaactagacaaagtcaatg | |
| | tcaaactgactagcacatctgctctcattacctatatcgttttgactatcatatctcttgtttttggt | |
| | atacttagcctgattctagcatgctacctaatgtacaagcaaaaggcgcaacaaaagacctt | |
| | attatggcttgggaataatactctagatcagatgagagccactacaaaaatgtgaacacaga | |
| | tgaggaacgaaggtttccctaatagtaatttgtgtgaaagttctggtagtctgtcagttcagag | |
| | agttaagaaaaaactaccggttgtagatgaccaaaggacgatatacgggtagaacggtaa | |
| | gagaggccgcccctcaattgcgagccaggcttcacaacctccgttctaccgcttcaccgac | |
| | aacagtcctcaatcatggaccgcgccgttagccaagttgcgttagagaatgatgaaagaga | |
| | ggcaaaaaatacatggcgcttgatattccggattgcaatcttattcttaacagtagtgaccttg | |
| | gctatatctgtagcctcccttttatatagcatgggggctagcacacctagcgatcttgtaggca | |
| | taccgactaggatttccagggcagaagaaaagattacatctacacttggttccaatcaagat | |
| | gtagtagataggatatataagcaagtggcccttgagtctccgttggcattgttaaatactgag | |
| | accacaattatgaacgcaataacatctctctcttatcagattaatggagctgcaaacaacagt | |
| | gggtggggggcacctatccatgacccagattatataggggggataggcaaagaactcatt | |
| | gtagatgatgctagtgatgtcacatcattctatccctctgcatttcaagaacatctgaattttatc | |
| | ccggcgcctactacaggatcaggttgcactcgaataccctcatttgacatgagtgctaccca | |
| | ttactgctacacccataatgtaatattgtctggatgcagagatcactcacattcatatcagtattt | |
| | agcacttggtgtgctccggacatctgcaacagggagggtattcttttctactctgcgttccatc | |
| | aacctggacgacacccaaaatcggaagtcttgcagtgtgagtgcaactcccctgggttgtg | |
| | atatgctgtgctcgaaagtcacggagacagaggaagaagattataactcagctgtccctac | |
| | gcggatggtacatgggaggttagggttcgacggccagtaccacgaaaaggacctagatgt | |
| | cacaacattattcggggactgggtggccaactacccaggagtagggggtggatcttttattg | |
| | acagccgcgtatggttctcagtctacggagggttaaaacccaattcacccagtgacactgta | |
| | caggaagggaaatatgtgatatacaagcgatacaatgacacatgcccagatgagcaagac | |
| | taccagattcgaatggccaagtcttcgtataagcctggacggtttggtgggaaacgcataca | |
| | gcaggctatcttatctatcaaggtgtcaacatccttaggcgaagacccggtactgactgtacc | |
| | gcccaacacagtcacactcatgggggccgaaggcagaattctcacagtagggacatctca | |
| | tttcttgtatcaacgagggtcatcatacttctctcccgcgttattatatcctatgacagtcagcaa | |
| | caaaacagccactcttcatagtccttatacattcaatgccttcactcggccaggtagtatccct | |
| | tgccaggcttcagcaagatgccccaactcgtgtgttactggagtctatacagatccatatccc | |
| | ctaatcttctatagaaaccacaccttgcgaggggtattcgggacaatgcttgatggtgtacaa | |
| | gcaagacttaaccctgcgtctgcagtattcgatagcacatcccgcagtcgcattactcgagt | |
| | gagttcaagcagtaccaaagcagcatacacaacatcaacttgttttaaagtggtcaagacta | |
| | ataagacctattgtctcagcattgctgaaatatctaatactctcttcggagaattcagaatcgtc | |
| | ccgttactagttgagatcctcaaagatgacggggttagagaagccaggtctggctagttga | |
| | gtcaattataaaggagttggaaagatggcattgtatcacctatcttctgcgacatcaagaatc | |
| | aaaccgaatgccggcgcgtgctcgaattccatgttgccagttgaccacaatcagccagtgc | |
| | tcatgcgatcagattaagccttgtcaAtaGtctcttgattaagaaaaaatgtaagtggcaatg | |
| | agatacaaggcaaaacagctcatggtTaaCaatacgggtaggacatggcgagctccggt | |
| | cctgaaagggcagagcatcagattatcctaccagagTcacacctgtcttcaccattggtca | |
| | agcacaaactactctattactggaaattaactgggctaccgcttcctgatgaatgtgacttcga | |
| | ccacctcattctcagccgacaatggaaaaaaatacttgaatcggcctctcctgatactgaga | |
| | gaatgataaaactcggaagggcagtacaccaaactcttaaccacaattccagaataaccgg | |
| | agtgctccaccccaggtgtttagaaGaactggctaatattgaggtcccagattcaaccaac | |
| | aaatttcggaagattgagaagaagatccaaattcacaacacgagatatggagaactgttcac | |
| | aaggctgtgtacgcatatagagaagaaactgctggggtcatcttggtctaacaatgtccccc | |
| | ggtcagaggagttcagcagcattcgtacggatccggcattctggtttcactcaaaatggtcc | |
| | acagccaagtttgcatggctccatataaaacagatccagaggcatctgatggtggcagcta | |
| | Ggacaaggtctgcggccaacaaattggtgatgctaacccataaggtaggccaagtctttgt | |
| | cactcctgaacttgtcgttgtgacgcatacgaatgagaacaagttcacatgtcttacccagga | |
| | acttgtattgatgtatgcagatatgatggagggcagagatatggtcaacataatatcaaccac | |
| | ggcggtgcatctcagaagcttatcagagaaaattgatgacattttgcggttaatagacgctct | |
| | ggcaaaagacttgggtaatcaagtctacgatgttgtatcactaatggagggatttgcatacg | |
| | gagctgtccagctactcgagccgtcaggtacatttgcaggagatttcttcgcattcaacctgc | |
| | aggagcttaaagacattctaattggcctcctccccaatgatatagcagaatccgtgactcatg | |
| | caatcgctactgtattctctggtttagaacagaatcaagcagctgagatgttgtgtctgttgcgt | |
| | ctgtggggtcacccactgcttgagtcccgtattgcagcaaaggcagtcaggagccaaatgt | |
| | gcgcaccgaaaatggtagactttgatatgatccttcaggtactgtctttcttcaagggaacaat | |
| | catcaacgggtacagaaagaagaatgcaggtgtgtggccgcgagtcaaagtggatacaat | |
| | atatgggaaggtcattgggcaactacatgcagattcagcagagatttcacacgatatcatgtt | |
| | gagagagtataagagtttatctgcacttgaatttgagccatgtatagaatatgaccctgtcacc | |
| | aacctgagcatgttcctaaaagacaaggcaatcgcacaccccaacgataattggcttgcct | |
| | cgtttaggcggaaccttctctccgaagaccagaagaaacatgtaaaagaagcaacttcgac | |
| | taatcgcctcttgatagagtttttagagtcaaatgattttgatccatataaagagatggaatatct | |
| | gacgacccttgagtaccttagagatgacaatgtggcagtatcatactcgctcaaggagaag | |
| | gaagtgaaagttaatggacggatcttcgctaagctgacaaagaagttaaggaactgtcagg | |
| | tgatggcggaagggatcctagccgatcagattgcacctttctttcagggaaatggagtcatt | |
| | caggatagcatatccttgaccaagagtatgctagcgatgagtcaactgtcttttaacagcaat | |
| | aagaaacgtatcactgactgtaaagaaagagtatcttcaaaccgcaatcatgatccgaaaa | |
| | gcaagaaccgtcggagagttgcaaccttcataacaactgacctgcaaaagtactgtcttaat | |
| | tggagatatcagacaatcaaattgttcgctcatgccatcaatcagttgatgggcctacctcact | |
| | tcttcgaatggattcacctaagactgatggacactacgatgttcgtaggagaccctttcaatc | |
| | ctccaagtgaccctactgactgtgacctctcaagagtccctaatgatgacatatatattgtcag | |
| | tgccagagggggtatcgaaggattatgccagaagctatggacaatgatctcaattgctgca | |
| | atccaacttgctgcagctagatcgcattgtcgtgttgcctgtatggtacagggtgataatcaa | |
| | gtaatagcagtaacgagagaggtaagatcagacgactctccggagatggtgttgacacag | |
| | ttgcatcaagccagtgataatttcttcaaggaattaattcatgtcaatcatttgattggccataat | |
| | ttgaaggatcgtgaaaccatcaggtcagacacattcttcatatacagcaaacgaatcttcaaa | |
| | gatggagcaatcctcagtcaagtcctcaaaaattcatctaaattagtgctagtgtcaggtgat | |
| | ctcagtgaaaacaccgtaatgtcctgtgccaacattgcctctactgtagcacggctatgcga | |
| | gaacgggcttcccaaagacttctgttactatttaaactatataatgagttgtgtgcagacatact | |
| | ttgactctgagttctccatcaccaacaattcgcaccccgatcttaatcagtcgtggattgagga | |
| | catctcttttgtgcactcatatgttctgactcctgcccaattagggggactgagtaaccttcaat | |
| | actcaaggctctacactagaaatatcggtgacccggggactactgcttttgcagagatcaag | |
| | cgactagaagcagtgggattactgagtcctaacattatgactaatatcttaactaggccgcct | |
| | gggaatggagattgggccagtctgtgcaacgacccatactctttcaattttgagactgttgca | |
| | agcccaaatattgttcttaagaaacatacgcaaagagtcctatttgaaacttgttcaaatccctt | |
| | attgtctggagtgcacacagaggataatgaggcagaagagaaggcattggctgaattcttg | |
| | cttaatcaagaggtgattcatccccgcgttgcgcatgccatcatggaggcaagctctgtagg | |
| | taggagaaagcaaattcaagggcttgttgacacaacaaacaccgtaattaagattgcgctta | |
| | ctaggaggccattaggcatcaagaggctgatgcggatagtcaattattctagcatgcatgca | |
| | atgctgtttagagacgatgttttttcctccagtagatccaaccaccccttagtctcttctaatatg | |
| | tgttctctgacactggcagactatgcacggaatagaagctggtcacctttgacgggaggca | |
| | ggaaaatactgggtgtatctaatcctgatacgatagaactcgtagagggtgagattcttagtg | |
| | taagcggagggtgtacaagatgtgacagcggagatgaacaatttacttggttccatcttcca | |
| | agcaatatagaattgaccgatgacaccagcaagaatcctccgatgagggtaccatatctcg | |
| | ggtcaaagacacaggagaggagagctgcctcacttgcaaaaatagctcatatgtcgccac | |
| | atgtaaaggctgccctaagggcatcatccgtgttgatctgggcttatggggataatgaagta | |
| | aattggactgctgctcttacgattgcaaaatctcggtgtaatgtaaacttagagtatcttcggtt | |
| | actgtcccctttacccacggctgggaatcttcaacatagactagatgatggtataactcagat | |
| | gacattcacccctgcatctctctacaggGtgtcaccttacattcacatatccaatgattctcaa | |
| | aggctgttcactgaagaaggagtcaaagaggggaatgtggtttaccaacagatcatgctctt | |
| | gggtttatctctaatcgaatcgatctttccaatgacaacaaccaggacatatgatgagatcac | |
| | actgcacctacatagtaaatttagttgctgtatcagagaagcacctgttgcggttcctttcgag | |
| | ctacttggggggtaccggaactgaggacagtgacctcaaataagtttatgtatgatcctagc | |
| | cctgtatcggagggagactttgcgagacttgacttagctatcttcaagagttatgagcttaatc | |
| | tggagtcatatcccacgatagagctaatgaacattctttcaatatccagcgggaagttgattg | |
| | gccagtctgtggtttcttatgatgaagatacctccataaagaatgacgccataatagtgtatga | |
| | caatacccgaaattggatcagtgaagctcagaattcagatgtggtccgcctatttgaatatgc | |
| | agcacttgaagtgctcctcgactgttcttaccaactctattacctgagagtaagaggcctGg | |
| | acaatattgtcttatatatgggtgatttatacaagaatatgccaggaattctactttccaacattg | |
| | cagctacaatatctcatcccgtcattcattcaaggttacatgcagtgggcctggtcaaccatg | |
| | acggatcacaccaacttgcagatacggattttatcgaaatgtctgcaaaactattagtatcttg | |
| | cacccgacgtgtgatctccggcttatattcaggaaataagtatgatctgctgttcccatctgtct | |
| | tagatgataacctgaatgagaagatgcttcagctgatatcccggttatgctgtctgtacacgg | |
| | tactctttgctacaacaagagaaatcccgaaaataagaggcttaactgcagaagagaaatgt | |
| | tcaatactcactgagtatttactgtcggatgctgtgaaaccattacttagccccgatcaagtga | |
| | gctctatcatgtctcctaacataattacattcccagctaatctgtactacatgtctcggaagagc | |
| | ctcaatttgatcagggaaagggaggacagggatactatcctggcgttgttgttcccccaaga | |
| | gccattattagagttcccttctgtgcaagatattggtgctcgagtgaaagatccattcacccga | |
| | caacctgcggcatttttgcaagagttagatttgagtgctccagcaaggtatgacgcattcaca | |
| | cttagtcagattcatcctgaactcacatctccaaatccggaggaagactacttagtacgatac | |
| | ttgttcagagggatagggactgcatcttcctcttggtataaggcatctcatctcctttctgtacc | |
| | cgaggtaagatgtgcaagacacgggaactccttatacttagctgaagggagcggagccat | |
| | catgagtcttctcgaactgcatgtaccacatgaaactatctattacaatacgctcttttcaaatg | |
| | agatgaaccccccgcaacgacatttcgggccgaccccaactcagtttttgaattcggttgttt | |
| | ataggaatctacaggcggaggtaacatgcaaagatggatttgtccaagagttccgtccatta | |
| | tggagagaaaatacagaggaaagCgacctgacctcagataaagTagtggggtatattac | |
| | atctgcagtgccctacagatctgtatcattgctgcattgtgacattgaaattcctccagggtcc | |
| | aatcaaagcttactagatcaactagctatcaatttatctctgattgccatgcattctgtaaggga | |
| | gggcggggtagtaatcatcaaagtgttgtatgcaatgggatactactttcatctactcatgaa | |
| | cttgtttgctccgtgttccacaaaaggatatattctctctaatggttatgcatgtcgaggagatat | |
| | ggagtgttacctggtatttgtcatgggttacctgggcgggcctacatttgtacatgaggtggt | |
| | gaggatggcGaaaactctggtgcagcggcacggtacgctTttgtctaaatcagatgagat | |
| | cacactgaccaggttattcacctcacagcggcagcgtgtgacagacatcctatccagtcctt | |
| | taccaagattaataaagtacttgaggaagaatattgacactgcgctgattgaagccggggg | |
| | acagcccgtccgtccattctgtgcggagagtctggtgagcacgctagcgaacataactcag | |
| | ataacccagatCatcgctagtcacattgacacagttatccggtctgtgatatatatggaagct | |
| | gagggtgatctcgctgacacagtatttctatttaccccttacaatctctctactgacgggaaaa | |
| | agaggacatcacttaAacagtgcacgagacagatcctagaggttacaatactaggtcttag | |
| | agtcgaaaatctcaataaaataggcgatataatcagcctagtgcttaaaggcatgatctccat | |
| | ggaggaccttatcccactaaggacatacttgaagcatagtacctgccctaaatatttgaagg | |
| | ctgtcctaggtattaccaaactcaaagaaatgtttacagacacttctgtaCtgtacttgactcg | |
| | tgctcaacaaaaattctacatgaaaactataggcaatgcagtcaaaggatattacagtaactg | |
| | tgactcttaacgaaaatcacatattaataggctccttttttggccaattgtattcttgttgatttaat | |
| | catattatgttagaaaaaagttgaaccctgactccttaggactcgaattcgaactcaaataaat | |
| | gtcttaaaaaaaggttgcgcacaattattcttgagtgtagtctcgtcattcaccaaatctttgttt | |
| | ggt | |

### 5.9 EMBODIMENTS

Exemplary embodiments are provided herein below.
1. A nucleic acid sequence comprising a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV APMV HN protein coding sequence; and (2) the nucleotide sequence encoding the NDV F protein has been replaced with a nucleotide sequence encoding a non-NDV APMV F protein, wherein NDV intergenic regions are before and after the non-NDV APMV F protein coding sequence, and wherein the non-NDV APMV HN protein and non-NDV APMV F protein are not NDV HN protein and F proteins, respectively.
2. The nucleic acid sequence of embodiment 1, wherein the non-NDV APMV F protein and non-NDV APMV HN protein are immunologically distinct from the NDF F protein and NDV HN protein, respectively.
3. The nucleic acid sequence of embodiment 1, wherein the non-NDV APMV HN is the HN protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07.
4. The nucleic acid sequence of embodiment 1 or 3, wherein the non-NDV APMV F is the F protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07.
5. The nucleic acid sequence of embodiment 1, wherein the non-NDV APMV HN protein is an HN protein from the subfamily Avulavirinae and the genus orthoavulavirus, metaavulavirus, or paraavulavirus.
6. The nucleic acid sequence of embodiment 1 or 5, wherein the non-NDV APMV F protein is an F protein from the subfamily Avulavirinae and the genus orthoavulavirus, metaavulavirus, or paraavulavirus.
7. The nucleic acid sequence of any one of embodiments 1 to 6, wherein the NDV genome comprises the NP gene, P gene, M gene, and L gene of NDV LaSota strain.
8. A nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV large polymerase (L), and (5) the nucleotide sequence of any one of SEQ ID NOS:1-14, or a nucleotide sequence that is at least 80%, at least 85%, at least 90%, at least 95%, or at least 98% identical to the nucleotide sequence of any one of SEQ ID NOS:1-14.
9. The nucleic acid sequence of embodiment 8, wherein the NDV nucleocapsid protein, NDV phosphoprotein, NDV matrix protein, and NDV large polymerase are of the NDV LaSota strain.
10. A nucleic acid sequence comprising the nucleotide sequence of SEQ ID NO:44, or SEQ ID NO:44 without the GFP coding sequence.
11. A nucleic acid sequence comprising a nucleotide sequence that is at least 80%, at least 85%, at least 90%, at least 95%, or at least 98% identical to the nucleotide sequence of SEQ ID NO:44, or SEQ ID NO:44 without the GFP coding sequence.
12. A nucleic acid sequence comprising the nucleotide sequence of SEQ ID NO:45, or SEQ ID NO:45 without the GFP coding sequence.
13. A nucleic acid sequence comprising a nucleotide sequence that is at least 80%, at least 85%, at least 90%, at least 95%, or at least 98% identical to the nucleotide sequence of SEQ ID NO:45, or SEQ ID NO:45 without the GFP coding sequence.
14. The nucleic acid sequence of any one of embodiments 1 to 13, which further comprises a transgene.
15. The nucleic acid sequence of any one of embodiments 1 to 13, which further comprises a transgene encoding an antigen.
16. The nucleic acid sequence of embodiment 14, wherein the antigen is viral, bacterial, fungal or protozoan antigen.
17. The nucleic acid sequence of embodiment 14, wherein the antigen comprises a SARS-CoV-2 spike protein or a fragment thereof.
18. The nucleic acid sequence of embodiment 17, wherein the fragment comprises the receptor binding domain of the SARS-CoV-2 spike protein.
19. The nucleic acid sequence of embodiment 17, wherein the fragment comprises the ectodomain of the SARS-CoV-2 spike protein.
20. The nucleic acid sequence of embodiment 15, wherein the antigen is a MERS-CoV antigen, respiratory syncytial virus antigen, human metapneumovirus antigen, a Lassa virus antigen, Ebola virus antigen, or Nipah virus antigen.
21. The nucleic acid sequence of embodiment 15, wherein the antigen is a cancer or tumor antigen.
22. A recombinant Newcastle disease virus (NDV) comprising a packaged genome, wherein the packaged genome comprises a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV AMPV HN protein coding sequence; and (2) the nucleotide sequence encoding the NDV F protein has been replaced with a nucleotide sequence encoding a non-NDV APMV F protein, wherein NDV intergenic regions are before and after the non-NDV AMPV F protein coding sequence, and wherein the non-NDV APMV HN protein and non-NDV APMV F protein are not NDV HN protein and F proteins, respectively.
23. The recombinant NDV of embodiment 22, wherein the non-NDV APMV F protein and non-NDV APMV HN protein are immunologically distinct from the NDF F protein and NDV HN protein, respectively.
24. The recombinant NDV of embodiment 22, wherein the non-NDV APMV HN protein is an HN protein from the subfamily Avulavirinae and the genus orthoavulavirus, metaavulavirus, or paraavulavirus.
25. The recombinant NDV of embodiment 22 or 23, wherein the non-NDV APMV F protein is an F protein from the subfamily Avulavirinae and the genus orthoavulavirus, metaavulavirus, or paraavulavirus.
26. The recombinant NDV of embodiment 22, wherein the non-NDV APMV HN is the HN protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07.
27. The recombinant NDV of embodiment 22 or 26, wherein the non-NDV APMV F is the F protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07.
28. A recombinant Newcastle disease virus (NDV) comprising a packaged genome, wherein the packaged genome comprises a nucleotide sequence of a Newcastle disease virus genome in which the nucleotide sequences encoding the NDV HN protein and NDV F protein are replaced with a nucleotide sequence comprising a negative sense RNA sequence transcribed from the cDNA sequence set forth in any one of SEQ ID NOs:1-14.
29. A recombinant Newcastle disease virus (NDV) comprising a packaged genome, wherein the packaged genome comprises a nucleotide sequence of a Newcastle disease virus genome in which the nucleotide sequences encoding the NDV HN protein and NDV F protein are replaced with a nucleotide sequence comprising a negative sense RNA sequence transcribed from a nucleotide sequence that is at least 80%, at least 85%, at least 90%, at least 95%, or at least 98% identical to the nucleotide sequence of any one of SEQ ID NOS:1-14.
30. The recombinant NDV of any one of embodiments 22 to 29, wherein the NDV genome comprises the NP gene, P gene, M gene, and L gene of NDV LaSota.
31. The recombinant NDV of any one of embodiments 22 to 30, wherein the packaged genome further comprises a transgene.
32. The recombinant NDV of embodiment 31, wherein the transgene comprises a nucleotide sequence encoding a viral, bacterial, fungal or protozoan antigen.
33. The recombinant NDV of embodiment 31, wherein the transgene comprises a nucleotide sequence encoding a SARS-CoV-2 antigen.
34. The recombinant NDV of embodiment 33, wherein the SARS-CoV-2 antigen comprises the SARS-CoV-2 spike protein or a fragment thereof.
35. The recombinant NDV of embodiment 34, wherein the fragment comprises the receptor binding domain of the SARS-CoV-2 spike protein.
36. The recombinant NDV of embodiment 31, wherein the transgene comprises a nucleotide sequence encoding a MERS-CoV antigen.
37. The recombinant NDV of embodiment 31, wherein the transgene comprises a nucleotide sequence encoding a respiratory syncytial virus antigen or human metapneumovirus antigen.
38. The recombinant NDV of embodiment 31, wherein the transgene comprises a nucleotide sequence encoding a Lassa virus antigen, Ebola virus antigen or Nipah virus antigen.
39. The recombinant NDV of embodiment 31, wherein the transgene comprises a nucleotide sequence encoding a cancer or tumor antigen.
40. An immunogenic composition comprising a first recombinant NDV, which is the recombinant NDV of any one of embodiments 22 to 31.
41. An immunogenic composition comprising a first recombinant NDV, which is the recombinant NDV of any one of embodiments 32 to 38.
42. An immunogenic composition comprising a first recombinant NDV, which is the recombinant NDV of embodiment 39.
43. A method for inducing an immune response to an antigen, comprising administering the immunogenic composition of embodiment 40, 41, or 42 to a subject.
44. A method for preventing an infectious disease, comprising administering the immunogenic composition of embodiment 40 or 41 to a subject.
45. A method for immunizing a subject against an infectious disease, comprising administering the immunogenic composition of embodiment 40 or 41 the subject.
46. A method for treating cancer, comprising administering the immunogenic composition of embodiment 40 or 42 to a subject.
47. The method of any one of embodiment 43 to 46, wherein the composition is administered to the subject intranasally.
48. The method of any one of embodiments 43 to 47, wherein the method further comprises administering a second recombinant NDV comprising a packaged genome, wherein the packaged genome of the second recombinant NDV comprises a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV AMPV HN protein coding sequence; and (2) the nucleotide sequence encoding the NDV F protein has been replaced with a nucleotide sequence encoding a non-NDV APMV F protein, wherein NDV intergenic regions are before and after the non-NDV AMPV F protein coding sequence, and wherein the second recombinant NDV is immunologically distinct than the first recombinant NDV.
49. The method of any one of embodiments 43 to 48, wherein the subject is a human.
50. A kit comprising the recombinant NDV of any one of embodiments 22 to 39.
51. A kit comprising the nucleic acid sequence of any one of embodiments 1 to 21.
52. An in vitro or ex vivo cell comprising the recombinant NDV of any one of embodiments 22 to 39.
53. A cell line or chicken embryonated egg comprising the recombinant NDV of any one of embodiments 22 to 39.
54. A method for propagating the recombinant NDV of any one of embodiments 22 to 39, the method comprising culturing the cell or embryonated egg of embodiment 52 or 53.
55. The method of embodiment 54, wherein the method further comprises isolating the recombinant NDV from the cell or embryonated egg.

### 6. EXAMPLES

### 6.1 Example 1: Chimeric Newcastle Disease Virus (NDV)-Avian Paramyxoviruses (APMV) Constructs

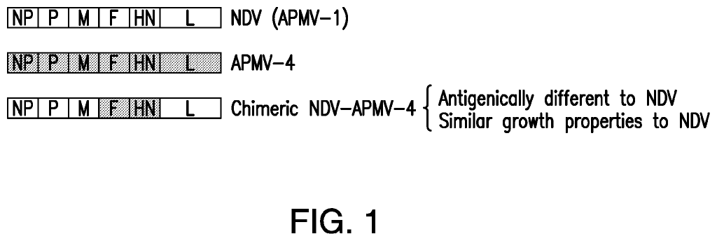

This example describes the production of chimeric NDV-APMV constructs. In this example the coding regions of the viral glycoproteins F and HN of NDV (avian paramyxovirus 1) are replaced with the coding regions of homologous glycoproteins (i.e., F and HN) from another avian paramyxoviruses (APMV) to generate a recombinant chimeric NDV-APMV vector (FIG. 1).

### 6.1.1 Materials & Methods

The chimeric NDV-APMV vectors are produced by reverse genetics using the protocol described in, e.g., Ayllon et al. Rescue of recombinant Newcastle disease virus from cDNA. J Vis Exp. 2013 Oct. 11; (80):50830. doi: 10.3791/50830.

6.1.1.1 Generation of Acceptor Plasmid pNDV-F-HNless

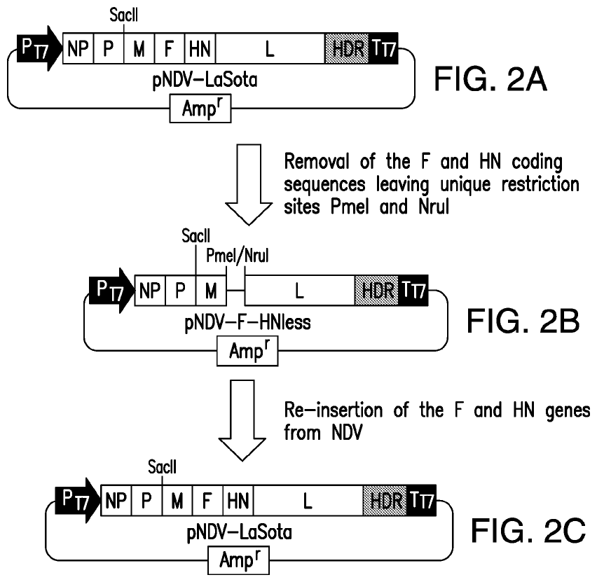

In brief, a 3.7 Kb region containing the F and HN coding sequences in a rescue plasmid, pNDV-LaSota, containing a full-length cDNA of the NDV genome under the control of the T7 RNA polymerase promoter is removed and replaced with a short sequence containing two new unique restriction sites (Pmel and Nrul) to generate an acceptor plasmid, pNDV-F-HNless (FIGS. 2A-2B). Synthetic inserts encoding the F and HN proteins of other APMVs are then inserted between the M and L genes of the acceptor plasmid pNDV-F-HNless. To show that inserting F and HN sequences into the acceptor plasmid (pNDV-F-HNless) results in a functional plasmid, a sequence coding for the F and HN protein of NDV was inserted in the cDNA of the acceptor plasmid pNDV-F-HNless between the M and L genes to generate a functional rescue plasmid pNDV-LaSota as shown in FIG. 2C. This functional rescue plasmid pNDV-LaSota successfully rescued a viable virus (data not shown).

### 6.1.1.2 Design of the APMV F and HN Sequence Inserts

Phylogenetic trees using the F and HN sequences from all the APMV full genomes available in GenBank were used to select the F and HN sequences to be cloned into the pNDV-F-HNless acceptor plasmids (FIGS. 3A and 3B). Fourteen (14) candidates were selected from the phylogenetic trees to represent the genetic diversity of the whole tree. For example, the F and HN sequences from the AMPV full genomes having GenBank accession numbers FJ177514, MK167211, EU910942, FJ231524, MK677433, EU622637, JQ886184, NC_034968, FJ215863, EU338414, EU782025, KC333050, LC168750, and NC_025349 were selected.

In order to synthesize the AMPV F-HN sequences, NDV intergenic regions were added before, in between, and after the F and HN open reading frames. The APMV F sequences (indicative of virulence) are checked for multi-basic cleavage sites and replaced, if necessary, by the closest non-virulent cleavage site available. Any SacII restriction sites in the APMV-F-HN sequences are removed by a silent point mutation since a unique SacII restriction site is used for the cloning of additional genes. Further, since the complete nucleotide size of any paramyxovirus genome must be a multiple of six, the APMV-F-HN sequences are checked for compliance with the rule of six and a second stop codon was added, if necessary, after the F open reading frame in order to comply with this requirement.

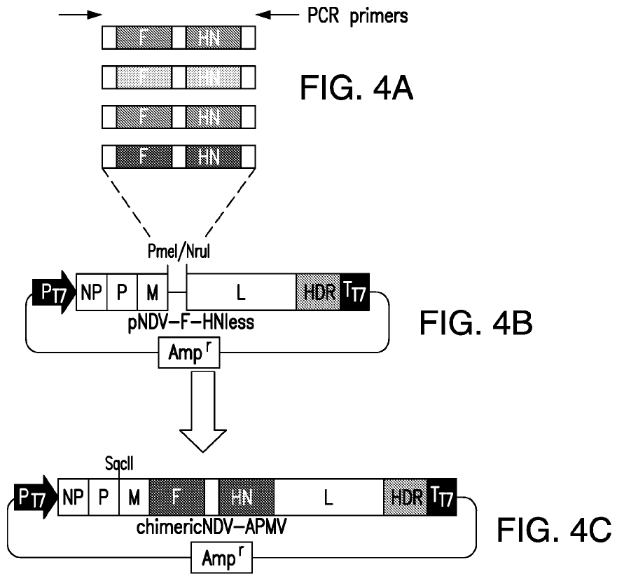

The AMPV F-HN sequences are synthesized by Genewiz (www.genewiz.com) and can be any one of, for example, SEQ ID NOs: 1-14. See Table 1. Since all the APMV F and HN sequence inserts have common NDV-derived sequences at both ends, such inserts can be amplified and cloned with the same primers. In brief, AMPV F-HN sequences are amplified by PCR using PCR primers designed for reconstitution of the NDV sequences flanking the F and HN open reading frames (FIGS. 4A-4C). Each AMPV F-HN sequence (or the PCR product) is then cloned into a pNDV-F-HNless acceptor plasmid between the M and L genes to generate a chimeric NDV-APMV plasmid. Each of the sequences in Table 1 has been cloned into a pNDV-F-HNless acceptor plasmid between the M and L genes to generate a chimeric NDV-APMV plasmid.

### 6.1.1.3 Assessment of Viability

The viability of rescued chimeric NDV-APMV is assessed by, e.g., a plaque assay. The chimeric NDV-APMV are tested to confirm that they are not neutralized by pre-existing NDV-specific humoral immunity using, e.g., a microneutralization assay.

### 6.1.2 Results

This example describes the production of chimeric vectors. Since F and HN are the main targets for the neutralizing antibody response, and different APMVs are antigenically different, the chimeric vectors are antigenically different and therefore are not neutralized by pre-existing NDV-specific humoral immunity. On the other hand, since growth properties are determined by the combined functions of all the viral proteins, and since all avian paramyxoviruses share a common replication strategy, the chimeric vectors are fully viable and replicate similarly to the parental NDV vector.

### 6.2 Example 2: APMV-4

This example provides data demonstrating that APMV-4 was found to be a more potent immune stimulator than NDV.

### 6.2.1 Materials & Methods

### Cell Lines, Antibodies and Other Reagents

Murine cancer cell lines B16-F10 (mouse skin melanoma cells; ATCC Cat #CRL-6475) and CT26.WT (mouse colon carcinoma cells; ATCC Cat #CRL-2638) were maintained in RPMI medium supplemented with 10% FBS (fetal bovine serum) and 2% penicillin and streptomycin. Human melanoma SK-MEL-2 (ATCC Cat #HTB-68TM) and colon carcinoma RKO-E6 cells (ATCC Cat #CRL-2578TM) were propagated using ATCC-formulated Eagle's Minimum Essential Medium. Master cancer cells-banks were created after purchase and early-passage cells were thawed in every experimental step. Once in culture, cells were maintained no longer than 8 weeks to guarantee genotypic stability and were monitored routinely by microscopy. Required IMPACT test for cancer cells involved in our in vivo experiments was performed by the Center for Comparative Medicine and Surgery at Icahn School of Medicine at Mt. Sinai (Mount Sinai Hospital, New York, NY). Reduced serum media Opti-MEM™ (Gibco™) was used for in vitro viral infection medium.

### Viruses

Modified Newcastle disease virus LaSota-L289A has been previously described (Vijayakumar G, Palese P, Goff PH. Oncolytic Newcastle disease virus expressing a checkpoint inhibitor as a radio enhancing agent for murine melanoma. EBioMedicine 2019; 49:96-105). APMV-4 Duck/Hong Kong/D3/1975 (175ADV0601) isolate was obtained from National Veterinary Services Laboratories, United States Department of Agriculture (USDA, Ames, IA). Viral stocks were propagated in 9 day-old embryonated chicken eggs and clear-purified from the allantoic fluid by discontinuous sucrose density gradient ultracentrifugation for resuspension and storage in PBS. Viral titers were calculated by indirect immuno-fluorescence on Vero cells.

### Transcription Analysis by RT-qPCR

Cancer cells were mock-treated or infected with specified virus at a MOI of 1 PFU/cell in 250 μl of OptiMEM-I. After allowing virus adsorption for 1 hour, the cells were incubated with an additional 750 μl of supplemented media. Total RNA was isolated using a Qiagen RNeasy Minikit (Cat #74106, Qiagen) at the indicated time post-infection. cDNA synthesis was performed using the Maxima First Strand cDNA Synthesis Kit for RT-qPCR (Cat #K1671, Thermo Scientific). Mean n-fold expression levels of cDNA from three individual biological samples were normalized to 18S rRNA levels and calibrated to mock-treated samples according to the 2-ΔΔCT method (Livak K J, Schmittgen T D. Analysis of relative gene expression data using real-time quantitative PCR and the 2(−Delta C(T)) Method. Methods 2001; 25:402-8). Heat maps were created using Morpheus, https://software.broadinstitute.org/morpheus. Human and murine primer sequences have been compiled in Table 2.

TABLE 2
RT-qPCR Primers' Sequences

| | FORWARD PRIMER | SEQ | REVERSE PRIMER | SEQ | |
|---|---|---|---|---|---|
| GEN | SEQUENCE | ID NO. | SEQUENCE | ID NO. | SPECIES |
| IFN-B | CAGCTCCAAGAAAGGACG | 16 | GGCAGTGTAACTCTTCTG | 17 | MOUSE |
| | AAC | | CAT | | |
| IFN-B | TCTGGCACAACAGGTAGT | 18 | GAGAAGCACAACAGGAGA | 19 | HUMAN |
| | AGGC | | GCAA | | |
| IL-6 | CTGCAAGAGACTTCCATC | 20 | AGTGGTATAGACAGGTCT | 21 | MOUSE |
| | CAG | | GTTGG | | |
| IL-6 | AGAGGCACTGGCAGAAAA | 22 | AGGCAAGTCTCCTCATTG | 23 | HUMAN |
| | CAAC | | AATCC | | |
| IL-1B | CTCGCCAGTGAAATGATG | 24 | GTCGGAGATTCGTAGCTG | 25 | HUMAN |
| | GCT | | GAT | | |
| IL-1B | TGGGCTGGACTGTTTCTA | 26 | TGTCTTGGCCGAGGACTA | 27 | MOUSE |
| | ATGC | | AGG | | |
| ISG-15 | GGTGTCCGTGACTAACTC | 28 | TGGAAAGGGTAAGACCGT | 29 | MOUSE |
| | CAT | | CCT | | |
| ISG-15 | TCCTGGTGAGGAATAACA | 30 | GTCAGCCAGAACAGGTCG | 31 | HUMAN |
| | AGGG | | TC | | |
| MX1 | GACCATAGGGGTCTTGAC | 32 | AGACTTGCTCTTTCTGAA | 33 | MOUSE |
| | CAA | | AAGCC | | |
| MX1 | GTTTCCGAAGTGGACATC | 34 | GAAGGGCAACTCCTGACA | 35 | HUMAN |
| | GCA | | GT | | |
| STAT-1 | TCACAGTGGTTCGAGCTT | 36 | GCAAACGAGACATCATAG | 37 | MOUSE |
| | CAG | | GCA | | |
| STAT-1 | ATGTCTCAGTGGTACGAA | 38 | TGTGCCAGGTACTGTCTG | 39 | HUMAN |
| | CTTCA | | ATT | | |
| N | GAACCATGTTGCCCTTGC | 40 | CCTCTCCAGGGTATCGGT | 41 | LS-L289A |
| | AG | | GA | | |
| N | ATCGGTCCTTAGCAGGAG | 42 | GGGTCCAGTCGTTGACAC | 43 | APMV-4 |
| | GA | | TT | | |

### Statistical Analysis

Data analysis was performed using GraphPad Prism 9. One-way ANOVA or two-way ANOVA were used to compare multiple groups with one or two independent variables, respectively. Results are expressed as mean value ±SEM or ±SD as indicated. Comparisons of survival curves were performed using the Log-rank (Mantel-Cox) test. p values >0.05 were considered statistically non-significant (ns); * p<0.05, ** p<0.01, *** p <0.001, **** p<0.0001.

### 6.2.2 Results

An infectious clone of APMV-4 (recombinant APMV-4) was generated by designing a plasmid-based rescue strategy modeled after the already established system for NDV and other paramyxoviruses (Ayllon J, Garcia-Sastre A, Martinez-Sobrido L. Rescue of recombinant Newcastle disease virus from cDNA. J Vis Exp 2013).

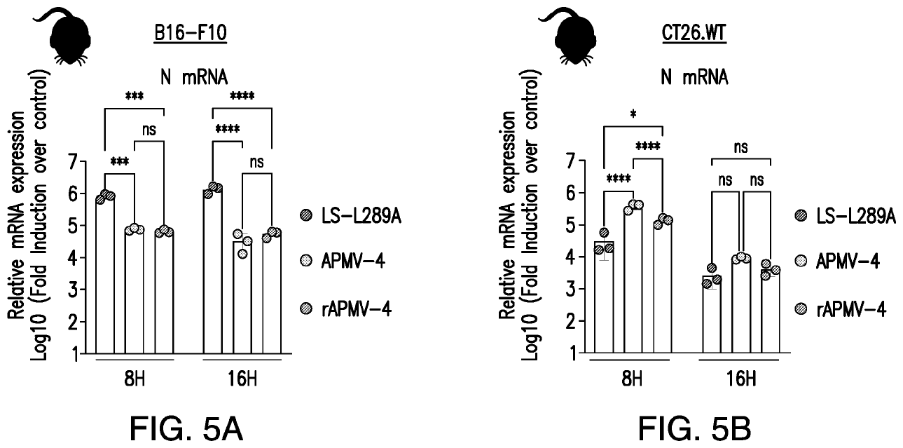

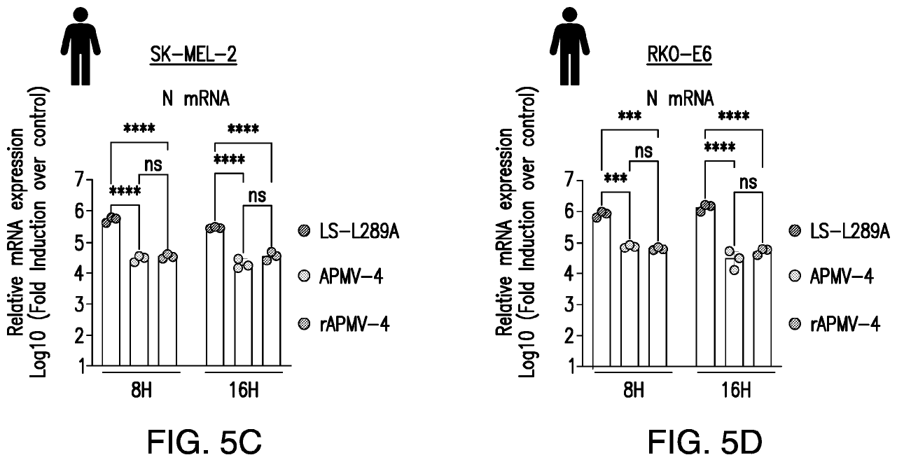

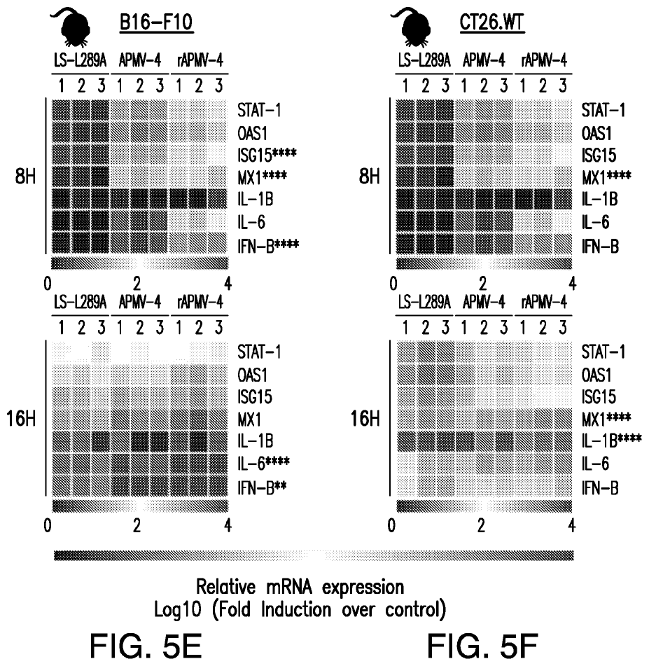

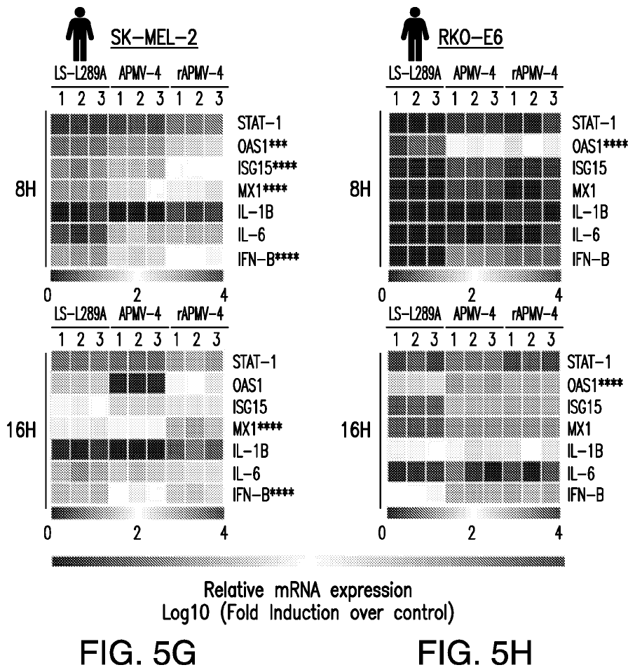

The proinflammatory response elicited by APMV-4 or recombinant APMV-4 (rAPMV-4) infected cancers was evaluated at 8- and 16-hours post-infection (FIG. 5E-5H). mRNA expression analysis by qPCR showed increased upregulation of INF-β, STAT-1, ISG15, OAS1 and MX1 genes by APMV-4 infected cells, when compared to the expression levels induced by LS-L289A at 8 hours post-infection. This earlier and stronger Type-I interferon signature was displayed by all cancer cell lines independently of their origin, and this signature was replicated by rAPMV-4 infection. At either 8 hours or 16 hours, significant differences between APMV-4 viruses and NDV were found in the expression of ISG-15 and MX-1. IL-6 was particularly upregulated in murine cancer cells, while OAS1 was significantly upregulated by human cancer cells. Analysis of mRNA expression levels of the viral nucleoprotein N (FIG. 5A-5D) did not show a direct association between the viral replication activity and the early immune signatures, with B16-F10 (FIG. 5A, 5E) and SK-MEL-2 melanoma cancer cells (FIG. 5C, 5G) showing higher levels of N mRNA of the LS-L289A virus, but a stronger immune stimulation in response to APMV-4 and rAPMV-4.

### 6.2.3 Discussion

APMV-4 Duck/Hong Kong/D3/1975 was the first identified APMV-4 virus and is considered the prototype strain of the species Avian paraavulavirus (Gogoi P, Ganar K, Kumar S. Avian Paramyxovirus: A Brief Review. Transbound Emerg Dis 2017; 64:53-67; Shortridge K F, Alexander DJ. Incidence and preliminary characterisation of a hitherto unreported, serologically distinct, avian paramyxovirus isolated in Hong Kong. Res Vet Sci 1978; 25:128-30). This isolate has typically been recovered from wild waterfowl worldwide, and occasionally from domestic ducks, geese and chickens, although no clinical signs of disease were ever reported in these infected animals (Alexander DJ. Newcastle disease and other avian paramyxoviruses. Rev Sci Tech 2000; 19:443-62; Warke A, Stallknecht D, Williams S M, Pritchard N, Mundt E. Comparative study on the pathogenicity and immunogenicity of wild bird isolates of avian paramyxovirus 2, 4, and 6 in chickens. Avian Pathol 2008; 37:429-34). This avirulent phenotype has been confirmed by experimental inoculations of birds and mammals (Samuel A S, Subbiah M, Shive H, Collins P L, Samal S K. Experimental infection of hamsters with avian paramyxovirus serotypes 1 to 9. Vet Res 2011; 42:38). Intranasal administration of a high dose of APMV-4 (107 PFU) did not compromise the health of inoculated mice (Data not shown). A complete genome sequence and molecular characterization of the Duck/Hong Kong/D3/1975 strain has been previously reported (Nayak B, Kumar S, Collins P L, Samal S K. Molecular characterization and complete genome sequence of avian paramyxovirus type 4 prototype strain duck/Hong Kong/D3/75. Virol J 2008; 5:124). APMV-4's RBP HN protein has hemagglutinin and neuroaminidase activities and is predicted to recognize sialic acids. Its F protein has a monobasic cleavage site (DIPQR↓F) that, although resembling those in avirulent lentogenic NDV strains, has been suggested to capacitate APMV-4 for multicycle replication in certain cell lines in vitro, despite not displaying a canonical furin cleavage site. In replication studies in cancer cells conducted by the inventors, only multicycle replication with the addition of exogenous TPCK-Trypsin to the infectious media was able to be followed. However, APMV-4 was observed to be able to reach higher titers than the LS-L289A virus while exhibiting similar growth kinetics (Data not shown). Considering all of the above, the distinct dependency of APMV4's F protein on proteolytic activation by either endogenous or secretory proteases could support these differences in viral fitness.

Additionally, APMV-4 has demonstrated its ability to trigger proinflammatory and death responses in infected cancer cells (see, FIG. 5A-5H). When compared with NDV, APMV-4 was found to be a more potent immune stimulator, leading to the host (Id.). When compared with NDV, APMV-4 was found to be a more potent immune stimulator, leading to an earlier and more robust upregulation of Type-I interferon responses. Interestingly, this effect was preserved among the different cancer cells tested (FIG. 5E-5H) and is independent of the levels of viral replication (FIG. 5A-5D).

### 6.3 Example 3: Chimeric Newcastle Disease Virus (NDV)-Avian Paramyxoviruses (APMV) Constructs NDV-APMV2 and NDV-APMV3

Avian paramyxoviruses (APMV) belong to the subfamily of Avulavirinae. APMVs comprise a high diversity of members that are antigenically different. APMVs are further categorized into the genera of Metaavulavirus, Orthoavulavirus and Paraavulavirus. Newcastle disease virus (NDV) belongs to the genus of Orthoavulavirus and is also known as AMPV serotype-1 (APMV-1) (FIG. 6A). To exploit the potential of NDV as vaccine vectors for different viral pathogens and overcome pre-existing immunity introduced by NDV-based vaccines, this example describes the generation of chimeric NDV-APMV2 and NDV-APMV3 viruses and provides data demonstrating that these viruses are antigenically distinct from the wild type NDV. In this example the coding regions of the viral glycoproteins F and HN of NDV (avian paramyxovirus 1) were replaced with the coding regions of homologous glycoproteins (i.e., F and HN) from another avian paramyxoviruses (APMV) to generate recombinant chimeric NDV-APMV viruses (FIG. 6B).

### 6.3.1 Materials & Methods

### Viruses

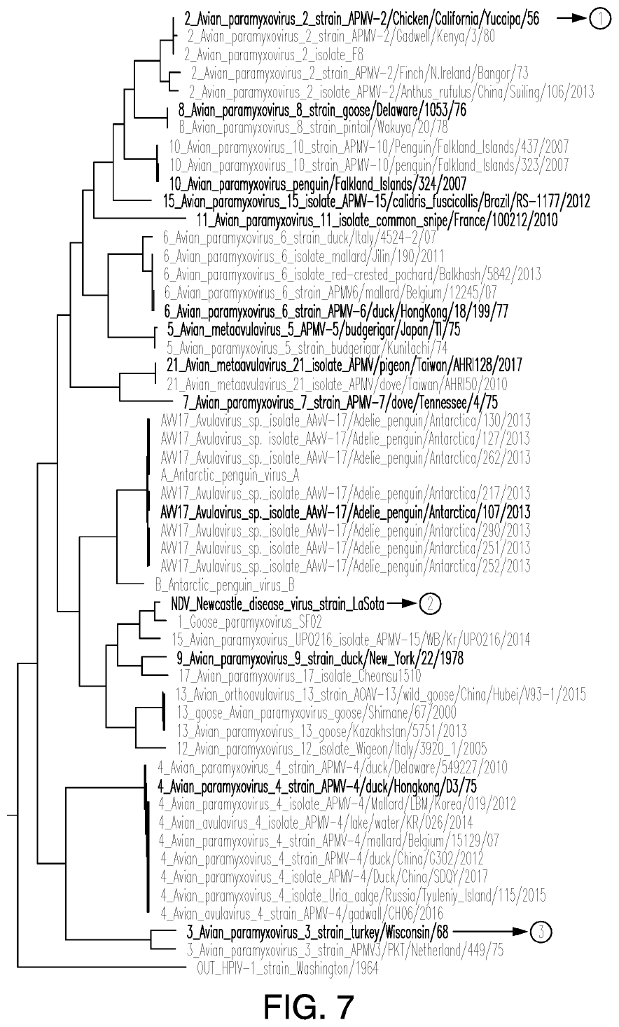

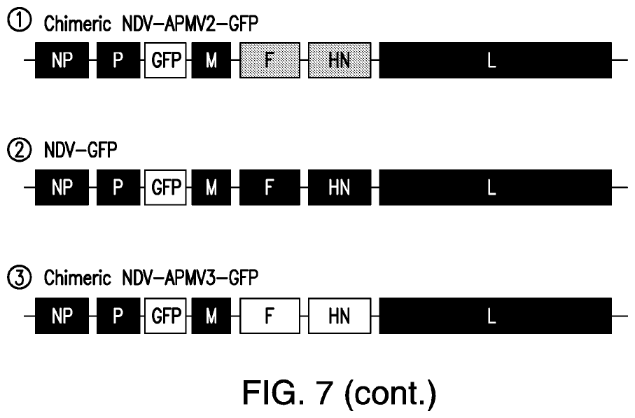

The chimeric NDV-APMV vectors were produced as describe in Example 1. Briefly, the F and HN sequences from APMV2/Chicken/California/Yucaipa/56 were cloned into the pNDV-F-HNless acceptor plasmids to make the chimeric NDV-APMV2 vector. The F and HN sequences from APMV3/Turkey/Wisconsin/68 were cloned into the pNDV-F-HNless acceptor plasmids to make the chimeric NDV-APMV3. As shown in the phylogenetic tree (FIG. 6A), APMV2 belonged to the genus of Metaavulavirus and AMPV3 belonged to the genus of Paraavulavirus. APMV2 and APMV3 were not only antigenically different from NDV (Orthoavulavirus), but also antigenically different from each other (FIG. 7).

As a biomarker and for imaging purpose, a gene of green fluorescent protein (GFP) was inserted between the P and M genes of chimeric NDV-APMV2 to produce the chimeric NDV-APMV2-GFP construct. See SEQ ID NO: 44 for the nucleotide sequence of the chimeric NDV-APMV2-GFP. Similarly, the GFP gene was inserted between the P and M genes of chimeric NDV-APMV3 to produce the chimeric NDV-APMV3-GFP construct (FIG. 7). See SEQ ID NO: 45 for the nucleotide sequence of the chimeric NDV-APMV3. Rescue of recombinant viruses was performed using standard techniques (see, e.g., J. Ayllon, A. Garcia-Sastre, L. Martinez-Sobrido, Rescue of recombinant Newcastle disease virus from cDNA. J Vis Exp, (2013)).

### Transgene Expression and Antigenicity

To demonstrate the expression of the transgene, chicken embryo fibroblasts (CEF) cells were infected with viruses of chimeric NDV-APMV2-GFP and chimeric NDV-APMV3-GFP, respectively. The expression of the transgene was verified by examining GFP expression at 18 hours post-infection using fluorescent microscopy. To investigate whether the chimeric NDV-APMV2-GFP and chimeric NDV-APMV3-GFP viruses were antigenically different from NDV, a hemagglutination inhibition (HI) assay was used because the HN protein of APMVs could agglutinate red blood cells. The HI assays were performed in rabbit sera that were raised against the wild type (WT) NDV viruses.

### 6.3.2 Results

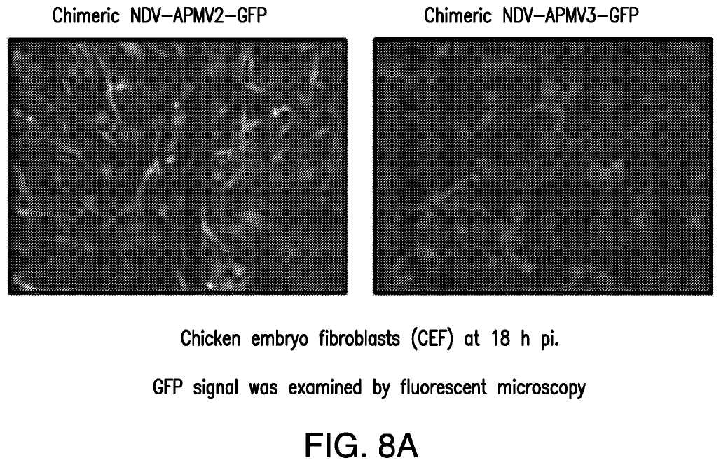

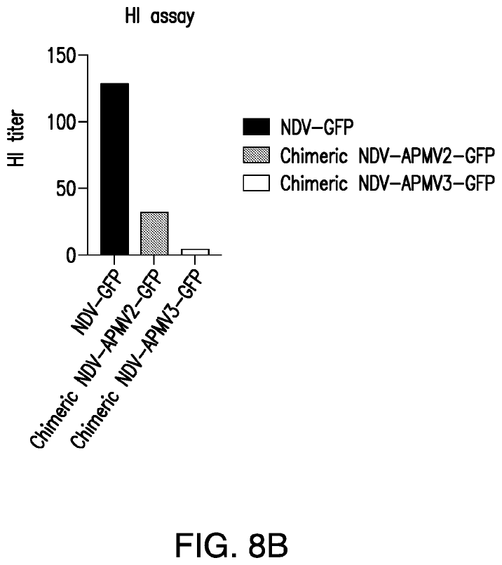

The expression of the transgene was demonstrated by GFP expression observed under fluorescent microscopy. As shown in FIG. 8A, the signal of GFP expression was observed in both chimeric NDV-APMV2-GFP and chimeric NDV-APMV3-GFP virus infected CEF cells at 18 hours post-infection. The results indicated that both chimeric NDV-APMV2-GFP and chimeric NDV-APMV3-GFP virus could express transgene.

The antigenic difference between the chimeric NDV-APMV viruses and WT NDV was assessed by HI assays. The results in FIG. 8B show that the HI activity of the rabbit serum was significantly reduced against both chimeric NDV-APMV-2-GFP and chimeric NDV-APMV-3-GFP constructs as compared to that against the NDV-GFP construct. The results indicate that both chimeric NDV-APMV viruses are antigenically distinct from wild-type NDV.

TABLE 3
Nucleotide Sequences of NDV-APMV2-GFP:
APMV2/Chicken/California/ Yucaipa/56 and NDV-APMV3-GFP:
APMV3/Turkey/Wisconsin/68

| Description | Sequence | SEQ ID NO. |
|---|---|---|
| NDV- | TCTTCCGCTTCCTCGCTCACTGACTCGCTGCGCTCGGTCGTTCGGCT | 44 |
| APMV2- | GCGGCGAGCGGTATCAGCTCACTCAAAGGCGGTAATACGGTTATCCA | |
| GFP: | CAGAATCAGGGGATAACGCAGGAAAGAACATGTGAGCAAAAGGCCAG | |
| APMV2/Chicken/ | CAAAAGGCCAGGAACCGTAAAAAGGCCGCGTTGCTGGCGTTTTTCCA | |
| California/ | TAGGCTCCGCCCCCCTGACGAGCATCACAAAAATCGACGCTCAAGTC | |
| Yucaipa/56 | AGAGGTGGCGAAACCCGACAGGACTATAAAGATACCAGGCGTTTCCC | |
| (GFP- | CCTGGAAGCTCCCTCGTGCGCTCTCCTGTTCCGACCCTGCCGCTTAC | |
| encoding | CGGATACCTGTCCGCCTTTCTCCCTTCGGGAAGCGTGGCGCTTTCTC | |
| sequence is | ATAGCTCACGCTGTAGGTATCTCAGTTCGGTGTAGGTCGTTCGCTCC | |
| in bold and | AAGCTGGGCTGTGTGCACGAACCCCCCGTTCAGCCCGACCGCTGCGC | |
| italics, | CTTATCCGGTAACTATCGTCTTGAGTCCAACCCGGTAAGACACGACT | |
| APMV2 F | TATCGCCACTGGCAGCAGCCACTGGTAACAGGATTAGCAGAGCGAGG | |
| protein- | TATGTAGGCGGTGCTACAGAGTTCTTGAAGTGGTGGCCTAACTACGG | |
| encoding | CTACACTAGAAGAACAGTATTTGGTATCTGCGCTCTGCTGAAGCCAG | |
| sequence is | TTACCTTCGGAAAAAGAGTTGGTAGCTCTTGATCCGGCAAACAAACC | |
| underline, | ACCGCTGGTAGCGGTGGTTTTTTTGTTTGCAAGCAGCAGATTACGCG | |
| and | CAGAAAAAAAGGATCTCAAGAAGATCCTTTGATCTTTTCTACGGGGT | |
| APMV2 | CTGACGCTCAGTGGAACGAAAACTCACGTTAAGGGATTTTGGTCATG | |
| HN- | AGATTATCAAAAAGGATCTTCACCTAGATCCTTTTAAATTAAAAATG | |
| encoding | AAGTTTTAAATCAATCTAAAGTATATATGAGTAAACTTGGTCTGACA | |
| sequence is | GTTACCAATGCTTAATCAGTGAGGCACCTATCTCAGCGATCTGTCTA | |
| double | TTTCGTTCATCCATAGTTGCCTGACTCCCCGTCGTGTAGATAACTAC | |
| underlined) | GATACGGGAGGGCTTACCATCTGGCCCCAGTGCTGCAATGATACCGC | |
| | GAGACCCACGCTCACCGGCTCCAGATTTATCAGCAATAAACCAGCCA | |
| | GCCGGAAGGGCCGAGCGCAGAAGTGGTCCTGCAACTTTATCCGCCTC | |
| | CATCCAGTCTATTAATTGTTGCCGGGAAGCTAGAGTAAGTAGTTCGC | |
| | CAGTTAATAGTTTGCGCAACGTTGTTGCCATTGCTACAGGCATCGTG | |
| | GTGTCACGCTCGTCGTTTGGTATGGCTTCATTCAGCTCCGGTTCCCA | |
| | ACGATCAAGGCGAGTTACATGATCCCCCATGTTGTGCAAAAAAGCGG | |
| | TTAGCTCCTTCGGTCCTCCGATCGTTGTCAGAAGTAAGTTGGCCGCA | |
| | GTGTTATCACTCATGGTTATGGCAGCACTGCATAATTCTCTTACTGT | |
| | CATGCCATCCGTAAGATGCTTTTCTGTGACTGGTGAGTACTCAACCA | |
| | AGTCATTCTGAGAATAGTGTATGCGGCGACCGAGTTGCTCTTGCCCG | |
| | GCGTCAATACGGGATAATACCGCGCCACATAGCAGAACTTTAAAAGT | |
| | GCTCATCATTGGAAAACGTTCTTCGGGGCGAAAACTCTCAAGGATCT | |
| | TACCGCTGTTGAGATCCAGTTCGATGTAACCCACTCGTGCACCCAAC | |
| | TGATCTTCAGCATCTTTTACTTTCACCAGCGTTTCTGGGTGAGCAAA | |
| | AACAGGAAGGCAAAATGCCGCAAAAAAGGGAATAAGGGCGACACGGA | |
| | AATGTTGAATACTCATACTCTTCCTTTTTCAATATTATTGAAGCATT | |
| | TATCAGGGTTATTGTCTCATGAGCGGATACATATTTGAATGTATTTA | |
| | GAAAAATAAACAAATAGGGGTTCCGCGCACATTTCCCCGAAAAGTGC | |
| | CACCTGACGTCTAAGAAACCATTATTATCATGACATTAACCTATAAA | |
| | AATAGGCGTATCACGAGGCCCTTTCGTCTCGCGCGTTTCGGTGATGA | |
| | CGGTGAAAACCTCTGACACATGCAGCTCCCGGAGACGGTCACAGCTT | |
| | GTCTGTAAGCGGATGCCGGGAGCAGACAAGCCCGTCAGGGCGCGTCA | |
| | GCGGGTGTTGGCGGGTGTCGGGGCTGGCTTAACTATGCGGCATCAGA | |
| | GCAGATTGTACTGAGAGTGCACCATAAAATTGTAAACGTTAATATTT | |
| | TGTTAAAATTCGCGTTAAATTTTTGTTAAATCAGCTCATTTTTTAAC | |
| | CAATAGGCCGAAATCGGCAAAATCCCTTATAAATCAAAAGAATAGCC | |
| | CGAGATAGGGTTGAGTGTTGTTCCAGTTTGGAACAAGAGTCCACTAT | |
| | TAAAGAACGTGGACTCCAACGTCAAAGGGCGAAAAACCGTCTATCAG | |
| | GGCGATGGCCCACTACGTGAACCATCACCCAAATCAAGTTTTTTGGG | |
| | GTCGAGGTGCCGTAAAGCACTAAATCGGAACCCTAAAGGGAGCCCCC | |
| | GATTTAGAGCTTGACGGGGAAAGCCGGCGAACGTGGCGAGAAAGGAA | |
| | GGGAAGAAAGCGAAAGGAGCGGGCGCTAGGGCGCTGGCAAGTGTAGC | |
| | GGTCACGCTGCGCGTAACCACCACACCCGCCGCGCTTAATGCGCCGC | |
| | TACAGGGCGCGTACTATGGTTGCTTTGACGTATGCGGTGTGAAATAC | |
| | CGCACAGATGCGTAAGGAGAAAATACCGCATCAGGCGCCATTCGCCA | |
| | TTCAGGCTGCGCAACTGTTGGGAAGGGCGATCGGTGCGGGCCTCTTC | |
| | GCTATTACGCCAGCTGGCGAAAGGGGGATGTGCTGCAAGGCGATTAA | |
| | GTTGGGTAACGCCAGGGTTTTCCCAGTCACGACGTTGTAAAACGACG | |
| | GCCAGTGCCAAGCTTTAATACGACTCACTATAGGGAGATTGGTCTGA | |
| | TGAGTCCGTGAGGACGAAACGGAGTCTAGACTCCGTCACCAAACAGA | |
| | GAATCCGTGAGTTACGATAAAAGGCGAAGGAGCAATTGAAGTCGCAC | |
| | GGGTAGAAGGTGTGAATCTCGAGTGCGAGCCCGAAGCACAAACTCGA | |
| | GAAAGCCTTCTGCCAACATGTCTTCCGTATTTGATGAGTACGAACAG | |
| | CTCCTCGCGGCTCAGACTCGCCCCAATGGAGCTCATGGAGGGGGAGA | |
| | AAAAGGGAGTACCTTAAAAGTAGACGTCCCGGTATTCACTCTTAACA | |
| | GTGATGACCCAGAAGATAGATGGAGCTTTGTGGTATTCTGCCTCCGG | |
| | ATTGCTGTTAGCGAAGATGCCAACAAACCACTCAGGCAAGGTGCTCT | |
| | CATATCTCTTTTATGCTCCCACTCACAGGTAATGAGGAACCATGTTG | |
| | CCCTTGCAGGGAAACAGAATGAAGCCACATTGGCCGTGCTTGAGATT | |
| | GATGGCTTTGCCAACGGCACGCCCCAGTTCAACAATAGGAGTGGAGT | |
| | GTCTGAAGAGAGAGCACAGAGATTTGCGATGATAGCAGGATCTCTCC | |
| | CTCGGGCATGCAGCAACGGAACCCCGTTCGTCACAGCCGGGGCCGAA | |
| | GATGATGCACCAGAAGACATCACCGATACCCTGGAGAGGATCCTCTC | |
| | TATCCAGGCTCAAGTATGGGTCACAGTAGCAAAAGCCATGACTGCGT | |
| | ATGAGACTGCAGATGAGTCGGAAACAAGGCGAATCAATAAGTATATG | |
| | CAGCAAGGCAGGGTCCAAAAGAAATACATCCTCTACCCCGTATGCAG | |
| | GAGCACAATCCAACTCACGATCAGACAGTCTCTTGCAGTCCGCATCT | |
| | TTTTGGTTAGCGAGCTCAAGAGAGGCCGCAACACGGCAGGTGGTACC | |
| | TCTACTTATTATAACCTGGTAGGGGACGTAGACTCATACATCAGGAA | |
| | TACCGGGCTTACTGCATTCTTCTTGACACTCAAGTACGGAATCAACA | |
| | CCAAGACATCAGCCCTTGCACTTAGTAGCCTCTCAGGCGACATCCAG | |
| | AAGATGAAGCAGCTCATGCGTTTGTATCGGATGAAAGGAGATAATGC | |
| | GCCGTACATGACATTACTTGGTGATAGTGACCAGATGAGCTTTGCGC | |
| | CTGCCGAGTATGCACAACTTTACTCCTTTGCCATGGGTATGGCATCA | |
| | GTCCTAGATAAAGGTACTGGGAAATACCAATTTGCCAGGGACTTTAT | |
| | GAGCACATCATTCTGGAGACTTGGAGTAGAGTACGCTCAGGCTCAGG | |
| | GAAGTAGCATTAACGAGGATATGGCTGCCGAGCTAAAGCTAACCCCA | |
| | GCAGCAAGGAGGGGCCTGGCAGCTGCTGCCCAACGGGTCTCCGAGGA | |
| | GACCAGCAGCATAGACATGCCTACTCAACAAGTCGGAGTCCTCACTG | |
| | GGCTTAGCGAGGGGGGGTCCCAAGCTCTACAAGGCGGATCGAATAGA | |
| | TCGCAAGGGCAACCAGAAGCCGGGGATGGGGAGACCCAATTCCTGGA | |
| | TCTGATGAGAGCGGTAGCAAATAGCATGAGGGAGGCGCCAAACTCTG | |
| | CACAGGGCACTCCCCAATCGGGGCCTCCCCCAACTCCTGGGCCATCC | |
| | CAAGATAACGACACCGACTGGGGGTATTGATGGACAAAACCCAGCCT | |
| | GCTTCCACAAAAACATCCCAATGCCCTCACCCGTAGTCGACCCCTCG | |
| | ATTTGCGGCTCTATATGACCACACCCTCAAACAAACATCCCCCTCTT | |
| | TCCTCCCTCCCCCTGCTGTACAACTACGTACGCCCTAGATACCACAG | |
| | GCACAATGCGGCTCACTAACAATCAAAACAGAGCCGAGGGAATTAGA | |
| | AAAAAGTACGGGTAGAAGAGGGATATTCAGAGATCAGGGCAAGTCTC | |
| | CCGAGTCTCTGCTCTCTCCTCTACCTGATAGACCAGGACAAACATGG | |
| | CCACCTTTACAGATGCAGAGATCGACGAGCTATTTGAGACAAGTGGA | |
| | ACTGTCATTGACAACATAATTACAGCCCAGGGTAAACCAGCAGAGAC | |
| | TGTTGGAAGGAGTGCAATCCCACAAGGCAAGACCAAGGTGCTGAGCG | |
| | CAGCATGGGAGAAGCATGGGAGCATCCAGCCACCGGCCAGTCAAGAC | |
| | AACCCCGATCGACAGGACAGATCTGACAAACAACCATCCACACCCGA | |
| | GCAAACGACCCCGCATGACAGCCCGCCGGCCACATCCGCCGACCAGC | |
| | CCCCCACCCAGGCCACAGACGAAGCCGTCGACACACAGCTCAGGACC | |
| | GGAGCAAGCAACTCTCTGCTGTTGATGCTTGACAAGCTCAGCAATAA | |
| | ATCGTCCAATGCTAAAAAGGGCCCATGGTCGAGCCCCCAAGAGGGGA | |
| | ATCACCAACGTCCGACTCAACAGCAGGGGAGTCAACCCAGTCGCGGA | |
| | AACAGTCAGGAAAGACCGCAGAACCAAGTCAAGGCCGCCCCTGGAAA | |
| | CCAGGGCACAGACGTGAACACAGCATATCATGGACAATGGGAGGAGT | |
| | CACAACTATCAGCTGGTGCAACCCCTCATGCTCTCCGATCAAGGCAG | |
| | AGCCAAGACAATACCCTTGTATCTGCGGATCATGTCCAGCCACCTGT | |
| | AGACTTTGTGCAAGCGATGATGTCTATGATGGAGGCGATATCACAGA | |
| | GAGTAAGTAAGGTTGACTATCAGCTAGATCTTGTCTTGAAACAGACA | |
| | TCCTCCATCCCTATGATGCGGTCCGAAATCCAACAGCTGAAAACATC | |
| | TGTTGCAGTCATGGAAGCCAACTTGGGAATGATGAAGATTCTGGATC | |
| | CCGGTTGTGCCAACATTTCATCTCTGAGTGATCTACGGGCAGTTGCC | |
| | CGATCTCACCCGGTTTTAGTTTCAGGCCCTGGAGACCCCTCTCCCTA | |
| | TGTGACACAAGGAGGCGAAATGGCACTTAATAAACTTTCGCAACCAG | |
| | TGCCACATCCATCTGAATTGATTAAACCCGCCACTGCATGCGGGCCT | |
| | GATATAGGAGTGGAAAAGGACACTGTCCGTGCATTGATCATGTCACG | |
| | CCCAATGCACCCGAGTTCTTCAGCCAAGCTCCTAAGCAAGTTAGATG | |
| | CAGCCGGGTCGATCGAGGAAATCAGGAAAATCAAGCGCCTTGCTCTA | |
| | AATGGCTAATTACTACTGCCACACGTAGCGGGTCCCTGTCCACTCGG | |
| | CATCACACGGAATCTGCACCGAGTTCCCCCCCGCGGTTAGAAAAAAT | |
| | ACGGGTAGAACCGCCACCATGGTGAGCAAGGGCGAGGAGCTGTTCAC | |
| | CGGGGTGGTGCCCATCCTGGTCGAGCTGGACGGCGACGTAAACGGCC | |
| | ACAAGTTCAGCGTGTCCGGCGAGGGCGAGGGCGATGCCACCTACGGC | |
| | AAGCTGACCCTGAAGTTCATCTGCACCACCGGCAAGCTGCCCGTGCC | |
| | CTGGCCCACCCTCGTGACCACCCTGACCTACGGCGTGCAGTGCTTCA | |
| | GCCGCTACCCCGACCACATGAAGCAGCACGACTTCTTCAAGTCCGCC | |
| | ATGCCCGAAGGCTACGTCCAGGAGCGCACCATCTTCTTCAAGGACGA | |
| | CGGCAACTACAAGACCCGCGCCGAGGTGAAGTTCGAGGGCGACACCC | |
| | TGGTGAACCGCATCGAGCTGAAGGGCATCGACTTCAAGGAGGACGGC | |
| | AACATCCTGGGGCACAAGCTGGAGTACAACTACAACAGCCACAACGT | |
| | CTATATCATGGCCGACAAGCAGAAGAACGGCATCAAGGTGAACTTCA | |
| | AGATCCGCCACAACATCGAGGACGGCAGCGTGCAGCTCGCCGACCAC | |
| | TACCAGCAGAACACCCCCATCGGCGACGGCCCCGTGCTGCTGCCCGA | |
| | CAACCACTACCTGAGCACCCAGTCCGCCCTGAGCAAAGACCCCAACG | |
| | AGAAGCGCGATCACATGGTCCTGCTGGAGTTCGTGACCGCCGCCGGG | |
| | ATCACTCTCGGCATGGACGAGCTGTACAAGTGACCCGCGGACCCAAG | |
| | GTCCAACTCTCCAAGCGGCAATCCTCTCTCGCTTCCTCAGCCCCACT | |
| | GAATGATCGCGTAACCGTAATTAATCTAGCTACATTTAAGATTAAGA | |
| | AAAAATACGGGTAGAATTGGAGTGCCCCAATTGTGCCAAGATGGACT | |
| | CATCTAGGACAATTGGGCTGTACTTTGATTCTGCCCATTCTTCTAGC | |
| | AACCTGTTAGCATTTCCGATCGTCCTACAAGACACAGGAGATGGGAA | |
| | GAAGCAAATCGCCCCGCAATATAGGATCCAGCGCCTTGACTTGTGGA | |
| | CTGATAGTAAGGAGGACTCAGTATTCATCACCACCTATGGATTCATC | |
| | TTTCAAGTTGGGAATGAAGAAGCCACCGTCGGCATGATCGATGATAA | |
| | ACCCAAGCGCGAGTTACTTTCCGCTGCGATGCTCTGCCTAGGAAGCG | |
| | TCCCAAATACCGGAGACCTTATTGAGCTGGCAAGGGCCTGTCTCACT | |
| | ATGATAGTCACATGCAAGAAGAGTGCAACTAATACTGAGAGAATGGT | |
| | TTTCTCAGTAGTGCAGGCACCCCAAGTGCTGCAAAGCTGTAGGGTTG | |
| | TGGCAAACAAATACTCATCAGTGAATGCAGTCAAGCACGTGAAAGCG | |
| | CCAGAGAAGATTCCCGGGAGTGGAACCCTAGAATACAAGGTGAACTT | |
| | TGTCTCCTTGACTGTGGTACCGAAGAGGGATGTCTACAAGATCCCAG | |
| | CTGCAGTATTGAAGGTTTCTGGCTCGAGTCTGTACAATCTTGCGCTC | |
| | AATGTCACTATTAATGTGGAGGTAGACCCGAGGAGTCCTTTGGTTAA | |
| | ATCTCTGTCTAAGTCTGACAGCGGATACTATGCTAACCTCTTCTTGC | |
| | ATATTGGACTTATGACCACTGTAGATAGGAAGGGGAAGAAAGTGACA | |
| | TTTGACAAGCTGGAAAAGAAAATAAGGAGCCTTGATCTATCTGTCGG | |
| | GCTCAGTGATGTGCTCGGGCCTTCCGTGTTGGTAAAAGCAAGAGGTG | |
| | CACGGACTAAGCTTTTGGCACCTTTCTTCTCTAGCAGTGGGACAGCC | |
| | TGCTATCCCATAGCAAATGCTTCTCCTCAGGTGGCCAAGATACTCTG | |
| | GAGTCAAACCGCGTGCCTGCGGAGCGTTAAAATCATTATCCAAGCAG | |
| | GTACCCAACGCGCTGTCGCAGTGACCGCCGACCACGAGGTTACCTCT | |
| | ACTAAGCTGGAGAAGGGGCACACCCTTGCCAAATACAATCCTTTTAA | |
| | GAAATAAGCTGCGTCTCTGAGATTGCGCTCCGCCCACTCACCCAGAT | |
| | CATCATGACACAAAAAACTAATCTGTCTTGATTatttacagttagtt | |
| | tacctgtctatcaagttagaaaaaacacgggtagaagattctggatc | |
| | ccggttggcgccctccaggtgcaagATGAATCAAGCACTCGTGATTT | |
| | TGTTGGTATCTTTCCAGCTCGGCGTTGCCTTAGATAACTCAGTGTTG | |
| | GCTCCAATAGGAGTAGCTAGCGCACAGGAGTGGCAACTGGCGGCATA | |
| | TACAACGACCCTCACAGGGACCATCGCAGTGAGATTTATCCCGGTCC | |
| | TGCCTGGGAACCTATCAACATGTGCACAGGAGACGCTGCAGGAATAT | |
| | AATAGAACTGTGACTAATATCTTAGGCCCGTTGAGAGAGAACTTGGA | |
| | TGCTCTCCTATCTGACTTCGATAAACCTGCATCGAGGTTCGTGGGCG | |
| | CCATCATTGGGTCGGTGGCCTTGGGGGTAGCAACAGCTGCACAAATC | |
| | ACAGCCGCCGTGGCTCTCAATCAAGCACAAGAGAATGCCCGGAATAT | |
| | ATGGCGTCTCAAGGAATCGATAAAGAAAACCAATGCGGCTGTGTTGG | |
| | AATTGAAGGATGGACTTGCAACGACTGCTATAGCTTTGGACAAAGTG | |
| | CAAAAGTTTATCAATGATGATATTATACCACAGATTAAGGACATTGA | |
| | CTGCCAGGTAGTTGCAAATAAATTAGGCGTCTACCTCTCCTTATACT | |
| | TAACAGAGCTTACAACTGTATTTGGTTCTCAGATCACTAATCCTGCA | |
| | TTATCAACGCTCTCTTACCAGGCGCTGTACAGCTTATGTGGAGGGGA | |
| | TATGGGAAAGCTAACTGAGCTGATCGGTGTCAATGCAAAGGATGTGG | |
| | GATCCCTCTACGAGGCTAACCTCATAACCGGCCAAATCGTTGGATAT | |
| | GACCCTGAACTACAGATAATCCTCATACAAGTATCTTACCCAAGTGT | |
| | GTCTGAAGTGACAGGAGTCCGGGCTACTGAGTTAGTCACTGTCAGTG | |
| | TCACTACACCAAAAGGAGAAGGGCAGGCAATTGTTCCGAGATATGTG | |
| | GCACAGAGTAGAGTGCTGACAGAGGAGTTGGATGTCTCGACTTGTAG | |
| | GTTTAGCAAAACAACTCTTTATTGTAGGTCGATTCTCACACGGCCCC | |
| | TACCAACTTTGATCGCCAGCTGCCTGTCAGGGAAGTACGACGATTGT | |
| | CAGTACACAACAGAGATAGGAGCGCTATCTTCGAGATTCATCACAGT | |
| | CAATGGTGGAGTCCTTGCAAACTGCAGAGCAATTGTGTGTAAGTGTG | |
| | TCTCACCCCCGCATATAATACCACAAAACGACATTGGCTCCGTAACA | |
| | GTTATTGACTCAAGTATATGCAAGGAAGTTGTCTTAGAGAGTGTGCA | |
| | GCTTAGGTTAGAAGGAAAGCTGTCATCCCAATACTTCTCCAACGTGA | |
| | CAATTGACCTTTCCCAAATCACAACGTCAGGGTCGCTGGATATAAGC | |
| | AGTGAAATTGGTAGCATTAACAACACAGTTAATCGGGTCGACGAGTT | |
| | AATCAAGGAATCCAACGAGTGGCTGAACGCTGTGAACCCCCGCCTTG | |
| | TGAACAATACGAGCATCATAGTCCTCTGTGTCCTTGCCGCCCTGATT | |
| | ATTGTCTGGCTAATAGCGCTGACAGTATGCTTCTGTTACTCCGCAAG | |
| | ATACTCAGCTAAGTCAAAACAGATGAGGGGCGCTATGACAGGGATCG | |
| | ATAATCCATATGTAATACAGAGTGCAACTAAGATGtgaacacagatg | |
| | aggaacgaaggtttccctaatagtaatttgtgtgaaagttctggtag | |
| | tctgtcagttcagagagttaagaaaaaactaccggttgtagatgacc | |
| | aaaggacgatatacgggtagaacggtaagagaggccgcccctcaatt | |
| | gcgagccaggcttcacaacctccgttctaccgcttcaccgacaacag | |
| | tcctcaatcATGGATTTCCCATCTAGGGAGAACCTGGCAGCAGGTGA | |
| | CATATCGGGGCGGAAGACTTGGAGATTACTGTTCCGGATCCTCACAT | |
| | TGAGCATAGGTGTGGTCTGTCTTGCCATCAATATTGCCACAATTGCA | |
| | AAATTGGATCACCTGGATAACATGGCTTCGAACACATGGACAACAAC | |
| | TGAGGCTGACCGTGTGATATCTAGCATCACGACTCCGCTCAAAGTCC | |
| | CTGTCAACCAGATTAATGACATGTTTCGGATTGTAGCGCTTGACCTA | |
| | CCTCTGCAGATGACATCATTACAGAAAGAAATAACATCCCAAGTCGG | |
| | GTTCTTGGCTGAAAGTATCAACAATGTTTTATCCAAGAATGGATCTG | |
| | CAGGCCTGGTTCTTGTTAATGACCCTGAATATGCAGGGGGGATCGCT | |
| | GTCAGCTTGTACCAAGGAGATGCATCTGCAGGCCTAAATTTCCAGCC | |
| | CATTTCTTTAATAGAACATCCAAGTTTTGTCCCTGGTCCTACTACTG | |
| | CTAAGGGCTGTATAAGGATCCCGACCTTCCATATGGGCCCTTCACAT | |
| | TGGTGTTACTCACATAACATCATTGCATCAGGTTGCCAGGATGCGAG | |
| | CCACTCCAGTATGTATATCTCTCTGGGGGTGCTGAAAGCATCGCAGA | |
| | CCGGGTCGCCTATCTTCTTGACAACGGCCAGCCATCTCGTGGATGAC | |
| | AACATCAACCGGAAGTCATGCAGCATCGTAGCCTCAAAATACGGTTG | |
| | TGATATCCTATGCAGTATTGTGATTGAAACAGAGAATGAGGATTATA | |
| | GGTCTGATCCGGCTACTAGCATGATTATAGGTAGGCTGTTCTTCAAC | |
| | GGGTCATACACAGAGAGCAAGATTAACACAGGGTCCATCTTCAGTCT | |
| | ATTCTCTGCTAACTACCCTGCGGTGGGGTCGGGTATTGTAGTCGGGG | |
| | ATGAAGCCGCATTCCCAATATATGGTGGGGTCAAGCAGAACACATGG | |
| | TTGTTCAACCAGCTCAAGGATTTTGGTTACTTCACCCATAATGATGT | |
| | GTACAAGTGCAATCGGACTGATATACAGCAAACTATCCTGGATGCAT | |
| | ACAGGCCACCTAAAATCTCAGGAAGGTTATGGGTACAAGGCATCCTA | |
| | TTGTGCCCAGTTTCACTGAGACCTGATCCTGGCTGTCGCTTAAAGGT | |
| | GTTCAATACCAGCAATGTGATGATGGGGGCAGAAGCGAGGTTGATCC | |
| | AAGTAGGCTCAACCGTGTATCTATACCAACGCTCATCCTCATGGTGG | |
| | GTGGTAGGACTGACTTACAAATTAGATGTGTCAGAAATAACTTCACA | |
| | GACAGGTAACACACTCAACCATGTAGACCCCATTGCCCATACAAAGT | |
| | TCCCAAGACCATCTTTCAGGCGAGATGCGTGTGCGAGGCCAAACATA | |
| | TGCCCTGCTGTCTGTGTCTCCGGAGTTTATCAGGACATTTGGCCGAT | |
| | CAGTACAGCCACCAATAACAGCAACATTGTGTGGGTTGGACAGTACT | |
| | TAGAAGCATTCTATTCCAGGAAAGACCCAAGAATAGGGATAGCAACC | |
| | CAGTATGAGTGGAAAGTCACCAACCAGCTGTTCAATTCGAATACTGA | |
| | GGGAGGGTACTCAACCACAACATGCTTCCGGAACACCAAACGGGACA | |
| | AGGCATATTGTGTAGTGATATCAGAGTACGCTGATGGGGTGTTCGGA | |
| | TCATACAGGATCGTTCCTCAGCTTATAGAGATTAGAACAACCACCGG | |
| | TAAATCTGAGtagttgagtcaattataaaggagttggaaagatggca | |
| | ttgtatcacctatcttctgcgacatcaagaatcaAACCGAATGCCGG | |
| | CGCGTGCTCGAATTCCATGTTGCCAGTTGACCACAATCAGCCAGTGC | |
| | TCATGCGATCAGATTAAGCCTTGTCAATAGTCTCTTGATTAAGAAAA | |
| | AATGTAAGTGGCAATGAGATACAAGGCAAAACAGCTCATGGTTAACA | |
| | ATACGGGTAGGACATGGCGAGCTCCGGTCCTGAAAGGGCAGAGCATC | |
| | AGATTATCCTACCAGAGTCACACCTGTCTTCACCATTGGTCAAGCAC | |
| | AAACTACTCTATTACTGGAAATTAACTGGGCTACCGCTTCCTGATGA | |
| | ATGTGACTTCGACCACCTCATTCTCAGCCGACAATGGAAAAAAATAC | |
| | TTGAATCGGCCTCTCCTGATACTGAGAGAATGATAAAACTCGGAAGG | |
| | GCAGTACACCAAACTCTTAACCACAATTCCAGAATAACCGGAGTGCT | |
| | CCACCCCAGGTGTTTAGAAGAACTGGCTAATATTGAGGTCCCAGATT | |
| | CAACCAACAAATTTCGGAAGATTGAGAAGAAGATCCAAATTCACAAC | |
| | ACGAGATATGGAGAACTGTTCACAAGGCTGTGTACGCATATAGAGAA | |
| | GAAACTGCTGGGGTCATCTTGGTCTAACAATGTCCCCCGGTCAGAGG | |
| | AGTTCAGCAGCATTCGTACGGATCCGGCATTCTGGTTTCACTCAAAA | |
| | TGGTCCACAGCCAAGTTTGCATGGCTCCATATAAAACAGATCCAGAG | |
| | GCATCTGATGGTGGCAGCTAGGACAAGGTCTGCGGCCAACAAATTGG | |
| | TGATGCTAACCCATAAGGTAGGCCAAGTCTTTGTCACTCCTGAACTT | |
| | GTCGTTGTGACGCATACGAATGAGAACAAGTTCACATGTCTTACCCA | |
| | GGAACTTGTATTGATGTATGCAGATATGATGGAGGGCAGAGATATGG | |
| | TCAACATAATATCAACCACGGCGGTGCATCTCAGAAGCTTATCAGAG | |
| | AAAATTGATGACATTTTGCGGTTAATAGACGCTCTGGCAAAAGACTT | |
| | GGGTAATCAAGTCTACGATGTTGTATCACTAATGGAGGGATTTGCAT | |
| | ACGGAGCTGTCCAGCTACTCGAGCCGTCAGGTACATTTGCAGGAGAT | |
| | TTCTTCGCATTCAACCTGCAGGAGCTTAAAGACATTCTAATTGGCCT | |
| | CCTCCCCAATGATATAGCAGAATCCGTGACTCATGCAATCGCTACTG | |
| | TATTCTCTGGTTTAGAACAGAATCAAGCAGCTGAGATGTTGTGTCTG | |
| | TTGCGTCTGTGGGGTCACCCACTGCTTGAGTCCCGTATTGCAGCAAA | |
| | GGCAGTCAGGAGCCAAATGTGCGCACCGAAAATGGTAGACTTTGATA | |
| | TGATCCTTCAGGTACTGTCTTTCTTCAAGGGAACAATCATCAACGGG | |
| | TACAGAAAGAAGAATGCAGGTGTGTGGCCGCGAGTCAAAGTGGATAC | |
| | AATATATGGGAAGGTCATTGGGCAACTACATGCAGATTCAGCAGAGA | |
| | TTTCACACGATATCATGTTGAGAGAGTATAAGAGTTTATCTGCACTT | |
| | GAATTTGAGCCATGTATAGAATATGACCCTGTCACCAACCTGAGCAT | |
| | GTTCCTAAAAGACAAGGCAATCGCACACCCCAACGATAATTGGCTTG | |
| | CCTCGTTTAGGCGGAACCTTCTCTCCGAAGACCAGAAGAAACATGTA | |
| | AAAGAAGCAACTTCGACTAATCGCCTCTTGATAGAGTTTTTAGAGTC | |
| | AAATGATTTTGATCCATATAAAGAGATGGAATATCTGACGACCCTTG | |
| | AGTACCTTAGAGATGACAATGTGGCAGTATCATACTCGCTCAAGGAG | |
| | AAGGAAGTGAAAGTTAATGGACGGATCTTCGCTAAGCTGACAAAGAA | |
| | GTTAAGGAACTGTCAGGTGATGGCGGAAGGGATCCTAGCCGATCAGA | |
| | TTGCACCTTTCTTTCAGGGAAATGGAGTCATTCAGGATAGCATATCC | |
| | TTGACCAAGAGTATGCTAGCGATGAGTCAACTGTCTTTTAACAGCAA | |
| | TAAGAAACGTATCACTGACTGTAAAGAAAGAGTATCTTCAAACCGCA | |
| | ATCATGATCCGAAAAGCAAGAACCGTCGGAGAGTTGCAACCTTCATA | |
| | ACAACTGACCTGCAAAAGTACTGTCTTAATTGGAGATATCAGACAAT | |
| | CAAATTGTTCGCTCATGCCATCAATCAGTTGATGGGCCTACCTCACT | |
| | TCTTCGAATGGATTCACCTAAGACTGATGGACACTACGATGTTCGTA | |
| | GGAGACCCTTTCAATCCTCCAAGTGACCCTACTGACTGTGACCTCTC | |
| | AAGAGTCCCTAATGATGACATATATATTGTCAGTGCCAGAGGGGGTA | |
| | TCGAAGGATTATGCCAGAAGCTATGGACAATGATCTCAATTGCTGCA | |
| | ATCCAACTTGCTGCAGCTAGATCGCATTGTCGTGTTGCCTGTATGGT | |
| | ACAGGGTGATAATCAAGTAATAGCAGTAACGAGAGAGGTAAGATCAG | |
| | ACGACTCTCCGGAGATGGTGTTGACACAGTTGCATCAAGCCAGTGAT | |
| | AATTTCTTCAAGGAATTAATTCATGTCAATCATTTGATTGGCCATAA | |
| | TTTGAAGGATCGTGAAACCATCAGGTCAGACACATTCTTCATATACA | |
| | GCAAACGAATCTTCAAAGATGGAGCAATCCTCAGTCAAGTCCTCAAA | |
| | AATTCATCTAAATTAGTGCTAGTGTCAGGTGATCTCAGTGAAAACAC | |
| | CGTAATGTCCTGTGCCAACATTGCCTCTACTGTAGCACGGCTATGCG | |
| | AGAACGGGCTTCCCAAAGACTTCTGTTACTATTTAAACTATATAATG | |
| | AGTTGTGTGCAGACATACTTTGACTCTGAGTTCTCCATCACCAACAA | |
| | TTCGCACCCCGATCTTAATCAGTCGTGGATTGAGGACATCTCTTTTG | |
| | TGCACTCATATGTTCTGACTCCTGCCCAATTAGGGGGACTGAGTAAC | |
| | CTTCAATACTCAAGGCTCTACACTAGAAATATCGGTGACCCGGGGAC | |
| | TACTGCTTTTGCAGAGATCAAGCGACTAGAAGCAGTGGGATTACTGA | |
| | GTCCTAACATTATGACTAATATCTTAACTAGGCCGCCTGGGAATGGA | |
| | GATTGGGCCAGTCTGTGCAACGACCCATACTCTTTCAATTTTGAGAC | |
| | TGTTGCAAGCCCAAATATTGTTCTTAAGAAACATACGCAAAGAGTCC | |
| | TATTTGAAACTTGTTCAAATCCCTTATTGTCTGGAGTGCACACAGAG | |
| | GATAATGAGGCAGAAGAGAAGGCATTGGCTGAATTCTTGCTTAATCA | |
| | AGAGGTGATTCATCCCCGCGTTGCGCATGCCATCATGGAGGCAAGCT | |
| | CTGTAGGTAGGAGAAAGCAAATTCAAGGGCTTGTTGACACAACAAAC | |
| | ACCGTAATTAAGATTGCGCTTACTAGGAGGCCATTAGGCATCAAGAG | |
| | GCTGATGCGGATAGTCAATTATTCTAGCATGCATGCAATGCTGTTTA | |
| | GAGACGATGTTTTTTCCTCCAGTAGATCCAACCACCCCTTAGTCTCT | |
| | TCTAATATGTGTTCTCTGACACTGGCAGACTATGCACGGAATAGAAG | |
| | CTGGTCACCTTTGACGGGAGGCAGGAAAATACTGGGTGTATCTAATC | |
| | CTGATACGATAGAACTCGTAGAGGGTGAGATTCTTAGTGTAAGCGGA | |
| | GGGTGTACAAGATGTGACAGCGGAGATGAACAATTTACTTGGTTCCA | |
| | TCTTCCAAGCAATATAGAATTGACCGATGACACCAGCAAGAATCCTC | |
| | CGATGAGGGTACCATATCTCGGGTCAAAGACACAGGAGAGGAGAGCT | |
| | GCCTCACTTGCAAAAATAGCTCATATGTCGCCACATGTAAAGGCTGC | |
| | CCTAAGGGCATCATCCGTGTTGATCTGGGCTTATGGGGATAATGAAG | |
| | TAAATTGGACTGCTGCTCTTACGATTGCAAAATCTCGGTGTAATGTA | |
| | AACTTAGAGTATCTTCGGTTACTGTCCCCTTTACCCACGGCTGGGAA | |
| | TCTTCAACATAGACTAGATGATGGTATAACTCAGATGACATTCACCC | |
| | CTGCATCTCTCTACAGGGTGTCACCTTACATTCACATATCCAATGAT | |
| | TCTCAAAGGCTGTTCACTGAAGAAGGAGTCAAAGAGGGGAATGTGGT | |
| | TTACCAACAGATCATGCTCTTGGGTTTATCTCTAATCGAATCGATCT | |
| | TTCCAATGACAACAACCAGGACATATGATGAGATCACACTGCACCTA | |
| | CATAGTAAATTTAGTTGCTGTATCAGAGAAGCACCTGTTGCGGTTCC | |
| | TTTCGAGCTACTTGGGGTGGTACCGGAACTGAGGACAGTGACCTCAA | |
| | ATAAGTTTATGTATGATCCTAGCCCTGTATCGGAGGGAGACTTTGCG | |
| | AGACTTGACTTAGCTATCTTCAAGAGTTATGAGCTTAATCTGGAGTC | |
| | ATATCCCACGATAGAGCTAATGAACATTCTTTCAATATCCAGCGGGA | |
| | AGTTGATTGGCCAGTCTGTGGTTTCTTATGATGAAGATACCTCCATA | |
| | AAGAATGACGCCATAATAGTGTATGACAATACCCGAAATTGGATCAG | |
| | TGAAGCTCAGAATTCAGATGTGGTCCGCCTATTTGAATATGCAGCAC | |
| | TTGAAGTGCTCCTCGACTGTTCTTACCAACTCTATTACCTGAGAGTA | |
| | AGAGGCCTGGACAATATTGTCTTATATATGGGTGATTTATACAAGAA | |
| | TATGCCAGGAATTCTACTTTCCAACATTGCAGCTACAATATCTCATC | |
| | CCGTCATTCATTCAAGGTTACATGCAGTGGGCCTGGTCAACCATGAC | |
| | GGATCACACCAACTTGCAGATACGGATTTTATCGAAATGTCTGCAAA | |
| | ACTATTAGTATCTTGCACCCGACGTGTGATCTCCGGCTTATATTCAG | |
| | GAAATAAGTATGATCTGCTGTTCCCATCTGTCTTAGATGATAACCTG | |
| | AATGAGAAGATGCTTCAGCTGATATCCCGGTTATGCTGTCTGTACAC | |
| | GGTACTCTTTGCTACAACAAGAGAAATCCCGAAAATAAGAGGCTTAA | |
| | CTGCAGAAGAGAAATGTTCAATACTCACTGAGTATTTACTGTCGGAT | |
| | GCTGTGAAACCATTACTTAGCCCCGATCAAGTGAGCTCTATCATGTC | |
| | TCCTAACATAATTACATTCCCAGCTAATCTGTACTACATGTCTCGGA | |
| | AGAGCCTCAATTTGATCAGGGAAAGGGAGGACAGGGATACTATCCTG | |
| | GCGTTGTTGTTCCCCCAAGAGCCATTATTAGAGTTCCCTTCTGTGCA | |
| | AGATATTGGTGCTCGAGTGAAAGATCCATTCACCCGACAACCTGCGG | |
| | CATTTTTGCAAGAGTTAGATTTGAGTGCTCCAGCAAGGTATGACGCA | |
| | TTCACACTTAGTCAGATTCATCCTGAACTCACATCTCCAAATCCGGA | |
| | GGAAGACTACTTAGTACGATACTTGTTCAGAGGGATAGGGACTGCAT | |
| | CTTCCTCTTGGTATAAGGCATCTCATCTCCTTTCTGTACCCGAGGTA | |
| | AGATGTGCAAGACACGGGAACTCCTTATACTTAGCTGAAGGGAGCGG | |
| | AGCCATCATGAGTCTTCTCGAACTGCATGTACCACATGAAACTATCT | |
| | ATTACAATACGCTCTTTTCAAATGAGATGAACCCCCCGCAACGACAT | |
| | TTCGGGCCGACCCCAACTCAGTTTTTGAATTCGGTTGTTTATAGGAA | |
| | TCTACAGGCGGAGGTAACATGCAAAGATGGATTTGTCCAAGAGTTCC | |
| | GTCCATTATGGAGAGAAAATACAGAGGAAAGCGACCTGACCTCAGAT | |
| | AAAGTAGTGGGGTATATTACATCTGCAGTGCCCTACAGATCTGTATC | |
| | ATTGCTGCATTGTGACATTGAAATTCCTCCAGGGTCCAATCAAAGCT | |
| | TACTAGATCAACTAGCTATCAATTTATCTCTGATTGCCATGCATTCT | |
| | GTAAGGGAGGGCGGGGTAGTAATCATCAAAGTGTTGTATGCAATGGG | |
| | ATACTACTTTCATCTACTCATGAACTTGTTTGCTCCGTGTTCCACAA | |
| | AAGGATATATTCTCTCTAATGGTTATGCATGTCGAGGAGATATGGAG | |
| | TGTTACCTGGTATTTGTCATGGGTTACCTGGGCGGGCCTACATTTGT | |
| | ACATGAGGTGGTGAGGATGGCGAAAACTCTGGTGCAGCGGCACGGTA | |
| | CGCTTTTGTCTAAATCAGATGAGATCACACTGACCAGGTTATTCACC | |
| | TCACAGCGGCAGCGTGTGACAGACATCCTATCCAGTCCTTTACCAAG | |
| | ATTAATAAAGTACTTGAGGAAGAATATTGACACTGCGCTGATTGAAG | |
| | CCGGGGGACAGCCCGTCCGTCCATTCTGTGCGGAGAGTCTGGTGAGC | |
| | ACGCTAGCGAACATAACTCAGATAACCCAGATCATCGCTAGTCACAT | |
| | TGACACAGTTATCCGGTCTGTGATATATATGGAAGCTGAGGGTGATC | |
| | TCGCTGACACAGTATTTCTATTTACCCCTTACAATCTCTCTACTGAC | |
| | GGGAAAAAGAGGACATCACTTAAACAGTGCACGAGACAGATCCTAGA | |
| | GGTTACAATACTAGGTCTTAGAGTCGAAAATCTCAATAAAATAGGCG | |
| | ATATAATCAGCCTAGTGCTTAAAGGCATGATCTCCATGGAGGACCTT | |
| | ATCCCACTAAGGACATACTTGAAGCATAGTACCTGCCCTAAATATTT | |
| | GAAGGCTGTCCTAGGTATTACCAAACTCAAAGAAATGTTTACAGACA | |
| | CTTCTGTACTGTACTTGACTCGTGCTCAACAAAAATTCTACATGAAA | |
| | ACTATAGGCAATGCAGTCAAAGGATATTACAGTAACTGTGACTCTTA | |
| | ACGAAAATCACATATTAATAGGCTCCTTTTTTGGCCAATTGTATTCT | |
| | TGTTGATTTAATCATATTATGTTAGAAAAAAGTTGAACCCTGACTCC | |
| | TTAGGACTCGAATTCGAACTCAAATAAATGTCTTAAAAAAAGGTTGC | |
| | GCACAATTATTCTTGAGTGTAGTCTCGTCATTCACCAAATCTTTGTT | |
| | TGGTGCGCGCGGCCGGCATGGTCCCAGCCTCCTCGCTGGCGCCGGCT | |
| | GGGCAACATTCCGAGGGGACCGTCCCCTCGGTAATGGCGAATGGGAC | |
| | GTCGACTGCTAACAAAGCCCGAAAGGAAGCTGAGTTGGCTGCTGCCA | |
| | CCGCTGAGCAATAACTAGCATAACCCCTTGGGGCCTCTAAACGGGTC | |
| | TTGAGGGGTTTTTTGCTGAAAGGAGGAACTATATGCGCGCAGATCTG | |
| | TCATGATGATCATTGCAATTGGATCCATATATAGGGCCCGGGTTATA | |
| | ATTACCTCAGGTCGACGTCCCATGGCCATTCGAATTCGTAATCATGG | |
| | TCATAGCTGTTTCCTGTGTGAAATTGTTATCCGCTCACAATTCCACA | |
| | CAACATACGAGCCGGAAGCATAAAGTGTAAAGCCTGGGGTGCCTAAT | |
| | GAGTGAGCTAACTCACATTAATTGCGTTGCGCTCACTGCCCGCTTTC | |
| | CAGTCGGGAAACCTGTCGTGCCAGCTGCATTAATGAATCGGCCAACG | |
| | CGCGGGGAGAGGCGGTTTGCGTATTGGGCGC | |
| NDV- | TCTTCCGCTTCCTCGCTCACTGACTCGCTGCGCTCGGTCGTTC | 45 |
| APMV3- | GGCTGCGGCGAGCGGTATCAGCTCACTCAAAGGCGGTAATACG | |
| GFP: | GTTATCCACAGAATCAGGGGATAACGCAGGAAAGAACATGTGA | |
| APMV3/ | GCAAAAGGCCAGCAAAAGGCCAGGAACCGTAAAAAGGCCGCGT | |
| Turkey/ | TGCTGGCGTTTTTCCATAGGCTCCGCCCCCCTGACGAGCATCA | |
| Wisconsin/68 | CAAAAATCGACGCTCAAGTCAGAGGTGGCGAAACCCGACAGGA | |
| (GFP- | CTATAAAGATACCAGGCGTTTCCCCCTGGAAGCTCCCTCGTGC | |
| encoding | GCTCTCCTGTTCCGACCCTGCCGCTTACCGGATACCTGTCCGC | |
| sequence is | CTTTCTCCCTTCGGGAAGCGTGGCGCTTTCTCATAGCTCACGC | |
| in bold and | TGTAGGTATCTCAGTTCGGTGTAGGTCGTTCGCTCCAAGCTGG | |
| italics, | GCTGTGTGCACGAACCCCCCGTTCAGCCCGACCGCTGCGCCTT | |
| APMV3 F | ATCCGGTAACTATCGTCTTGAGTCCAACCCGGTAAGACACGAC | |
| protein- | TTATCGCCACTGGCAGCAGCCACTGGTAACAGGATTAGCAGAG | |
| encoding | CGAGGTATGTAGGCGGTGCTACAGAGTTCTTGAAGTGGTGGCC | |
| sequence is | TAACTACGGCTACACTAGAAGAACAGTATTTGGTATCTGCGCT | |
| underline, | CTGCTGAAGCCAGTTACCTTCGGAAAAAGAGTTGGTAGCTCTT | |
| and | GATCCGGCAAACAAACCACCGCTGGTAGCGGTGGTTTTTTTGT | |
| APMV3 | TTGCAAGCAGCAGATTACGCGCAGAAAAAAAGGATCTCAAGAA | |
| HN- | GATCCTTTGATCTTTTCTACGGGGTCTGACGCTCAGTGGAACG | |
| encoding | AAAACTCACGTTAAGGGATTTTGGTCATGAGATTATCAAAAAG | |
| sequence is | GATCTTCACCTAGATCCTTTTAAATTAAAAATGAAGTTTTAAA | |
| double | TCAATCTAAAGTATATATGAGTAAACTTGGTCTGACAGTTACC | |
| underlined) | AATGCTTAATCAGTGAGGCACCTATCTCAGCGATCTGTCTATT | |
| | TCGTTCATCCATAGTTGCCTGACTCCCCGTCGTGTAGATAACT | |
| | ACGATACGGGAGGGCTTACCATCTGGCCCCAGTGCTGCAATGA | |
| | TACCGCGAGACCCACGCTCACCGGCTCCAGATTTATCAGCAAT | |
| | AAACCAGCCAGCCGGAAGGGCCGAGCGCAGAAGTGGTCCTGCA | |
| | ACTTTATCCGCCTCCATCCAGTCTATTAATTGTTGCCGGGAAG | |
| | CTAGAGTAAGTAGTTCGCCAGTTAATAGTTTGCGCAACGTTGT | |
| | TGCCATTGCTACAGGCATCGTGGTGTCACGCTCGTCGTTTGGT | |
| | ATGGCTTCATTCAGCTCCGGTTCCCAACGATCAAGGCGAGTTA | |
| | CATGATCCCCCATGTTGTGCAAAAAAGCGGTTAGCTCCTTCGG | |
| | TCCTCCGATCGTTGTCAGAAGTAAGTTGGCCGCAGTGTTATCA | |
| | CTCATGGTTATGGCAGCACTGCATAATTCTCTTACTGTCATGC | |
| | CATCCGTAAGATGCTTTTCTGTGACTGGTGAGTACTCAACCAA | |
| | GTCATTCTGAGAATAGTGTATGCGGCGACCGAGTTGCTCTTGC | |
| | CCGGCGTCAATACGGGATAATACCGCGCCACATAGCAGAACTT | |
| | TAAAAGTGCTCATCATTGGAAAACGTTCTTCGGGGCGAAAACT | |
| | CTCAAGGATCTTACCGCTGTTGAGATCCAGTTCGATGTAACCC | |
| | ACTCGTGCACCCAACTGATCTTCAGCATCTTTTACTTTCACCA | |
| | GCGTTTCTGGGTGAGCAAAAACAGGAAGGCAAAATGCCGCAAA | |
| | AAAGGGAATAAGGGCGACACGGAAATGTTGAATACTCATACTC | |
| | TTCCTTTTTCAATATTATTGAAGCATTTATCAGGGTTATTGTC | |
| | TCATGAGCGGATACATATTTGAATGTATTTAGAAAAATAAACA | |
| | AATAGGGGTTCCGCGCACATTTCCCCGAAAAGTGCCACCTGAC | |
| | GTCTAAGAAACCATTATTATCATGACATTAACCTATAAAAATA | |
| | GGCGTATCACGAGGCCCTTTCGTCTCGCGCGTTTCGGTGATGA | |
| | CGGTGAAAACCTCTGACACATGCAGCTCCCGGAGACGGTCACA | |
| | GCTTGTCTGTAAGCGGATGCCGGGAGCAGACAAGCCCGTCAGG | |
| | GCGCGTCAGCGGGTGTTGGCGGGTGTCGGGGCTGGCTTAACTA | |
| | TGCGGCATCAGAGCAGATTGTACTGAGAGTGCACCATAAAATT | |
| | GTAAACGTTAATATTTTGTTAAAATTCGCGTTAAATTTTTGTT | |
| | AAATCAGCTCATTTTTTAACCAATAGGCCGAAATCGGCAAAAT | |
| | CCCTTATAAATCAAAAGAATAGCCCGAGATAGGGTTGAGTGTT | |
| | GTTCCAGTTTGGAACAAGAGTCCACTATTAAAGAACGTGGACT | |
| | CCAACGTCAAAGGGCGAAAAACCGTCTATCAGGGCGATGGCCC | |
| | ACTACGTGAACCATCACCCAAATCAAGTTTTTTGGGGTCGAGG | |
| | TGCCGTAAAGCACTAAATCGGAACCCTAAAGGGAGCCCCCGAT | |
| | TTAGAGCTTGACGGGGAAAGCCGGCGAACGTGGCGAGAAAGGA | |
| | AGGGAAGAAAGCGAAAGGAGCGGGCGCTAGGGCGCTGGCAAGT | |
| | GTAGCGGTCACGCTGCGCGTAACCACCACACCCGCCGCGCTTA | |
| | ATGCGCCGCTACAGGGCGCGTACTATGGTTGCTTTGACGTATG | |
| | CGGTGTGAAATACCGCACAGATGCGTAAGGAGAAAATACCGCA | |
| | TCAGGCGCCATTCGCCATTCAGGCTGCGCAACTGTTGGGAAGG | |
| | GCGATCGGTGCGGGCCTCTTCGCTATTACGCCAGCTGGCGAAA | |
| | GGGGGATGTGCTGCAAGGCGATTAAGTTGGGTAACGCCAGGGT | |
| | TTTCCCAGTCACGACGTTGTAAAACGACGGCCAGTGCCAAGCT | |
| | TTAATACGACTCACTATAGGGAGATTGGTCTGATGAGTCCGTG | |
| | AGGACGAAACGGAGTCTAGACTCCGTCACCAAACAGAGAATCC | |
| | GTGAGTTACGATAAAAGGCGAAGGAGCAATTGAAGTCGCACGG | |
| | GTAGAAGGTGTGAATCTCGAGTGCGAGCCCGAAGCACAAACTC | |
| | GAGAAAGCCTTCTGCCAACATGTCTTCCGTATTTGATGAGTAC | |
| | GAACAGCTCCTCGCGGCTCAGACTCGCCCCAATGGAGCTCATG | |
| | GAGGGGGAGAAAAAGGGAGTACCTTAAAAGTAGACGTCCCGGT | |
| | ATTCACTCTTAACAGTGATGACCCAGAAGATAGATGGAGCTTT | |
| | GTGGTATTCTGCCTCCGGATTGCTGTTAGCGAAGATGCCAACA | |
| | AACCACTCAGGCAAGGTGCTCTCATATCTCTTTTATGCTCCCA | |
| | CTCACAGGTAATGAGGAACCATGTTGCCCTTGCAGGGAAACAG | |
| | AATGAAGCCACATTGGCCGTGCTTGAGATTGATGGCTTTGCCA | |
| | ACGGCACGCCCCAGTTCAACAATAGGAGTGGAGTGTCTGAAGA | |
| | GAGAGCACAGAGATTTGCGATGATAGCAGGATCTCTCCCTCGG | |
| | GCATGCAGCAACGGAACCCCGTTCGTCACAGCCGGGGCCGAAG | |
| | ATGATGCACCAGAAGACATCACCGATACCCTGGAGAGGATCCT | |
| | CTCTATCCAGGCTCAAGTATGGGTCACAGTAGCAAAAGCCATG | |
| | ACTGCGTATGAGACTGCAGATGAGTCGGAAACAAGGCGAATCA | |
| | ATAAGTATATGCAGCAAGGCAGGGTCCAAAAGAAATACATCCT | |
| | CTACCCCGTATGCAGGAGCACAATCCAACTCACGATCAGACAG | |
| | TCTCTTGCAGTCCGCATCTTTTTGGTTAGCGAGCTCAAGAGAG | |
| | GCCGCAACACGGCAGGTGGTACCTCTACTTATTATAACCTGGT | |
| | AGGGGACGTAGACTCATACATCAGGAATACCGGGCTTACTGCA | |
| | TTCTTCTTGACACTCAAGTACGGAATCAACACCAAGACATCAG | |
| | CCCTTGCACTTAGTAGCCTCTCAGGCGACATCCAGAAGATGAA | |
| | GCAGCTCATGCGTTTGTATCGGATGAAAGGAGATAATGCGCCG | |
| | TACATGACATTACTTGGTGATAGTGACCAGATGAGCTTTGCGC | |
| | CTGCCGAGTATGCACAACTTTACTCCTTTGCCATGGGTATGGC | |
| | ATCAGTCCTAGATAAAGGTACTGGGAAATACCAATTTGCCAGG | |
| | GACTTTATGAGCACATCATTCTGGAGACTTGGAGTAGAGTACG | |
| | CTCAGGCTCAGGGAAGTAGCATTAACGAGGATATGGCTGCCGA | |
| | GCTAAAGCTAACCCCAGCAGCAAGGAGGGGCCTGGCAGCTGCT | |
| | GCCCAACGGGTCTCCGAGGAGACCAGCAGCATAGACATGCCTA | |
| | CTCAACAAGTCGGAGTCCTCACTGGGCTTAGCGAGGGGGGGTC | |
| | CCAAGCTCTACAAGGCGGATCGAATAGATCGCAAGGGCAACCA | |
| | GAAGCCGGGGATGGGGAGACCCAATTCCTGGATCTGATGAGAG | |
| | CGGTAGCAAATAGCATGAGGGAGGCGCCAAACTCTGCACAGGG | |
| | CACTCCCCAATCGGGGCCTCCCCCAACTCCTGGGCCATCCCAA | |
| | GATAACGACACCGACTGGGGGTATTGATGGACAAAACCCAGCC | |
| | TGCTTCCACAAAAACATCCCAATGCCCTCACCCGTAGTCGACC | |
| | CCTCGATTTGCGGCTCTATATGACCACACCCTCAAACAAACAT | |
| | CCCCCTCTTTCCTCCCTCCCCCTGCTGTACAACTACGTACGCC | |
| | CTAGATACCACAGGCACAATGCGGCTCACTAACAATCAAAACA | |
| | GAGCCGAGGGAATTAGAAAAAAGTACGGGTAGAAGAGGGATAT | |
| | TCAGAGATCAGGGCAAGTCTCCCGAGTCTCTGCTCTCTCCTCT | |
| | ACCTGATAGACCAGGACAAACATGGCCACCTTTACAGATGCAG | |
| | AGATCGACGAGCTATTTGAGACAAGTGGAACTGTCATTGACAA | |
| | CATAATTACAGCCCAGGGTAAACCAGCAGAGACTGTTGGAAGG | |
| | AGTGCAATCCCACAAGGCAAGACCAAGGTGCTGAGCGCAGCAT | |
| | GGGAGAAGCATGGGAGCATCCAGCCACCGGCCAGTCAAGACAA | |
| | CCCCGATCGACAGGACAGATCTGACAAACAACCATCCACACCC | |
| | GAGCAAACGACCCCGCATGACAGCCCGCCGGCCACATCCGCCG | |
| | ACCAGCCCCCCACCCAGGCCACAGACGAAGCCGTCGACACACA | |
| | GCTCAGGACCGGAGCAAGCAACTCTCTGCTGTTGATGCTTGAC | |
| | AAGCTCAGCAATAAATCGTCCAATGCTAAAAAGGGCCCATGGT | |
| | CGAGCCCCCAAGAGGGGAATCACCAACGTCCGACTCAACAGCA | |
| | GGGGAGTCAACCCAGTCGCGGAAACAGTCAGGAAAGACCGCAG | |
| | AACCAAGTCAAGGCCGCCCCTGGAAACCAGGGCACAGACGTGA | |
| | ACACAGCATATCATGGACAATGGGAGGAGTCACAACTATCAGC | |
| | TGGTGCAACCCCTCATGCTCTCCGATCAAGGCAGAGCCAAGAC | |
| | AATACCCTTGTATCTGCGGATCATGTCCAGCCACCTGTAGACT | |
| | TTGTGCAAGCGATGATGTCTATGATGGAGGCGATATCACAGAG | |
| | AGTAAGTAAGGTTGACTATCAGCTAGATCTTGTCTTGAAACAG | |
| | ACATCCTCCATCCCTATGATGCGGTCCGAAATCCAACAGCTGA | |
| | AAACATCTGTTGCAGTCATGGAAGCCAACTTGGGAATGATGAA | |
| | GATTCTGGATCCCGGTTGTGCCAACATTTCATCTCTGAGTGAT | |
| | CTACGGGCAGTTGCCCGATCTCACCCGGTTTTAGTTTCAGGCC | |
| | CTGGAGACCCCTCTCCCTATGTGACACAAGGAGGCGAAATGGC | |
| | ACTTAATAAACTTTCGCAACCAGTGCCACATCCATCTGAATTG | |
| | ATTAAACCCGCCACTGCATGCGGGCCTGATATAGGAGTGGAAA | |
| | AGGACACTGTCCGTGCATTGATCATGTCACGCCCAATGCACCC | |
| | GAGTTCTTCAGCCAAGCTCCTAAGCAAGTTAGATGCAGCCGGG | |
| | TCGATCGAGGAAATCAGGAAAATCAAGCGCCTTGCTCTAAATG | |
| | GCTAATTACTACTGCCACACGTAGCGGGTCCCTGTCCACTCGG | |
| | CATCACACGGAATCTGCACCGAGTTCCCCCCCGCGGTTAGAAA | |
| | AAATACGGGTAGAACCGCCACCATGGTGAGCAAGGGCGAGGAG | |
| | CTGTTCACCGGGGTGGTGCCCATCCTGGTCGAGCTGGACGGCG | |
| | ACGTAAACGGCCACAAGTTCAGCGTGTCCGGCGAGGGCGAGGG | |
| | CGATGCCACCTACGGCAAGCTGACCCTGAAGTTCATCTGCACC | |
| | ACCGGCAAGCTGCCCGTGCCCTGGCCCACCCTCGTGACCACCC | |
| | TGACCTACGGCGTGCAGTGCTTCAGCCGCTACCCCGACCACAT | |
| | GAAGCAGCACGACTTCTTCAAGTCCGCCATGCCCGAAGGCTAC | |
| | GTCCAGGAGCGCACCATCTTCTTCAAGGACGACGGCAACTACA | |
| | AGACCCGCGCCGAGGTGAAGTTCGAGGGCGACACCCTGGTGAA | |
| | CCGCATCGAGCTGAAGGGCATCGACTTCAAGGAGGACGGCAAC | |
| | ATCCTGGGGCACAAGCTGGAGTACAACTACAACAGCCACAACG | |
| | TCTATATCATGGCCGACAAGCAGAAGAACGGCATCAAGGTGAA | |
| | CTTCAAGATCCGCCACAACATCGAGGACGGCAGCGTGCAGCTC | |
| | GCCGACCACTACCAGCAGAACACCCCCATCGGCGACGGCCCCG | |
| | TGCTGCTGCCCGACAACCACTACCTGAGCACCCAGTCCGCCCT | |
| | GAGCAAAGACCCCAACGAGAAGCGCGATCACATGGTCCTGCTG | |
| | GAGTTCGTGACCGCCGCCGGGATCACTCTCGGCATGGACGAGC | |
| | TGTACAAGTGACCCGCGGACCCAAGGTCCAACTCTCCAAGCGG | |
| | CAATCCTCTCTCGCTTCCTCAGCCCCACTGAATGATCGCGTAA | |
| | CCGTAATTAATCTAGCTACATTTAAGATTAAGAAAAAATACGG | |
| | GTAGAATTGGAGTGCCCCAATTGTGCCAAGATGGACTCATCTA | |
| | GGACAATTGGGCTGTACTTTGATTCTGCCCATTCTTCTAGCAA | |
| | CCTGTTAGCATTTCCGATCGTCCTACAAGACACAGGAGATGGG | |
| | AAGAAGCAAATCGCCCCGCAATATAGGATCCAGCGCCTTGACT | |
| | TGTGGACTGATAGTAAGGAGGACTCAGTATTCATCACCACCTA | |
| | TGGATTCATCTTTCAAGTTGGGAATGAAGAAGCCACCGTCGGC | |
| | ATGATCGATGATAAACCCAAGCGCGAGTTACTTTCCGCTGCGA | |
| | TGCTCTGCCTAGGAAGCGTCCCAAATACCGGAGACCTTATTGA | |
| | GCTGGCAAGGGCCTGTCTCACTATGATAGTCACATGCAAGAAG | |
| | AGTGCAACTAATACTGAGAGAATGGTTTTCTCAGTAGTGCAGG | |
| | CACCCCAAGTGCTGCAAAGCTGTAGGGTTGTGGCAAACAAATA | |
| | CTCATCAGTGAATGCAGTCAAGCACGTGAAAGCGCCAGAGAAG | |
| | ATTCCCGGGAGTGGAACCCTAGAATACAAGGTGAACTTTGTCT | |
| | CCTTGACTGTGGTACCGAAGAGGGATGTCTACAAGATCCCAGC | |
| | TGCAGTATTGAAGGTTTCTGGCTCGAGTCTGTACAATCTTGCG | |
| | CTCAATGTCACTATTAATGTGGAGGTAGACCCGAGGAGTCCTT | |
| | TGGTTAAATCTCTGTCTAAGTCTGACAGCGGATACTATGCTAA | |
| | CCTCTTCTTGCATATTGGACTTATGACCACTGTAGATAGGAAG | |
| | GGGAAGAAAGTGACATTTGACAAGCTGGAAAAGAAAATAAGGA | |
| | GCCTTGATCTATCTGTCGGGCTCAGTGATGTGCTCGGGCCTTC | |
| | CGTGTTGGTAAAAGCAAGAGGTGCACGGACTAAGCTTTTGGCA | |
| | CCTTTCTTCTCTAGCAGTGGGACAGCCTGCTATCCCATAGCAA | |
| | ATGCTTCTCCTCAGGTGGCCAAGATACTCTGGAGTCAAACCGC | |
| | GTGCCTGCGGAGCGTTAAAATCATTATCCAAGCAGGTACCCAA | |
| | CGCGCTGTCGCAGTGACCGCCGACCACGAGGTTACCTCTACTA | |
| | AGCTGGAGAAGGGGCACACCCTTGCCAAATACAATCCTTTTAA | |
| | GAAATAAGCTGCGTCTCTGAGATTGCGCTCCGCCCACTCACCC | |
| | AGATCATCATGACACAAAAAACTAATCTGTCTTGATTatttac | |
| | agttagtttacctgtctatcaagttagaaaaaacacgggtaga | |
| | agattctggatcccggttggcgccctccaggtgcaagATGGCC | |
| | TCCCCAATGGTCCCACTACTCATCATAACGGTAGTACCCGCAC | |
| | TCATTTCAAGTCAATCAGCTAATATTGATAAGCTCATTCAAGC | |
| | AGGGATTATCATGGGCTCAGGGAAGGAACTCCACATTTATCAA | |
| | GAATCTGGCTCTCTTGATTTGTATCTTAGACTATTGCCAGTTA | |
| | TCCCTTCAAATCTTTCTCATTGCCAGAGTGAAGTAATAACACA | |
| | ATATAACTCGACTGTAACGAGACTATTATCACCAATTGCAAAA | |
| | AATCTAAACCATTTGCTACAACCGAGACCGTCTGGCAGGTTAT | |
| | TTGGCGCTGTAATTGGATCGATTGCCTTAGGGGTAGCTACATC | |
| | CGCACAGATTTCAGCTGCTATAGCATTGGTCCGTGCTCAACAG | |
| | AATGCAAACGATATCCTCGCTCTTAAAGCTGCAATACAATCTA | |
| | GTAATGAGGCAATAAAACAACTTACTTATGGCCAAGAAAAGCA | |
| | ACTACTAGCAATATCAAAAATACAAAAAGCCGTAAATGAACAA | |
| | GTAATCCCTGCATTGACTGCACTTGACTGTGCAGTTCTTGGAA | |
| | ATAAACTAGCTGCACAACTGAACCTCTACCTCATTGAAATGAC | |
| | GACTATTTTTGGTGACCAAATAAATAACCCAGTCCTAACTCCA | |
| | ATACCACTCAGTTATCTCCTGCGGTTGACAGGCTCTGAGTTAA | |
| | ATGATGTATTATTACAACAGACTCGATCCTCTTTGAGCCTAAT | |
| | CCACCTTGTCTCTAAAGGCTTATTAAGTGGTCAGATTATAGGA | |
| | TATGACCCTTCAGTACAAGGCATCATTATCAGAATAGGACTGA | |
| | TCAGGACTCAAAGAATAGATCGGTCACTAGTTTTCCaACCTTA | |
| | CGTATTACCAATTACTATTAGTTCTAACATAGCCACACCAATT | |
| | ATACCCGACTGTGTGGTCAAGAAGGGAGTAATAATTGAGGGAA | |
| | TGCTTAAGAGTAATTGTATAGAATTGGAACGAGATATAATTTG | |
| | CAAGACTATCAACACATACCAAATAACTAAGGAAACTAGAGCA | |
| | TGCTTACAAGGTAATATAACAATGTGTAAGTACCAGCAGTCCA | |
| | GGACACAGTTGAGCACCCCCTTTATTACATATAATGGAGTTGT | |
| | AATTGCAAATTGTGATTTGGTATCATGCCGATGCATAAGACCC | |
| | CCTATGATTATCACACAAGTAAAAGGTTACCCTCTGACAATTA | |
| | TAAATAGGAATTTATGTACCGAGTTGTCGGTGGATAATTTAAT | |
| | TTTAAATATTGAAACAAACCATAACTTTTCATTAAACCCTACT | |
| | ATTATAGATTCACAATCCCGGCTTATAGCTACTAGTCCATTAG | |
| | AAATAGATGCCCTTATTCAAGATGCGCAACATCACGCGGCTGC | |
| | GGCCCTTCTTAAAGTAGAAGAAAGCAATGCTCACTTATTAAGA | |
| | GTTACAGGGCTGGGCTCATCAAGTTGGCACATCATACTTATAT | |
| | TAACATTGCTTGTATGCACCATAGCATGGCTCATTGGTTTATC | |
| | TATTTATGTCTGCCGCATTAAAAATGATGACTCGACCGACAAA | |
| | GAACCTACAACCCAATCATCGAACCGaGGCATTGGGGTTGGAT | |
| | CTATACAATATATGACATGAtgaacacagatgaggaacgaagg | |
| | tttccctaatagtaatttgtgtgaaagttctggtagtctgtca | |
| | gttcagagagttaagaaaaaactaccggttgtagatgaccaaa | |
| | ggacgatatacgggtagaacggtaagagaggccgcccctcaat | |
| | tgcgagccaggcttcacaacctccgttctaccgcttcaccgac | |
| | aacagtcctcaatcATGGAGCCGACAGGATCAAAAGTTGACAT | |
| | TGTCCCTTCCCAAGGTACCAAGAGAACATGTCGAACCTTTTAT | |
| | CGCCTCTTAATTCTTATTTTGAATCTTATTATAATTATATTAA | |
| | CAATTATCAGTATTTATGTCTCTATCTCAACAGATCAACACAA | |
| | ATTGTGCAATAATGAGGCTGACTCACTTTTACACTCAATAGTA | |
| | GAACCCATAACAGTCCCCCTAGGAACAGACTCGGATGTTGAGG | |
| | ATGAATTACGTGAGATTCGACGTGATACAGGCATAAATATTCC | |
| | TATCCAAATTGACAACACAGAGAACATCATATTAACTACATTA | |
| | GCAAGTATCAACTCTAACATTGCACGCCTTCATAACGCCACCG | |
| | ATGAAAGCCCAACATGCCTGTCACCAGTTAATGATCCCAGGTT | |
| | TATAGCAGGGATTAATAAGATAACCAAAGGGTCGATGATATAT | |
| | AGGAATTTCAGCAATTTGATAGAACATGTTAACTTTATACCAT | |
| | CTCCAACGACATTATCAGGCTGTACAAGAATTCCATCTTTTTC | |
| | ACTATCTAAAACACATTGGTGTTACTCGCATAATGTAATATCT | |
| | ACTGGTTGTCAAGACCATGCTGCGAGTTCACAGTATATTTCCA | |
| | TAGGAATAGTAGATACAGGATTGAATAATGAGCCCTATTTGCG | |
| | TACAATGTCTTCACGCTTGCTAAATGATGGCCTAAATAGAAAG | |
| | AGCTGCTCTGTCACAGCCGGCGCTGGTGTCTGTTGGCTATTGT | |
| | GTAGTGTTGTAACAGAAAGTGAATCAGCTGACTACAGATCAAG | |
| | AGCCCCCACTGCAATGATTCTCGGAAGGTTCAATTTTTATGGT | |
| | GATTACACTGAATCCCCTGTTCCTGCATCTTTGTTCAGCGGTC | |
| | GTTTCACTGCTAATTACCCTGGAGTTGGCTCAGGAACCCAATT | |
| | AAATGGGACCCTTTATTTTCCAATATATGGGGGTGTTGTTAAC | |
| | GACTCTGATATTGAGTTATCGAACCGAGGGAAGTCATTCAGAC | |
| | CTAGGAACCCTACAAACCCATGTCCAGATCCTGAGGTGACCCA | |
| | AAGTCAGAGGGCTCAGGCAAGTTACTATCCGACAAGGTTTGGC | |
| | AGGCTGCTCATACAACAAGCAATACTAGCTTGTCGTATTAGTG | |
| | ACACTACATGCACTGATTATTATCTTCTATACTTTGATAATAA | |
| | TCAAGTCATGATGGGTGCAGAAGCCCGAATTTATTATTTAAAC | |
| | AATCAGATGTACTTATATCAAAGATCTTCGAGTTGGTGGCCGC | |
| | ATCCGCTTTTTTACAGATTCTCACTGCCTCATTGTGAACCTAT | |
| | GTCTGTCTGTATGATCACCGATACACACTTAATATTGACATAT | |
| | GCTACCTCACGCCCTGGCACTTCAATTTGTACAGGGGCCTCGC | |
| | GATGTCCTAATAACTGTGTTGATGGTGTCTATACAGACGTTTG | |
| | GCCCTTGACTGAGGGTACAACACAAGATCCAGATTCCTACTAC | |
| | ACAGTATTCCTCAACAGTCCCAACCGCAGGATCAGTCCTACAA | |
| | TTAGCATTTACAGCTACAACCAGAAGATTAGCTCTCGTCTGGC | |
| | TGTAGGAAGTGAAATAGGAGCTGCTTACACGACCAGTACATGT | |
| | TTTAGCAGGACAGACACTGGGGCACTATACTGCATCACTATAA | |
| | TAGAAGCTGTAAACACAATCTTTGGACAATACCGAATAGTACC | |
| | GATCCTTGTTCAACTAATTAGTGACtagttgagtcaattataa | |
| | aggagttggaaagatggcattgtatcacctatcttctgcgaca | |
| | tcaagaatcaAACCGAATGCCGGCGCGTGCTCGAATTCCATGT | |
| | TGCCAGTTGACCACAATCAGCCAGTGCTCATGCGATCAGATTA | |
| | AGCCTTGTCAATAGTCTCTTGATTAAGAAAAAATGTAAGTGGC | |
| | AATGAGATACAAGGCAAAACAGCTCATGGTTAACAATACGGGT | |
| | AGGACATGGCGAGCTCCGGTCCTGAAAGGGCAGAGCATCAGAT | |
| | TATCCTACCAGAGTCACACCTGTCTTCACCATTGGTCAAGCAC | |
| | AAACTACTCTATTACTGGAAATTAACTGGGCTACCGCTTCCTG | |
| | ATGAATGTGACTTCGACCACCTCATTCTCAGCCGACAATGGAA | |
| | AAAAATACTTGAATCGGCCTCTCCTGATACTGAGAGAATGATA | |
| | AAACTCGGAAGGGCAGTACACCAAACTCTTAACCACAATTCCA | |
| | GAATAACCGGAGTGCTCCACCCCAGGTGTTTAGAAGAACTGGC | |
| | TAATATTGAGGTCCCAGATTCAACCAACAAATTTCGGAAGATT | |
| | GAGAAGAAGATCCAAATTCACAACACGAGATATGGAGAACTGT | |
| | TCACAAGGCTGTGTACGCATATAGAGAAGAAACTGCTGGGGTC | |
| | ATCTTGGTCTAACAATGTCCCCCGGTCAGAGGAGTTCAGCAGC | |
| | ATTCGTACGGATCCGGCATTCTGGTTTCACTCAAAATGGTCCA | |
| | CAGCCAAGTTTGCATGGCTCCATATAAAACAGATCCAGAGGCA | |
| | TCTGATGGTGGCAGCTAGGACAAGGTCTGCGGCCAACAAATTG | |
| | GTGATGCTAACCCATAAGGTAGGCCAAGTCTTTGTCACTCCTG | |
| | AACTTGTCGTTGTGACGCATACGAATGAGAACAAGTTCACATG | |
| | TCTTACCCAGGAACTTGTATTGATGTATGCAGATATGATGGAG | |
| | GGCAGAGATATGGTCAACATAATATCAACCACGGCGGTGCATC | |
| | TCAGAAGCTTATCAGAGAAAATTGATGACATTTTGCGGTTAAT | |
| | AGACGCTCTGGCAAAAGACTTGGGTAATCAAGTCTACGATGTT | |
| | GTATCACTAATGGAGGGATTTGCATACGGAGCTGTCCAGCTAC | |
| | TCGAGCCGTCAGGTACATTTGCAGGAGATTTCTTCGCATTCAA | |
| | CCTGCAGGAGCTTAAAGACATTCTAATTGGCCTCCTCCCCAAT | |
| | GATATAGCAGAATCCGTGACTCATGCAATCGCTACTGTATTCT | |
| | CTGGTTTAGAACAGAATCAAGCAGCTGAGATGTTGTGTCTGTT | |
| | GCGTCTGTGGGGTCACCCACTGCTTGAGTCCCGTATTGCAGCA | |
| | AAGGCAGTCAGGAGCCAAATGTGCGCACCGAAAATGGTAGACT | |
| | TTGATATGATCCTTCAGGTACTGTCTTTCTTCAAGGGAACAAT | |
| | CATCAACGGGTACAGAAAGAAGAATGCAGGTGTGTGGCCGCGA | |
| | GTCAAAGTGGATACAATATATGGGAAGGTCATTGGGCAACTAC | |
| | ATGCAGATTCAGCAGAGATTTCACACGATATCATGTTGAGAGA | |
| | GTATAAGAGTTTATCTGCACTTGAATTTGAGCCATGTATAGAA | |
| | TATGACCCTGTCACCAACCTGAGCATGTTCCTAAAAGACAAGG | |
| | CAATCGCACACCCCAACGATAATTGGCTTGCCTCGTTTAGGCG | |
| | GAACCTTCTCTCCGAAGACCAGAAGAAACATGTAAAAGAAGCA | |
| | ACTTCGACTAATCGCCTCTTGATAGAGTTTTTAGAGTCAAATG | |
| | ATTTTGATCCATATAAAGAGATGGAATATCTGACGACCCTTGA | |
| | GTACCTTAGAGATGACAATGTGGCAGTATCATACTCGCTCAAG | |
| | GAGAAGGAAGTGAAAGTTAATGGACGGATCTTCGCTAAGCTGA | |
| | CAAAGAAGTTAAGGAACTGTCAGGTGATGGCGGAAGGGATCCT | |
| | AGCCGATCAGATTGCACCTTTCTTTCAGGGAAATGGAGTCATT | |
| | CAGGATAGCATATCCTTGACCAAGAGTATGCTAGCGATGAGTC | |
| | AACTGTCTTTTAACAGCAATAAGAAACGTATCACTGACTGTAA | |
| | AGAAAGAGTATCTTCAAACCGCAATCATGATCCGAAAAGCAAG | |
| | AACCGTCGGAGAGTTGCAACCTTCATAACAACTGACCTGCAAA | |
| | AGTACTGTCTTAATTGGAGATATCAGACAATCAAATTGTTCGC | |
| | TCATGCCATCAATCAGTTGATGGGCCTACCTCACTTCTTCGAA | |
| | TGGATTCACCTAAGACTGATGGACACTACGATGTTCGTAGGAG | |
| | ACCCTTTCAATCCTCCAAGTGACCCTACTGACTGTGACCTCTC | |
| | AAGAGTCCCTAATGATGACATATATATTGTCAGTGCCAGAGGG | |
| | GGTATCGAAGGATTATGCCAGAAGCTATGGACAATGATCTCAA | |
| | TTGCTGCAATCCAACTTGCTGCAGCTAGATCGCATTGTCGTGT | |
| | TGCCTGTATGGTACAGGGTGATAATCAAGTAATAGCAGTAACG | |
| | AGAGAGGTAAGATCAGACGACTCTCCGGAGATGGTGTTGACAC | |
| | AGTTGCATCAAGCCAGTGATAATTTCTTCAAGGAATTAATTCA | |
| | TGTCAATCATTTGATTGGCCATAATTTGAAGGATCGTGAAACC | |
| | ATCAGGTCAGACACATTCTTCATATACAGCAAACGAATCTTCA | |
| | AAGATGGAGCAATCCTCAGTCAAGTCCTCAAAAATTCATCTAA | |
| | ATTAGTGCTAGTGTCAGGTGATCTCAGTGAAAACACCGTAATG | |
| | TCCTGTGCCAACATTGCCTCTACTGTAGCACGGCTATGCGAGA | |
| | ACGGGCTTCCCAAAGACTTCTGTTACTATTTAAACTATATAAT | |
| | GAGTTGTGTGCAGACATACTTTGACTCTGAGTTCTCCATCACC | |
| | AACAATTCGCACCCCGATCITAATCAGTCGTGGATTGAGGACA | |
| | TCTCTTTTGTGCACTCATATGTTCTGACTCCTGCCCAATTAGG | |
| | GGGACTGAGTAACCTTCAATACTCAAGGCTCTACACTAGAAAT | |
| | ATCGGTGACCCGGGGACTACTGCTTTTGCAGAGATCAAGCGAC | |
| | TAGAAGCAGTGGGATTACTGAGTCCTAACATTATGACTAATAT | |
| | CTTAACTAGGCCGCCTGGGAATGGAGATTGGGCCAGTCTGTGC | |
| | AACGACCCATACTCTTTCAATTTTGAGACTGTTGCAAGCCCAA | |
| | ATATTGTTCTTAAGAAACATACGCAAAGAGTCCTATTTGAAAC | |
| | TTGTTCAAATCCCTTATTGTCTGGAGTGCACACAGAGGATAAT | |
| | GAGGCAGAAGAGAAGGCATTGGCTGAATTCTTGCTTAATCAAG | |
| | AGGTGATTCATCCCCGCGTTGCGCATGCCATCATGGAGGCAAG | |
| | CTCTGTAGGTAGGAGAAAGCAAATTCAAGGGCTTGTTGACACA | |
| | ACAAACACCGTAATTAAGATTGCGCTTACTAGGAGGCCATTAG | |
| | GCATCAAGAGGCTGATGCGGATAGTCAATTATTCTAGCATGCA | |
| | TGCAATGCTGTTTAGAGACGATGTTTTTTCCTCCAGTAGATCC | |
| | AACCACCCCTTAGTCTCTTCTAATATGTGTTCTCTGACACTGG | |
| | CAGACTATGCACGGAATAGAAGCTGGTCACCTTTGACGGGAGG | |
| | CAGGAAAATACTGGGTGTATCTAATCCTGATACGATAGAACTC | |
| | GTAGAGGGTGAGATTCTTAGTGTAAGCGGAGGGTGTACAAGAT | |
| | GTGACAGCGGAGATGAACAATTTACTTGGTTCCATCTTCCAAG | |
| | CAATATAGAATTGACCGATGACACCAGCAAGAATCCTCCGATG | |
| | AGGGTACCATATCTCGGGTCAAAGACACAGGAGAGGAGAGCTG | |
| | CCTCACTTGCAAAAATAGCTCATATGTCGCCACATGTAAAGGC | |
| | TGCCCTAAGGGCATCATCCGTGTTGATCTGGGCTTATGGGGAT | |
| | AATGAAGTAAATTGGACTGCTGCTCTTACGATTGCAAAATCTC | |
| | GGTGTAATGTAAACTTAGAGTATCTTCGGTTACTGTCCCCTTT | |
| | ACCCACGGCTGGGAATCTTCAACATAGACTAGATGATGGTATA | |
| | ACTCAGATGACATTCACCCCTGCATCTCTCTACAGGGTGTCAC | |
| | CTTACATTCACATATCCAATGATTCTCAAAGGCTGTTCACTGA | |
| | AGAAGGAGTCAAAGAGGGGAATGTGGTTTACCAACAGATCATG | |
| | CTCTTGGGTTTATCTCTAATCGAATCGATCTTTCCAATGACAA | |
| | CAACCAGGACATATGATGAGATCACACTGCACCTACATAGTAA | |
| | ATTTAGTTGCTGTATCAGAGAAGCACCTGTTGCGGTTCCTTTC | |
| | GAGCTACTTGGGGTGGTACCGGAACTGAGGACAGTGACCTCAA | |
| | ATAAGTTTATGTATGATCCTAGCCCTGTATCGGAGGGAGACTT | |
| | TGCGAGACTTGACTTAGCTATCTTCAAGAGTTATGAGCTTAAT | |
| | CTGGAGTCATATCCCACGATAGAGCTAATGAACATTCTTTCAA | |
| | TATCCAGCGGGAAGTTGATTGGCCAGTCTGTGGTTTCTTATGA | |
| | TGAAGATACCTCCATAAAGAATGACGCCATAATAGTGTATGAC | |
| | AATACCCGAAATTGGATCAGTGAAGCTCAGAATTCAGATGTGG | |
| | TCCGCCTATTTGAATATGCAGCACTTGAAGTGCTCCTCGACTG | |
| | TTCTTACCAACTCTATTACCTGAGAGTAAGAGGCCTGGACAAT | |
| | ATTGTCTTATATATGGGTGATTTATACAAGAATATGCCAGGAA | |
| | TTCTACTTTCCAACATTGCAGCTACAATATCTCATCCCGTCAT | |
| | TCATTCAAGGTTACATGCAGTGGGCCTGGTCAACCATGACGGA | |
| | TCACACCAACTTGCAGATACGGATTTTATCGAAATGTCTGCAA | |
| | AACTATTAGTATCTTGCACCCGACGTGTGATCTCCGGCTTATA | |
| | TTCAGGAAATAAGTATGATCTGCTGTTCCCATCTGTCTTAGAT | |
| | GATAACCTGAATGAGAAGATGCTTCAGCTGATATCCCGGTTAT | |
| | GCTGTCTGTACACGGTACTCTTTGCTACAACAAGAGAAATCCC | |
| | GAAAATAAGAGGCTTAACTGCAGAAGAGAAATGTTCAATACTC | |
| | ACTGAGTATTTACTGTCGGATGCTGTGAAACCATTACTTAGCC | |
| | CCGATCAAGTGAGCTCTATCATGTCTCCTAACATAATTACATT | |
| | CCCAGCTAATCTGTACTACATGTCTCGGAAGAGCCTCAATTTG | |
| | ATCAGGGAAAGGGAGGACAGGGATACTATCCTGGCGTTGTTGT | |
| | TCCCCCAAGAGCCATTATTAGAGTTCCCTTCTGTGCAAGATAT | |
| | TGGTGCTCGAGTGAAAGATCCATTCACCCGACAACCTGCGGCA | |
| | TTTTTGCAAGAGTTAGATTTGAGTGCTCCAGCAAGGTATGACG | |
| | CATTCACACTTAGTCAGATTCATCCTGAACTCACATCTCCAAA | |
| | TCCGGAGGAAGACTACTTAGTACGATACTTGTTCAGAGGGATA | |
| | GGGACTGCATCTTCCTCTTGGTATAAGGCATCTCATCTCCTTT | |
| | CTGTACCCGAGGTAAGATGTGCAAGACACGGGAACTCCTTATA | |
| | CTTAGCTGAAGGGAGCGGAGCCATCATGAGTCTTCTCGAACTG | |
| | CATGTACCACATGAAACTATCTATTACAATACGCTCTTTTCAA | |
| | ATGAGATGAACCCCCCGCAACGACATTTCGGGCCGACCCCAAC | |
| | TCAGTTTTTGAATTCGGTTGTTTATAGGAATCTACAGGCGGAG | |
| | GTAACATGCAAAGATGGATTTGTCCAAGAGTTCCGTCCATTAT | |
| | GGAGAGAAAATACAGAGGAAAGCGACCTGACCTCAGATAAAGT | |
| | AGTGGGGTATATTACATCTGCAGTGCCCTACAGATCTGTATCA | |
| | TTGCTGCATTGTGACATTGAAATTCCTCCAGGGTCCAATCAAA | |
| | GCTTACTAGATCAACTAGCTATCAATTTATCTCTGATTGCCAT | |
| | GCATTCTGTAAGGGAGGGCGGGGTAGTAATCATCAAAGTGTTG | |
| | TATGCAATGGGATACTACTTTCATCTACTCATGAACTTGTTTG | |
| | CTCCGTGTTCCACAAAAGGATATATTCTCTCTAATGGTTATGC | |
| | ATGTCGAGGAGATATGGAGTGTTACCTGGTATTTGTCATGGGT | |
| | TACCTGGGCGGGCCTACATTTGTACATGAGGTGGTGAGGATGG | |
| | CGAAAACTCTGGTGCAGCGGCACGGTACGCTTTTGTCTAAATC | |
| | AGATGAGATCACACTGACCAGGTTATTCACCTCACAGCGGCAG | |
| | CGTGTGACAGACATCCTATCCAGTCCTTTACCAAGATTAATAA | |
| | AGTACTTGAGGAAGAATATTGACACTGCGCTGATTGAAGCCGG | |
| | GGGACAGCCCGTCCGTCCATTCTGTGCGGAGAGTCTGGTGAGC | |
| | ACGCTAGCGAACATAACTCAGATAACCCAGATCATCGCTAGTC | |
| | ACATTGACACAGTTATCCGGTCTGTGATATATATGGAAGCTGA | |
| | GGGTGATCTCGCTGACACAGTATTTCTATTTACCCCTTACAAT | |
| | CTCTCTACTGACGGGAAAAAGAGGACATCACTTAAACAGTGCA | |
| | CGAGACAGATCCTAGAGGTTACAATACTAGGTCTTAGAGTCGA | |
| | AAATCTCAATAAAATAGGCGATATAATCAGCCTAGTGCTTAAA | |
| | GGCATGATCTCCATGGAGGACCTTATCCCACTAAGGACATACT | |
| | TGAAGCATAGTACCTGCCCTAAATATTTGAAGGCTGTCCTAGG | |
| | TATTACCAAACTCAAAGAAATGTTTACAGACACTTCTGTACTG | |
| | TACTTGACTCGTGCTCAACAAAAATTCTACATGAAAACTATAG | |
| | GCAATGCAGTCAAAGGATATTACAGTAACTGTGACTCTTAACG | |
| | AAAATCACATATTAATAGGCTCCTTTTTTGGCCAATTGTATTC | |
| | TTGTTGATTTAATCATATTATGTTAGAAAAAAGTTGAACCCTG | |
| | ACTCCTTAGGACTCGAATTCGAACTCAAATAAATGTCTTAAAA | |
| | AAAGGTTGCGCACAATTATTCTTGAGTGTAGTCTCGTCATTCA | |
| | CCAAATCTTTGTTTGGTGCGCGCGGCCGGCATGGTCCCAGCCT | |
| | CCTCGCTGGCGCCGGCTGGGCAACATTCCGAGGGGACCGTCCC | |
| | CTCGGTAATGGCGAATGGGACGTCGACTGCTAACAAAGCCCGA | |
| | AAGGAAGCTGAGTTGGCTGCTGCCACCGCTGAGCAATAACTAG | |
| | CATAACCCCTTGGGGCCTCTAAACGGGTCTTGAGGGGTTTTTT | |
| | GCTGAAAGGAGGAACTATATGCGCGCAGATCTGTCATGATGAT | |
| | CATTGCAATTGGATCCATATATAGGGCCCGGGTTATAATTACC | |
| | TCAGGTCGACGTCCCATGGCCATTCGAATTCGTAATCATGGTC | |
| | ATAGCTGTTTCCTGTGTGAAATTGTTATCCGCTCACAATTCCA | |
| | CACAACATACGAGCCGGAAGCATAAAGTGTAAAGCCTGGGGTG | |
| | CCTAATGAGTGAGCTAACTCACATTAATTGCGTTGCGCTCACT | |
| | GCCCGCTTTCCAGTCGGGAAACCTGTCGTGCCAGCTGCATTAA | |
| | TGAATCGGCCAACGCGCGGGGAGAGGCGGTTTGCGTATTGGGC | |
| | GC | |

The invention is not to be limited in scope by the specific embodiments described herein. Indeed, various modifications of the invention in addition to those described will become apparent to those skilled in the art from the foregoing description and accompanying Figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed:

1. A nucleic acid sequence comprising a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV APMV HN protein coding sequence; and (2) the nucleotide sequence encoding the NDV F protein has been replaced with a nucleotide sequence encoding a non-NDV APMV F protein, wherein NDV intergenic regions are before and after the non-NDV APMV F protein coding sequence, and wherein the non-NDV APMV HN protein and non-NDV APMV F protein are not NDV HN protein and F proteins, respectively.

2. The nucleic acid sequence of claim 1, wherein the non-NDV APMV F protein and non-NDV APMV HN protein are immunologically distinct from the NDF F protein and NDV HN protein, respectively.

3. The nucleic acid sequence of claim 1, wherein the non-NDV APMV HN is the HN protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07.

4. The nucleic acid sequence of claim 1 or 3, wherein the non-NDV APMV F is the F protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07.

5. The nucleic acid sequence of claim 1, wherein the non-NDV APMV HN protein is an HN protein from the subfamily Avulavirinae and the genus orthoavulavirus, metaavulavirus, or paraavulavirus.

6. The nucleic acid sequence of claim 1 or 5, wherein the non-NDV APMV F protein is an F protein from the subfamily Avulavirinae and the genus orthoavulavirus, metaavulavirus, or paraavulavirus.

7. The nucleic acid sequence of any one of claims 1 to 6, wherein the NDV genome comprises the NP gene, P gene, M gene, and L gene of NDV LaSota strain.

8. A nucleic acid sequence comprising: (1) a transcription unit encoding a NDV nucleocapsid (N) protein, (2) a transcription unit encoding a NDV phosphoprotein (P), (3) a transcription unit encoding a NDV matrix (M) protein, (4) a transcription unit encoding a NDV large polymerase (L), and (5) the nucleotide sequence of any one of SEQ ID NOS:1-14, or a nucleotide sequence that is at least 80%, at least 85%, at least 90%, at least 95%, or at least 98% identical to the nucleotide sequence of any one of SEQ ID NOS:1-14.

9. The nucleic acid sequence of claim 8, wherein the NDV nucleocapsid protein, NDV phosphoprotein, NDV matrix protein, and NDV large polymerase are of the NDV LaSota strain.

10. A nucleic acid sequence comprising the nucleotide sequence of SEQ ID NO:44, or SEQ ID NO:44 without the GFP coding sequence.

11. A nucleic acid sequence comprising a nucleotide sequence that is at least 80%, at least 85%, at least 90%, at least 95%, or at least 98% identical to the nucleotide sequence of SEQ ID NO:44, or SEQ ID NO:44 without the GFP coding sequence.

12. A nucleic acid sequence comprising the nucleotide sequence of SEQ ID NO:45, or SEQ ID NO:45 without the GFP coding sequence.

13. A nucleic acid sequence comprising a nucleotide sequence that is at least 80%, at least 85%, at least 90%, at least 95%, or at least 98% identical to the nucleotide sequence of SEQ ID NO:45, or SEQ ID NO:45 without the GFP coding sequence.

14. The nucleic acid sequence of any one of claims 1 to 13, which further comprises a transgene.

15. The nucleic acid sequence of any one of claims 1 to 13, which further comprises a transgene encoding an antigen.

16. The nucleic acid sequence of claim 14, wherein the antigen is viral, bacterial, fungal or protozoan antigen.

17. The nucleic acid sequence of claim 14, wherein the antigen comprises a SARS-CoV-2 spike protein or a fragment thereof.

18. The nucleic acid sequence of claim 17, wherein the fragment comprises the receptor binding domain of the SARS-CoV-2 spike protein.

19. The nucleic acid sequence of claim 17, wherein the fragment comprises the ectodomain of the SARS-CoV-2 spike protein.

20. The nucleic acid sequence of claim 15, wherein the antigen is a MERS-CoV antigen, respiratory syncytial virus antigen, human metapneumovirus antigen, a Lassa virus antigen, Ebola virus antigen, or Nipah virus antigen.

21. The nucleic acid sequence of claim 15, wherein the antigen is a cancer or tumor antigen.

22. A recombinant Newcastle disease virus (NDV) comprising a packaged genome, wherein the packaged genome comprises a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV AMPV HN protein coding sequence; and (2) the nucleotide sequence encoding the NDV F protein has been replaced with a nucleotide sequence encoding a non-NDV APMV F protein, wherein NDV intergenic regions are before and after the non-NDV AMPV F protein coding sequence, and wherein the non-NDV APMV HN protein and non-NDV APMV F protein are not NDV HN protein and F proteins, respectively.

23. The recombinant NDV of claim 22, wherein the non-NDV APMV F protein and non-NDV APMV HN protein are immunologically distinct from the NDF F protein and NDV HN protein, respectively.

24. The recombinant NDV of claim 22, wherein the non-NDV APMV HN protein is an HN protein from the subfamily Avulavirinae and the genus orthoavulavirus, metaavulavirus, or paraavulavirus.

25. The recombinant NDV of claim 22 or 23, wherein the non-NDV APMV F protein is an F protein from the subfamily Avulavirinae and the genus orthoavulavirus, metaavulavirus, or paraavulavirus.

26. The recombinant NDV of claim 22, wherein the non-NDV APMV HN is the HN protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07.

27. The recombinant NDV of claim 22 or 26, wherein the non-NDV APMV F is the F protein of APMV4/duck/Hongkong/D3/75, APMV17/Antarctica/107/13, APMV9/duck/New York/22/78, APMV7/Dove/Tennessee/4/75, APMV21/pigeon/Taiwan/AHRI128/17, APMV6/duck/HongKong/18/199/77, APMV11/common_snipe/France/100212/10, APMV15/calidris_fuscicollis/Brazil/RS-1177/12, APMV8/Goose/Delaware/1053/76, APMV2/Chicken/California/Yucaipa/56, APMV3/Turkey/Wisconsin/68, APMV12/Wigeon/Italy/3920_1/05, APMV5/budgerigar/Japan/TI/75, or APMV10/penguin/Falkland Islands/324/07.

28. A recombinant Newcastle disease virus (NDV) comprising a packaged genome, wherein the packaged genome comprises a nucleotide sequence of a Newcastle disease virus genome in which the nucleotide sequences encoding the NDV HN protein and NDV F protein are replaced with a nucleotide sequence comprising a negative sense RNA sequence transcribed from the cDNA sequence set forth in any one of SEQ ID NOs:1-14.

29. A recombinant Newcastle disease virus (NDV) comprising a packaged genome, wherein the packaged genome comprises a nucleotide sequence of a Newcastle disease virus genome in which the nucleotide sequences encoding the NDV HN protein and NDV F protein are replaced with a nucleotide sequence comprising a negative sense RNA sequence transcribed from a nucleotide sequence that is at least 80%, at least 85%, at least 90%, at least 95%, or at least 98% identical to the nucleotide sequence of any one of SEQ ID NOS:1-14.

30. The recombinant NDV of any one of claims 22 to 29, wherein the NDV genome comprises the NP gene, P gene, M gene, and L gene of NDV LaSota.

31. The recombinant NDV of any one of claims 22 to 30, wherein the packaged genome further comprises a transgene.

32. The recombinant NDV of claim 31, wherein the transgene comprises a nucleotide sequence encoding a viral, bacterial, fungal or protozoan antigen.

33. The recombinant NDV of claim 31, wherein the transgene comprises a nucleotide sequence encoding a SARS-CoV-2 antigen.

34. The recombinant NDV of claim 33, wherein the SARS-CoV-2 antigen comprises the SARS-CoV-2 spike protein or a fragment thereof.

35. The recombinant NDV of claim 34, wherein the fragment comprises the receptor binding domain of the SARS-CoV-2 spike protein.

36. The recombinant NDV of claim 31, wherein the transgene comprises a nucleotide sequence encoding a MERS-CoV antigen.

37. The recombinant NDV of claim 31, wherein the transgene comprises a nucleotide sequence encoding a respiratory syncytial virus antigen or human metapneumovirus antigen.

38. The recombinant NDV of claim 31, wherein the transgene comprises a nucleotide sequence encoding a Lassa virus antigen, Ebola virus antigen or Nipah virus antigen.

39. The recombinant NDV of claim 31, wherein the transgene comprises a nucleotide sequence encoding a cancer or tumor antigen.

40. An immunogenic composition comprising a first recombinant NDV, which is the recombinant NDV of any one of claims 22 to 31.

41. An immunogenic composition comprising a first recombinant NDV, which is the recombinant NDV of any one of claims 32 to 38.

42. An immunogenic composition comprising a first recombinant NDV, which is the recombinant NDV of claim 39.

43. A method for inducing an immune response to an antigen, comprising administering the immunogenic composition of claim 40, 41, or 42 to a subject.

44. A method for preventing an infectious disease, comprising administering the immunogenic composition of claim 40 or 41 to a subject.

45. A method for immunizing a subject against an infectious disease, comprising administering the immunogenic composition of claim 40 or 41 the subject.

46. A method for treating cancer, comprising administering the immunogenic composition of claim 40 or 42 to a subject.

47. The method of any one of claim 43 to 46, wherein the composition is administered to the subject intranasally.

48. The method of any one of claims 43 to 47, wherein the method further comprises administering a second recombinant NDV comprising a packaged genome, wherein the packaged genome of the second recombinant NDV comprises a nucleotide sequence of a Newcastle disease virus genome in which (1) the nucleotide sequence encoding the NDV HN protein has been replaced with a nucleotide sequence encoding a non-NDV APMV HN protein, wherein NDV intergenic regions are before and after the non-NDV AMPV HN protein coding sequence; and (2) the nucleotide sequence encoding the NDV F protein has been replaced with a nucleotide sequence encoding a non-NDV APMV F protein, wherein NDV intergenic regions are before and after the non-NDV AMPV F protein coding sequence, and wherein the second recombinant NDV is immunologically distinct than the first recombinant NDV.

49. The method of any one of claims 43 to 48, wherein the subject is a human.

50. A kit comprising the recombinant NDV of any one of claims 22 to 39.

51. A kit comprising the nucleic acid sequence of any one of claims 1 to 21.

52. An in vitro or ex vivo cell comprising the recombinant NDV of any one of claims 22 to 39.

53. A cell line or chicken embryonated egg comprising the recombinant NDV of any one of claims 22 to 39.

54. A method for propagating the recombinant NDV of any one of claims 22 to 39, the method comprising culturing the cell or embryonated egg of claim 52 or 53.

55. The method of claim 54, wherein the method further comprises isolating the recombinant NDV from the cell or embryonated egg.
